SEQUENCE LISTING (58 sequences)

SEQ ID NO: 1 - DNA, 1554 bp, Homo sapiens
  CDS (247)..(1323)
ggccgcctttgcaaggttgctggacagatggaactggaagggcagccgtctgccgcccacgaacaccttctcaagcactttgagtgaccacggcttgcaagctggtggctggccccccgagtcccgggctctgaggcacggccgtcgacttaagcgttgcatcctgttacctggagaccctctgagctctcacctgctacttctgccgctgcttctgcacagagcccgggcgaggacccctccaggatgcaggtcccgaacagcaccggcccggacaacgcgacgctgcagatgctgcggaacccggcgatcgcggtggccctgcccgtggtgtactcgctggtggcggcggtcagcatcccgggcaacctcttctctctgtgggtgctgtgccggcgcatggggcccagatccccgtcggtcatcttcatgatcaacctgagcgtcacggacctgatgctggccagcgtgttgcctttccaaatctactaccattgcaaccgccaccactgggtattcggggtgctgctttgcaacgtggtgaccgtggccttttacgcaaacatgtattccagcatcctcaccatgacctgtatcagcgtggagcgcttcctgggggtcctgtacccgctcagctccaagcgctggcgccgccgtcgttacgcggtggccgcgtgtgcagggacctggctgctgctcctgaccgccctgtccccgctggcgcgcaccgatctcacctacccggtgcacgccctgggcatcatcacctgcttcgacgtcctcaagtggacgatgctccccagcgtggccatgtgggccgtgttcctcttcaccatcttcatcctgctgttcctcatcccgttcgtgatcaccgtggcttgttacacggccaccatcctcaagctgttgcgcacggaggaggcgcacggccgggagcagcggaggcgcgcggtgggcctggccgcggtggtcttgctggcctttgtcacctgcttcgcccccaacaacttcgtgctcctggcgcacatcgtgagccgcctgttctacggcaagagctactaccacgtgtacaagctcacgctgtgtctcagctgcctcaacaactgtctggacccgtttgtttattactttgcgtcccgggaattccagctgcgcctgcgggaatatttgggctgccgccgggtgcccagagacaccctggacacgcgccgcgagagcctcttctccgccaggaccacgtccgtgcgctccgaggccggtgcgcaccctgaagggatggagggagccaccaggcccggcctccagaggcaggagagtgtgttctccctgctgacatcgtcccttagttgtggttctggccttctccattctcctccaggggttctggtctccgtagcccggtgcacgccgaaatttctgtttatttcactcaggggcactgtggttgctgtggttggaattcttctttcagaggagcgcctggggctcctgcaagtcagctactctccgtgcccacttcccctcacacacacacccccctcgtgccgaattctt

SEQ ID NO: 2 - protein, 359 aa, Homo sapiens
MQVPNSTGPDNATLQMLRNPAIAVALPVVYSLVAAVSIPGNLFSLWVLCRRMGPRSPSVIFMINLSVTDLMLASVLPFQIYYHCNRHHWVFGVLLCNVVTVAFYANMYSSILTMTCISVERFLGVLYPLSSKRWRRRRYAVAACAGTWLLLLTALSPLARTDLTYPVHALGIITCFDVLKWTMLPSVAMWAVFLFTIFILLFLIPFVITVACYTATILKLLRTEEAHGREQRRRAVGLAAVVLLAFVTCFAPNNFVLLAHIVSRLFYGKSYYHVYKLTLCLSCLNNCLDPFVYYFASREFQLRLREYLGCRRVPRDTLDTRRESLFSARTTSVRSEAGAHPEGMEGATRPGLQRQESVF

SEQ ID NO: 3 - DNA, 19 bp, Homo sapiens
gatccccgtcggtcatctt

SEQ ID NO: 4 - DNA, 19 bp, Homo sapiens
ggtcaccacgttgcaaagc

SEQ ID NO: 5 - DNA, 25 bp, Artificial Sequence
  Synthesized Oligonucleotide.
acaggaugcaacgcuuaagucgacg

SEQ ID NO: 6 - DNA, 25 bp, Artificial Sequence
  Synthesized Oligonucleotide.
agauuuggaaaggcaacacgcuggc

SEQ ID NO: 7 - DNA, 25 bp, Artificial Sequence
  Synthesized Oligonucleotide.
cuugaggacgucgaagcaggugaug

SEQ ID NO: 8 - DNA, 25 bp, Artificial Sequence
  Synthesized Oligonucleotide.
gccuccuccgugcgcaacagcuuga

SEQ ID NO: 9 - DNA, 25 bp, Artificial Sequence
  Synthesized Oligonucleotide.
cugauacaggucauggugaggaugc

SEQ ID NO: 10 - DNA, 16 bp, Homo sapiens
cccggtggaccacgaa

SEQ ID NO: 11 - DNA, 19 bp, Homo sapiens
ggctcgcctcttccatgtc

SEQ ID NO: 12 - DNA, 25 bp, Homo sapiens
aacccgggacttggagaagcactgc

SEQ ID NO: 13 - DNA, 24 bp, Homo sapiens
gaggatgaggagagctatgacaca

SEQ ID NO: 14 - DNA, 22 bp, Homo sapiens
ccctttgcactcataacgtcag

SEQ ID NO: 15 - DNA, 29 bp, Homo sapiens
aaacacacagtcatcatagggcagctcgt

SEQ ID NO: 16 - DNA, 2448 bp, Homo sapiens
  CDS (359)..(1651)
ccacgcgtccgggcgccaggaccctagcgtggcgctccagcaccccagaccgtggcggcgcctcgccttagggaagagcaagggaagaactttatttgaaccgcgaacattttttggtcactgagatcgagtctcccagtgctttggcttcccgcctctttatcttgggtttgatccctgagctgctctcctttcccgaacctcccggggtgcagcctagagccctcccgcgcggctgactccagagtagaggaagggaggcggcctccggctggtcccccgaagccctcgctgccccgcagatgcggatggccagccagtagcgggcggtggccccgcgtcccgggagcgcacagcaatgcaggcgcttaacattaccccggagcagttctctcggctgctgcgggaccacaacctgacgcgggagcagttcatcgctctgtaccggctgcgaccgctcgtctacaccccagagctgccgggacgcgccaagctggccctcgtgctcaccggcgtgctcatcttcgccctggcgctctttggcaatgctctggtgttctacgtggtgacccgcagcaaggccatgcgcaccgtcaccaacatctttatctgctccttggcgctcagtgacctgctcatcaccttcttctgcattcccgtcaccatgctccagaacatttccgacaactggctggggggtgctttcatttgcaagatggtgccatttgtccagtctaccgctgttgtgacagaaatcctcactatgacctgcattgctgtggaaaggcaccagggacttgtgcatccttttaaaatgaagtggcaatacaccaaccgaagggctttcacaatgctaggtgtggtctggctggtggcagtcatcgtaggatcacccatgtggcacgtgcaacaacttgagatcaaatatgacttcctatatgaaaaggaacacatctgctgcttagaagagtggaccagccctgtgcaccagaagatctacaccaccttcatccttgtcatcctcttcctcctgcctcttatggtgatgcttattctgtacagtaaaattggttatgaactttggataaagaaaagagttggggatggttcagtgcttcgaactattcatggaaaagaaatgtccaaaatagccaggaagaagaaacgagctgtcattatgatggtgacagtggtggctctctttgctgtgtgctgggcaccattccatgttgtccatatgatgattgaatacagtaattttgaaaaggaatatgatgatgtcacaatcaagatgatttttgctatcgtgcaaattattggattttccaactccatctgtaatcccattgtctatgcatttatgaatgaaaacttcaaaaaaaatgttttgtctgcagtttgttattgcatagtaaataaaaccttctctccagcacaaaggcatggaaattcaggaattacaatgatgcggaagaaagcaaagttttccctcagagagaatccagtggaggaaaccaaaggagaagcattcagtgatggcaacattgaagtcaaattgtgtgaacagacagaggagaagaaaaagctcaaacgacatcttgctctctttaggtctgaactggctgagaattctcctttagacagtgggcattaattataacaatatcttcataattaatgcccttcagattgtaacccaaagagaaaattattttgagcaaaggtcaaatactctttttattcttaagatgatgacaagaagaaaacaaatcatgtttccattaaaaaatgacacgaggctagtccaagtgcagtgatgtttacaaccaattgatcacaatcatttaacagatttctgtgttccttctcattcccactgcttcacttgactagccttaaaaaagcaacatggaaggccaggcacggtggctcatgcctgtaatcccagcactttgggaggcctagacgggcggatcacgaggtcaggagatcaaaaccatcctggctaacacggtgaaaccccatctctgctaaaaatacaaaaattagccgggcgtggtggcgggcacctgtagtcccagctacttgggagcctcaggcgggagaatggtgtgaacccgggaggcggagcttgcagtgatccgagatcgtgccactgcactccagcctgggcgaaagagcgagactccccgtctcaaaaaaaatttttttgaaaaattcgtaaaccatacttttaagattatttcagtggatttttaaaaatcttgtacagaaatcagggttcttagctagcagtttttctcccacgcagtcactgtaatgtgactatgtattgctagattgaataagaaaataaaataatatcttcttccttgaaaaaaaaaaaaaaaaaaaaaaaaaaaaaaaaaaggccggccgctctagaggatccctcgaggggcccaagct

SEQ ID NO: 17 - protein, 431 aa, Homo sapiens
MQALNITPEQFSRLLRDHNLTREQFIALYRLRPLVYTPELPGRAKLALVLTGVLIFALALFGNALVFYVVTRSKAMRTVTNIFICSLALSDLLITFFCIPVTMLQNISDNWLGGAFICKMVPFVQSTAVVTEILTMTCIAVERHQGLVHPFKMKWQYTNRRAFTMLGVVWLVAVIVGSPMWHVQQLEIKYDFLYEKEHICCLEEWTSPVHQKIYTTFILVILFLLPLMVMLILYSKIGYELWIKKRVGDGSVLRTIHGKEMSKIARKKKRAVIMMVTVVALFAVCWAPFHVVHMMIEYSNFEKEYDDVTIKMIFAIVQIIGFSNSICNPIVYAFMNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 18 - DNA, 21 bp, Homo sapiens
tttctggatcgtcagcttgct

SEQ ID NO: 19 - DNA, 21 bp, Homo sapiens
acagggctggtccactcttct

SEQ ID NO: 20 - DNA, 33 bp, Homo sapiens
cccaagcttatgcaggcgcttaacattaccccg

SEQ ID NO: 21 - DNA, 32 bp, Homo sapiens
cgggatccttaatgccactgtctaaaggaaga

SEQ ID NO: 22 - DNA, 68 bp, Homo sapiens
cgggatccttacttgtcgtcgtcgtccttgtagtccatatgcccactgtctaaaggagaattctcaac

SEQ ID NO: 23 - DNA, 35 bp, Homo sapiens
gcagcagcggccgcatgcgcaccgtcaccaacatc

SEQ ID NO: 24 - DNA, 37 bp, Homo sapiens
gcagcagtcgacatgcccactgtctaaaggagaattc

SEQ ID NO: 25 - DNA, 39 bp, Homo sapiens
gcagcagcggccgcatgcaggcgcttaacattaccccgg

SEQ ID NO: 26 - DNA, 34 bp, Homo sapiens
gcagcagtcgacatattccttttcaaaattactg

SEQ ID NO: 27 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 28 - DNA, 19 bp, Homo sapiens
caccaaccgaagggctttc

SEQ ID NO: 29 - DNA, 21 bp, Homo sapiens
ccacatgggtgatcctacgat

SEQ ID NO: 30 - DNA, 25 bp, Homo sapiens
actgccaccagccagaccacaccta

SEQ ID NO: 31 - DNA, 17 bp, Homo sapiens
catccgccttattacat

SEQ ID NO: 32 - DNA, 23 bp, Homo sapiens
catccgccttattacatcttttt

SEQ ID NO: 33 - DNA, 17 bp, Homo sapiens
catgcggggcagcgagg

SEQ ID NO: 34 - DNA, 24 bp, Homo sapiens
catgcggggcagcgagggcttcgg

SEQ ID NO: 35 - protein, 15 aa, Artificial Sequence
  Synthetic Peptide.
GDFWYEACESSCAFW

SEQ ID NO: 36 - protein, 15 aa, Artificial Sequence
  Synthetic Peptide.
LEWGSDVFYDVYDCC

SEQ ID NO: 37 - protein, 15 aa, Artificial Sequence
  Synthetic Peptide.
CLRSGTGCAFQLYRF

SEQ ID NO: 38 - protein, 15 aa, Artificial Sequence
  Synthetic Peptide.
FAGQIIWYDALDTLM

SEQ ID NO: 39 - protein, 397 aa, Homo sapiens
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 40 - protein, 374 aa, Homo sapiens
MKALIFAAAGLLLLLPTFCQSGMENDTNNLAKPTLPIKTFRGAPPNSFEEFPFSALEGWTGATITVKIKCPEESASHLHVKNATMGYLTSSLSTKLIPAIYLLVFVVGVPANAVTLWMLFFRTRSICTTVFYTNLAIADFLFCVTLPFKIAYHLNGNNWVFGEVLCRATTVIFYGNMYCSILLLACISINRYLAIVHPFTYRGLPKHTYALVTCGLVWATVFLYMLPFFILKQEYYLVQPDITTCHDVHNTCESSSPFQLYYFISLAFFGFLIPFVLIIYCYAAIIRTLNAYDHRWLWYVKASLLILVIFTICFAPSNIILIIHHANYYYNNTDGLYFIYLIALCLGSLNSCLDPFLYFLMSKTRNHSTAYLTK

SEQ ID NO: 41 - protein, 420 aa, Xenopus laevis
MMELRVLLLLLLLTLLGAMGSLCLANSDTQAKGAHSNNMTIKTFRIFDDSESEFEEIPWDELDESGEGSGDQAPVSRSARKPIRRNITKEAEQYLSSQWLTKFVPSLYTVVFIVGLPLNLLAIIIFLFKMKVRKPAVVYMLNLAIADVFFVSVLPFKIAYHLSGNDWLFGPGMCRIVTAIFYCNMYCSVLLIASISVDRFLAVVYPMHSLSWRTMSRAYMACSFIWLISIASTIPLLVTEQTQKIPRLDITTCHDVLDLKDLKDFYIYYFSSFCLLFFFVPFIITTICYIGIIRSLSSSSIENSCKKTRALFLAVVVLCVFIICFGPTNVLFLTHYLQEANEFLYFAYILSACVGSVSCCLDPLIYYYASSQCQRYLYSLLCCRKVSEPGSSTGQLMSTAMKNDNCSTNAKSSIYKKLLA

SEQ ID NO: 42 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLVLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISSCIDPLIYYYASSECQRYVYSILCCKESSDPSSYNSSGQLMASKMDTCSSNLNNSIYKKLLT

SEQ ID NO: 43 - protein, 385 aa, Homo sapiens
MWGRLLLWPLVLGFSLSGGTQTPSVYDESGSTGGGDDSTPSILPAPRGYPGQVCANDSDTLELPDSSRALLLGWVPTRLVPALYGLVLVVGLPANGLALWVLATQAPRLPSTMLLMNLATADLLLALALPPRIAYHLRGQRWPFGEAACRLATAALYGHMYGSVLLLAAVSLDRYLALVHPLRARALRGRRLALGLCMAAWLMAAALALPLTLQRQTFRLARSDRVLCHDALPLDAQASHWQPAFTCLALLGCFLPLLAMLLCYGATLHTLAASGRRYGHALRLTAVVLASAVAFFVPSNLLLLLHYSDPSPSAWGNLYGAYVPSLALSTLNSCVDPFIYYYVSAEFRDKVRAGLFQRSPGDTVASKASAEGGSRGMGTHSSLLQ

SEQ ID NO: 44 - protein, 370 aa, Homo sapiens
MGDRRFIDFQFQDSNSSLRPRLGNATANNTCIVDDSFKYNLNGAVYSVVFILGLITNSVSLFVFCFRMKMRSETAIFITNLAVSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTTCFEGFSKRVWKTYLSKITIFIEVVGFIIPLILNVSCSSVVLRTLRKPATLSQIGTNKKKVLKMITVHMAVFVVCFVPYNSVLFLYALVRSQAITNCFLERFAKIMYPITLCLATLNCCFDPFIYYFTLESFQKSFYINAHIRMESLFKTETPLTTKPSLPAIQEEVSDQTTNNGGELMLESTF

SEQ ID NO: 45 - protein, 444 aa, Homo sapiens
MSGTKLEDSPPCRNWSSASELNETQEPFLNPTDYDDEEFLRYLWREYLHPKEYEWVLIAGYIIVFVVALIGNVLVCVAVWKNHHMRTVTNYFIVNLSLADVLVTITCLPATLVVDITETWFFGQSLCKVIPYLQTVSVSVSVLTLSCIALDRWYAICHPLMFKSTAKRARNSIVIIWIVSCIIMIPQAIVMECSTVFPGLANKTTLFTVCDERWGGEIYPKMYHICFFLVTYMAPLCLMVLAYLQIFRKLWCRQIPGTSSVVQRKWKPLQPVSQPRGPGQPTKSRMSAVAAEIKQIRARRKTARMLMVVLLVFAICYLPISILNVLKRVFGMFAHTEDRETVYAWFTFSHWLVYANSAANPIIYNFLSGKFREEFKAAFSCCCLGVHHRQEDRLTRGRTSTESRKSLTTQISNFDNISKLSEQVVLTSISTLPAANGAGPLQNW

SEQ ID NO: 46 - protein, 460 aa, Rattus norvegicus
MSSTKLEDSLPRRNWSSASELNETQEPFLNPTDYDDEEFLRYLWREYLHPKEYEWVLIAGYIIVFVVALIGNVLVCVAVWKNHHMRTVTNYFIVNLSLADVLVTITCLPATLVVDITETWFFGQSLCKVIPYLQTVSVSVSVLTLSCIALDRWYAICHPLMFKSTAKRARNSIVVIWIVSCIIMIPQAIVMERSSMLPGLANKTTLFTVCDERWGGEVYPKMYHICFFLVTYMAPLCLMVLAYLQIFRKLWCRQIPGTSSVVQRKWKQPQPVSQPRGSGQQSKARISAVAAEIKQIRARRKTARMLMVVLLVFAICYLPISILNVLKRVFGMFTHTEDRETVYAWFTFSHWLVYANSAANPIIYNFLSGKFREEFKAAFSCCLGVHRRQGDRLARGRTSTESRKSLTTQISNFDNVSKLSEHVALTSISTLPAANGAGPLQNWYLQQGVPSSLLSTWLEV

SEQ ID NO: 47 - protein, 375 aa, Mus musculus
MNTSHFLAPLFPGSLQGKNGTNPLDSPYNFSDGCQDSAELLAFIITTYSIETILGVLGNLCLIFVTTRQKEKSNVTNLLIANLAFSDFLMCLICQPLTVTYTIMDYWIFGEVLCKMLTFIQCMSVTVSILSLVLVALERHQLIINPTGWKPSIFQAYLGIVVIWFISCFLSLPFLANSTLNDLFHYNHSKVVEFLEDKVVCFVSWSSDHHRLIYTTFLLLFQYCIPLAFILVCYIRIYQRLQRQKHVFHAHACSSRAGQMKRINSMLMTMVTAFAVLWLPLHVFNTLEDWYQEAIPACHGNLIFLMCHLLAMASTCVNPFIYGFLNINFKKDIKALVLTCHCRSPQGESEHLPLSTVHTDLSKGSMRMGSKSNFI

SEQ ID NO: 48 - protein, 375 aa, Rattus norvegicus
MNTSHLMASLSPAFLQGKNGTNPLDSLYNLSDGCQDSADLLAFIITTYSVETVLGVLGNLCLIFVTTRQKEKSNVTNLLIANLAFSDFLMCLICQPLTVTYTIMDYWIFGEVLCKMLTFIQCMSVTVSILSLVLVALERHQLIINPTGWKPSISQAYLGIVVIWFISCFLSLPFLANSILNDLFHYNHSKVVEFLEDKVVCFVSWSSDHHRLIYTTFLLLFQYCVPLAFILVCYMRIYQRLQRQRRAFHTHTCSSRVGQMKRINGMLMAMVTAFAVLWLPLHVFNTLEDWYQEAIPACHGNLIFLMCHLFAMASTCVNPFIYGFLNINFKKDIKALVLTCRCRPPQGEPEPLPLSTVHTDLSKGSMRMGSKSNVM

SEQ ID NO: 49 - protein, 371 aa, Oryctolagus cuniculus
MEVSLNDPASNKTSAKSNSSAFFYFESCQSPSLALLLLLIAYTVVLIMGICGNLSLITIIFKKQREAQNVTNILIANLSLSDILVCVMCIPFTAIYTLMDRWIFGNTMCKLTSYVQSVSISVSIFSLVLIAIERYQLIVNPRGWKPSASHAYWGIMLIWLFSLLLSIPLLLSYHLTDEPFRNLSLPTDLYSHHVVCVEHWPSKTNQLLYSTSLIMLQYFVPLGFMFICYLKIVICLHKRNSKIDRRRENESRLTENKRINTMLISIVVTFAACWLPLNTFNVIFDWYHEVLMSCHHDLVFAICHLVAMVSTCINPLFYGFLNRNFQKDLVVLIHHCLCFALRERYENIAISTLHTDESKGSLRVAHIPAGI

SEQ ID NO: 50 - protein, 383 aa, Cavia porcellus
MNSTSFSQLENHSVHYNLSEEKPSFFAFENDDCHLPLAVIFTLALAYGAVIILGVSGNLALILIILKQKEMRNVTNILIVNLSFSDLLVAIMCLPFTFVYTLMDHWIFGEIMCKLNPFVQCVSITVSIFSLVLIAVERHQLIINPRGWRPNNRHAYIGIAVIWVLAVASSLPFMIYQVLTDEPFQNVTLDAFKDKLVCFDQFPSDSHRLSYTTLLLVLQYFGPLCFIFICYFKIYIRLKRRNNMMDKMRDSKYRSSESKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPLAFKKISCVENEKI

SEQ ID NO: 51 - protein, 375 aa, Danio rerio
MERSHLNNSSWLLEDPTCPASLSSTTFLIVAYSTMLAVGLVGNTCLVVVITRQKEMRNVTNIFIVNLSCSDILVCLVCLPVTIIYTLMDRWILGEALCKVTPFVQCMSVTVSIFSMVLIALERHQLIIHPTGWKPVVRHSYLAVAVIWIIACFISLPFLSFNILTNSPFHNLSLPFNPFSDHFICIEQWPSEGNRLTYTTTLLLCQYCLPLALILVCYFRIFLRLSRRKDMVERARGGRQKKAKGSKRVNAMLASIVAAFALCWLPLNVFNTIFDWNHEAIPVCQHDAIFSACHLTAMASTCVNPVIYGFLNNNFQKELKSLLSRCRCWGPAESYESFPLSTVSTGITKGSILSNGSASTYQPHKKNSLEQKESI

SEQ ID NO: 52 - protein, 381 aa, Homo sapiens
MGPIGAEADENQTVEEMKVEQYGPQTTPRGELVPDPEPELIDSTKLIEVQVVLILAYCSIILLGVIGNSLVIHVVIKFKSMRTVTNFFIANLAVADLLVNTLCLPFTLTYTLMGEWKMGPVLCHLVPYAQGLAVQVSTITLTVIALDRHRCIVYHLESKISKRISFLIIGLAWGISALLASPLAIFREYSLIEIIPDFEIVACTEKWPGEEKSIYGTVYSLSSLLILYVLPLGIISFSYTRIWSKLKNHVSPGAANDHYHQRRQKTTKMLVCVVVVFAVSWLPLHAFQLAVDIDSQVLDLKEYKLIFTVFHIIAMCSTFANPLLYGWMNSNYRKAFLSAFRCEQRLDAIHSEVSVTFKAKKNLEVRKNSGPNDSFTEATNV

SEQ ID NO: 53 - protein, 522 aa, Homo sapiens
MNSFFGTPAASWCLLESDVSSAPDKEAGRERRALSVQQRGGPAWSGSLEWSRQSAGDRRRLGLSRQTAKSSWSRSRDRTCCCRRAWWILVPAADRARRERFIMNEKWDTNSSENWHPIWNVNDTKHHLYSDINITYVNYYLHQPQVAAIFIISYFLIFFLCMMGNTVVCFIVMRNKHMHTVTNLFILNLAISDLLVGIFCMPITLLDNIIAGWPFGNTMCKISGLVQGISVAASVFTLVAIAVDRFQCVVYPFKPKLTIKTAFVIIMIIWVLAITIMSPSAVMLHVQEEKYYRVRLNSQNKTSPVYWCREDWPNQEMRKIYTTVLFANIYLAPLSLIVIMYGRIGISLFRAAVPHTGRKNQEQWHVVSRKKQKIIKMLLIVALLFILSWLPLWTLMMLSDYADLSPNELQIINIYIYPFAHWLAFGNSSVNPIIYGFFNENFRRGFQEAFQLQLCQKRAKPMEAYTLKAKSHVLINTSNQLVQESTFQNPHGETLLYRKSAEKPQQELVMEELKETTNSSEI

SEQ ID NO: 54 - protein, 348 aa, Mus musculus
MELAMVNLSEGNGSDPEPPAPESRPLFGIGVENFITLVVFGLIFAMGVLGNSLVITVLARSKPGKPRSTTNLFILNLSIADLAYLLFCIPFQATVYALPTWVLGAFICKFIHYFFTVSMLVSIFTLAAMSVDRYVAIVHSRRSSSLRVSRNALLGVGFIWALSIAMASPVAYHQRLFHRDSNQTFCWEQWPNKLHKKAYVVCTFVFGYLLPLLLICFCYAKVLNHLHKKLKNMSKKSEASKKKTAQTVLVVVVVFGISWLPHHVVHLWAEFGAFPLTPASFFFRITAHCLAYSNSSVNPIIYAFLSENFRKAYKQVFKCHVCDESPRSETKENKSRMDTPPSTNCTHV

SEQ ID NO: 55 - DNA, 38 bp, Homo sapiens
gcagcagcggccgccggcgcatggggcccagatccccg

SEQ ID NO: 56 - DNA, 37 bp, Homo sapiens
gcagcagtcgacgaacacactctcctgcctctggagg

SEQ ID NO: 57 - DNA, 39 bp, Homo sapiens
gcagcagcggccgcatgcaggtcccgaacagcaccggcc

SEQ ID NO: 58 - DNA, 37 bp, Homo sapiens
gcagcagtcgaccttgtacacgtggtagtagctcttg

(19) United States
(12) Patent Application Publication
(10) Pub. No.: US 2003/0064438 A1
(43) Pub. Date: Apr. 3, 2003

(54) G-protein coupled receptor nucleic acids, polypeptides, antibodies and uses thereof

(76) Inventors: John N. Feder, Belle Mead, NJ (US); Chandra S. Ramanathan, Wallingford, CT (US); Thomas C. Nelson, Lawrenceville, NJ (US); Gabriel A. Mintier, Hightstown, NJ (US); Angela Cacace, Clinton, CT (US); Lauren E. Barber, Jewett City, CT (US); Michael Kornacker, Princeton, NJ (US); David Bol, Langhorne, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/081,810

(22) Filed: Feb. 22, 2002

Related U.S. Application Data
(60) Provisional application No. 60/270,793, filed on Feb. 23, 2001.
(60) Provisional application No. 60/270,792, filed on Feb. 23, 2001.
(60) Provisional application No. 60/296,427, filed on Jun. 6, 2001.

(51) Int. Cl. C12P 21/02; C12N 5/06; C07H 21/04; C07K 14/435

(52) U.S. Cl. 435/69.1; 536/23.5; 530/350; 435/320.1; 435/325

(57) ABSTRACT

The invention provides isolated HGPRBMY1 and HGPRBMY2 nucleic acid molecules and polypeptide molecules. The invention also provides antisense nucleic acid molecules, expression vectors containing the nucleic acid molecules of the invention, host cells into which the expression vectors have been introduced, and non-human transgenic animals in which a nucleic acid molecule of the invention has been introduced or disrupted. The invention still further provides isolated polypeptides, fusion polypeptides, antigenic peptides and antibodies. Diagnostic, screening and therapeutic methods utilizing compositions of the invention are also provided.

This application claims benefit to provisional application U.S. Serial No. 60/270,793, filed Feb. 23, 2001; to provisional application U.S. Serial No. 60/270,792, filed Feb. 23, 2001; and to provisional application U.S. Serial No. 60/296,427, filed Jun. 6, 2001. The teachings of the referenced applications are incorporated herein by reference in their entirety.

### 1. INTRODUCTION

Many transmembrane proteins are receptors that bind a ligand and transduce an intracellular signal, leading to a variety of cellular responses. The identification and characterization of such a receptor enables one to identify both the ligands which bind to the receptor and the intracellular molecules and signal transduction pathways associated with the receptor, permitting one to identify or design modulators of receptor activity, e.g., receptor agonists or antagonists and modulators of signal transduction.

The present invention relates to the discovery and characterization of nucleic acid molecules that encode a G-protein coupled receptor (GPCR), a receptor that participates in signal transduction in eukaryotic cells. More specifically, the present invention relates to a novel GPCR that is particularly expressed in bone marrow and spleen tissue, referred to herein as HGPRBMY1. The invention features GPCR nucleic acid molecules, host cell expression systems, GPCRs, fusion polypeptides, peptides, antibodies to the receptor, transgenic animals that express a GPCR transgene, or recombinant knock-out animals that do not express the GPCR, antagonists and agonists of the receptor, and other compounds that modulate GPCR gene expression or GPCR activity that can be used for diagnosis, drug screening, clinical trial monitoring, and/or as pharmaceutical compositions the treatment of immune related diseases and disorders, particularly proliferative immune and autoimmune disorders, specifically p27 and/or IkB defects.

The present invention relates to the discovery and characterization of nucleic acid molecules that encode a G-protein coupled receptor (GPCR), a receptor that participates in signal transduction in eukaryotic cells. More specifically, the present invention relates to a novel GPCR that is particularly expressed in heart and brain tissue, referred to herein as HGPRBMY2. The invention encompasses GPCR nucleic acid molecules, host cell expression systems, GPCR polypeptides, fusion polypeptides, peptides, antibodies to the receptor, transgenic animals that express a GPCR transgene, or recombinant knock-out animals that do not express the GPCR, antagonists and agonists of the receptor, and other compounds that modulate GPCR gene expression or GPCR activity that can be used for diagnosis, drug screening, clinical trial monitoring, and/or as pharmaceutical compositions the treatment of cardiovascular and/or neural diseases and disorders.

### 2. BACKGROUND OF THE INVENTION

G-protein coupled receptors (GPCRs) belong to one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also important signaling molecules in subjects with depression, schizophrenia, sleeplessness, hypertension, anxiety, stress, renal failure and in several other cardiovascular, metabolic, neuro, oncology and immune disorders (Horn and Vriend, J. Mol. Med. 76:464-468, 1998). They have also been shown to play a role in HIV infection (Feng et al., (1996) Science 272:872-877).

GPCRs are integral membrane proteins characterized by the presence of seven hydrophobic transmembrane domains which span the plasma membrane and form a bundle of antiparallel alpha helices. The transmembrane domains account for structural and functional features of the receptor. In most cases, the bundle of helices forms a binding pocket; however, when the binding site must accommodate more bulky molecules, the extracellular N-terminal segment or one or more of the three extracellular loops participate in binding and in subsequent induction of conformational change in intracellular portions of the receptor. The activated receptor, in turn, interacts with an intracellular heterotrimeric G-protein complex which mediates further intracellular signaling activities, generally interaction with guanine nucleotide binding (G) proteins and the production of second messengers such as cyclic AMP (cAMP), phospholipase C, inositol triphosphate or ion channel proteins (Baldwin, J. M. (1994) Curr. Opin. Cell Biol. 6:180-190). The activity of the receptors are then modulated by modification, such as phosphorylation, or by binding to a regulatory molecule, such as by the negative regulatory molecule arrestin, or by internalization wherein the receptor is degraded in a lyzosome (see generally Hu, L. A., et al., (2000) J. Biol. Chem.. 275:38659-38666).

The amino-terminus of the GPCR is extracellular, of variable length and often glycosylated, while the carboxy-terminus is cytoplasmic. Extracellular loops of the GPCR alternate with intracellular loops and link the transmembrane domains. The most conserved domains of GPCRs are the transmembrane domains and the first two cytoplasmic loops. GPCRs range in size from under 400 to over 1000 amino acids (Coughlin, S. R. (1994) Curr. Opin. Cell Biol. 6:191-197).

GPCRs respond to a diverse array of ligands including lipid analogs, amino acids and their derivatives, peptides, cytokines, and specialized stimuli such as light, taste, and odor. GPCRs function in physiological processes including vision (the rhodopsins), smell (the olfactory receptors), neurotransmission (muscarinic acetylcholine, dopamine, and adrenergic receptors), and hormonal response (luteinizing hormone and thyroid-stimulating hormone receptors).

GPCR mutations, both of the loss-of-function and of the activating variety, have been associated with numerous human diseases (Coughlin, supra). For instance, retinitis pigmentosa may arise from either loss-of-function or activating mutations in the rhodopsin gene. Somatic activating mutations in the thyrotropin receptor cause hyperfunctioning thyroid adenomas (Parma, J. et al. (1993) Nature 365:649-651). Parma et al. suggest that certain G-protein-coupled receptors susceptible to constitutive activation may behave as proto-oncogenes.

Characterization of the HGPRBMY1 polypeptide of the present invention led to the determination that it is involved in the modulation of the cyclin p27 protein, in addition to, the apoptosis regulatory protein IkB, either directly or indirectly. The present invention represents the first association between HGPRBMY1 to cell cycle and apoptosis regulation.

Critical transitions through the cell cycle are highly regulated by distinct protein kinase complexes, each composed of a cyclin regulatory and a cyclin-dependent kinase (cdk) catalytic subunit (for review see Draetta, 1994). These proteins regulate the cell's progression through the stages of the cell cycle and are in turn regulated by numerous proteins, including p53, p21, p16, p27, and cdc25. Downstream targets of cyclin-cdk complexes include pRb and E2F. The cell cycle often is dysregulated in neoplasia due to alterations either in oncogenes that indirectly affect the cell cycle or in tumor suppressor genes or oncogenes that directly impact cell cycle regulation, such as pRb, p53, p16, cyclin D1, or mdm-2 (for review see Lee and Yang, 2001, Schafer, 1998).

P27, also known as CDNK1B (cyclin-dependent kinase inhibitor 1B) or KIP1, shares a limited similarity with the CDK inhibitor CDKN1A/p21. The encoded protein binds to and prevents the activation of cyclinE-CDK2 or cyclinD-CDK4 complexes. Therefore it mainly blocks the cell cycle progression at the G1- and S-phases (for review see Desdouets and Brechot, 2000).

Reduction in levels of p27 and increased expression of cyclin E also occur and carry a poor prognostic significance in many common forms of cancer. The inhibition of protein activities leading to an upregulation of p27 might therefore be a possibility to decrease the progression of cancer and increase patient survival rates (for review see Sgambato, 2000).

Recently, Medema et al. (2000) demonstrated that p27 is a major transcriptional target of forkhead transcription factors FKHRL1, AFX, or FKHR. Overexpression of these proteins causes growth suppression in a variety of cell lines, including a Ras-transformed cell line and a cell line lacking the tumor suppressor PTEN integrating signals from PI3K/PKB signaling and RAS/RAL signaling to regulate transcription of p27(KIP1). Expression of AFX blocked cell cycle progression at phase G1, independent of functional retinoblastoma protein but dependent on the cell cycle inhibitor p27 (KIP1). This is further supported by the fact that AFX activity inhibits p27 −/− knockout mouse cells significantly less than their p27 +/+ counterparts.

The connection between the PTEN pathway and the activation of p27 via forkhead-like transcription factors implies that genes whose inhibition leads to p27 upregulation might be involved in this pathway. Therefore the identification of genes whose knockout leads to an upregulation of p27 might be useful drug targets, as inhibition of such genes should result in the upregulation of p27 and therefore be useful for the treatment and/or amelioration of cancer and increase a cancer patients prolonged outlook and survival.

The fate of a cell in multicellular organisms often requires choosing between life and death. This process of cell suicide, known as programmed cell death or apoptosis, occurs during a number of events in an organisms life cycle, such as for example, in development of an embryo, during the course of an immunological response, or in the demise of cancerous cells after drug treatment, among others. The final outcome of cell survival versus apoptosis is dependent on the balance of two counteracting events, the onset and speed of caspase cascade activation (essentially a protease chain reaction), and the delivery of antiapoptotic factors which block the caspase activity (Aggarwal B. B. Biochem. Pharmacol. 60, 1033-1039, (2000); Thornberry, N. A. and Lazebnik, Y. Science 281, 1312-1316, (1998)).

The production of antiapoptotic proteins is controlled by the transcriptional factor complex NF-kB. For example, exposure of cells to the protein tumor necrosis factor (TNF) can signal both cell death and survival, an event playing a major role in the regulation of immunological and inflammatory responses (Ghosh, S., May, M. J., Kopp, E. B. Annu. Rev. hnmunol. 16, 225-260, (1998); Silverman, N. and Maniatis, T., Genes & Dev. 15, 2321-2342, (2001); Baud, V. and Karin, M., Trends Cell Biol. 11, 372-377, (2001)). The anti-apoptotic activity of NF-kB is also crucial to oncogenesis and to chemo- and radio-resistance in cancer (Baldwin, A. S., J. Clin. Inves. 107, 241-246, (2001)).

Nuclear Factor-kB (NF-kB), is composed of dimeric complexes of p50 (NF-kB1) or p52 (NF-kB2) usually associated with members of the Rel family (p65, c-Rel, Rel B) which have potent transactivation domains. Different combinations of NF-kB/Rel proteins bind distinct kB sites to regulate the transcription of different genes. Early work involving NF-kB suggested its expression was limited to specific cell types, particularly in stimulating the transcription of genes encoding kappa immunoglobulins in B lymphocytes. However, it has been discovered that NF-kB is, in fact, present and inducible in many, if not all, cell types and that it acts as an intracellular messenger capable of playing a broad role in gene regulation as a mediator of inducible signal transduction. Specifically, it has been demonstrated that NF-kB plays a central role in regulation of intercellular signals in many cell types. For example, NF-kB has been shown to positively regulate the human beta-interferon (beta-IFN) gene in many, if not all, cell types. Moreover, NF-kB has also been shown to serve the important function of acting as an intracellular transducer of external influences.

The transcription factor NF-kB is sequestered in an inactive form in the cytoplasm as a complex with its inhibitor, IkB, the most prominent member of this class being IkBa. A number of factors are known to serve the role of stimulators of NF-kB activity, such as, for example, TNF. After TNF exposure, the inhibitor is phosphorylated and proteolytically removed, releasing NF-kB into the nucleus and allowing its transcriptional activity. Numerous genes are upregulated by this transcription factor, among them IkBa. The newly synthezised IkBa protein inhibits NF-kB, effectively shutting down further transcriptional activation of its downstream effectors. However, as mentioned above, the IkBa protein may only inhibit NF-kB in the absence of IkBa stimuli, such as TNF stimulation, for example. Other agents that are known to stimulate NF-kB release, and thus NF-kB activity, are bacterial lipopolysaccharide, extracellular polypeptides, chemical agents, such as phorbol esters, which stimulate intracellular phosphokinases, inflammatory cytokines, IL-1, oxidative and fluid mechanical stresses, and Ionizing Radiation (Basu, S., Rosenzweig, K, R., Youmell, M., Price, B, D, Biochem, Biophys, Res, Commun., 247(1):79-83, (1998)). Therefore, as a general rule, the stronger the insulting stimulus, the stronger the resulting NF-kB activation, and the higher the level of IkBa transcription. As a consequence, measuring the level of IkBa RNA can be used as a marker for antiapoptotic events, and indirectly, for the onset and strength of pro-apoptotic events.

The upregulation of IkBa due to the downregulation of HGPRBMY1 places this GPCR protein into a signalling pathway potentially involved in apoptotic events. This gives the opportunity to regulate downstream events via the activity of the protein HGPRBMY1 with antisense polynucleotides, polypeptides or low molecular chemicals with the potential of achieving a therapeutic effect in cancer, and autoimmune diseases. In addition to cancer and immunological disorders, NF-kB has significant roles in other diseases (Baldwin, A. S., J. Clin Invest. 107, :3-6 (2001)). NF-kB is a key factor in the pathophysiology of ischemia-reperfusion injury and heart failure (Valen, G., Yan. Z Q, Hansson, G K, J. Am. Coll. Cardiol. 38, 307-14 (2001)). Furthermore, NF-kB has been found to be activated in experimental renal disease (Guijarro C, Egido J., Kidney Int. 59, 415-425 (2001)). As HGPRBMY1 is highly expressed in bone marrow and spleen and there is the potential of an involvement in immune diseases.

The discovery of a new human G-protein coupled receptor as described herein, and the nucleic acids encoding it satisfies a need in the art by providing new compositions which are useful in the diagnosis, prevention and treatment of immune disorders, and particularly those G-protein coupled receptors that modulate the p27 and/or NFkB pathways.

### 3. SUMMARY OF THE INVENTION

HGPRBMY1 is a putative G-protein coupled receptor (GPCR) that is expressed in tissues, in particular immune system tissues such as bone marrow, spleen and thymus. More specifically, HGPRBMY1 comprises the amino acid sequences depicted in FIG. 2 which is encoded by the nucleic acid sequence depicted in FIG. 1.

HGPRBMY2 is predicted to be a G-protein coupled receptor (GPCR) that is expressed in heart and brain tissue. More specifically, HGPRBMY2 comprises the amino acid sequences depicted in FIG. 7 which is encoded by the nucleic acid sequence depicted in FIG. 6. The clone encoding the HGPRBMY2 polypeptide was deposited with the ATCC as ATCC Deposit Number XXXXX on XXXXX.

As HGPRBMY1 and HGPRBMY2 have homology to GPCRs, they are likely seven transmembrane proteins located at the membrane of a cell. Signal transduction from GPCRs is triggered by the binding of agonists or antagonists to the receptor. Secondary regulation of the receptor may occur through post-stimulatory modification of the polypeptide (e.g., phosphorylation) and/or by binding to a secondary regulatory molecule, particularly on a cytoplasmic domain of the receptor (e.g., arrestin).

HGPRBMY1 mRNA has been detected in the bone marrow, spleen and thymus. Thus, neutralization of HGPRBMY1 agonists or antagonists, removal of HGPRBMY1 agonists or antagonists, or interference with binding to HGPRBMY1 may result in improvement or prevention of immune related disease.

HGPRBMY2 mRNA has been detected in the heart, and various tissues of the brain. Thus, neutralization of HGPRBMY2 agonists or antagonists, removal of HGPRBMY2 agonists or antagonists, or interference with binding to HGPRBMY2 may result in improvement or prevention of cardiovascular and/.or neurological diseases.

The invention features the use of HGPRBMY1 nucleic acid molecules, HGPRBMY1 polypeptides and peptides, fusion polypeptides or fusion peptides (e.g., fusions to heterologous sequences), as well as antibodies to the HGPRBMY1 (which can, for example, act as HGPRBMY1 agonists or antagonists), antagonists that inhibit receptor activity or expression, or agonists that activate receptor activity or increase its expression in the diagnosis and treatment of immune system or immune response diseases and/or disorders including, but not limited to immune system diseases or disorders in animals, including humans, particularly proliferative immune disorders, and autoimmune disorders.

The invention features the use of HGPRBMY2 nucleic acid molecules, HGPRBMY2 polypeptides and peptides, fusion polypeptides or fusion peptides (e.g., fusions to heterologous sequences), as well as antibodies to the HGPRBMY2 (which can, for example, act as HGPRBMY2 agonists or antagonists), antagonists that inhibit receptor activity or expression, or agonists that activate receptor activity or increase its expression in the diagnosis and treatment of the cardiovascular system diseases or disorders, in addition to neural disorders, in animals, including humans.

The diagnosis of an HGPRBMY1 abnormality in a patient, or an abnormality in the HGPRBMY1 signal transduction pathway, will assist in devising a proper treatment or therapeutic regimen for immune disorders. In addition, HGPRBMY1 nucleic acid molecules and HGPRBMY1 polypeptides are useful for the identification of compounds effective in the treatment of immune disorders regulated by the HGPRBMY1, particularly proliferative immune disorders, and autoimmune disorders.

The diagnosis of an HGPRBMY2 abnormality in a patient, or an abnormality in the HGPRBMY2 signal transduction pathway, will assist in devising a proper treatment or therapeutic regimen for heart failure. In addition, HGPRBMY2 nucleic acid molecules and HGPRBMY2 polypeptides are useful for the identification of compounds effective in the treatment of cardiovascular and/or neural disorders regulated by the HGPRBMY2.

In particular, the invention described in the subsections below features HGPRBMY1, polypeptides or peptides corresponding to functional domains of the HGPRBMY1 (e.g., extracellular domain (ECD), transmembrane domain (TM) or cytoplasmic domain (CD)), mutated, truncated or deleted HGPRBMY1 (e.g., an HGPRBMY1 with one or more functional domains or portions thereof deleted, such as ΔTM and/or ΔCD), HGPRBMY1 fusion polypeptides (e.g., an HGPRBMY1 or a functional domain of HGPRBMY1, such as the ECD, fused to an unrelated polypeptide or peptide such as an immunoglobulin constant region, i.e., Ig-Fc), nucleic acid sequences encoding such products, and host cell expression systems that can produce such HGPRBMY1 products.

The invention also features antibodies and anti-idiotypic antibodies (including Fab fragments), antagonists and agonists of the HGPRBMY1, as well as compounds or nucleic acid constructs that inhibit expression of the HGPRBMY1 gene (transcription factor inhibitors, antisense and ribozyme molecules, or gene or regulatory sequence replacement constructs), or promote expression of HGPRBMY1 (e.g., expression constructs in which HGPRBMY1 coding sequences are operatively associated with expression control elements such as promoters, promoter/enhancers, etc.). The invention also relates to host cells and animals genetically engineered to express the human HGPRBMY1 (or mutants thereof) or to inhibit or “knock-out” expression of the animal's endogenous HGPRBMY1.

The HGPRBMY1 polypeptides or peptides, HGPRBMY1 fusion polypeptides, HGPRBMY1 nucleic acid sequences, antibodies, antagonists and agonists can be useful for the detection of mutant HGPRBMY1 or inappropriately expressed HGPRBMY1 for the diagnosis of immune disorders. The HGPRBMY1 polypeptides or peptides, HGPRBMY1 fusion polypeptides, HGPRBMY1 nucleic acid sequences, host cell expression systems, antibodies, antagonists, agonists and genetically engineered cells and animals can be used for screening for drugs effective in the treatment of such immune disorders. The use of engineered host cells and/or animals may offer an advantage in that such systems allow not only for the identification of compounds that bind to the ECD of the HGPRBMY1, but can also identify compounds that affect the signal transduced by the activated HGPRBMY1.

The HGPRBMY2 polypeptides or peptides, HGPRBMY2 fusion polypeptides, HGPRBMY2 nucleic acid sequences, antibodies, antagonists and agonists can be useful for the detection of mutant HGPRBMY2 or inappropriately expressed HGPRBMY2 for the diagnosis of heart disease or neural disorders. The HGPRBMY2 polypeptides or peptides, HGPRBMY2 fusion polypeptides, HGPRBMY2 nucleic acid sequences, host cell expression systems, antibodies, antagonists, agonists and genetically engineered cells and animals can be used for screening for drugs effective in the treatment of such heart disease or immune disorders. The use of engineered host cells and/or animals may offer an advantage in that such systems allow not only for the identification of compounds that bind to the ECD of the HGPRBMY2, but can also identify compounds that affect the signal transduced by the activated HGPRBMY2.

The HGPRBMY1 polypeptide products (especially soluble derivatives such as peptides corresponding to the HGPRBMY1 ECD, or soluble polypeptides lacking one or more TM domains (“ΔTM”)), fusion polypeptides (especially HGPRBMY1-Ig fusion polypeptides, i.e., fusions of the HGPRBMY1 or a domain of the HGPRBMY1, e.g., ECD, ΔTM or CD to a heterologous sequence such as IgFc), antibodies and anti-idiotypic antibodies (including Fab fragments), antagonists or agonists (including compounds that modulate signal transduction which may act on downstream targets in the HGPRBMY1 signal transduction pathway) can be used for therapy of such diseases. For example, the administration of an effective amount of a pharmaceutical composition comprising soluble HGPRBMY1 ECD, ΔTM HGPRBMY1 or an ECD-IgFc fusion polypeptide or an anti-idiotypic antibody (or its Fab) that mimics the HGPRBMY1 ECD would modulate endogenous HGPRBMY1 agonists or antagonists, and prevent or reduce binding and receptor activation, leading to prevention of immune disorders.

For example, the administration of an effective amount of a pharmaceutical composition comprising a fusion polypeptide or an anti-idiotypic antibody, or fragment thereof, that mimics HGPRBMY1 would modulate endogenous HGPRBMY1 binding to signaling partners, leading to treatment of immune disorders, particulalry proliferative immune disorders, and autoimmune disorders.

For example, the administration of an effective amount of a pharmaceutical composition comprising soluble HGPRBMY2 ECD, ΔTM HGPRBMY2 or an ECD-IgFc fusion polypeptide or an anti-idiotypic antibody (or its Fab) that mimics the HGPRBMY2 ECD would modulate endogenous HGPRBMY2 agonists or antagonists, and prevent or reduce binding and receptor activation, leading to prevention of heart failure.

Nucleic acid constructs encoding such HGPRBMY1 products can be used to genetically engineer host cells to express such HGPRBMY1 products in vivo; these genetically engineered cells, when placed in the body, deliver a continuous supply of HGPRBMY1 polypeptides or peptides, that modulate HGPRBMY1 activity. Nucleic acid constructs encoding functional HGPRBMY1, mutant HGPRBMY1, or antisense and ribozyme molecules can be used in gene therapy approaches for the modulation of HGPRBMY1 activity in the treatment of immune disorders, particulalry proliferative immune disorders, and autoimmune disorders.

Nucleic acid constructs encoding such HGPRBMY2 products can be used to genetically engineer host cells to express such HGPRBMY2 products in vivo; these genetically engineered cells deliver a continuous supply of soluble HGPRBMY2 peptide, ECD or ΔTM or HGPRBMY2 fusion polypeptide that will modulate activation of HGPRBMY2 by agonists or antagonists. Nucleic acid constructs encoding functional HGPRBMY2, mutant HGPRBMY2, as well as antisense and ribozyme molecules can be used in “gene therapy” approaches for the modulation of HGPRBMY2 expression and/or activity in the treatment of heart disease or neural disorders.

The invention also features HGPRBMY1 pharmaceutical formulations and methods for treating immune disorders, particulalry proliferative immune disorders, and autoimmune disorders.

Thus, the invention also encompasses HGPRBMY2 pharmaceutical formulations and methods for treating heart or neural diseases.

The invention further relates to a method of identifying a compound that modulates the biological activity of HGPRBMY1 or HGPRBMY2, comprising the steps of, (a) combining a candidate modulator compound with HGPRBMY1 or HGPRBMY2 having the sequence set forth in one or more of SEQ ID NO:2; and measuring an effect of the candidate modulator compound on the activity of HGPRBMY1 or HGPRBMY2.

The invention further relates to a method of identifying a compound that modulates the biological activity of a GPCR, comprising the steps of, (a) combining a candidate modulator compound with a host cell expressing HGPRBMY1 or HGPRBMY2 having the sequence as set forth in SEQ ID NO:2; and, (b) measuring an effect of the candidate modulator compound on the activity of the expressed HGPRBMY1 or HGPRBMY2.

The invention further relates to a method of identifying a compound that modulates the biological activity of HGPRBMY1 or HGPRBMY2, comprising the steps of, (a) combining a candidate modulator compound with a host cell containing a vector described herein, wherein HGPRBMY1 or HGPRBMY2 is expressed by the cell; and, (b) measuring an effect of the candidate modulator compound on the activity of the expressed HGPRBMY1 or HGPRBMY2.

The invention further relates to a method of screening for a compound that is capable of modulating the biological activity of HGPRBMY1 or HGPRBMY2, comprising the steps of: (a) providing a host cell described herein; (b) determining the biological activity of HGPRBMY1 or HGPRBMY2 in the absence of a modulator compound; (c) contacting the cell with the modulator compound; and (d) determining the biological activity of HGPRBMY1 or HGPRBMY2 in the presence of the modulator compound; wherein a difference between the activity of HGPRBMY1 or HGPRBMY2 in the presence of the modulator compound and in the absence of the modulator compound indicates a modulating effect of the compound.

The invention further relates to a recombinant host cell comprising a vector comprising all or a portion of the polynucleotide of SEQ ID NO:1 or SEQ ID NO:13, NFAT/CRE, and/or NFAT G alpha 15 wherein said host cell exhibits low levels of HGPRBMY1 or HGPRBMY2 expression. Such host cells are particularly useful in methods of screening for agonists of the HGPRBMY1 or HGPRBMY2 polypeptide.

The invention further relates to a recombinant host cell comprising a vector comprising all or a portion of the polynucleotide of SEQ ID NO:1 or SEQ ID NO:13, NFAT/CRE, and/or NFAT G alpha 15 wherein said host cell exhibits intermediate levels of HGPRBMY1 or HGPRBMY2 expression. Such host cells are particularly useful in methods of screening for modulators of the HGPRBMY1 or HGPRBMY2 polypeptide.

The invention further relates to a recombinant host cell comprising a vector comprising all or a portion of the polynucleotide of SEQ ID NO:1 or SEQ ID NO:13, NFAT/CRE, and/or NFAT G alpha 15 wherein said host cell exhibits high levels of HGPRBMY1 or HGPRBMY2 expression. Such host cells are particularly useful in methods of screening for antagonists of the HGPRBMY1 or HGPRBMY2 polypeptide.

The invention further relates to a method of screening for candidate compounds capable of modulating activity of a G-protein coupled receptor-encoding polypeptide, comprising the steps of contacting a test compound with a cell or tissue expressing all or a portion of the polynucleotide of SEQ ID NO:1 or SEQ ID NO:13, NFAT/CRE, and/or NFAT G alpha 15 wherein said cell or tissue exhibits low, intermediate, or high HGPRBMY1 or HGPRBMY2 expression levels, and selecting as candidate modulating compounds those test compounds that modulate activity of the the HGPRBMY1 or HGPRBMY2 polypeptide.

The invention relates to a method of preventing, treating, or ameliorating a medical condition, comprising administering to a mammalian subject a therapeutically effective amount of the polypeptide of SEQ ID NO:2 or the polynucleotide of SEQ ID NO:1, wherein the medical condition is a proliferative disorder.

More preferably, the invention relates to a method of preventing, treating, or ameliorating a medical condition, comprising administering to a mammalian subject a therapeutically effective amount of an antagonist of the polypeptide of SEQ ID NO:2 or the polynucleotide of SEQ ID NO:1, wherein the medical condition is a proliferative disorder.

More preferably, the invention relates to a method of preventing, treating, or ameliorating a medical condition, comprising administering to a mammalian subject a therapeutically effective amount of an antagonist of the polypeptide of SEQ ID NO:2 or the polynucleotide of SEQ ID NO:1, wherein the medical condition is a disoder related to aberrant apoptosis regulation.

Alternatively, the invention relates to a method of preventing, treating, or ameliorating a medical condition, comprising administering to a mammalian subject a therapeutically effective amount of an agonist of the polypeptide of SEQ ID NO:2 or the polynucleotide of SEQ ID NO:1, wherein the medical condition is a proliferative disorder.

More preferably, the invention relates to a method of preventing, treating, or ameliorating a medical condition, comprising administering to a mammalian subject a therapeutically effective amount of an agonist of the polypeptide of SEQ ID NO:2 or the polynucleotide of SEQ ID NO:1, wherein the medical condition is a disoder related to aberrant apoptosis regulation.

The invention further relates to peptides that bind to the HGPRBMY1 or HGPRBMY2 polypeptide. More preferred are peptides that modulate the activity of HGPRBMY1 or HGPRBMY2 activity.

The invention further relates to a method for identifying compounds that regulate immune-related disorders, comprising the step of contacting a test compound with a cell which expresses a nucleic acid of SEQ ID NO:1, and determining whether the test compound modulates HGPRBMY1 activity.

The invention further relates to a method for identifying compounds that regulate immune-related disorders comprising the step of contacting a test compound with a nucleic acid of SEQ ID NO:1; and determining whether the test compound interacts with the nucleic acid of SEQ ID NO:1.

The invention further relates to a method for identifying compounds that regulate immune-related disorders, comprising the step of contacting a test compound with a cell or cell lysate containing a reporter gene operatively associated with a HGPRBMY1 regulatory element; and detecting expression of the reporter gene product.

The invention further relates to a method for identifying compounds that regulate immune-related disorders comprising the step of contacting a test compound with a cell or cell lysate containing HGPRBMY1 transcripts; and detecting the translation of the HGPRBMY1 transcript.

The invention further relates to a method for modulating immune-related disorders in a subject, comprising administering to the subject a therapeutically effective amount of a HGPRBMY1 polypeptide.

The invention further relates to a method for modulating immune-related disorders in a subject, comprising administering to the subject a therapeutically effective amount of a HGPRBMY1 polypeptide wherein the HGPRBMY1 polypeptide is HGPRBMY1 or a functionally equivalent derivative thereof, preferably wherein the subject is a human.

The invention further relates to a method for modulating immune-related disorders in a subject, comprising administering to the subject a therapeutically effective amount of a HGPRBMY1 polypeptide wherein the HGPRBMY1 polypeptide is HGPRBMY1 or a functionally equivalent derivative thereof, preferably wherein the subject is a human, wherein the HGPRBMY1 polypeptide is contained in a pharmaceutical composition.

The invention further relates to a method for the treatment of immune-related disorders, comprising modulating the activity of a HGPRBMY1 polypeptide.

The invention further relates to a method for the treatment of immune-related disorders, comprising modulating the activity of a HGPRBMY1 polypeptide, wherein the HGPRBMY1 polypeptide is HGPRBMY1 or a functionally equivalent derivative thereof.

The invention further relates to a method for the treatment of immune-related disorders, comprising modulating the activity of a HGPRBMY1 polypeptide, wherein the HGPRBMY1 polypeptide is HGPRBMY1 or a functionally equivalent derivative thereof, wherein the method comprises administering an effective amount of a compound that agonizes or antagonizes the activity of the HGPRBMY1 polypeptide.

The invention further relates to a method for the treatment of immune-related disorders, comprising administering an effective amount of a compound that decreases expression of a HGPRBMY1 gene.

The invention further relates to a method for the treatment of immune-related disorders, comprising administering an effective amount of a compound that decreases expression of a HGPRBMY1 gene, wherein the compound is an oligonucleotide encoding an antisense or ribozyme molecule that targets HGPRBMY1 transcripts and inhibits translation.

The invention further relates to a method for the treatment of immune-related disorders, comprising administering an effective amount of a compound that decreases expression of a HGPRBMY1 gene, wherein the compound is an oligonucleotide that forms a triple helix with the promoter of the HGPRBMY1 gene and inhibits transcription.

The invention further relates to a method for the treatment of immune-related disorders, comprising administering an effective amount of a compound that increases expression of a HGPRBMY1 gene.

The invention further relates to a pharmaceutical formulation for the treatment of immune-related disorders, comprising a compound that activates or inhibits HGPRBMY1 activity, mixed with a pharmaceutically acceptable carrier.

The invention further relates to a method for identifying compounds that modulate the activity of a G-protein coupled receptor comprising the step of (a)contacting a test compound to a cell that expresses a HGPRBMY1 gene and the G-protein coupled receptor, and measuring activity; (b) contacting a test compound to a cell that expresses a HGPRBMY1 gene but does not express the G-protein coupled receptor, and measuring activity; and (c) comparing activity obtained in (b) with the activity obtained in (a); such that if the level obtained in (b) differs from that obtained in (b), a compound that modulates G-protein coupled receptor activity is identified.

The invention further relates to a method for identifying compounds that regulate heart-related disorders, comprising the step of contacting a test compound with a cell which expresses a nucleic acid of SEQ ID NO:13, and determining whether the test compound modulates HGPRBMY2 activity.

The invention further relates to a method for identifying compounds that regulate heart-related disorders comprising the step of contacting a test compound with a nucleic acid of SEQ ID NO:13; and determining whether the test compound interacts with the nucleic acid of SEQ ID NO:13.

The invention further relates to a method for identifying compounds that regulate heart-related disorders, comprising the step of contacting a test compound with a cell or cell lysate containing a reporter gene operatively associated with a HGPRBMY2 regulatory element; and detecting expression of the reporter gene product.

The invention further relates to a method for identifying compounds that regulate heart-related disorders comprising the step of contacting a test compound with a cell or cell lysate containing HGPRBMY2 transcripts; and detecting the translation of the HGPRBMY2 transcript.

The invention further relates to a method for modulating heart-related disorders in a subject, comprising administering to the subject a therapeutically effective amount of a HGPRBMY2 polypeptide.

The invention further relates to a method for modulating heart-related disorders in a subject, comprising administering to the subject a therapeutically effective amount of a HGPRBMY2 polypeptide wherein the HGPRBMY2 polypeptide is HGPRBMY2 or a functionally equivalent derivative thereof, preferably wherein the subject is a human.

The invention further relates to a method for modulating heart-related disorders in a subject, comprising administering to the subject a therapeutically effective amount of a HGPRBMY2 polypeptide wherein the HGPRBMY2 polypeptide is HGPRBMY2 or a functionally equivalent derivative thereof, preferably wherein the subject is a human, wherein the HGPRBMY2 polypeptide is contained in a pharmaceutical composition.

The invention further relates to a method for the treatment of heart-related disorders, comprising modulating the activity of a HGPRBMY2 polypeptide.

The invention further relates to a method for the treatment of heart-related disorders, comprising modulating the activity of a HGPRBMY2 polypeptide, wherein the HGPRBMY2 polypeptide is HGPRBMY2 or a functionally equivalent derivative thereof.

The invention further relates to a method for the treatment of heart-related disorders, comprising modulating the activity of a HGPRBMY2 polypeptide, wherein the HGPRBMY2 polypeptide is HGPRBMY2 or a functionally equivalent derivative thereof, wherein the method comprises administering an effective amount of a compound that agonizes or antagonizes the activity of the HGPRBMY2 polypeptide.

The invention further relates to a method for the treatment of heart-related disorders, comprising administering an effective amount of a compound that decreases expression of a HGPRBMY2 gene.

The invention further relates to a method for the treatment of heart-related disorders, comprising administering an effective amount of a compound that decreases expression of a HGPRBMY2 gene, wherein the compound is an oligonucleotide encoding an antisense or ribozyme molecule that targets HGPRBMY2 transcripts and inhibits translation.

The invention further relates to a method for the treatment of heart-related disorders, comprising administering an effective amount of a compound that decreases expression of a HGPRBMY2 gene, wherein the compound is an oligonucleotide that forms a triple helix with the promoter of the HGPRBMY2 gene and inhibits transcription.

The invention further relates to a method for the treatment of heart-related disorders, comprising administering an effective amount of a compound that increases expression of a HGPRBMY2 gene.

The invention further relates to a pharmaceutical formulation for the treatment of heart-related disorders, comprising a compound that activates or inhibits HGPRBMY2 activity, mixed with a pharmaceutically acceptable carrier.

The invention further relates to a method for identifying compounds that modulate the activity of a G-protein coupled receptor comprising the step of (a)contacting a test compound to a cell that expresses a HGPRBMY2 gene and the G-protein coupled receptor, and measuring activity; (b) contacting a test compound to a cell that expresses a HGPRBMY2 gene but does not express the G-protein coupled receptor, and measuring activity; and (c) comparing activity obtained in (b) with the activity obtained in (a); such that if the level obtained in (b) differs from that obtained in (b), a compound that modulates G-protein coupled receptor activity is identified.

3.1 Definitions

The term “derivative” as used herein refers to a polypeptide that comprises an amino acid sequence of a GPCR polypeptide or peptide as described herein that has been altered by the introduction of amino acid residue substitutions, deletions or additions. The term “derivative” as used herein also refers to a GPCR polypeptide or peptide that has been modified, i.e., by the covalent attachment of any type of molecule to the polypeptide. For example, but not by way of limitation, a GPCR polypeptide or peptide may be modified, e.g., by glycosylation, acetylation, pegylation, phosphorylation, amidation, derivatization by known protecting/blocking groups, proteolytic cleavage, linkage to a cellular ligand or other polypeptide, etc. A derivative of a GPCR polypeptide or peptide may be modified by chemical modifications using techniques known to those of skill in the art, including, but not limited to specific chemical cleavage, acetylation, formylation, metabolic synthesis of tunicamycin, etc. Further, a derivative of a GPCR polypeptide or peptide may contain one or more non-classical amino acids. A polypeptide derivative possesses a similar or identical function as a GPCR polypeptide or peptide described herein.

An “isolated” or “purified” polypeptide or polypeptide complex of the invention is substantially free of cellular material or other contaminating polypeptides from the cell or tissue source from which the polypeptide is derived, or substantially free of chemical precursors or other chemicals when chemically synthesized. The language “substantially free of cellular material” includes preparations of a polypeptide or polypeptide complex in which the polypeptide or polypeptide complex is separated from cellular components of the cells from which it is isolated or recombinantly produced. Thus, a polypeptide or polypeptide complex that is substantially free of cellular material includes preparations of polypeptide or polypeptide complex having less than about 30%, 20%, 10%, or 5% (by dry weight) of a heterologous polypeptide (also referred to herein as a “contaminating polypeptide”). When the polypeptide or polypeptide complex is recombinantly produced, it is also preferably substantially free of culture medium, ie., culture medium represents less than about 20%, 10%, or 5% of the volume of the polypeptide preparation. When the polypeptide or polypeptide complex is produced by chemical synthesis, it is preferably substantially free of chemical precursors or other chemicals, i.e., it is separated from chemical precursors or other chemicals which are involved in the synthesis of the polypeptide. Accordingly such preparations of the polypeptide or polypeptide complex have less than about 30%, 20%, 10%, 5% (by dry weight) of chemical precursors or compounds other than the polypeptide or polypeptide complex of interest. In a preferred embodiment, polypeptides or polypeptide complexes or peptides of the invention are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

The term “fusion polypeptide” as used herein refers to a polypeptide that comprises an amino acid sequence of a polypeptide or peptide and an amino acid sequence of another polypeptide or peptide (e.g., GPCR fused to an epitope tag such as a hexa-histidine motif, or a GPCR domain fused to another GPCR domain, such as two or more extracellular domains in tandem).

The term “GPCR antigen” refers to a GPCR polypeptide or peptide to which an antibody or antibody fragment immunospecifically binds. A GPCR antigen also refers to an analog or derivative of a GPCR polypeptide or peptide to which an antibody or antibody fragment immunospecifically binds.

The term “antibodies or antibody fragments that immunospecifically bind to a GPCR antigen” as used herein refers to antibodies, Fab's of antibodies, or other binding portions of antibodies, that specifically bind to a either a native and/or denatured GPCR polypeptide or a GPCR peptide and do not non-specifically bind to other polypeptides. Antibodies, or Fab portions thereof, that immunospecifically bind to a GPCR polypeptide or peptide may have cross-reactivity with other antigens. Preferably, antibodies or fragments that immunospecifically bind to a GPCR polypeptide or peptide do not cross-react with other antigens. Antibodies or fragments that immunospecifically bind to a GPCR polypeptide can be identified, for example, by immunoassays or other techniques known to those of skill in the art.

The term “patient in need thereof” refers to a human with, or at risk of, a disease or disorder associated with the gene or gene product of the invention. Further this term includes in certain embodiments immunocompromised patients. For research purposes, an animal model, for example a mouse model or monkey model, can be utilized to simulate such a patient in some circumstances.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleic acids at corresponding amino acid positions or nucleic acid positions are then compared. When a position in the first sequence is occupied by the same amino acid residue or nucleic acid as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleic acid searches can be performed with the NBLAST nucleic acid program parameters set, e.g., for score=100, wordlength=12 to obtain nucleic acid sequences homologous to a nucleic acid molecules of the present invention. BLAST polypeptide searches can be performed with the XBLAST program parameters set, e.g., to score−50, wordlength=3 to obtain amino acid sequences homologous to a polypeptide molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (e.g., http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### 4. DESCRIPTION OF THE FIGURES

The file of this patent contains at least one Figure executed in color. Copies of this patent with color Figure(s) will be provided by the Patent and Trademark Office upon request and payment of the necessary fee.

FIG. 1: Nucleic acid sequence of the coding region of HGPRBMY1. The 5′ untranslated region is the first group of sequences, the second group of sequences is the open reading frame of HGPRBMY1 and the third set is the 3′ untranslated region.

FIG. 2: Theoretical translation of the open reading frame of the cDNA of FIG. 1, resulting in the polypeptide sequence of HGPRBMY1.

FIG. 3: The shaded sequences in the polypeptide sequence in the upper half of the figure reflect the transmembrane regions. The bottom of the figure depicts a hydropathy plot of the polypeptide sequence of FIG. 2.

FIG. 4: Sequence alignment of HGPRBMY1 and related G-protein coupled receptors. The GCG pileup program was used to generate the alignment. The blackened areas represent identical amino acids in more than half of the listed sequences and the grey highlighted areas represent similar amino acids.

FIG. 5: Expression profile of HGPRBMY1 in various tissues as measured by PCR. The PCR data was converted into a relative assessment of the difference in transcript abundance amongst the tissues tested. Transcripts corresponding to the orphan GPCR, HGPRBMY1, are expressed most highly in bone marrow, spleen and thymus.

FIG. 6: Nucleic acid sequence of the coding region of HGPRBMY2. The 5′ untranslated region is the first group of sequences, the second group of sequences is the open reading frame of HGPRBMY2 and the third set is the 3′ untranslated region.

FIG. 7: Theoretical translation of the open reading frame of the cDNA of FIG. 6, resulting in the polypeptide sequence of HGPRBMY2.

FIG. 8: The shaded sequences in the polypeptide sequence in the upper half of the figure reflect the transmembrane regions. The bottom of the figure depicts a Hydropathy plot of the polypeptide sequence of FIG. 7.

FIG. 9: Sequence alignment of HGPRBMY2 and related G-protein coupled receptors. The GCG pileup program was used to generate the alignment. The blackened areas represent identical amino acids in more than half of the listed sequences and the grey highlighted areas represent similar amino acids.

FIG. 10: Expression profile of HGPRBMY2 in various tissues as measured by PCR. The PCR data was converted into a relative assessment of the difference in transcript abundance amongst the tissues tested. Transcripts corresponding to the orphan GPCR, HGPRBMY2, are expressed most highly in testis, heart, and thymus.

FIG. 11: Untransfected Cho NFAT-CRE cell line FACS profile. Control Cho-NFAT/CRE (Nuclear Factor Activator of Transcription (NFAT)/cAMP response element (CRE)) cell lines were incubated with 10 nM PMA and 1 uM Thapsigargin/10 uM Forskolin, respectively, in the absence of the pcDNA3.1 Hygro™/HGPRBMY2 mammalian expression vector transfection, as described herein. The stimulated cells were sorted via FACS (Fluorescent Assisted Cell Sorter) according to their wavelength emission at 518 nM (Channel R3—Green Cells), and 447 nM (Channel R2—Blue Cells). As shown, the vast majority of cells emit at 518 nM, with minimal emission observed at 447 nM. The latter is expected since the NFAT/CRE response elements remain dormant in the absence of an activated G-protein dependent signal transduction pathway (e.g., pathways mediated by Gq/11 or Gs coupled receptors). As a result, the cell permeant, CCF2/AM™ (Aurora Biosciences; Zlokarnik, et al., 1998) substrate remains intact and emits light at 518 nM.

FIG. 12: Overexpression Of BMY2 Constitutively Couples Through The NFAT/CRE Response Element. Cho-NFAT/CRE cell lines transfected with the pcDNA3.1 Hygro™/HGPRBMY2 mammalian expression vector were incubated with 10 nM PMA and 1 uM Thapsigargin/10 uM Forskolin, respectively, as described herein. The stimulated cells were sorted via FACS according to their wavelength emission at 518 nM (Channel R3—Green Cells), and 447 nM (Channel R2—Blue Cells). As shown, overexpression of HGPRBMY2 results in functional coupling and subsequent activation of beta lactamase gene expression, as evidenced by the significant number of cells with fluorescent emission at 447 nM relative to the non-transfected control Cho-NFAT/CRE cells (shown in FIG. 11).

FIG. 13: HGPRBMY2 Does Not Couple Through The cAMP Response Element. HEK-CRE cell lines transfected with the pcDNA3.1 Hygro™/HGPRBMY2 mammalian expression vector were incubated with 10 nM PMA and 10 uM Forskolin, as described herein. The stimulated cells were sorted via FACS according to their wavelength emission at 518 nM (Channel R3—Green Cells), and 447 nM (Channel R2—Blue Cells). As shown, overexpression of HGPRBMY2 in te HEK-CRE cells did not result in functional coupling, as evidenced by the insignificant background level of cells with fluorescent emission at 447 nM.

FIG. 14: Expressed HGPRBMY2 Localizes To The Plasma Membrane. Cho-NFAT/CRE cell lines transfected with the pcDNA3.1 Hygro™/HGPRBMY2-FLAG mammalian expression vector were subjected to immunocytochemistry using an FITC conjugated Anti Flag monoclonal antibody, as described herein. Panel A shows the transfected Cho-NFAT/CRE cells under visual wavelengths, and panel B shows the fluorescent emission of the same cells at 530 nm after illumination with a laser at 447 nm. The plasma membrane localization is clearly evident in panel B, and is consistent with the HGPRBMY2 polypeptide representing a member of the GPCR family.

FIG. 15: Transfected Cho-NFAT/CRE cell lines With Intermediate and High Beta Lactamase Expression Levels Useful In Screens to Identify HGPRBMY2 Agonists and/or Antagonists. Several Cho-NFAT/CRE cell lines transfected with the pcDNA3.1 Hygro™/HGPRBMY2 mammalian expression vector were isolated via FACS that had either intermediate or high beta lactamase expression levels post stimulation with 10 nM PMA and 1 uM Thapsigargin/10 uM Forskolin, as described herein. Panel A shows HGPRBMY2 transfected Cho-NFAT/CRE cells prior to stimulation with 10 nM PMA and 1 uM Thapsigargin/10 uM Forskolin (−P/T/F). Panel B shows HGPRBMY2 transfected Cho-NFAT/CRE cells after stimulation with 10 nM PMA and 1 uM Thapsigargin/10 uM Forskolin (+P/T/F). Panel C shows HGPRBMY2 transfected Cho-NFAT/CRE cells after stimulation with 10 nM PMA and 1 uM Thapsigargin/10 uM Forskolin (+P/T/F) that have an intermediate level of beta lactamase expression. Panel D shows HGPRBMY2 transfected Cho-NFAT/CRE cells after stimulation with 10 nM PMA and 1 uM Thapsigargin/10 uM Forskolin (+P/T/F) that have a high level of beta lactamase expression.

FIG. 16: Expanded Expression Profile Of The Novel Human G-Protein Coupled Receptor, HGPRBMY2. The figure illustrates the relative expression level of HGPRBMY2 amongst various mRNA tissue sources. As shown, the HGPRBMY2 polypeptide was predominately expressed in the heart, with highest expression in the left ventricle, significantly in tissues of the posterior hypothalamus (1000-fold greater than most other tissues), the DRG, and to a lesser extent in tissues throughout the brain in addition to other tissues as shown. Expression data was obtained by measuring the steady state HGPRBMY2 mRNA levels by quantitative PCR using the PCR primer pair provided as SEQ ID NO:25 and 26, and Taqman probe (SEQ ID NO:27) as described herein.

### 5. DETAILED DESCRIPTION OF THE INVENTION

HGPRBMY1 is a novel receptor expressed in bone marrow, spleen and thymus. The present invention use of HGPRBMY1 nucleic acids, HGPRBMY1 polypeptides and peptides, as well as antibodies to the HGPRBMY1 (which can, for example, act as HGPRBMY1 agonists or antagonists), antagonists that inhibit receptor activity or expression, or agonists that activate receptor activity or increase its expression in the diagnosis and treatment of immune disorders, including, but not limited to immune disorders in animals, including humans. The diagnosis of abnormality associated with HGPRBMY1 in a patient, or an abnormality in the HGPRBMY1 signal transduction pathway, will assist in devising a proper treatment or therapeutic regimen. In addition, HGPRBMY1 nucleic acids and HGPRBMY1 polypeptides are useful for the identification of compounds effective in the treatment of immune disorders regulated by HGPRBMY1.

The invention features HGPRBMY1 polypeptides or portions of the full length polypeptide, i.e., peptides, which can be designed to correspond to functional domains of the HGPRBMY1 (e.g., full length polypeptide, ECD, TM or CD), or mutated, truncated or deleted HGPRBMY1 (e.g. an HGPRBMY1 with one or more functional domains or portions thereof deleted, such as ΔTM and/or ΔCD), or HGPRBMY1 fusion polypeptides (e.g. an HGPRBMY1 or a functional domain of HGPRBMY1, such as an ECD fused to an unrelated polypeptide or peptide such as an immunoglobulin constant region, i.e., IgFc), nucleic acid sequences encoding such products, and host cell expression systems that can produce such HGPRBMY1 products.

The invention also features antibodies and anti-idiotypic antibodies (including antibody fragments), antagonists and agonists of the HGPRBMY1, as well as compounds or nucleic acid constructs that inhibit expression of the HGPRBMY1 gene (transcription factor inhibitors, antisense and ribozyme molecules, or gene or regulatory sequence replacement constructs), or promote expression of HGPRBMY1 (e.g., expression constructs in which HGPRBMY1 coding sequences are operatively associated with expression control elements such as promoters, promoter/enhancers, etc.).

The invention also features host cells or animals genetically engineered to express exogenous HGPRBMY1 (or mutants thereof), cells or animals engineered to increase expression of the endogenous HGPRBMY1, cells or animals engineered to express a mutated HGPRBMY1, or cells or animals engineered to inhibit expression of either an animal's endogenous HGPRBMY1.

The HGPRBMY1 polypeptides, HGPRBMY1 fusion polypeptides, HGPRBMY1 nucleic acid sequences, antibodies, antagonists and agonists can be useful for the detection of mutant HGPRBMY1 or inappropriately expressed HGPRBMY1, particularly for the diagnosis of immune disorders either related to HGPRBMY1 expression, activation or down regulation, or wherein HGPRBMY1 serves as an indicator of an immune disorder. The HGPRBMY1 polypeptides, HGPRBMY1 fusion polypeptides, HGPRBMY1 nucleic acid sequences, host cell expression systems, antibodies, antagonists, agonists and genetically engineered cells and animals can be used for screening for drugs effective in the treatment of such immune disorders. The use of engineered host cells and/or animals may offer an advantage in that such systems allow not only for the identification of compounds that bind to the ECD or to the CD of the HGPRBMY1, and/or can be used to identify compounds that modulate the signal transduced by the activated HGPRBMY1.

Finally, the HGPRBMY1 polypeptide products (especially derivatives such as peptides corresponding to a HGPRBMY1 ECD, or truncated polypeptides lacking a hydrophobic TM domain, which are soluble under normal physiological conditions) and fusion polypeptide products (especially HGPRBMY1-Ig fusion polypeptides, i.e., fusions of a domain of HGPRBMY1, e.g., ECD, ΔTM or CD to a heterologous sequence such as IgFc), antibodies (including fragments thereof), antagonists or agonists (including compounds that modulate signal transduction which may act on downstream targets in the HGPRBMY1 signal transduction pathway) can be used for therapy of such diseases. For example, the administration of an effective amount of a pharmaceutical composition comprising a soluble ECD, CD, ΔTM, CD-IgFc fusion, ECD-IgFc fusion polypeptide or an antibody (or fragment thereof) that mimics the HGPRBMY1 ECD would modulate HGPRBMY1 activity, leading to prevention or treatment of an immune disorder.

Nucleic acid constructs encoding the HGPRBMY1 products above can be used to engineer host cells to express such HGPRBMY1 products in vivo. These implanted cells, when implanted into a host, deliver a continuous supply of a soluble ECD or a fusion polypeptide that modulates HGPRBMY1 activity. Nucleic acid constructs encoding functional HGPRBMY1, mutant HGPRBMY1, as well as antisense and ribozyme molecules can be used in gene therapy for the modulation of HGPRBMY1 expression and/or activity in the treatment of immune disorders. Thus, the invention features pharmaceutical formulations and methods for treating immune disorders.

The strong homology to human G-protein coupled receptors, combined with the predominate localized expression in bone marrow and spleen, in conjunction with the p27 and IkB association, suggests the HGPRBMY1 polynucleotides and polypeptides may be useful in treating, diagnosing, prognosing, and/or preventing immune diseases and/or disorders. Representative uses are described elsewhere herein. Briefly, the strong expression in immune tissue indicates a role in regulating the proliferation; survival; differentiation; and/or activation of hematopoietic cell lineages, including blood stem cells.

The HGPRBMY1 polypeptide may also be useful as a preventative agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, and scieroderma. The HGPRBMY1 polypeptide may be useful for modulating cytokine production, antigen presentation, or other processes, such as for boosting immune responses, etc.

Moreover, the protein may represent a factor that influences the differentiation or behavior of other blood cells, or that recruits hematopoietic cells to sites of injury. Thus, this gene product is thought to be useful in the expansion of stem cells and committed progenitors of various blood lineages, and in the differentiation and/or proliferation of various cell types. Furthermore, the protein may also be used to determine biological activity, raise antibodies, as tissuemarkers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Various aspects of the invention are described in greater detail in the subsections below.

HGPRBMY2, described for the first time herein, is a novel receptor protein expressed in heart, brain tissues, testis, and thymus tissues. The invention encompasses the use of HGPRBMY2 nucleic acids, HGPRBMY2 proteins and peptides, as well as antibodies to the HGPRBMY2 (which can, for example, act as HGPRBMY2 agonists or antagonists), antagonists that inhibit receptor activity or expression, or agonists that activate receptor activity or increase its expression in the diagnosis and treatment of cardiovascular disorders, including, but not limited to heart disease in animals, including humans. The diagnosis of an HGPRBMY2 abnormality in a patient, or an abnormality in the HGPRBMY2 signal transduction pathway, will assist in devising a proper treatment or therapeutic regimen. In addition, HGPRBMY2 nucleic acids and HGPRBMY2 proteins are useful for the identification of compounds effective in the treatment of cardiovascular disorders regulated by the HGPRBMY2.

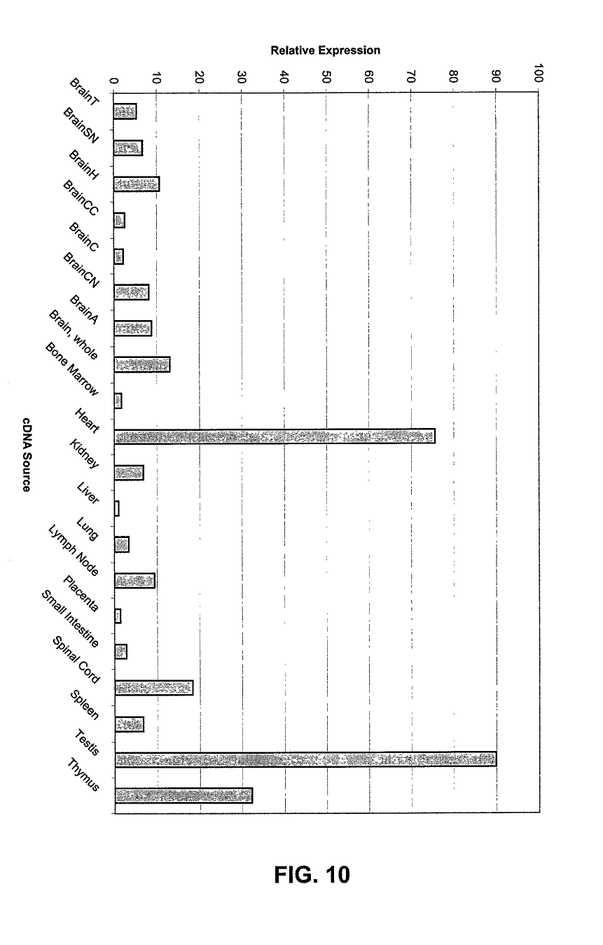

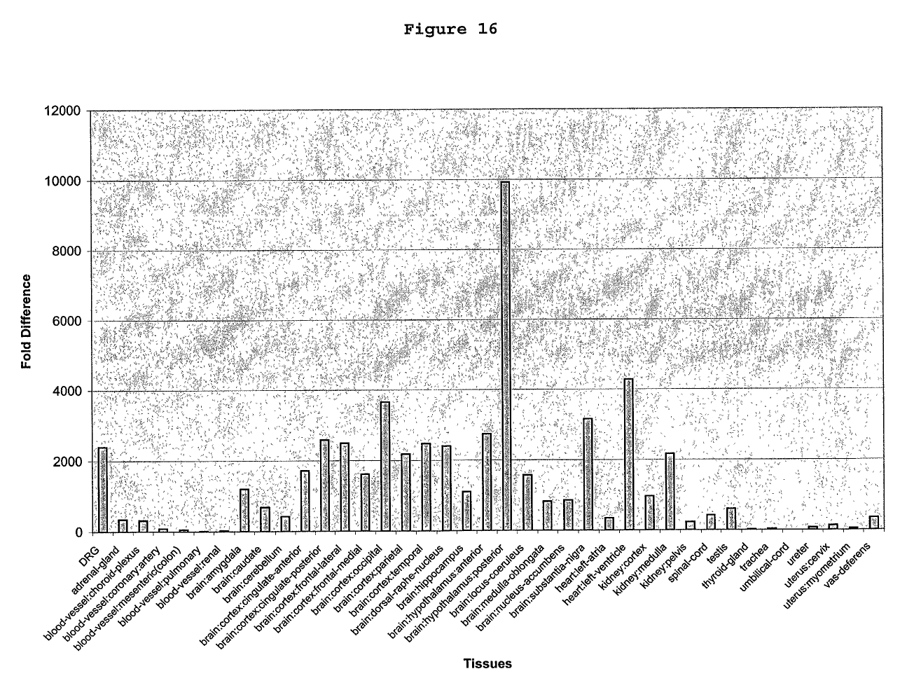

Expanded analysis of HGPRBMY2 expression levels by TaqMan™ quantitative PCR (see FIG. 16) confirmed that the HGPRBMY2 polypeptide is expressed at very low levels in heart and testis, with relatively low-level expression in the brain sub regions tested as shown using the SYBR green experiments (see FIG. 10). HGPRBMY2 mRNA was expression predominately in heart, with the highest concentration in the left ventricle, and the posterior hypothalamus; significantly in the DRG and other tissues throughout the brain, and to a lesser extent in the spinal cord in addition to other tissues as shown. These data suggest that HGPRBMY2 may be useful for the treatment and/or amelioration of metabolic disorders, mainly obesity, and for the treatment of pain disorders.

The strong homology to human G-protein coupled receptors, combined with the predominate localized expression in heart tissue suggests the HGPRBMY2 polynucleotides and polypeptides may be useful in treating, diagnosing, prognosing, and/or preventing cardiovascular diseases and/or disorders, which include, but are not limited to: myocardio infarction, congestive heart failure, arrthymias, cardiomyopathy, atherosclerosis, arterialsclerosis, microvascular disease, embolism, thromobosis, pulmonary edema, palpitation, dyspnea, angina, hypotension, syncope, heart murmer, aberrant ECG, hypertrophic cardiomyopathy, the Marfan syndrome, sudden death, prolonged QT syndrome, congenital defects, cardiac viral infections, valvular heart disease, and hypertension.

Similarly, HGPRBMY2 polynucleotides and polypeptides may be useful for ameliorating cardiovascular diseases and symptoms which result indirectly from various non-cardiavascular effects, which include, but are not limited to, the following, obesity, smoking, Down syndrome (associated with endocardial cushion defect); bony abnormalities of the upper extremities (associated with atrial septal defect in the Holt-Oram syndrome); muscular dystrophies (associated with cardiomyopathy); hemochromatosis and glycogen storage disease (associated with myocardial infiltration and restrictive cardiomyopathy); congenital deafness (associated with prolonged QT interval and serious cardiac arrhythrnias); Raynaud's disease (associated with primary pulmonary hypertension and coronary vasospasm); connective tissue disorders, i.e., the Marfan syndrome, Ehlers-Danlos and Hurler syndromes, and related disorders of mucopolysaccharide metabolism (aortic dilatation, prolapsed mitral valve, a variety of arterial abnormalities); acromegaly (hypertension, accelerated coronary atherosclerosis, conduction defects, cardiomyopathy); hyperthyroidism (heart failure, atrial fibrillation); hypothyroidism (pericardial effusion, coronary artery disease); rheumatoid arthritis (pericarditis, aortic valve disease); scleroderma (cor pulmonale, myocardial fibrosis, pericarditis); systemic lupus erythematosus (valvulitis, myocarditis, pericarditis); sarcoidosis (arrhythmias, cardiomyopathy); postmenopausal effects, Chlamydial infections, polycystic ovary disease, thyroid disease, alcoholism, diet, and exfoliative dermatitis (high-output heart failure), for example.

Moreover, polynucleotides and polypeptides, including fragments and/or antagonists thereof, have uses which include, directly or indirectly, treating, preventing, diagnosing, and/or prognosing the following, non-limiting, cardiovascular infections: blood stream invasion, bacteremia, sepsis, Streptococcus pneumoniae infection, group a streptococci infection, group b streptococci infection, Enterococcus infection, nonenterococcal group D streptococci infection, nonenterococcal group C streptococci infection, nonenterococcal group G streptococci infection, Streptoccus viridans infection, Staphylococcus aureus infection, coagulase-negative staphylococci infection, gram-negative Bacilli infection, Enterobacteriaceae infection, Psudomonas spp. Infection, Acinobacter spp. Infection, Flavobacterium meningosepticum infection, Aeromonas spp. Infection, Stenotrophomonas maltophilia infection, gram-negative coccobacilli infection, Haemophilus influenza infection, Branhamella catarrhalis infection, anaerobe infection, Bacteriodes fragilis infection, Clostridium infection, fungal infection, Candida spp. Infection, non-albicans Candida spp. Infection, Hansenula anomala infection, Malassezia furfur infection, nontuberculous Mycobacteria infection, Mycobacterium avium infection, Mycobacterium chelonae infection, Mycobacterium fortuitum infection, spirochetal infection, Borrelia burgdorferi infection, in addition to any other cardiovascular disease and/or disorder (e.g., non-sepsis) implicated by the causative agents listed above or elsewhere herein.

The strong homology to human G-protein coupled receptor proteins, combined with the localized expression in various brain tissues suggests HGPRBMY2 polynucleotides and polypeptides may be useful in treating, diagnosing, prognosing, and/or preventing neurodegenerative disease states, behavioral disorders, or inflammatory conditions. Representative uses are described in the section 5.6c below, in the Examples, and elsewhere herein. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates it plays a role in normal neural function. Potentially, this gene product is involved in synapse formation, neurotransmission, learning, cognition, homeostasis, or neuronal differentiation or survival. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Alternatively, the strong homology to G-protein coupled receptors, combined with the predominate localized expression in testis tissue suggests the potential utility for HGPRBMY2 polynucleotides and polypeptides in treating, diagnosing, prognosing, and/or preventing testicular, in addition to reproductive disorders.

In preferred embodiments, HGPRBMY2 polynucleotides and polypeptides including agonists and fragments thereof, have uses which include treating, diagnosing, prognosing, and/or preventing the following, non-limiting, diseases or disorders of the testis: spermatogenesis, infertility, Klinefelter's syndrome, XX male, epididymitis, genital warts, germinal cell aplasia, cryptorchidism, varicocele, immotile cilia syndrome, and viral orchitis. The HGPRBMY2 polynucleotides and polypeptides including agonists and fragments thereof, may also have uses related to modulating testicular development, embryogenesis, reproduction, and in ameliorating, treating, and/or preventing testicular proliferative disorders (e.g., cancers, which include, for example, choriocarcinoma, Nonseminoma, seminona, and testicular germ cell tumors).

Likewise, the predominate localized expression in testis tissue also emphasizes the potential utility for HGPRBMY2 polynucleotides and polypeptides in treating, diagnosing, prognosing, and/or preventing metabolic diseases and disorders which include the following, not limiting examples: premature puberty, incomplete puberty, Kallman syndrome, Cushing's syndrome, hyperprolactinemia, hemochromatosis, congenital adrenal hyperplasia, FSH deficiency, and granulomatous disease, for example.

This gene product may also be useful in assays designed to identify binding agents, as such agents (antagonists) are useful as male contraceptive agents. The testes are also a site of active gene expression of transcripts that is expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications.

The strong homology to G-protein coupled receptors, combined with the localized expression in thymus tissue suggests the HGPRBMY2 polynucleotides and polypeptides may be useful in treating, diagnosing, prognosing, and/or preventing immune diseases and/or disorders. The strong expression in immune tissue indicates a role in regulating the proliferation; survival; differentiation; and/or activation of hematopoietic cell lineages, including blood stem cells.

The HGPRBMY2 polypeptide may also be useful as a preventative agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, and scleroderma. The HGPRBMY2 polypeptide may be useful for modulating cytokine production, antigen presentation, or other processes, such as for boosting immune responses, etc.

Moreover, the protein may represent a factor that influences the differentiation or behavior of other blood cells, or that recruits hematopoietic cells to sites of injury. Thus, this gene product is thought to be useful in the expansion of stem cells and committed progenitors of various blood lineages, and in the differentiation and/or proliferation of various cell types. Furthermore, the protein may also be used to determine biological activity, raise antibodies, as tissuemarkers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

The invention features HGPRBMY2 polypeptides or portions of the full length polypeptide, i.e., peptides, which can be designed to correspond to functional domains of the HGPRBMY2 (e.g., full length protein, ECD, TM or CD), or mutated, truncated or deleted HGPRBMY2 (e.g. an HGPRBMY2 with one or more functional domains or portions thereof deleted, such as ΔTM and/or ΔCD), or HGPRBMY2 fusion polypeptides (e.g. an HGPRBMY2 or a functional domain of HGPRBMY2, such as an ECD fused to an unrelated polypeptide or peptide such as an immunoglobulin constant region, i.e., IgFc), nucleic acid sequences encoding such products, and host cell expression systems that can produce such HGPRBMY2 products.

The invention also features antibodies and anti-idiotypic antibodies (including Fab fragments), antagonists and agonists of the HGPRBMY2, as well as compounds or nucleic acid constructs that inhibit expression of the HGPRBMY2 gene (transcription factor inhibitors, antisense and ribozyme molecules, or gene or regulatory sequence replacement constructs), or promote expression of HGPRBMY2 (e.g., expression constructs in which HGPRBMY2 coding sequences are operatively associated with expression control elements such as promoters, promoter/enhancers, etc.). The invention also relates to host cells and animals genetically engineered to express the human HGPRBMY2 (or mutants thereof) or to inhibit or “knock-out” expression of the animal's endogenous HGPRBMY2.

The HGPRBMY2 polypeptides or peptides, HGPRBMY2 fusion polypeptides, HGPRBMY2 nucleic acid sequences, antibodies, antagonists and agonists can be useful for the detection of mutant HGPRBMY2 or inappropriately expressed HGPRBMY2 for the diagnosis of cardiovascular disorders. The HGPRBMY2 polypeptides or peptides, HGPRBMY2 fusion polypeptides, HGPRBMY2 nucleic acid sequences, host cell expression systems, antibodies, antagonists, agonists and genetically engineered cells and animals can be used for screening for drugs effective in the treatment of such cardiovascular disorders. The use of engineered host cells and/or animals may offer an advantage in that such systems allow not only for the identification of compounds that bind to the ECD of the HGPRBMY2, but can also identify compounds that affect the signal transduced by the activated HGPRBMY2.

Finally, the HGPRBMY2 protein products (especially soluble derivatives such as peptides corresponding to a HGPRBMY2 ECD, or truncated polypeptides lacking a hydrophobic TM domain) and fusion polypeptide products (especially HGPRBMY2-Ig fusion polypeptides, i.e., fusions of the HGPRBMY2 or a domain of the HGPRBMY2, e.g., ECD, ΔTM, or CD to a heterologous sequence such as IgFc), antibodies and anti-idiotypic antibodies (including Fab fragments), antagonists or agonists (including compounds that modulate signal transduction which may act on downstream targets in the HGPRBMY2 signal transduction pathway) can be used for therapy of such diseases. For example, the administration of an effective amount of a pharmaceutical composition comprising a soluble HGPRBMY2 ECD, ΔTM HGPRBMY2 or an ECD-IgFc fusion polypeptide or an anti-idiotypic antibody (or its Fab) that mimics the HGPRBMY2 ECD would modulate activation of the GPCR by endogenous agonist or antagonist, and prevent or reduce binding and receptor activation, leading to heart failure.

Nucleic acid constructs encoding such HGPRBMY2 products can be used to genetically engineer host cells to express such HGPRBMY2 products in vivo; these genetically engineered cells function in the body delivering a continuous supply of the HGPRBMY2, HGPRBMY2 peptide, soluble ECD or ΔTM or HGPRBMY2 fusion polypeptide that will modulate agonist or antagonist. Nucleic acid constructs encoding functional HGPRBMY2, mutant HGPRBMY2, as well as antisense and ribozyme molecules can be used in “gene therapy” approaches for the modulation of HGPRBMY2 expression and/or activity in the treatment of cardiovascular disorders. Thus, the invention also encompasses pharmaceutical formulations and methods for treating cardiovascular disorders.

The invention is based, in part, on the surprising discovery of a receptor for agonist or antagonist expressed at significant concentration in heart and thymus. Various aspects of the invention are described in greater detail in the subsections below.

5.1. HGPRBMY1 Nucleic Acids

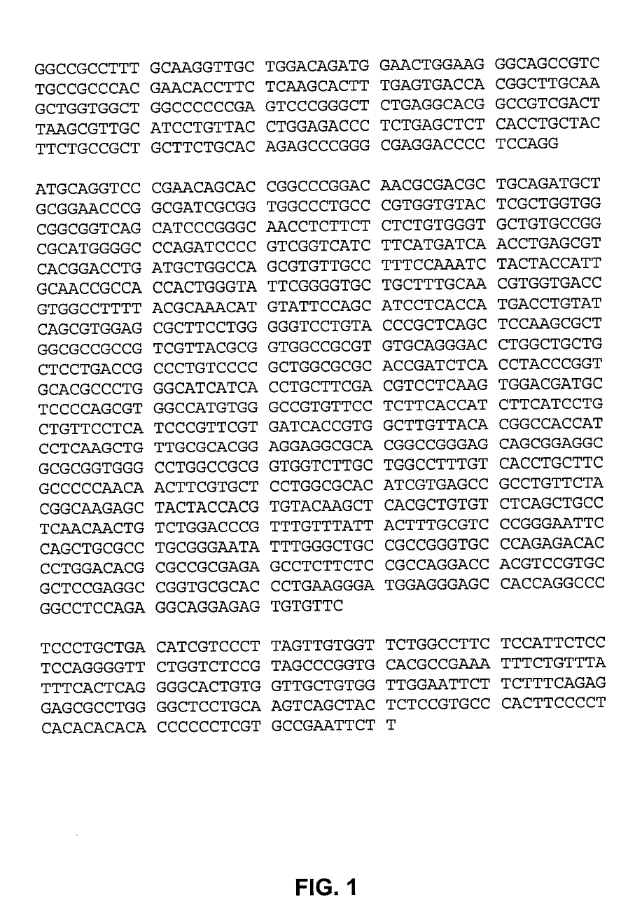

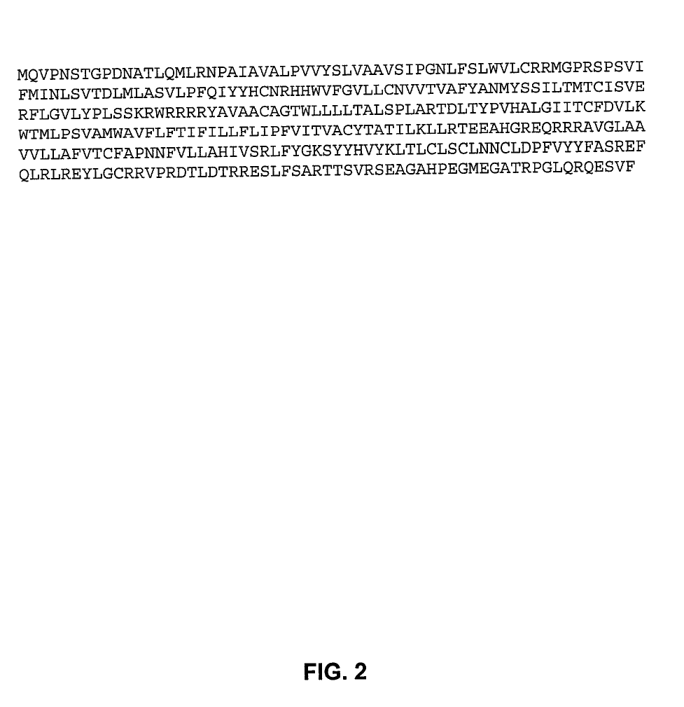

The cDNA sequence of HGPRBMY1 (SEQ ID NO:1) is 1554 base pairs long and is shown in FIG. 1. The first set of sequence is the 5′ untranslated, the second set is the open reading frame and the third set is the 5′ untranslated. The open reading frame extends from nucleotides 247 to 1323 of SEQ ID NO:1. The deduced amino acid sequence encoded by the open reading frame of the cDNA of HGPRBMY1 is 359 amino acids (SEQ ID NO:2) and is shown in FIG. 2.

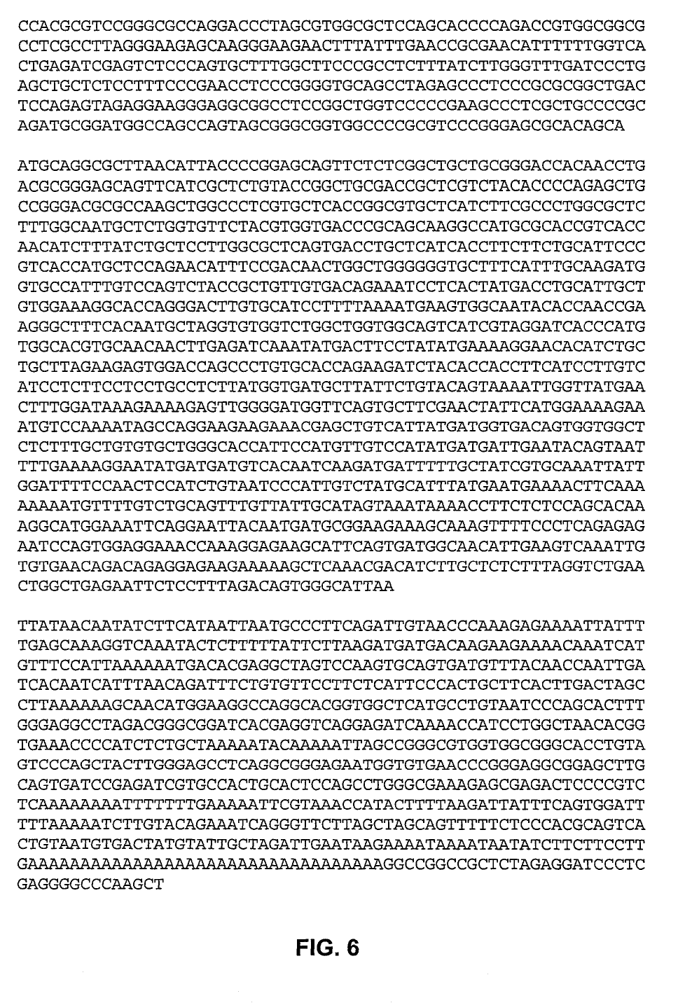

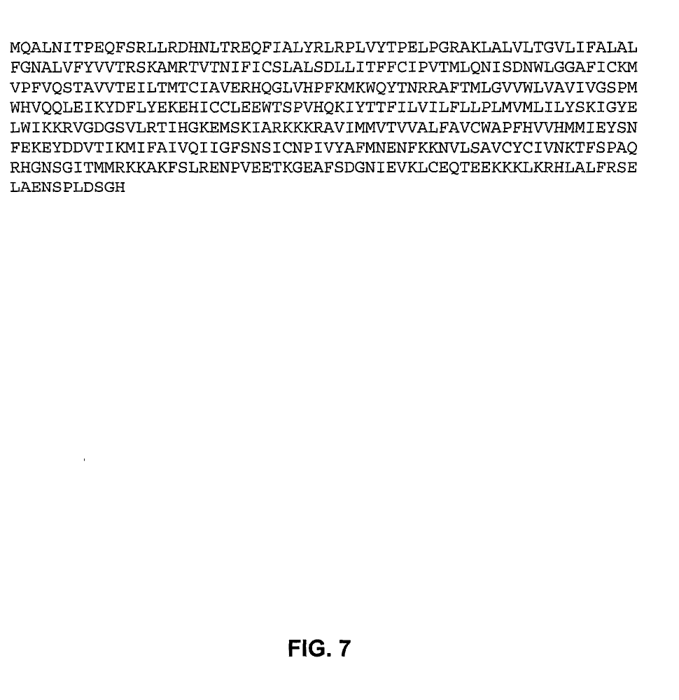

The cDNA sequence of HGPRBMY2 (SEQ ID NO:13) is 2448 base pairs long and is shown in FIG. 6. The first set of sequence is the 5′ untranslated, the second set is the open reading frame and the third set is the 5′ untranslated. The open reading frame extends from nucleotides 359 to 1651 of SEQ ID NO:13. The deduced amino acid sequence encoded by the open reading frame of the cDNA of HGPRBMY2 is 431 amino acids (SEQ ID NO:14) and is shown in FIG. 7.

HGPRBMY1 nucleic acid sequences of the invention include: (a) the DNA sequence shown in SEQ ID NO:1; (b) nucleic acid sequence that encodes the polypeptide shown in SEQ ID NO:2; (c) any nucleic acid sequence that hybridizes to the complement of the DNA sequence shown in SEQ ID NO:1 under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & sons, Inc., New York, at p. 2.10.3) and encodes a functionally equivalent gene product; and (d) any nucleic acid sequence that hybridizes to the complement of the DNA sequences that encode the amino acid sequence shown in SEQ ID NO:2 contained in cDNA clone as deposited with the ATCC® under less stringent conditions, such as moderately stringent conditions, e.g., washing in 0.2×SSC/0.1% SDS at 42° C. (Ausubel et al., 1989, supra), yet which still encodes a functionally equivalent HGPRBMY1 gene product.

HGPRBMY2 nucleic acid sequences of the invention include: (a) the DNA sequence shown in SEQ ID NO:13; (b) nucleic acid sequence that encodes the polypeptide shown in SEQ ID NO:14; (c) any nucleic acid sequence that hybridizes to the complement of the DNA sequence shown in SEQ ID NO:13 under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product; and (d) any nucleic acid sequence that hybridizes to the complement of the DNA sequences that encode the amino acid sequence shown in SEQ ID NO:14 contained in cDNA clone as deposited with the ATCC® under less stringent conditions, such as moderately stringent conditions, e.g., washing in 0.2×SSC/0.1% SDS at 42° C. (Ausubel et al., 1989, supra), yet which still encodes a functionally equivalent HGPRBMY2 gene product.

Functional equivalents of the HGPRBMY1 include naturally occurring HGPRBMY1 present in other species, ie., orthologs, and mutant HGPRBMY1 whether naturally occurring or engineered. The invention also includes degenerate variants of sequences (a) through (d), supra. The invention also includes nucleic acid molecules, preferably DNA molecules, that hybridize to, and are therefore the complements of, the nucleic acid sequences (a) through (d), in the preceding paragraph.

Functional equivalents of the HGPRBMY2 include naturally occurring HGPRBMY2 present in other species, i.e., orthologs, and mutant HGPRBMY2 whether naturally occurring or engineered. The invention also includes degenerate variants of sequences (a) through (d), supra. The invention also includes nucleic acid molecules, preferably DNA molecules, that hybridize to, and are therefore the complements of, the nucleic acid sequences (a) through (d), in the preceding paragraph.

Hybridization conditions may be highly stringent or less highly stringent. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), an 60° C. (for 23-base oligos). These nucleic acid molecules may encode or act as HGPRBMY1 or HGPRBMY2 antisense molecules, useful, for example, in HGPRBMY1 or HGPRBMY2 gene regulation (for and/or as antisense primers in amplification reactions of HGPRBMY1 or HGPRBMY2 gene nucleic acid sequences).

The invention features nucleic acids that are similar to the HGPRBMY1 nucleic acid sequences of the invention. A nucleic acid that has a similar sequence refers to a nucleic acid that satisfies at least one of the following: (a) a nucleic acid having a sequence that is at least 40%, at least 45%, at least 50%, at least 55%, at least 60%, at least 65%, at least 70%, at least 75%, at least 80%, at least 85%, at least 90%, at least 95% or at least 99% identical to the nucleic acid sequence of a GPCR as described herein; (b) a nucleic acid as described herein of at least 100 nucleotides, or at least 125, 150, 200, 250, 300, 400, 500, 600, 700, 800, 900, 1000, 1100, 1250, 1350, 1500, 1650, 1750, 1850, 2000, 2150, 2250 or 2400 contiguous nucleotides in length; and (c) a nucleic acid that is at least 40%, at least 45%, at least 50%, at least 55%, at least 60%, at least 65%, at least 70%, at least 75%, at least 80%, at least 85%, at least 90%, at least 95% or at least 99% identical to the nucleic acid sequence encoding a GPCR polypeptide or peptide as described herein.

The invention features nucleic acids that are similar to the HGPRBMY2 nucleic acid sequences of the invention. A nucleic acid that has a similar sequence refers to a nucleic acid that satisfies at least one of the following: (a) a nucleic acid having a sequence that is at least 40%, at least 45%, at least 50%, at least 55%, at least 60%, at least 65%, at least 70%, at least 75%, at least 80%, at least 85%, at least 90%, at least 95% or at least 99% identical to the nucleic acid sequence of a GPCR as described herein; (b) a nucleic acid as described herein of at least 100 nucleotides, or at least 125, 150, 200, 250, 300, 400, 500, 600, 700, 800, 900, 1000, 1100, 1250, 1350, 1500, 1650, 150, 1850, 2000, 2150, 2250 or 2400 contiguous nucleotides in length; and (c) a nucleic acid that is at least 40%, at least 45%, at least 50%, at least 55%, at least 60%, at least 65%, at least 70%, at least 75%, at least 80%, at least 85%, at least 90%, at least 95% or at least 99% identical to the nucleic acid sequence encoding a GPCR polypeptide or peptide as described herein.

The invention also features allelic variants, i.e., functional equivalents of the HGPRBMY1 or HGPRBMY2 nucleic acid sequence which are naturally occurring and appear in the same genetic locus.

Nucleic acids of HGPRBMY1 or HGPRBMY2 can also be used to identify species orthologs of the sequence, e.g., in monkeys, mice, cats, dogs, cows, fruit flies, zebrafish or other animals. The identification of orthologs of HGPRBMY1 or HGPRBMY2 in other species can be useful for developing animal model systems more closely related to humans for purposes of drug discovery. For example, expression libraries of cDNAs synthesized from bone marrow mRNA derived from the organism of interest can be screened using labeled agonist derived from that species, e.g., an alkaline phosphatase (AP)-agonist fusion polypeptide.

Sequences of the invention may be used as part of ribozyme and/or triple helix sequences, also useful for HGPRBMY1 gene regulation. Still further, such molecules may be used as components of diagnostic methods whereby, for example, the presence of a particular HGPRBMY1 allele responsible for causing an immune disorder, such as immunodeficiency, may be detected.

Sequences of the invention may be used as part of ribozyme and/or triple helix sequences, also useful for HGPRBMY2 gene regulation. Still further, such molecules may be used as components of diagnostic methods whereby, for example, the presence of a particular HGPRBMY2 allele responsible for causing a heart disorder, such as heart failure, may be detected.

In addition to the HGPRBMY1 nucleic acid sequences described above, full length HGPRBMY1 cDNA or gene sequences present in the same species and/or homologues of the HGPRBMY1 gene present in other species can be identified and readily isolated, without undue experimentation, by molecular biological techniques well known in the art. The identification of homologues of HGPRBMY1 in related species can be useful for developing animal model systems more closely related to humans for purposes of drug discovery. For example, expression libraries of cDNAs synthesized from spleen or bone marrow mRNA derived from the organism of interest can be screened using labeled agonist derived from that species, e.g., an AP-agonist fusion polypeptide.

In addition to the HGPRBMY2 nucleic acid sequences described above, full length HGPRBMY2 cDNA or gene sequences present in the same species and/or homologues of the HGPRBMY2 gene present in other species can be identified and readily isolated, without undue experimentation, by molecular biological techniques well known in the art. The identification of homologues of HGPRBMY2 in related species can be useful for developing animal model systems more closely related to humans for purposes of drug discovery. For example, expression libraries of cDNAs synthesized from heart mRNA derived from the organism of interest can be screened using labeled agonist derived from that species, e.g., an AP-agonist fusion polypeptide.

Alternatively, such cDNA libraries, or genomic DNA libraries derived from the organism of interest can be screened by hybridization using the nucleic acids described herein as hybridization or amplification probes. Furthermore, genes at other genetic loci within the genome that encode proteins which have extensive homology to one or more domains of the HGPRBMY1 or HGPRBMY2 gene product can also be identified via similar techniques. In the case of cDNA libraries, such screening techniques can identify clones derived from alternatively spliced transcripts in the same or different species.

Screening can be by filter hybridization, using duplicate filters. The labeled probe can contain at least 15-30 base pairs of the HGPRBMY1 or HGPRBMY2 nucleic acid sequence, as shown in FIG. 1 or FIG. 6. The hybridization washing conditions used should be of a lower stringency when the cDNA library is derived from an organism different from the type of organism from which the labeled sequence was derived. With respect to the cloning of a human HGPRBMY1 or HGPRBMY2 homolog, using murine HGPRBMY1 or HGPRBMY2 probes, for example, hybridization can, for example, be performed at 65° C. overnight in Church's buffer (7% SDS, 250 mM NaHPO₄, 2 mM EDTA, 1% BSA). Washes can be done with 2×SSC, 0.1% SDS at 65° C. and then at 0.1×SSC, 0.1% SDS at 65° C.

Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Alternatively, the labeled HGPRBMY1 or HGPRBMY2 nucleic acid probe may be used to screen a genomic library derived from the organism of interest, again, using appropriately stringent conditions. The identification and characterization of human genomic clones is helpful for designing diagnostic tests and clinical protocols for treating cardiovascular disorders in human patients. For example, sequences derived from regions adjacent to the intron/exon boundaries of the human gene can be used to design primers for use in amplification assays to detect mutations within the exons, introns, splice sites (e.g. splice acceptor and/or donor sites), etc., that can be used in diagnostics.

Further, an HGPRBMY1 or HGPRBMY2 gene homologue may be isolated from nucleic acid of the organism of interest by performing PCR using two degenerate oligonucleotide primer pools designed on the basis of amino acid sequences within the HGPRBMY1 or HGPRBMY2 gene product disclosed herein. The template for the reaction may be cDNA obtained by reverse transcription of mRNA prepared from, for example, human or non-human cell lines or tissue, such as bone marrow, known or suspected to express an HGPRBMY1 or HGPRBMY2 gene allele.

The PCR product may be subcloned and sequenced to ensure that the amplified sequences represent the sequences of an HGPRBMY1 or HGPRBMY2 gene. The PCR fragment may then be used to isolate a full length cDNA clone by a variety of methods. For example, the amplified fragment may be labeled and used to screen a cDNA library, such as a bacteriophage cDNA library. Alternatively, the labeled fragment may be used to isolate genomic clones via the screening of a genomic library.

PCR technology may also be utilized to isolate full length cDNA sequences. For example, RNA may be isolated, following standard procedures, from an appropriate cellular or tissue source (i.e., one known, or suspected, to express the HGPRBMY1 gene, such as, for example, spleen or bone marrow). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. (For a review of cloning strategies which may be used, see e.g., Sambrook et al., 1989, supra.).

PCR technology may also be utilized to isolate full length cDNA sequences. For example, RNA may be isolated, following standard procedures, from an appropriate cellular or tissue source (i.e., one known, or suspected, to express the HGPRBMY2 gene, such as, for example, heart tissues). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. (For a review of cloning strategies which may be used, see e.g., Sambrook et al., 1989, supra.)

The HGPRBMY1 gene sequences may additionally be used to isolate mutant HGPRBMY1 gene alleles. Such mutant alleles may be isolated from individuals either known or proposed to have a genotype which contributes to the symptoms of immnue disorders. Mutant alleles and mutant allele products may then be utilized in the therapeutic and diagnostic systems described below. Additionally, such HGPRBMY1 gene sequences can be used to detect HGPRBMY1 gene regulatory (e.g., promoter or promotor/enhancer) defects which can affect immune function.

The HGPRBMY2 gene sequences may additionally be used to isolate mutant HGPRBMY2 gene alleles. Such mutant alleles may be isolated from individuals either known or proposed to have a genotype which contributes to the symptoms of cardiovascular disorders. Mutant alleles and mutant allele products may then be utilized in the therapeutic and diagnostic systems described below. Additionally, such HGPRBMY2 gene sequences can be used to detect HGPRBMY2 gene regulatory (e.g., promoter or promotor/enhancer) defects which can affect cardiovascular function.

A cDNA of a mutant HGPRBMY1 or HGPRBMY2 gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant HGPRBMY1 or HGPRBMY2 allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant HGPRBMY1 or HGPRBMY2 allele to that of the normal HGPRBMY1 or HGPRBMY2 allele, the mutation(s) responsible for the loss or alteration of function of the mutant HGPRBMY1 or HGPRBMY2 gene product can be ascertained.

Alternatively, a genomic library can be constructed using DNA obtained from an individual suspected of or known to carry the mutant HGPRBMY1 or HGPRBMY2 allele, or a cDNA library can be constructed using RNA from a tissue known, or suspected, to express the mutant HGPRBMY1 or HGPRBMY2 allele. The normal HGPRBMY1 or HGPRBMY2 gene or any suitable fragment thereof may then be labeled and used as a probe to identify the corresponding mutant HGPRBMY1 or HGPRBMY2 allele in such libraries. Clones containing the mutant HGPRBMY1 or HGPRBMY2 gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant HGPRBMY1 or HGPRBMY2 allele in an individual suspected of or known to carry such a mutant allele. In this manner, gene products made by the putatively mutant tissue may be expressed and screened using standard antibody screening techniques in conjunction with antibodies raised against the normal HGPRBMY1 or HGPRBMY2 gene product, as described, below, in Section 5.3. (For screening techniques, see, for example, Harlow, E. and Lane, eds., 1988, “Antibodies: A Laboratory Manual”, Cold Spring Harbor Press, Cold Spring Harbor.) Additionally, screening can be accomplished by screening with labeled agonist or antagonist fusion polypeptides, such as, for example, AP-GPCR or GPCR-AP fusion polypeptides. In cases where an HGPRBMY1 or HGPRBMY2 mutation results in an expressed gene product with altered function (e.g., as a result of a missense or a frameshift mutation), a polyclonal set of antibodies to HGPRBMY1 or HGPRBMY2 are likely to cross-react with the mutant HGPRBMY1 or HGPRBMY2 gene product. Library clones detected via their reaction with such labeled antibodies can be purified and subjected to sequence analysis according to methods well known to those of skill in the art.

HGPRBMY1 or HGPRBMY2 nucleic acids can also be utilized for chromosomal mapping, or as chromosomal markers, e.g., in radiation hybrid mapping.

The invention also features identifying detecting or diagnosing cells or tissues which express a mRNA or HGPRBMY1 or HGPRBMY2. The invention also features nucleic acid sequences that encode mutant HGPRBMY1 or HGPRBMY2 polypeptides, peptides of the HGPRBMY1 or HGPRBMY2, truncated HGPRBMY1 or HGPRBMY2, and HGPRBMY1 or HGPRBMY2 fusion polypeptides. These include, but are not limited to nucleic acid sequences encoding mutant HGPRBMY1 or HGPRBMY2 described in section 5.2 infra; polypeptides or peptides corresponding to the ECD, TM and/or CD domains of the HGPRBMY1 or HGPRBMY2 or portions of these domains; truncated HGPRBMY1 or HGPRBMY2 in which one or two of the domains are deleted, e.g., a soluble HGPRBMY1 or HGPRBMY2 lacking the TM or both the TM and CD regions, or a truncated, nonfunctional HGPRBMY1 or HGPRBMY2 lacking all or a portion of the CD region. Nucleotides encoding fusion polypeptides may include by are not limited to full length HGPRBMY1 or HGPRBMY2, HGPRBMY1 or HGPRBMY2 peptides, or HGPRBMY1 or HGPRBMY2 polypeptides or peptides fused to an unrelated polypeptide or peptide, such as for example, a transmembrane sequence, which anchors the HGPRBMY1 or HGPRBMY2 ECD to the cell membrane; an Ig-Fc domain which increases the stability and half life of the resulting fusion polypeptide (e.g., HGPRBMY1 or HGPRBMY2-Ig) in the bloodstream; or an enzyme, fluorescent polypeptide, luminescent polypeptide which can be used as a marker.

The invention also encompasses (a) DNA vectors that contain any of the foregoing HGPRBMY1 or HGPRBMY2 coding sequences and/or their complements (i.e., antisense); (b) DNA expression vectors that contain any of the foregoing HGPRBMY1 or HGPRBMY2 coding sequences operatively associated with a regulatory element that directs the expression of the coding sequences; and (c) genetically engineered host cells that contain any of the foregoing HGPRBMY1 or HGPRBMY2 coding sequences operatively associated with a regulatory element that directs the expression of the coding sequences in the host cell.

As used herein, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat polypeptide, the promoter for 3-phosphoglycerate kinase, the promoters of acid phosphatase, and the promoters of the yeast □-mating factors.

These expression and cloning methods include, for example, in vitro recombinant DNA techniques, synthetic techniques, and in vivo genetic recombination. (For example, Sambrook et al., 1989, supra, and Ausubel et al., 1989, supra). Alternatively, RNA capable of encoding HGPRBMY1 nucleic acid sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

As referenced elsewhere herein, characterization of the HGPRBMY1 polypeptide of the present invention led to the determination that it is involved in the modulation of the cyclin p27 protein, in addition to, the apoptosis regulatory protein IkB, either directly or indirectly.

In preferred embodiments, HGPRBMY1 polynucleotides and polypeptides, including fragments thereof, are useful for treating, diagnosing, and/or ameliorating cell cycle defects, disorders related to aberrant phosphorylation, disorders related to aberrant signal transduction, proliferating disorders, and/or cancers.

In preferred embodiments, antagonists directed to HGPRBMY1 are useful for decreasing cellular proliferation, decreasing cellular proliferation in rapidly proliferating cells, increasing the number of cells in the G1 phase of the cell cycle, and decreasing the number of cells that progress to the S phase of the cell cycle.

Moreover, agonists directed against HGPRBMY1 are useful for increasing cellular proliferation, increasing cellular proliferation in rapidly proliferating cells, decreasing the number of cells in the G1 phase of the cell cycle, and increasing the number of cells that progress to the S phase of the cell cycle. Such agonists would be particularly useful for transforming normal cells into immortalized cell lines, stimulating hematopoietic cells to grow and divide, increasing recovery rates of cancer patients that have undergone chemotherapy or other therapeutic regimen, by boosting their immune responses, etc.

In preferred embodiments, HGPRBMY1 polynucleotides and polypeptides, including fragments thereof, are useful for treating, diagnosing, and/or ameliorating proliferative disorders, cancers, ischemia-reperfusion injury, heart failure, immuno compromised conditions, HIV infection, and renal diseases.

Moreover, HGPRBMY1 polynucleotides and polypeptides, including fragments thereof, are useful for increasing NF-kB activity, decreasing apoptotic events, and/or decreasing I□B□ expression or activity levels.

In preferred embodiments, antagonists directed against HGPRBMY1 are useful for treating, diagnosing, and/or ameliorating autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, and HIV propagation in cells infected with other viruses.

Moreover, antagonists directed against HGPRBMY1 are useful for decreasing NF-kB activity, increasing apoptotic events, and/or increasing I□B□ expression or activity levels.

In preferred embodiments, agonists directed against HGPRBMY1 are useful for treating, diagnosing, and/or ameliorating autoimmune diorders, disorders related to hyper immune activity, hypercongenital conditions, birth defects, necrotic lesions, wounds, disorders related to aberrant signal transduction, immuno compromised conditions, HIV infection, proliferating disorders, Alzheimer's, and/or cancers.

Moreover, agonists directed against HGPRBMY1 are useful for increasing NF-kB activity, decreasing apoptotic events, and/or decreasing I□B□ expression or activity levels.

5.2. HGPRBMY1 and HGPRBMY2 Polypeptides

The term “peptides” as used herein is meant to comprise a small number of amino acids connected by peptide bonds. The term “polypeptide” generally refers to longer chains of amino acids but does not refer to a specific length, thus as used herein, polypeptides include proteins (a term usually reserved for a functional unit which may consist of either a single polypeptide or several polypeptides).

The invention features polypeptides and/or peptides that are similar to the sequence of HGPRBMY1. A polypeptide or peptide that has a similar amino acid sequence refers to a polypeptide or peptide sequence that satisfies at least one of the following: (a) a polypeptide having an amino acid sequence that is at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a GPCR polypeptide or peptide as described herein; (b) a polypeptide or peptide encoded by a nucleic acid sequence that hybridizes under stringent conditions to a nucleic acid sequence encoding a GPCR as described herein of at least 20 amino acid residues, at least 25, at least 40, at least 50, at least 60, at least 70, at least 80, at least 90, at least 100, at least 125, at least 150, at least 200, at least 225, at least 250, at least 275, at least 300 or at least 350 amino acids; and (c) a polypeptide encoded by a nucleic acid sequence that is at least 40%, at least 45%, at least 50%, at least 55%, at least 60%, at least 65%, at least 70%, at least 75%, at least 80%, at least 85%, at least 90%, at least 95% or at least 99% identical to the nucleic acid sequence encoding a GPCR polypeptide or peptide as described herein.

The invention features polypeptides and/or peptides that are similar to the sequence of HGPRBMY2. A polypeptide or peptide that has a similar amino acid sequence refers to a polypeptide or peptide sequence that satisfies at least one of the following: (a) a polypeptide having an amino acid sequence that is at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a GPCR polypeptide or peptide as described herein; (b) a polypeptide or peptide encoded by a nucleic acid sequence that hybridizes under stringent conditions to a nucleic acid sequence encoding a GPCR as described herein of at least 20 amino acid residues, at least 25, at least 40, at least 50, at least 60, at least 70, at least 80, at least 90, at least 100, at least 125, at least 150, at least 200, at least 225, at least 250, at least 275, at least 300 or at least 350 amino acids; and (c) a polypeptide encoded by a nucleic acid sequence that is at least 40%, at least 45%, at least 50%, at least 55%, at least 60%, at least 65%, at least 70%, at least 75%, at least 80%, at least 85%, at least 90%, at least 95% or at least 99% identical to the nucleic acid sequence encoding a GPCR polypeptide or peptide as described herein.

A polypeptide with similar structure and/or function to a GPCR polypeptide or as described herein refers to a polypeptide that has a similar secondary, tertiary or quaternary structure of a GPCR polypeptide, e.g., a protein or a fusion protein, as described herein. The structure of a polypeptide can determined by methods known to those skilled in the art, including but not limited to, X-ray crystallography, nuclear magnetic resonance, and crystallographic electron microscopy.

HGPRBMY1 polypeptides and peptides, mutations, truncations and/or HGPRBMY1 fusion polypeptides of any of the foregoing can be used for, but not limited to, the generation of antibodies, as reagents in diagnostic assays, the identification of other cellular gene products involved in the regulation of immune function, as reagents in assays for screening for compounds that can be used in the treatment of immune disorders, and as pharmaceutical reagents useful in the treatment of immune disorders related to the HGPRBMY1.

In preferred embodiments, the following N-terminal HGPRBMY1 deletion polypeptides are encompassed by the present invention: M1-F359, Q2-F359, V3-F359, P4-F359, N5-F359, S6-F359, T7-F359, G8-F359, P9-F359, D10-F359, N11-F359, A12-F359, T13-F359, L14-F359, Q15-F359, M16-F359, L17-F359, R18-F359, N19-F359, P20-F359, A21-F359, I22-F359, A23-F359, V24-F359, A25-F359, L26-F359, P27-F359, V28-F359, V29-F359, Y30-F359, S31-F359, L32-F359, V33-F359, A34-F359, A35-F359, V36-F359, S37-F359, I38-F359, P39-F359, G40-F359, N41-F359, L42-F359, F43-F359, S44-F359, L45-F359, W46-F359, V47-F359, L48-F359, C49-F359, R50-F359, R51-F359, M52-F359, G53-F359, P54-F359, R55-F359, S56-F359, P57-F359, S58-F359, V59-F359, 160-F359, F61-F359, M62-F359, 163-F359, N64-F359, L65-F359, S66-F359, V67-F359, T68-F359, D69-F359, L70-F359, M71-F359, L72-F359, A73-F359, S74-F359, V75-F359, L76-F359, P77-F359, F78-F359, Q79-F359, I80-F359, Y81-F359, Y82-F359, H83-F359, C84-F359, N85-F359, R86-F359, H87-F359, H88-F359, W89-F359, V90-F359, F91-F359, G92-F359, V93-F359, L94-F359, L95-F359, C96-F359, N97-F359, V98-F359, V99-F359, T100-F359, V101-F359, A102-F359, F103-F359, Y104-F359, A105-F359, N106-F359, M107-F359, Y108-F359, S109-F359, S110-F359, I111-F359, L112-F359, T113-F359, M114-F359, T115-F359, C116-F359, I117-F359, S118-F359, V119-F359, E120-F359, R121-F359, F122-F359, L123-F359, G124-F359, V125-F359, L126-F359, Y127-F359, P128-F359, L129-F359, S130-F359, S131-F359, K132-F359, R133-F359, W134-F359, R135-F359, R136-F359, R137-F359, R138-F359, Y139-F359, A140-F359, V141-F359, A142-F359, A143-F359, C144-F359, A145-F359, G146-F359, T147-F359, W148-F359, L149-F359, L150-F359, L151-F359, L152-F359, T153-F359, A154-F359, L155-F359, S156-F359, P157-F359, L158-F359, A159-F359, R160-F359, T161-F359, D162-F359, L163-F359, T164-F359, Y165-F359, P166-F359, V167-F359, H168-F359, A169-F359, L170-F359, G171-F359, I172-F359, I173-F359, T174-F359, C175-F359, F176-F359, D177-F359, V178-F359, L179-F359, K180-F359, W181-F359, T182-F359, M183-F359, L184-F359, P185-F359, S186-F359, V187-F359, A188-F359, M189-F359, W190-F359, A191-F359, V192-F359, F193-F359, L194-F359, F195-F359, T196-F359, I197-F359, F198-F359, I199-F359, L200-F359, L201-F359, F202-F359, L203-F359, I204-F359, P205-F359, F206-F359, V207-F359, I208-F359, T209-F359, V210-F359, A211-F359, C212-F359, Y213-F359, T214-F359, A215-F359, T216-F359, I217-F359, L218-F359, K219-F359, L220-F359, L221-F359, R222-F359, T223-F359, E224-F359, E225-F359, A226-F359, H227-F359, G228-F359, R229-F359, E230-F359, Q231-F359, R232-F359, R233-F359, R234-F359, A235-F359, V236-F359, G237-F359, L238-F359, A239-F359, A240-F359, V241-F359, V242-F359, L243-F359, L244-F359, A245-F359, F246-F359, V247-F359, T248-F359, C249-F359, F250-F359, A251-F359, P252-F359, N253-F359, N254-F359, F255-F359, V256-F359, L257-F359, L258-F359, A259-F359, H260-F359, I261-F359, V262-F359, S263-F359, R264-F359, L265-F359, F266-F359, Y267-F359, G268-F359, K269-F359, S270-F359, Y271-F359, Y272-F359, H273-F359, V274-F359, Y275-F359, K276-F359, L277-F359, T278-F359, L279-F359, C280-F359, L281-F359, S282-F359, C283-F359, L284-F359, N285-F359, N286-F359, C287-F359, L288-F359, D289-F359, P290-F359, F291-F359, V292-F359, Y293-F359, Y294-F359, F295-F359, A296-F359, S297-F359, R298-F359, E299-F359, F300-F359, Q301-F359, L302-F359, R303-F359, L304-F359, R305-F359, E306-F359, Y307-F359, L308-F359, G309-F359, C310-F359, R311-F359, R312-F359, V313-F359, P314-F359, R315-F359, D316-F359, T317-F359, L318-F359, D319-F359, T320-F359, R321-F359, R322-F359, E323-F359, S324-F359, L325-F359, F326-F359, S327-F359, A328-F359, R329-F359, T330-F359, T331-F359, S332-F359, V333-F359, R334-F359, S335-F359, E336-F359, A337-F359, G338-F359, A339-F359, H340-F359, P341-F359, E342-F359, G343-F359, M344-F359, E345-F359, G346-F359, A347-F359, T348-F359, R349-F359, P350-F359, G351-F359, L352-F359, and/or Q353-F359 of SEQ ID NO:2. Polynucleotide sequences encoding these polypeptides are also provided. The present invention also encompasses the use of these N-terminal HGPRBMY1 deletion polypeptides as immunogenic and/or antigenic epitopes as described elsewhere herein.

In preferred embodiments, the following C-terminal HGPRBMY1 deletion polypeptides are encompassed by the present invention: M1-F359, M1-V358, M1-S357, M1-E356, M1-Q355, M1-R354, M1-Q353, M1-L352, M1-G351, M1-P350, M1-R349, M1-T348, M1-A347, M1-G346, M1-E345, M1-M344, M1-G343, M1-E342, M1-P341, M1-H340, M1-A339, M1-G338, M1-A337, M1-E336, M1-S335, M1-R334, M1-V333, M1-S332, M1-T331, M1-T330, M1-R329, M1-A328, M1-S327, M1-F326, M1-L325, M1-S324, M1-E323, M1-R322, M1-R321, M1-T320, M1-D319, M1-L318, M1-T317, M1-D316, M1-R315, M1-P314, M1-V313, M1-R312, M1-R311, M1-C310, M1-G309, M1-L308, M1-Y307, M1-E306, M1-R305, M1-L304, M1-R303, M1-L302, M1-Q301, M1-F300, M1-E299, M1-R298, M1-S297, M1-A296, M1-F295, M1-Y294, M1-Y293, M1-V292, M1-F291, M1-P290, M1-D289, M1-L288, M1-C287, M1-N286, M1-N285, M1-L284, M1-C283, M1-S282, M1-L281, M1-C280, M1-L279, M1-T278, M1-L277, M1-K276, M1-Y275, M1-V274, M1-H273, M1-Y272, M1-Y271, M1-S270, M1-K269, M1-G268, M1-Y267, M1-F266, M1-L265, M1-R264, M1-S263, M1-V262, M1-1261, M1-H260, M1-A259, M1-L258, M1-L257, M1-V256, M1-F255, M1-N254, M1-N253, M1-P252, M1-A251, M1-F250, M1-C249, M1-T248, M1-V247, M1-F246, M1-A245, M1-L244, M1-L243, M1-V242, M1-V241, M1-A240, M1-A239, M1-L238, M1-G237, M1-V236, M1-A235, M1-R234, M1-R233, M1-R232, M1-Q231, M1-E230, M1-R229, M1-G228, M1-H227, M1-A226, M1-E225, M1-E224, M1-T223, M1-R222, M1-L221, M1-L220, M1-K219, M1-L218, M1-I217, M1-T216, M1-A215, M1-T214, M1-Y213, M1-C212, M1-A211, M1-V210, M1-T209, M1-1208, M1-V207, M1-F206, M1-P205, M1-I204, M1-L203, M1-F202, M1-L201, M1-L200, M1-I199, M1-F198, M1-I197, M1-T196, M1-F195, M1-L194, M1-F193, M1-V192, M1-A191, M1-W190, M1-M189, M1-A188, M1-V187, M1-S186, M1-P185, M1-L184, M1-M183, M1-T182, M1-W181, M1-K180, M1-L179, M1-V178, M1-D177, M1-F176, M1-C175, M1-T174, M1-I173, M1-I172, M1-G171, M1-L170, M1-A169, M1-H168, M1-V167, M1-P166, M1-Y165, M1-T164, M1-L163, M1-D162, M1-T161, M1-R160, M1-A159, M1-L158, M1-P157, M1-S156, M1-L155, M1-A154, M1-T153, M1-L152, M1-L151, M1-L150, M1-L149, M1-W148, M1-T147, M1-G146, M1-A145, M1-C144, M1-A143, M1-A142, M1-V141, M1-A140, M1-Y139, M1-R138, M1-R137, M1-R136, M1-R135, M1-W134, M1-R133, M1-K132, M1-S131, M1-S130, M1-L129, M1-P128, M1-Y127, M1-L126, M1-V125, M1-G124, M1-L123, M1-F122, M1-R121, M1-E120, M1-V119, M1-S118, M1-I117, M1-C116, M1-T115, M1-M114, M1-T113, M1-L112, M1-I111, M1-S110, M1-S109, M1-Y108, M1-M107, M1-N106, M1-A105, M1-Y104, M1-F103, M1-A102, M1-V101, M1-T100, M1-V99, M1-V98, M1-N97, M1-C96, M1-L95, M1-L94, M1-V93, M1-G92, M1-F91, M1-V90, M1-W89, M1-H88, M1-H87, M1-R86, M1-N85, M1-C84, M1-H83, M1-Y82, M1-Y81, M1-I80, M1-Q79, M1-F78, M1-P77, M1-L76, M1-V75, M1-S74, M1-A73, M1-L72, M1-M71, M1-L70, M1-D69, M1-T68, M1-V67, M1-S66, M1-L65, M1-N64, M1-I63, M1-M62, M1-F61, M1-I60, M1-V59, M1-S58, M1-P57, M1-S56, M1-R55, M1-P54, M1-G53, M1-M52, M1-R51, M1-R50, M1-C49, M1-L48, M1-V47, M1-W46, M1-L45, M1-S44, M1-F43, M1-L42, M1-N41, M1-G40, M1-P39, M1-I38, M1-S37, M1-V36, M1-A35, M1-A34, M1-V33, M1-L32, M1-S31, M1-Y30, M1-V29, M1-V28, M1-P27, M1-L26, M1-A25, M1-V24, M1-A23, M1-I22, M1-A21, M1-P20, M1-N19, M1-R18, M1-L17, M1-M16, M1-Q15, M1-L14, M1-T13, M1-A12, M1-N11, M1-D10, M1-P9, M1-G8, and/or M1-T7 of SEQ ID NO:2. Polynucleotide sequences encoding these polypeptides are also provided. The present invention also encompasses the use of these C-terminal HGPRBMY1 deletion polypeptides as immunogenic and/or antigenic epitopes as described elsewhere herein.

HGPRBMY2 polypeptides and peptides, mutated, truncated or deleted forms of the HGPRBMY2 and/or HGPRBMY2 fusion polypeptides can be prepared for a variety of uses, including but not limited to the generation of antibodies, as reagents in diagnostic assays, the identification of other cellular gene products involved in the regulation of cardiovascular, as reagents in assays for screening for compounds that can be used in the treatment of cardiovascular disorders, and as pharmaceutical reagents useful in the treatment of cardiovascular disorders related to the HGPRBMY2.

The deduced amino acid sequence encoded by the open reading frame of HGPRBMY2 is 431 amino acids (SEQ ID NO:14) and is shown in FIG. 7. The extracellular domains (“ECD”) of HGPRBMY2 extend from about amino acid residues 1 to about 45, about 105 to about 119, about 182 to about 212, and about 293 to about 311 of SEQ ID NO:14; the transmembrane domains of HGPRBMY2 extend from about amino acid residues 46 to about 69, about 82 to about 104, about 119 to about 141, about 162 to about 181, about 213 to about 233, about 272 to about 292, and about 312 to about 335 of SEQ ID NO:14; and the cytoplasmic domains of HGPRBMY2 extend from about amino acid residue 69 to about 81, about 142 to about 161, about 234 to about 271, and about 336 to about 431 of SEQ ID NO:14.

In preferred embodiments, the following N-terninal HGPRBMY2 deletion polypeptides are encompassed by the present invention: M1-H431, Q2-H431, A3-H431, L4-H431, N5-H431, I6-H431, T7-H431, P8-H431, E9-H431, Q10-H431, F11-H431, S12-H431, R13-H431, L14-H431, L15-H431, R16-H431, D17-H431, H18-H431, N19-H431, L20-H431, T21-H431, R22-H431, E23-H431, Q24-H431, F25-H431, I26-H431, A27-H431, L28-H431, Y29-H431, R30-H431, L31-H431, R32-H431, P33-H431, L34-H431, V35-H431, Y36-H431, T37-H431, P38-H431, E39-H431, L40-H431, P41-H431, G42-H431, R43-H431, A44-H431, K45-H431, L46-H431, A47-H431, L48-H431, V49-H431, L50-H431, T51-H431, G52-H431, V53-H431, L54-H431, I55-H431, F56-H431, A57-H431, L58-H431, A59-H431, L60-H431, F61-H431, G62-H431, N63-H431, A64-H431, L65-H431, V66-H431, F67-H431, Y68-H431, V69-H431, V70-H431, T71-H431, R72-H431, S73-H431, K74-H431, A75-H431, M76-H431, R77-H431, T78-H431, V79-H431, T80-H431, N81-H431, I82-H431, F83-H431, I84-H431, C85-H431, S85-H431, L87-H431, A88-H431, L89-H431, S90-H431, D91-H431, L92-H431, L93-H431, I94-H431, T95-H431, F96-H431, F97-H431, C98-H431, I99-H431, P100-H431, V101-H431, T102-H431, M103-H431, L104-H431, Q105-H431, N106-H431, I107-H431, S108-H431, D109-H431, N110-H431, W111-H431, L112-H431, G113-H431, G114-H431, A115-H431, F116-H431, I117-H431, C118-H431, K119-H431, M120-H431, V121-A128-H431, V129-H431, V130-H431, T131-H431, E132-H431, I133-H431, L134-H431, T135-H431, M136-H431, T137-H431, C138-H431, I139-H431, A140-H431, V141-H431, E142-H431, R143-H431, H144-H431, Q145-H431, G146-H431, L147-H431, V148-H431, H149-H431, P150-H431, F151-H431, K152-H431, M153-H431, K154-H431, W155-H431, Q156-H431, Y157-H431, T158-H431, N159-H431, R160-H431, R161-H431, A162-H431, F163-H431, T164-H431, M165-H431, L166-H431, G167-H431, V168-H431, V169-H431, W170-H431, L171-H431, V172-H431, A173-H431, V174-H431, I175-H431, V176-H431, G177-H431, S178-H431, P179-H431, M180-H431, W181-H431, H182-H431, V183-H431, Q184-H431, Q185-H431, L186-H431, E187-H431, I188-H431, K189-H431, Y190-H431, D191-H431, F192-H431, L193-H431, Y194-H431, E195-H431, K196-H431, E197-H431, H198-H431, I199-H431, C200-H431, C201-H431, L202-H431, E203-H431, E204-H431, W205-H431, T206-H431, S207-H431, P208-H431, V209-H431, H210-H431, Q211-H431, K212-H431, I213-H431, Y214-H431, T215-H431, T216-H431, F217-H431, I218-H431, L219-H431, V220-H431, I221-H431, L222-H431, F223-H431, L224-H431, L225-H431, P226-H431, L227-H431, M228-H431, V229-H431, M230-H431, L231-H431, I232-H431, L233-H431, Y234-H431, S235-H431, K236-H431, I237-H431, G238-H431, Y239-H431, E240-H431, L241-H431, W242-H431, I243-H431, K244-H431, K245-H431, R246-H431, V247-H431, G248-H431, D249-H431, G250-H431, S251-H431, V252-H431, L253-H431, R254-H431, T255-H431, I256-H431, H257-H431, G258-H431, K259-H431, E260-H431, M261-H431, S262-H431, K263-H431, I264-H431, A265-H431, R266-H431, K267-H431, K268-H431, K269-H431, R270-H431, A271-H431, V272-H431, I273-H431, M274-H431, M275-H431, V276-H431, T277-H431, V278-H431, V279-H431, A280-H431, L281-H431, F282-H431, A283-H431, V284-H431, C285-H431, W286-H431, A287-H431, P288-H431, F289-H431, H290-H431, V291-H431, V292-H431, H293-H431, M294-H431, M295-H431, I296-H431, E297-H431, Y298-H431, S299-H431, N300-H431, F301-H431, E302-H431, K303-H431, E304-H431, Y305-H431, D306-H431, D307-H431, V308-H431, T309-H431, I310-H431, K311-H431, M312-H431, I313-H431, F314-H431, A315-H431, I316-H431, V317-H431, Q318-H431, I319-H431, I320-H431, G321-H431, F322-H431, S323-H431, N324-H431, S325-H431, I326-H431, C327-H431, N328-H431, P329-H431, I330-H431, V331-H431, Y332-H431, A333-H431, F334-H431, M335-H431, N336-H431, E337-H431, N338-H431, F339-H431, K340-H431, K341-H431, N342-H431, V343-H431, L344-H431, S345-H431, A346-H431, V347-H431, C348-H431, Y349-H431, C350-H431, I351-H431, V352-H431, N353-H431, K354-H431, T355-H431, F356-H431, S357-H431, P358-H431, A359-H431, Q360-H431, R361-H431, H362-H431, G363-H431, N364-H431, S365-H431, G366-H431, I367-H431, T368-H431, M369-H431, M370-H431, R371-H431, K372-H431, K373-H431, A374-H431, K375-H431, F376-H431, S377-H431, L378-H431, R379-H431, E380-H431, N381-H431, P382-H431, V383-H431, E384-H431, E385-H431, T386-H431, K387-H431, G388-H431, E389-H431, A390-H431, F391-H431, S392-H431, D393-H431, G394-H431, N395-H431, I396-H431, E397-H431, V398-H431, K399-H431, L400-H431, C401-H431, E402-H431, Q403-H431, T404-H431, E405-H431, E406-H431, K407-H431, K408-H431, K409-H431, L410-H431, K411-H431, R412-H431, H413-H431, L414-H431, A415-H431, L416-H431, F417-H431, R418-H431, S419-H431, E420-H431, L421-H431, A422-H431, E423-H431, N424-H431, and/or S425-H431 of SEQ ID NO:14. Polynucleotide sequences encoding these polypeptides are also provided. The present invention also encompasses the use of these N-terminal HGPRBMY2 deletion polypeptides as immunogenic and/or antigenic epitopes as described elsewhere herein.

In preferred embodiments, the following C-terminal HGPRBMY2 deletion polypeptides are encompassed by the present invention: M1-H431, M1-G430, M1-S429, M1-D428, M1-L427, M1-P426, M1-S425, M1-N424, M1-E423, M1-A422, M1-L421, M1-E420, M1-S419, M1-R418, M1-F417, M1-L416, M1-A415, M1-L414, M1-H413, M1-R412, M1-K411, M1-L410, M1-K409, M1-K408, M1-K407, M1-E406, M1-E405, M1-T404, M1-Q403, M1-E402, M1-C401, M1-L400, M1-K399, M1-V398, M1-E397, M1-I396, M1-N395, M1-G394, M1-D393, M1-S392, M1-F391, M1-A390, M1-E389, M1-G388, M1-K387, M1-T386, M1-E385, M1-E384, M1-V383, M1-P382, M1-N381, M1-E380, M1-R379, M1-L378, M1-S377, M1-F376, M1-K375, M1-A374, M1-K373, M1-K372, M1-R371, M1-M370, M1-M369, M1-T368, M1-I367, M1-G366, M1-S365, M1-N364, M1-G363, M1-H362, M1-R361, M1-Q360, M1-A359, M1-P358, M1-S357, M1-F356, M1-T355, M1-K354, M1-N353, M1-V352, M1-I351, M1-C350, M1-Y349, M1-C348, M1-V347, M1-A346, M1-S345, M1-L344, M1-V343, M1-N342, M1-K341, M1-K340, M1-F339, M1-N338, M1-E337, M1-N336, M1-M335, M1-F334, M1-A333, M1-Y332, M1-V331, M1-I330, M1-P329, M1-N328, M1-C327, M1-I326, M1-S325, M1-N324, M1-S323, M1-F322, M1-G321, M1-I320, M1-I319, M1-Q318, M1-V317, M1-I316, M1-A315, M1-F314, M1-I313, M1-M312, M1-K311, M1-I310, M1-T309, M1-V308, M1-D307, M1-D306, M1-Y305, M1-E304, M1-K303, M1-E302, M1-F301, M1-N300, M1-S299, M1-Y298, M1-E297, M1-I296, M1-M295, M1-M294, M1-H293, M1-V292, M1-V291, M1-H290, M1-F289, M1-P288, M1-A287, M1-W286, M1-C285, M1-V284, M1-A283, M1-F282, M1-L281, M1-A280, M1-V279, M1-V278, M1-T277, M1-V276, M1-M275, M1-M274, M1-I273, M1-V272, M1-A271, M1-R270, M1-K269, M1-K268, M1-K267, M1-R266, M1-A265, M1-I264, M1-K263, M1-S262, M1-M261, M1-E260, M1-K259, M1-G258, M1-H257, M1-1256, M1-T255, M1-R254, M1-L253, M1-V252, M1-S251, M1-G250, M1-D249, M1-G248, M1-V247, M1-R246, M1-K245, M1-K244, M1-I243, M1-W242, M1-L241, M1-E240, M1-Y239, M1-G238, M1-I237, M1-K236, M1-S235, M1-Y234, M1-L233, M1-I232, M1-L231, M1-M230, M1-V229, M1-M228, M1-L227, M1-P226, M1-L225, M1-L224, M1-F223, M1-L222, M1-I221, M1-V220, M1-L219, M1-I218, M1-F217, M1-T216, M1-T215, M1-Y214, M1-I213, M1-K212, M1-Q211, M1-H210, M1-V209, M1-P208, M1-S207, M1-T206, M1-W205, M1-E204, M1-E203, M1-L202, M1-C201, M1-C200, M1-I199, M1-H198, M1-E197, M1-K196, M1-E195, M1-Y194, M1-L193, M1-F192, M1-D191, M1-Y190, M1-K189, M1-I188, M1-E187, M1-L186, M1-Q185, M1-Q184, M1-V183, M1-H182, M1-W181, M1-M180, M1-P179, M1-S178, M1-G177, M1-V176, M1-I175, M1-V174, M1-A173, M1-V172, M1-L171, M1-W170, M1-V169, M1-V168, M1-G167, M1-L166, M1-M165, M1-T164, M1-F163, M1-A162, M1-R161, M1-R160, M1-N159, M1-T158, M1-Y157, M1-Q156, M1-W155, M1-K154, M1-M153, M1-K152, M1-F151, M1-P150, M1-H149, M1-V148, M1-L147, M1-G146, M1-Q145, M1-H144, M1-R143, M1-E142, M1-V141, M1-A140, M1-I139, M1-C138, M1-T137, M1-M136, M1-T135, M1-L134, M1-I133, M1-E132, M1-T131, M1-V130, M1-V129, M1-A128, M1-T127, M1-S126, M1-Q125, M1-V124, M1-F123, M1-P122, M1-V121, M1-M120, M1-K119, M1-C118, M1-I117, M1-F116, M1-A115, M1-G114, M1-G113, M1-L112, M1-W111, M1-N110, M1-D109, M1-S108, M1-I107, M1-N106, M1-Q105, M1-L104, M1-M103, M1-T102, M1-V101, M1-P100, M1-I99, M1-C98, M1-F97, M1-F96, M1-T95, M1-I94, M1-L93, M1-L92, M1-D91, M1-S90, M1-L89, M1-A88, M1-L87, M1-S86, M1-C85, M1-I84, M1-F83, M1-I82, M1-N81, M1-T80, M1-V79, M1-T78, M1-R77, M1-M76, M1-A75, M1-K74, M1-S73, M1-R72, M1-T71, M1-V70, M1-V69, M1-Y68, M1-F67, M1-V66, M1-L65, M1-A64, M1-N63, M1-G62, M1-F61, M1-L60, M1-A59, M1-L58, M1-A57, M1-F56, M1-I55, M1-L54, M1-V53, M1-G52, M1-T51, M1-L50, M1-V49, M1-L48, M1-A47, M1-L46, M1-K45, M1-A44, M1-R43, M1-G42, M1-P41, M1-L40, M1-E39, M1-P38, M1-T37, M1-Y36, M1-V35, M1-L34, M1-P33, M1-R32, M1-L31, M1-R30, M1-Y29, M1-L28, M1-A27, M1-I26, M1-F25, M1-Q24, M1-E23, M1-R22, M1-T21, M1-L20, M1-N19, M1-H18, M1-D17, M1-R16, M1-L15, M1-L14, M1-R13, M1-S12, M1-F11, M1-Q10, M1-E9, M1-P8, and/or M1-T7 of SEQ ID NO:14. Polynucleotide sequences encoding these polypeptides are also provided. The present invention also encompasses the use of these C-terminal HGPRBMY2 deletion polypeptides as immunogenic and/or antigenic epitopes as described elsewhere herein.

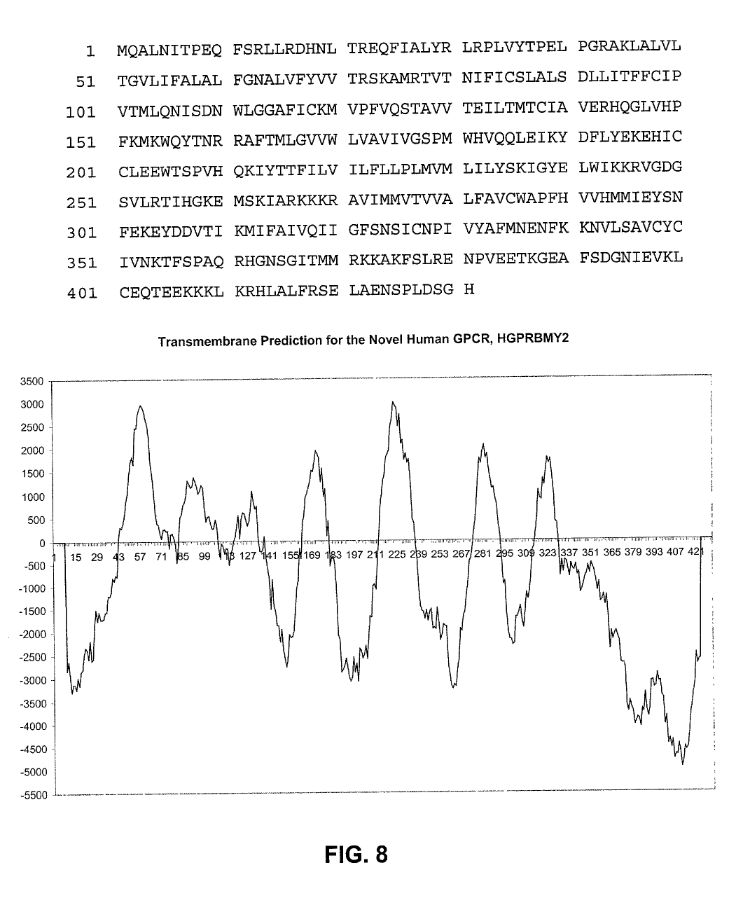

FIG. 8 depicts the putative transmembrane regions of the HGPRBMY2 polypeptide as shaded areas of the sequence, and also presents a hydropathy plot which was used to predict the hydrophobic and hydrophilic regions of the full length polypeptide.

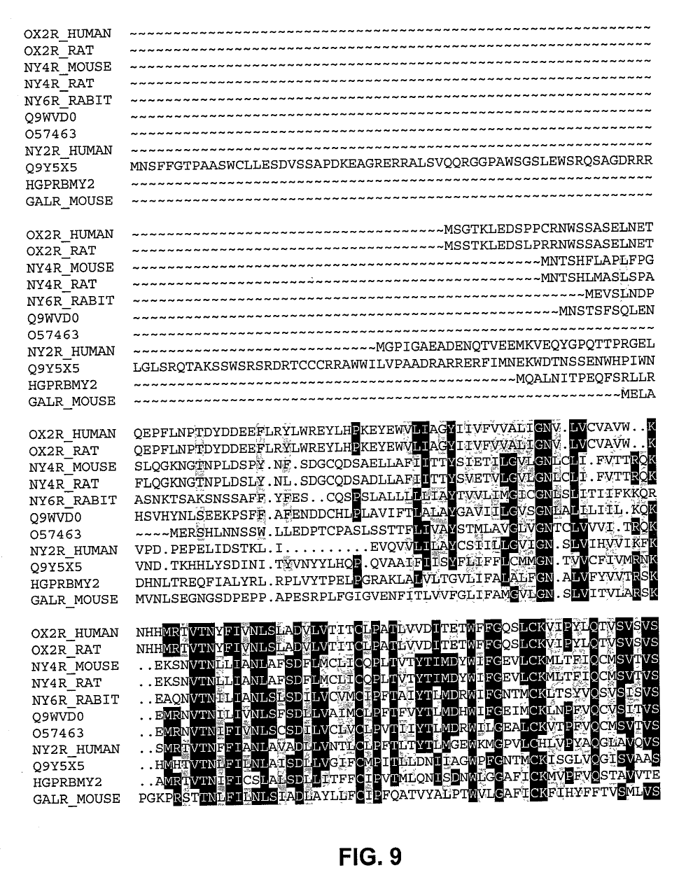

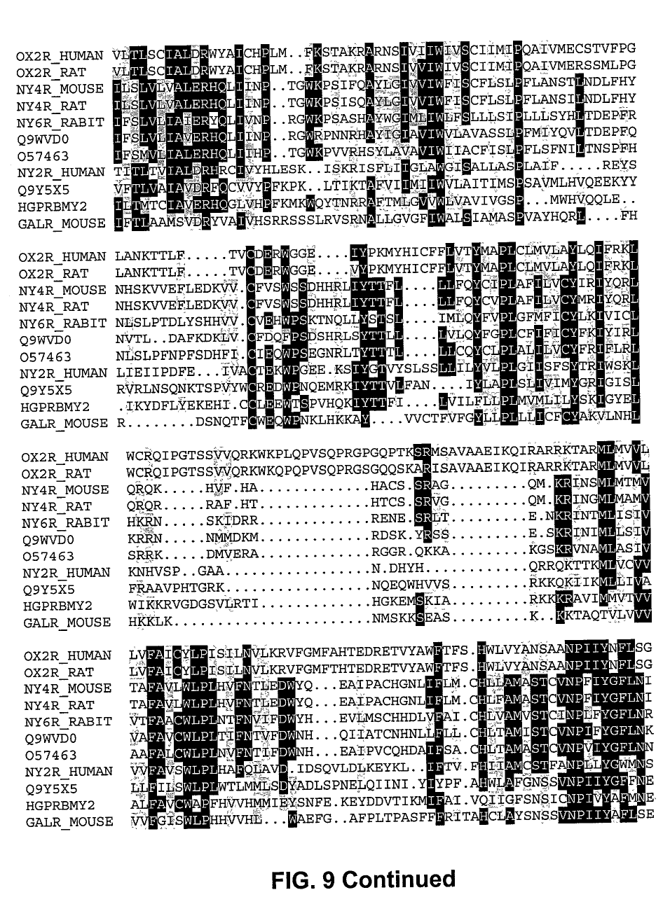

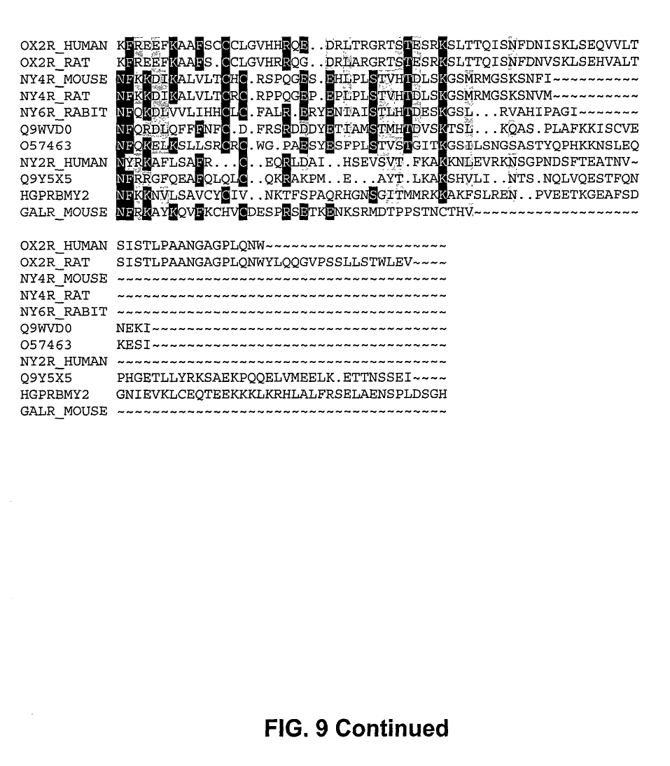

The HGPRBMY2 sequence begins with a methionine in a DNA sequence context consistent with a translation initiation site. An alignment between the HGPRBMY2 polypeptide with neuropeptide, orexin and galanin receptor sequences is shown in FIG. 9 (par2_human, Genbank Accession No. gi|18560788, SEQ ID NO:36; par3_{'}human, Genbank Accession No. NP_{—}004092, SEQ ID NO:37; thrombin_Xeno, Genbank Accession No. gi|2134162, SEQ ID NO:38; thrombin_human, Genbank Accession No. NP_{—}001983, SEQ ID NO:39; par4_human, Genbank Accession No. NP_{—}003941, SEQ ID NO:40; and p2y9_human, Genbank Accession No. gi|17426979, SEQ ID NO:41). Although the overall amino acid sequence identity between these molecules is low, there are numerous residues which are conserved across all of the GPCRs in the alignment suggesting a functional importance for that residue.

The HGPRBMY1 amino acid sequences of the invention include the amino acid sequence shown in FIG. 2 (SEQ ID NO:2). The cDNA sequence (SEQ ID NO:1) described in Section 5.1 encodes the amino acid sequence of HGPRBMY1 (359 amino acids; SEQ ID NO:2). The extracellular domains (“ECD”) of HGPRBMY1 extend from about amino acid residues 1 to about 27, about 85 to about 88, about 161 to about 186, and about 259 to about 276 of SEQ ID NO:2; the transmembrane domains (“TM”) of HGPRBMY1 extend from about amino acid residues 28 to about 49, about 60 to about 84, about 89 to about 105, about 139 to about 160, about 187 to about 200, about 235 to about 258, and about 277 to about 297 of SEQ ID NO:2; and the cytoplasmic domains (“CD”) of HGPRBMY1 extend from about amino acid residue 50 to about 59, about 106 to about 138, about 201 to about 234, and about 298 to about 359 of SEQ ID NO:2.

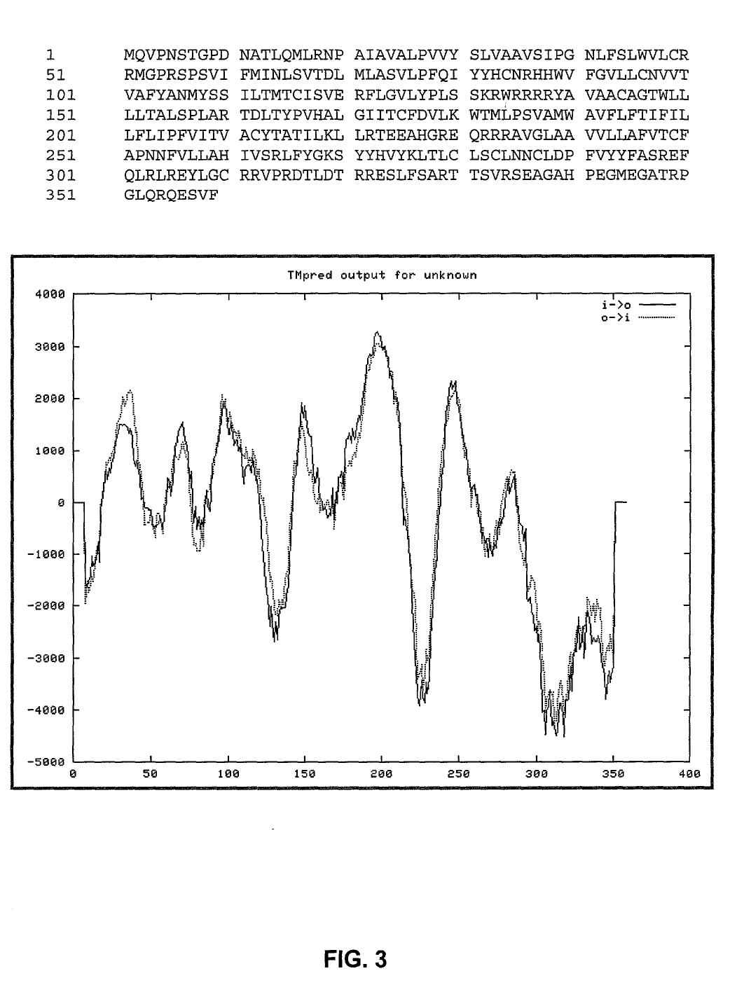

FIG. 3 depicts the putative transmembrane regions of the HGPRBMY1 polypeptide as shaded areas of the sequence, and also presents a hydropathy plot which was used to predict the hydrophobic and hydrophilic regions of the full length polypeptide.

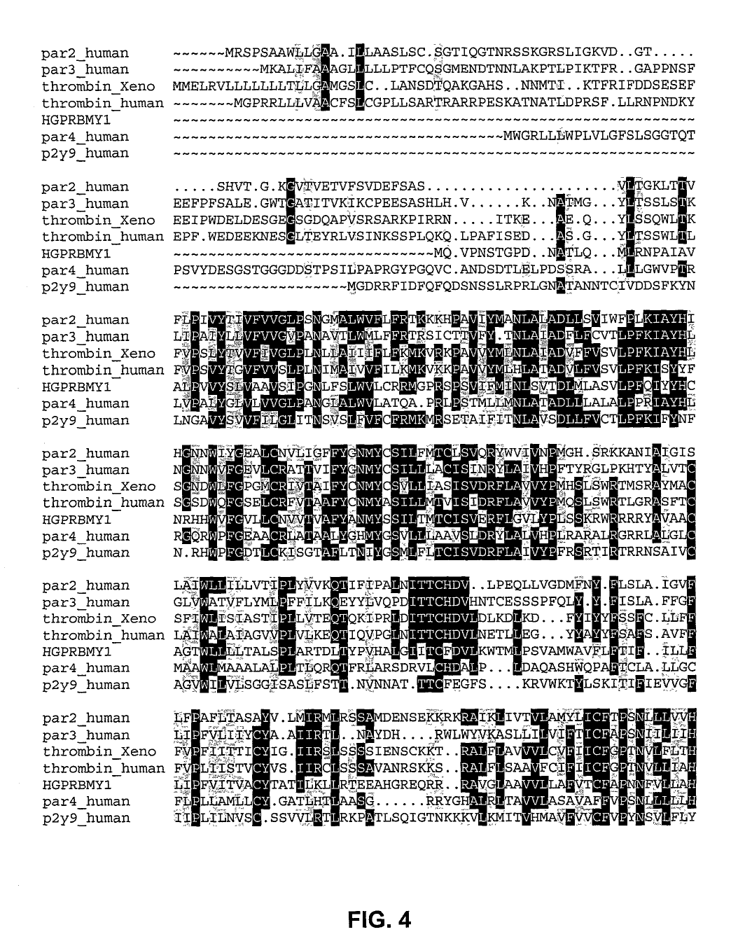

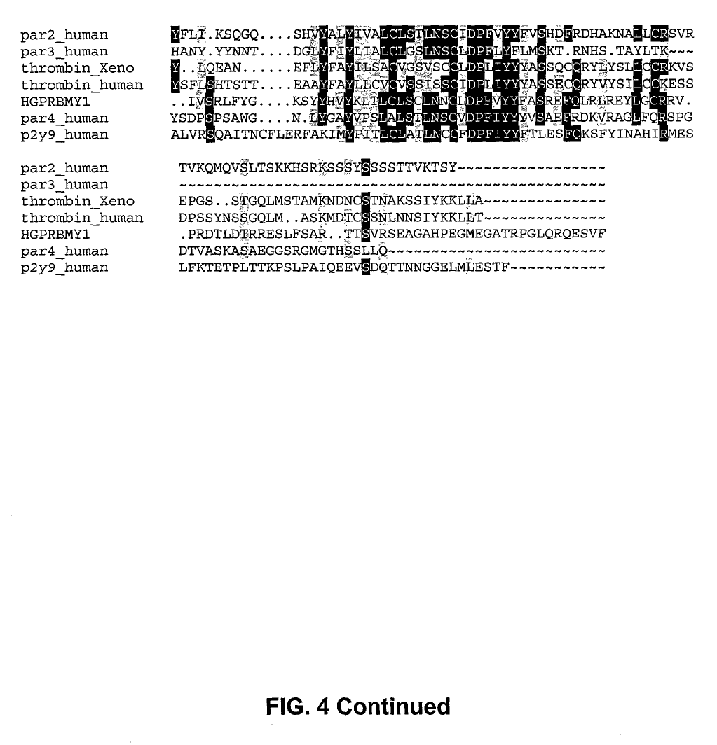

The HGPRBMY1 sequence begins with a methionine in a DNA sequence context consistent with a translation initiation site. An alignment between the HGPRBMY1 polypeptide with thrombin receptor, protease activated receptor (par) and P2Y9-like receptor sequences is shown in FIG. 4 (OX2R_HUMAN, Genbank Accession No. gi|17978555, SEQ ID NO:42; OX2R_RAT, Genbank Accession No. gi|6981020, SEQ ID NO:43;. NY4R_MOUSE, Genbank Accession No. gi|587693, SEQ ID NO:44; NY4R_RAT, Genbank Accession No. gi|2494992, SEQ ID NO:45; NY6R_RABIT, Genbank Accession No. gi|3024242, SEQ ID NO:46; Q9WVD0, Genbank Accession No. gi|5410446, SEQ ID NO:47; 057463, Genbank Accession No. gi|2739141, SEQ ID NO:48; NY2R_HUMAN, Genbank Accession No. NP_{—}000901, SEQ ID NO:49; Q9Y5X5, Genbank Accession No. gi|4530469, SEQ ID NO:50; and GALR_MOUSE, Genbank Accession No. gi|3023827, SEQ ID NO:51. Although the overall amino acid sequence identity between these molecules is low, there are numerous residues which are conserved across all of the GPCRs in the alignment suggesting a functional importance for that residue.

Peptides and polypeptides of HGPRBMY1 or HGPRBMY2 or mutants thereof can also be chemically synthesized (e.g., see Creighton, 1983, Proteins: Structures and Molecular Principles, W. H. Freeman & Co., N.Y.). In addition, polypeptides and peptides of the invention may be produced by recombinant DNA technology using techniques well known in the art for expressing nucleic acid containing HGPRBMY1 or HGPRBMY2 gene sequences and/or coding sequences. Such methods can be used to construct expression vectors containing various HGPRBMY1 or HGPRBMY2 nucleic acid sequences, including those described in Section 5.1, and appropriate transcriptional and translational control signals.

These constructs can be designed to encode and express polypeptides or peptides corresponding to one or more functional domains of the HGPRBMY1 or HGPRBMY2 (e.g., an ECD, a TM and/or a CD) in any order, truncated or deleted HGPRBMY1 or HGPRBMY2 (e.g., HGPRBMY1 or HGPRBMY2 in which one or more TM and/or CD are deleted) as well as fusion polypeptides in which the HGPRBMY1 or HGPRBMY2 or truncation/deletion mutant of HGPRBMY1 or HGPRBMY2 is fused to an unrelated polypeptide (i.e., linked to a heterologous carrier polypeptide) and can be designed on the basis of the HGPRBMY1 or HGPRBMY2 nucleic acid and HGPRBMY1 or HGPRBMY2 amino acid sequences disclosed in this Section and in Section 5. 1, above.

The HGPRBMY1 or HGPRBMY2 polypeptide or peptide may be a soluble derivative, e.g., HGPRBMY1 or HGPRBMY2 domains corresponding to one or more of the CD or ECD (e.g., the four ECD constructed in frame and in tandem without linkers, or likewise the four CD in tandem, or any combination of soluble domains of the polypeptide of the invention); one or more of the ECD or CD linked via a hydrophillic peptide linker sequence and/or a flexible linker sequence (e.g., such as GGSGG); or a truncated or deleted HGPRBMY1 or HGPRBMY2 in which the TM are deleted, the TM and CD are deleted or the TM and ECD are deleted, wherein the peptide or polypeptide can be recovered from the culture, i.e., from the host cell in cases where the HGPRBMY1 or HGPRBMY2 peptide or polypeptide is not secreted, and from the culture media in cases where the HGPRBMY1 or HGPRBMY2 peptide or polypeptide is secreted by the cells. In a preferred embodiment, these polypeptides are soluble in normal physiological conditions.

Fusion polypeptides comprising HGPRBMY1 or HGPRBMY2 polypeptide or peptide sequences fused to heterologous sequences can include, but are not limited to, epitope tagged polypeptides or peptides, e.g., GST fusions, Myc-tag, hemagglutinin-tag, histidine-tag, FLAG-tag, etc.; Ig-Fc fusions which stabilize the HGPRBMY1 or HGPRBMY2 polypeptide or peptide and prolong half-life in vivo; or fusions to any amino acid sequence that allows the fusion polypeptide to be anchored to the cell membrane, allowing the HGPRBMY1 or HGPRBMY2 domain to be exhibited on the cell surface. The fusion polypeptide can also be constructed with a protease cleavage site between the HGPRBMY1 or HGPRBMY2 and the heterologous sequences in order to allow release from the foreign sequences, e.g., thrombin site or factor Xa.

The polypeptides or peptides of the invention can also be conjugated or fused to a compound, such as an enzyme, fluorescent polypeptide, or luminescent polypeptide which provide a marker function. Examples of suitable marker compounds include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride or phycoerythrin, luminol, luciferase, luciferin, aequorin, ¹²⁵I, ¹³¹I, ³⁵S or ³H.

Further, a polypeptide or peptide of the invention may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In addition, a fusion polypeptide or peptide of the invention may be a conjugate or fusion with a drug moiety, which is not to be construed as limited to classical chemical therapeutic agents. For example, the drug moiety may be a polypeptide or polypeptide possessing a desired biological activity. Such polypeptides may include, for example, a toxin such as abrin, ricin A, pseudomonas exotoxin, or diphtheria toxin; a polypeptide such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-4 (“IL-4”), interleukin-6 (“IL-6”), interleukin-7 (“IL-7”), granulocyte macrophage colony stimulating factor (“GM-CSF”), granulocyte colony stimulating factor (“G-CSF”), interleukin-10 (“IL-10”), interleukin-12 (“IL-12”), interleukin-17 (“IL-15”), interleukin-17 (“IL-17”), interferon-γ (“IFN-γ”), interferon-α (“IFN-α”), or other immune factors or growth factors.

Further, HGPRBMY1 or HGPRBMY2 polypeptides of other species are encompassed by the invention. In fact, any HGPRBMY1 or HGPRBMY2 polypeptide encoded by the HGPRBMY1 or HGPRBMY2 nucleic acid sequences described in Section 5. 1, above, are within the scope of the invention.

In another embodiment, polypeptides that are functionally equivalent to the HGPRBMY1 encoded by the nucleic acid sequences described in Section 5.1, as judged by any of a number of criteria, including but not limited to the ability to bind agonist or antagonist, the binding affinity for agonist or antagonist, the resulting biological effect of agonist or antagonist binding, e.g., signal transduction, a change in cellular metabolism (e.g., ion flux) or change in phenotype when the HGPRBMY1 equivalent is present in an appropriate cell type (such as the amelioration, prevention or delay of an immune disorder such as rheumatoid arthritis, leukemia or an immunodeficiency); by its ability to bind or compete with antibodies to HGPRBMY1 receptors; or by its ability to elicit antibodies that immunospecifically bind to the HGPRBMY1 receptor; etc.

The invention also encompasses polypeptides that are functionally equivalent to the HGPRBMY2 encoded by the nucleic acid sequences described in Section 5.1, as judged by any of a number of criteria, including but not limited to the ability to bind an antibody, an agonist or an antagonist, the binding affinity for agonist or antagonist, the resulting biological effect of agonist or antagonist binding, e.g., signal transduction, a change in cellular metabolism (e.g., ion flux, tyrosine phosphorylation) or change in phenotype when the HGPRBMY2 equivalent is present in an appropriate cell type (such as the amelioration, prevention or delay of congestive heart failure); by its ability to bind or compete with antibodies to HGPRBMY2 receptors; or by its ability to elicit antibodies that immunospecifically bind to the HGPRBMY2 receptor; etc.

Such functionally equivalent HGPRBMY1 polypeptides include but are not limited to additions or substitutions of amino acid residues within the amino acid sequence encoded by the HGPRBMY1 nucleic acid sequences described, above, in Section 5.1, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Regional charge in the polypeptide can be determined analytically with computer programs, for example as shown in FIG. 3, which depicts a hydropathy plot of the polypeptide sequence of FIG. 2.

Such functionally equivalent HGPRBMY2 polypeptides include but are not limited to additions or substitutions of amino acid residues within the amino acid sequence encoded by the HGPRBMY2 nucleic acid sequences described, above, in Section 5.1, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Regional charges in the polypeptide can be determined analytically with computer programs, for example as shown in FIG. 8, which depicts a hydropathy plot of the polypeptide sequence of FIG. 7.

While random mutations can be made to HGPRBMY1 or HGPRBMY2 DNA and the resulting mutant HGPRBMY1 or HGPRBMY2 tested for activity, site-directed mutations of the HGPRBMY1 or HGPRBMY2 coding sequence can be engineered using site-directed mutagenesis techniques known to those skilled in the art to generate a mutant HGPRBMY1 or HGPRBMY2 with modulated function, e.g., higher binding affinity for agonist or antagonist, and/or changed signaling capacity, e.g., lower binding affinity for agonist or antagonist.

For example, the alignment of HGPRBMY1 and orexin is shown in FIG. 4 in which identical amino acid residues are indicated by a black background. Mutant HGPRBMY1 can be engineered so that regions of identity (indicated by black background in FIG. 4) are maintained, whereas the variable residues (white background in FIG. 4) are altered, e.g., by deletion or insertion of an amino acid residue(s) or by substitution of one or more different amino acid residues. Conservative alterations at the variable positions can be engineered in order to produce a mutant HGPRBMY1 that retains function; e.g., agonist or antagonist binding affinity or signal transduction capability or both. Non-conservative changes can be engineered at these variable positions to alter function, e.g., agonist or antagonist binding affinity or signal transduction capability, or both.

For example, the alignment of HGPRBMY2 and orexin is shown in FIG. 9 in which identical amino acid residues are indicated by a black background. Mutant HGPRBMY2 can be engineered so that regions of identity (indicated by black background in FIG. 9) are maintained, whereas the variable residues (white background in FIG. 9) are altered, e.g., by deletion or insertion of an amino acid residue(s) or by substitution of one or more different amino acid residues. Conservative alterations at the variable positions can be engineered in order to produce a mutant HGPRBMY2 that retains function; e.g., agonist or antagonist binding affinity or signal transduction capability or both. Non-conservative changes can be engineered at these variable positions to alter function, e.g., agonist or antagonist binding affinity or signal transduction capability, or both.

In addition, mutation by deletion or non-conservative alteration of the conserved regions can be engineered where modulation of function is desired (i.e., identical amino acids indicated by stars in FIG. 4 or FIG. 9). For example, deletion or non-conservative alterations (substitutions or insertions) of the agonist binding domain, can be engineered to produce a mutant HGPRBMY1 or HGPRBMY2 that binds agonist or antagonist but is signaling-incompetent. Non-conservative alterations to the residues with a black background in the ECD shown in FIG. 4 or FIG. 9 can be engineered to produce mutant HGPRBMY1 or HGPRBMY2 with altered binding affinity for agonist or antagonist.

Other mutations to the HGPRBMY1 or HGPRBMY2 coding sequence can be made to generate HGPRBMY1 or HGPRBMY2 that are better suited for expression in host cells, e.g., reduced toxicity, increased solubility, scale up, etc. in host cells. For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions of any one or more of the glycosylation recognition sequences which occur in an ECD (N-X-S or N-X-T), and/or an amino acid deletion at the second position of any one or more such recognition sequences in the ECD will prevent glycosylation of the HGPRBMY1 or HGPRBMY2 at the modified tripeptide sequence. (See, e.g., Miyajima et al., 1986, EMBO J. 5(6): 1193-1197). In addition, the nucleic acid construct can be designed to be polycistronic with alternative splice sites in order to increase production of polypeptides or peptides of the invention per cell, thus increasing yield.

The expression systems also encompass engineered host cells that express the HGPRBMY1 or HGPRBMY2 or functional equivalents in situ, i.e., anchored in the cell membrane. Purification or enrichment of the HGPRBMY1 or HGPRBMY2 from such expression systems can be accomplished using appropriate detergents and lipid micelles and methods well known to those skilled in the art. However, such engineered host cells themselves may be used in situations where it is important not only to retain the structural and functional characteristics of the HGPRBMY1 or HGPRBMY2, but to assess biological activity, e.g., in drug screening assays.

The expression systems that may be used for purposes of the invention include but are not limited to microorganisms such as bacteria (e.g., E. coli, B. subtilis) transformed with recombinant bacteriophage DNA, plasmid DNA or cosmid DNA expression vectors containing HGPRBMY1 or HGPRBMY2 nucleic acid sequences; yeast (e.g., Saccharomyces, Pichia) transformed with recombinant yeast expression vectors containing the HGPRBMY1 or HGPRBMY2 nucleic acid sequences; insect cell systems infected with recombinant virus expression vectors (e.g., baculovirus) containing the HGPRBMY1 or HGPRBMY2 sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing HGPRBMY1 or HGPRBMY2 nucleic acid sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the HGPRBMY1 or HGPRBMY2 gene product being expressed. For example, when a large quantity of such a polypeptide is to be produced, for the generation of pharmaceutical compositions of HGPRBMY1 or HGPRBMY2 polypeptide or for raising antibodies to the HGPRBMY1 or HGPRBMY2 polypeptide, for example, vectors which direct the expression of high levels of fusion polypeptide products that are readily purified may be desirable. For example, pGEX vectors may also be used to express foreign polypeptides as fusion polypeptides with glutathione S-transferase (GST). The pGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. These recombinant viruses are then used to infect Spodoptera frugiperda cells in which the inserted gene is expressed. (e.g., see Smith et al., 1983, J. Virol. 46: 584; Smith, U.S. Pat. No. 4,215,051).

In mammalian host cells, a number of viral-based expression systems may be utilized. In adenovirus, the HGPRBMY1 or HGPRBMY2 nucleic acid sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the HGPRBMY1 or HGPRBMY2 gene product in infected hosts (e.g., See Logan & Shenk, 1984, Proc. Natl. Acad. Sci. USA 81:3655-3659).

In cases where an entire HGPRBMY1 or HGPRBMY2 gene or cDNA, including its own initiation codon and adjacent sequences, is inserted into the appropriate expression vector, no additional translational control signals may be needed. However, in cases where only a portion of the HGPRBMY1 or HGPRBMY2 coding sequence is inserted, exogenous translational control signals, including, perhaps, the ATG initiation codon, must be provided. Furthermore, the initiation codon must be correctly oriented in the reading frame of the desired coding sequence to ensure translation of the insert in the correct reading frame. These exogenous translational control signals and initiation codons can be of a variety of origins, both natural and synthetic. Intronic sequences and polyadenylation signals can also be included to increase the efficiency of expression.

In addition, a host cell strain may be chosen which modulates the expression of the inserted sequences, or modifies and processes the gene product in the specific fashion desired. Such modifications (e.g., glycosylation) and processing (e.g., cleavage) of polypeptide products may be important for the function of the polypeptide. Different host cells have characteristic and specific mechanisms for the post-translational processing and modification of polypeptides and gene products. Appropriate cell lines or host systems can be chosen to ensure the correct modification and processing of the foreign polypeptide expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, and in particular, bone marrow cell lines such as lymphocyte lineage (for example, monocyte, B-cell or T-cell, such as K562, WEHI 7.1 or WEHI-3 cell lines) or erythrocyte lineage cell lines.

For long-term, high-yield production of recombinant polypeptides, stable expression is preferred. For example, cell lines which stably express the HGPRBMY1 or HGPRBMY2 sequences described above may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the HGPRBMY1 or HGPRBMY2 gene product. Such engineered cell lines may be particularly useful in screening and evaluation of compounds that affect the endogenous activity of the HGPRBMY1 or HGPRBMY2 gene product.

A number of selection systems may be used, including but not limited to the herpes simplex virus thymidine kinase (tk) (Wigler, et al., 1977, Cell 11:223), hypoxanthine-guanine phosphoribosyltransferase (hgprt) (Szybalska & Szybalski, 1962, Proc. Natl. Acad. Sci. USA 48:2026), and adenine phosphoribosyltransferase (aprt) (Lowy, et al., 1980, Cell 22:817) genes can be employed in tk⁻, hgprt⁻ or aprt^{'} cells, respectively. Also, antimetabolite resistance can be used as the basis of selection for the following genes: Dihydrofolate Reductase (DHFR), which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

The polypeptides of the invention can, for example, include modifications that can increase such attributes as stability, half-life, ability to enter cells and aid in administration, e.g., in vivo administration of the polypeptides of the invention. For example, polypeptides of the invention can comprise a polypeptide transduction domain of the HIV TAT polypeptide as described in Schwarze, et al. (1999 Science 285:1569-1572), thereby facilitating delivery of polypeptides of the invention into cells.

Alternatively, any fusion polypeptide may be readily purified by utilizing an antibody specific for the fusion polypeptide being expressed. For example, a system described by Janknecht et al. allows for the ready purification of non-denatured fusion polypeptides expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged polypeptides are selectively eluted with imidazole-containing buffers.

The HGPRBMY1 or HGPRBMY2 gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate HGPRBMY1 or HGPRBMY2 transgenic animals.

Any technique known in the art may be used to introduce the HGPRBMY1 or HGPRBMY2 transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229, which is incorporated by reference herein in its entirety.

The present invention provides for transgenic animals that carry the HGPRBMY1 or HGPRBMY2 transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., 1992, Proc. Natl. Acad. Sci. USA 89: 6232-6236). The regulatory sequences required for such a cell-type specific activation will depend upon the particular cell type of interest, and will be apparent to those of skill in the art. When it is desired that the HGPRBMY1 or HGPRBMY2 gene transgene be integrated into the chromosomal site of the endogenous HGPRBMY1 or HGPRBMY2 gene, gene targeting is preferred. Briefly, when such a technique is to be utilized, vectors containing some nucleic acid sequences homologous to the endogenous HGPRBMY1 or HGPRBMY2 gene are designed for the purpose of integrating, via homologous recombination with chromosomal sequences, into and disrupting the function of the nucleic acid sequence of the endogenous HGPRBMY1 or HGPRBMY2 gene. The transgene may also be selectively introduced into a particular cell type, thus inactivating the endogenous HGPRBMY1 or HGPRBMY2 gene in only that cell type, by following, for example, the teaching of Gu et al. (Gu, et al., 1994, Science 265: 103-106). The regulatory sequences required for such a cell-type specific inactivation will depend upon the particular cell type of interest, and will be apparent to those of skill in the art.

Once transgenic animals have been generated, the expression of the recombinant HGPRBMY1 or HGPRBMY2 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of HGPRBMY1 or HGPRBMY2 gene-expressing tissue, may also be evaluated immunocytochemically using antibodies specific for the HGPRBMY1 or HGPRBMY2 transgene product.

5.3. Antibodies to HGPRBMY1

Antibodies that specifically recognize one or more epitopes of HGPRBMY1 or HGPRBMY2, or epitopes of conserved variants of HGPRBMY1 or HGPRBMY2 polypeptides or peptides, are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of the HGPRBMY1 in a biological sample and may, therefore, be utilized as part of a diagnostic or prognostic technique whereby patients may be tested for abnormal amounts of HGPRBMY1. Such antibodies may also be utilized in conjunction with, for example, compound screening schemes, as described, below, in Section 5.5, for the evaluation of the effect of test compounds on expression and/or activity of the HGPRBMY1 gene product. Additionally, such antibodies can be used in conjunction with the gene therapy techniques described, below, in Section 5.6, to, for example, evaluate the normal and/or engineered HGPRBMY1-expressing cells prior to their introduction into the patient. Such antibodies may additionally be used as a method for the inhibition of abnormal HGPRBMY1 activity. Thus, such antibodies may, therefore, be utilized as part of immune disorder treatment methods.

The antibodies of the invention may be used, for example, in the detection of the HGPRBMY2 in a biological sample and may, therefore, be utilized as part of a diagnostic or prognostic technique whereby patients may be tested for abnormal amounts of HGPRBMY2. Such antibodies may also be utilized in conjunction with, for example, compound screening schemes, as described, below, in Section 5.5, for the evaluation of the effect of test compounds on expression and/or activity of the HGPRBMY2 gene product. Additionally, such antibodies can be used in conjunction with the gene therapy techniques described, below, in Section 5.6, to, for example, evaluate the normal and/or engineered HGPRBMY2-expressing cells prior to their introduction into the patient. Such antibodies may additionally be used as a method for the inhibition of abnormal HGPRBMY2 activity. Thus, such antibodies may, therefore, be utilized as part of heart disorder treatment methods.

In a particular embodiment, HGPRBMY1 expression can be utilized as a marker (e.g., an in situ marker) for specific tissues (e.g., bone marrow, spleen or thymus) and/or cells (e.g., lymphocytes) in which HGPRBMY1 is expressed.

In a particular embodiment, HGPRBMY2 expression can be utilized as a marker (e.g., an in situ marker) for specific tissues (e.g., heart, brain, etc.) and/or cells (e.g., cells shown in FIGS. 10 and 16) in which HGPRBMY2 is expressed.

An isolated polypeptide or peptide of the invention can be used as an immunogen to generate antibodies using standard techniques for polyclonal and monoclonal antibody preparation. The full-length polypeptide or a functional domain of the polypeptide, either native or denatured, can be used or, alternatively, the invention provides antigenic polypeptides or peptides for use as immunogens. The antigenic peptide of a polypeptide of the invention comprises at least 8 (preferably 10, 15, 20, or 30) amino acid residues of the amino acid sequence of SEQ ID NO:2 or SEQ ID NO:14 or a variant thereof, and features an epitope of the polypeptide such that an antibody raised against the peptide forms a specific immune complex with the polypeptide, and alternatively with a native polypeptide.

Preferred epitopes encompassed by the antigenic peptide are regions that are located on the surface of the polypeptide, e.g., hydrophilic regions, for example, as shown in hydrophilic regions in FIG. 3 or FIG. 8. In certain embodiments, the nucleic acid molecules of the invention are present as part of nucleic acid molecules comprising nucleic acid sequences that contain or encode heterologous (e.g., vector, expression vector, or fusion polypeptide) sequences. These nucleotides can then be used to express polypeptides which can be used as immunogens to generate an immune response, or more particularly, to generate polyclonal or monoclonal antibodies specific to the expressed polypeptide.

An immunogen typically is used to prepare antibodies by immunizing a suitable subject, (e.g., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human polypeptides such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a polypeptide or peptide of the invention can be selected for (e.g., partially purified) or purified by, e.g., affinity chromatography. For example, a recombinantly expressed and purified (or partially purified) polypeptide of the invention is produced as described herein, and covalently or non-covalently coupled to a solid support such as, for example, a chromatography column. The column can then be used to affinity purify antibodies specific for the polypeptides of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired polypeptide or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired polypeptide or peptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the polypeptide (e.g., in a cellular lysate or cell supernatant) in order to evaluate the abundance and pattern of expression of the polypeptide. The antibodies can also be used diagnostically to monitor polypeptide levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In addition, the HGPRBMY1 or HGPRBMY2 gene sequences and gene products, including polypeptides, peptides, fusion polypeptides or peptides, and antibodies directed against said gene products and peptides, have applications for purposes independent of the role of the gene products. For example, HGPRBMY1 or HGPRBMY2 gene products, including polypeptides or peptides, as well as specific antibodies thereto, can be used for construction of fusion polypeptides to facilitate recovery, detection, or localization of another polypeptide of interest. In addition, HGPRBMY1 or HGPRBMY2 genes and gene products can be used for genetic mapping. Finally, HGPRBMY1 or HGPRBMY2 nucleic acids and gene products have generic uses, such as supplemental sources of nucleic acids, polypeptides and amino acids for food additives or cosmetic products.

Further, an antibody of the invention (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In addition, polypeptides, agonists or antagonists which bind a polypeptide of the invention can also be conjugated to the foregoing, thereby targeting a toxin to cells expressing HGPRBMY1 or HGPRBMY2.

The conjugates of the invention can be used for modifying a given biological response, the drug moiety is not to be construed as limited to classical chemical therapeutic agents. For example, the drug moiety may be a polypeptide or peptide possessing a desired biological activity. Such polypeptides may include, for example, a toxin such as abrin, ricin A, pseudomonas exotoxin, or diphtheria toxin; a polypeptide such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin- 1 (“IL-1”), interleukin-2 (“IL-2”), interleukin-4 (“IL-4”), interleukin-6 (“IL-6”), interleukin-7 (“IL-7”), granulocyte macrophage colony stimulating factor (“GM-CSF”), granulocyte colony stimulating factor (“G-CSF”), interleukin-10 (“IL-10”), interleukin-12 (“IL-12”), interleukin-17 (“IL-15”), interleukin-17 (“IL-17”), interferon-γ (“IFN-γ”), interferon-α (“IFN-α”), or other immune factors or growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

An antibody with or without a therapeutic moiety conjugated to it can be used as a therapeutic that is administered alone or in combination with chemotherapeutic agents.

Alternatively, an antibody of the invention can be conjugated to a second antibody to form an “antibody heteroconjugate” as described by Segal in U.S. Pat. No. 4,676,980 or alternatively, the antibodies can be conjugated to form an “antibody heteropolymer” as described in Taylor et al., in U.S. Pat. Nos. 5,470,570 and 5,487,890.

An antibody with or without a therapeutic moiety conjugated to it can be used as a therapeutic that is administered alone or in combination with cytotoxic factor(s) and/or cytokine(s).

In yet a further aspect, the invention provides substantially purified antibodies or fragments thereof, including human or non-human antibodies or fragments thereof, which antibodies or fragments specifically bind to a polypeptide of the invention comprising an amino acid sequence of SEQ ID NO:2 or SEQ ID NO:14 or a variant thereof. In various embodiments, the substantially purified antibodies of the invention, or fragments thereof, can be human, non-human, chimeric and/or humanized antibodies.

In another aspect, the invention provides human or non-human antibodies or fragments thereof, which antibodies or fragments specifically bind to a polypeptide comprising an amino acid sequence of SEQ ID NO:2 or SEQ ID NO:14 or a variant thereof. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide of the invention comprising an amino acid sequence of SEQ ID NO:2 or SEQ ID NO:14 or a variant thereof. The monoclonal antibodies can be human, humanized, chimeric and/or non-human antibodies.

The substantially purified antibodies or fragments thereof specifically bind to a signal peptide, a secreted sequence, an extracellular domain, a transmembrane or a cytoplasmic domain cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence, or alternatively, to an extracellular domain of the amino acid sequence of the invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes HGPRBMY1 or HGPRBMY2, the method comprising immunizing a mammal with a polypeptide. After immunization, a sample is collected from the mammal that contains an antibody that specifically recognizes the immunogen. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against HGPRBMY1 or HGPRBMY2 gene products. Single chain antibodies are formed by linking the heavy and light chain fragments of the Fv region via an amino acid bridge, resulting in a single chain polypeptide.

Antibodies to the HGPRBMY1 can, in turn, be utilized to generate anti-idiotype antibodies that “mimic” the HGPRBMY1, using techniques well known to those skilled in the art (See, e.g., Greenspan & Bona, 1993, FASEB J 7(5):437-444; and Nissinoff, 1991, J. Immunol. 147(8):2429-2438). For example antibodies which bind to the HGPRBMY1 ECD and competitively inhibit the binding of agonist or antagonist to the HGPRBMY1 can be used to generate anti-idiotypes that “mimic” the ECD and, therefore, bind and neutralize agonist or antagonist. Such neutralizing anti-idiotypes or Fab fragments of such anti-idiotypes can be used in therapeutic regimens to neutralize agonist or antagonist and prevent immune disorders.

Antibodies to the HGPRBMY2 can, in turn, be utilized to generate anti-idiotype antibodies that “mimic” the HGPRBMY2, using techniques well known to those skilled in the art. (See, e.g., Greenspan & Bona, 1993, FASEB J 7(5):437-444; and Nissinoff, 1991, J. Immunol. 147(8):2429-2438). For example antibodies which bind to the HGPRBMY2 ECD and competitively inhibit the binding of agonist or antagonist to the HGPRBMY2 can be used to generate anti-idiotypes that “mimic” the ECD and, therefore, bind and neutralize agonist or antagonist. Such neutralizing anti-idiotypes or Fab fragments of such anti-idiotypes can be used in therapeutic regimens to neutralize agonist or antagonist and prevent heart failure or neural disorders.

5.4. Diagnosis of Immune Disorders

A variety of methods can be employed for the diagnostic and prognostic evaluation of immune disorders and for the identification of subjects having a predisposition to such disorders.

Immune system disorders occur when the immune response is inappropriate, excessive, or lacking. Immunodeficiency disorders occur when the immune system fails to fight tumors or invading substances. This causes persistent or recurrent infections, severe infections by organisms that are normally mild, incomplete recovery from illness or poor response to treatment, and increased incidence of cancer and other tumors. Opportunistic infections are widespread infections by microorganisms that are usually controllable.

People are said to be “immunosuppressed” when they experience immunodeficiency that is caused by medications such as corticosteroids or immunosuppressant (chemotherapy) medications. This is a desired part of treatment for disorders such as autoimmune disorders. It is used after organ transplantation to prevent transplant rejection. Acquired immunodeficiency may be a complication of diseases such as HIV, infection and AIDS (acquired immunodeficiency syndrome), or from malnutrition.

Various immune disorders include, but are not limited to: congenital immunodeficiency, Anemia, Antiphospholipid Syndrome (APS), Blue Rubber Bleb Nevus Syndrome, Gout, Hemophilia, Leukemia, Myeloproliferative Disorders, Sickle Cell Disease, and Thalassemia. Additionally, diseases which affect immune function are contemplated, for example those that cause immunodeficiency such as AIDS/HIV.

Examples of congenital immunodeficiency disorders of antibody production (B lymphocyte abnormalities) include hypo-gammaglobulinemia (lack of one or more specific antibodies), which usually causes repeated mild respiratory infections, and agammaglobulinemia (lack of all or most antibody production), which results in frequent severe infections and is often fatal. Congenital disorders affecting the T lymphocytes may cause increased susceptibility to fungi, resulting in repeated Candida (yeast) infections. Inherited combined immunodeficiency affects both T lymphocytes and B lymphocytes.

The following conditions and diseases often result in an immunodeficient state: ataxia-telangiectasia, DiGeorge syndrome, Chediak-Higashi syndrome, Job syndrome, leukocyte adhesion defects, panhypogammaglobulinemia, Bruton disease, congenital agammaglobulinemia, selective deficiency of IgA, combined immunodeficiency disease, Wiscott-Aldrich syndrome, and complement deficiencies.

Suppression of the immune system may be desired in the treatment of certain disorders, or it may be a side effect of some treatments, for example in organ or bone marrow transplantation.

Immune deficiency is identified partly by poor response to treatment, delayed or incomplete recovery from illness, the presence of certain types of cancers (such as Kaposi's sarcoma), opportunistic infections (such as widespread Pneumocystis carinii infection or recurrent fungal/yeast infections).

Autoimmune disorders occur when the normal control process is disrupted. They may also occur if normal body tissue is altered so that it is no longer recognized as “self.” Because autoimmune disorders and allergy are both caused by hypersensitivity reactions, it is believed that a history of allergy indicates increased risk for autoimmune disorders.

Examples of autoimmune (or autoimmune-related) disorders include but are not limited to: Hashimoto's thyroiditis, pernicious anemia, Addison's disease, diabetes mellitus, rheumatoid arthritis, systemic lupus erythematosus, dermatomyositis, Sjogren's syndrome, dermatomyositis, lupus erythematosus, multiple sclerosis, myasthenia gravis, Reiter's syndrome, and Graves disease.

Additional immune disorders include: Giant Lymph Node Hyperplasia, Castleman disease, Small Bowel Nodules, Immunoblastic Lymphadenopathy, Immunoproliferative Small Intestinal Disease, myelodysplasia syndrome 1, Still's syndrome, Lymphangiomyoma, Lymphoma, Abdominal Visceral Lymphoma, Bilaterally Large Multifocal Kidneys, Marek's Disease, Sezary Syndrome, Mycosis Fungoides and Tumor Lysis Syndrome.

Organs and tissues commonly affected by autoimmune disorders include blood components such as red blood cells, blood vessels, connective tissues, endocrine glands such as the thyroid or pancreas, muscles, joints, and skin. A person may experience more than one autoimmune disorder at the same time. Some disorders have multiple interrelated causes, one of which is autoimmunity.

Leukemias are defined generally as a group of usually fatal diseases of the reticuloendothelial system involving uncontrolled proliferation of white blood cells (leukocytes) such as: chronic myelogenous leukemia (CML), hairy cell leukemia, chronic lymphocytic leukemia (CLL), acute lymphocytic leukemia, acute nonlymphocytic leukemia (AML), and chronic myelomonocytic leukemia.

Moreover, as the compositions of the invention relate to bone marrow, it is also contemplated that BMPRBMY1 can be targeted for modulation of anemia. Anemias which can be treated by methods of the invention include but are not limited to: anemia of B12 deficiency, anemia of chronic disease, anemia of folate deficiency, drug-induced immune hemolytic anemia, hemolytic anemia, hemolytic anemia due to g6pd deficiency, idiopathic aplastic anemia, idiopathic autoimmune hemolytic anemia, immune hemolytic anemia, iron deficiency anemia, megaloblastic anemia, pernicious anemia, secondary aplastic anemia, and sickle cell anemia.

Other HGPRBMY1 associated disorders can include TNF related disorders (e.g., acute myocarditis, myocardial infarction, congestive heart failure, T cell disorders (e.g., dermatitis, fibrosis)), immunological differentiative and apoptotic disorders (e.g., hyper-proliferative syndromes such as systemic lupus erythematosus (lupus)), and disorders related to angiogenesis (e.g., tumor formation and/or metastasis, cancer). Modulators of HGPRBMY1 expression and/or activity can be used to treat such disorders.

Methods of diagnosing or detecting immune disorders may, for example, utilize reagents such as the HGPRBMY1 nucleic acid sequences described in Section 5.1, and HGPRBMY1 antibodies, as described, in Section 5.3. Specifically, such reagents may be used, for example, for: (1) the detection of the presence of HGPRBMY1 gene mutations, or the detection of either over- or under-expression of HGPRBMY1 mRNA relative to the non-immune related disorder state; (2) the detection of either an over- or an under-abundance of HGPRBMY1 gene product relative to the non-immune related disorder state; and (3) the detection of perturbations or abnormalities in the signal transduction pathway mediated by HGPRBMY1.

The methods described herein may be performed, for example, by utilizing pre-packaged diagnostic kits comprising at least one specific HGPRBMY1 nucleic acid sequence or HGPRBMY1 antibody reagent described herein, which may be conveniently used, e.g., in clinical settings, to diagnose patients exhibiting immune related disorder abnormalities.

For the detection of HGPRBMY1 mutations, any nucleated cell can be used as a starting source for genomic nucleic acid. For the detection of HGPRBMY1 gene expression or HGPRBMY1 gene products, any cell type or tissue in which the HGPRBMY1 gene is expressed, such as, for example, immune cells, may be utilized.

Nucleic acid-based detection techniques are described, below, in Section 5.4.1. Peptide detection techniques are described, below, in Section 5.4.2.

5.4b. Diagnosis of Cardiovascular Disorders

A variety of methods can be employed for the diagnostic and prognostic evaluation of HGPRBMY2-related cardiovascular disorders and for the identification of subjects having a predisposition to such disorders. Various forms of heart disease include: cardiomyopathy, aortic valve prolapse; aortic valve stenosis; arrhythmia; cardiogenic shock; congenital heart disease; heart attack; heart failure; heart tumor; heart valve pulmonary stenosis; idiopathic cardiomyopathy; ischemic cardiomyopathy; mitral regurgitation (acute); mitral regurgitation (chronic); mitral stenosis; mitral valve prolapse; stable angina; hypotension; hypertension; acute heart failure; angina pectoris; and tricuspid regurgitation.

Congestive heart failure may affect either the right side, left side, or both sides of the heart. As pumping action is lost, blood may back up into other areas of the body, including the liver, gastrointestinal tract, and extremities (right-sided heart failure), or the lungs (left-sided heart failure).

Structural or functional causes of heart failure include high blood pressure (hypertension), heart valve disease, congenital heart diseases, cardiomyopathy, heart tumor, and other heart diseases. Precipitating factors include infections with high fever or complicated infections, use of negative inotropic drugs (such as β-blocker and calcium channel blocker), anemia, irregular heartbeats (arrhythmia), hyperthyroidism, and kidney disease.

Furthermore, cardiomyopathy is a disease affecting the heart muscle (myocardium); this disease usually results in the inadequate heart pumping. Causes, incidence, and risk factors for cardiomyopathy include: viral infections; heart attacks; alcoholism; long-term, severe high blood pressure (hypertension); or for other reasons not yet known. Specific types of cardiomyopathy include: ischemic cardiomyopathy; idiopathic cardiomyopathy; hypertrophic cardiomyopathy; alcoholic cardiomyopathy; peripartum cardiomyopathy; dilated cardiomyopathy; and restrictive cardiomyopathy. Cardiomyopathy is not common but can be severely disabling or fatal. Extreme cardiomyopathy with heart failure may require a heart transplant.

Methods of diagnosing or detecting heart diseases may, for example, utilize reagents such as the HGPRBMY2 nucleic acid sequences described in Section 5.1, and HGPRBMY2 antibodies, as described, in Section 5.3. Specifically, such reagents may be used, for example, for: (1) the detection of the presence of HGPRBMY2 gene mutations, or the detection of either over- or under-expression of HGPRBMY2 mRNA relative to the non-cardiovascular disorder state; (2) the detection of either an over- or an under-abundance of HGPRBMY2 gene product relative to the non-cardiovascular disorder state; and (3) the detection of perturbations or abnormalities in the signal transduction pathway mediated by HGPRBMY2.

The methods described herein may be performed, for example, by utilizing pre-packaged diagnostic kits comprising at least one specific HGPRBMY2 nucleic acid sequence or HGPRBMY2 antibody reagent described herein, which may be conveniently used, e.g., in clinical settings, to diagnose patients exhibiting cardiovascular disorder abnormalities.

For the detection of HGPRBMY2 mutations, any nucleated cell can be used as a starting source for genomic nucleic acid. For the detection of HGPRBMY2 gene expression or HGPRBMY2 gene products, any cell type or tissue in which the HGPRBMY2 gene is expressed, such as, for example, heart cells, may be utilized.

Nucleic acid-based detection techniques are described, below, in Section 5.4.1. Peptide detection techniques are described, below, in Section 5.4.2.

5.4.1. Detection of the HGPRBMY1 and HGPRBMY2 Gene and Transcripts

Mutations within the HGPRBMY1 or HGPRBMY2 gene can be detected by utilizing a number of techniques. Nucleic acid from any nucleated cell can be used as the starting point for such assay techniques, and may be isolated according to standard nucleic acid preparation procedures which are well known to those of skill in the art.

DNA may be used in hybridization or amplification assays of biological samples to detect abnormalities involving HGPRBMY1 or HGPRBMY2 gene structure, including point mutations, insertions, deletions and chromosomal rearrangements. Such assays may include, but are not limited to, Southern analyses, single stranded conformational polymorphism analyses (SSCP), and PCR analyses.

Such diagnostic methods for the detection of HGPRBMY1 or HGPRBMY2 gene-specific mutations can involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described in Section 5.1, under conditions favorable for the specific annealing of these reagents to their complementary sequences within the HGPRBMY1 or HGPRBMY2 gene. Preferably, the lengths of these nucleic acid reagents are at least 15 to 30 nucleotides. After incubation, all non-annealed nucleic acids are removed from the nucleic acid:HGPRBMY1 or HGPRBMY2 molecule hybrid.

The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents of the type described in Section 5.1 are easily removed. Detection of the remaining, annealed, labeled HGPRBMY1 or HGPRBMY2 nucleic acid reagents is accomplished using standard techniques well-known to those in the art. The HGPRBMY1 or HGPRBMY2 gene sequences to which the nucleic acid reagents have annealed can be compared to the annealing pattern expected from a normal HGPRBMY1 or HGPRBMY2 gene sequence in order to determine whether an HGPRBMY1 or HGPRBMY2 gene mutation is present.

Alternative diagnostic methods for the detection of HGPRBMY1 or HGPRBMY2 gene specific nucleic acid molecules, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the HGPRBMY1 or HGPRBMY2 gene in order to determine whether an HGPRBMY1 or HGPRBMY2 gene mutation exists.

Additionally, well-known genotyping techniques can be performed to identify individuals carrying HGPRBMY1 or HGPRBMY2 gene mutations. Such techniques include, for example, the use of restriction fragment length polymorphisms (RFLPs), which involve sequence variations in one of the recognition sites for the specific restriction enzyme used.

Additionally, improved methods for analyzing DNA polymorphisms which can be utilized for the identification of HGPRBMY1 or HGPRBMY2 gene mutations have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)n-(dG-dT)n short tandem repeats. The average separation of (dC-dA)n-(dG-dT)n blocks. is estimated to be 30,000-60,000 bp. Markers which are so closely spaced exhibit a high frequency co-inheritance, and are extremely useful in the identification of genetic mutations, such as, for example, mutations within the HGPRBMY1 or HGPRBMY2 gene, and the diagnosis of diseases and disorders related to HGPRBMY1 or HGPRBMY2 mutations.

A DNA profiling assay for detecting short tri and tetra nucleotide repeat sequences has been described (U.S. Pat. No. 5,364,759, which is incorporated herein by reference in its entirety). This process includes extracting the DNA of interest, such as the HGPRBMY1 or HGPRBMY2 gene, amplifying the extracted DNA, and labeling the repeat sequences to form a genotypic map of the individual's DNA.

The level of HGPRBMY1 or HGPRBMY2 gene expression can also be assayed by detecting and measuring HGPRBMY1 or HGPRBMY2 transcription. For example, RNA from a cell type or tissue known, or suspected to express the HGPRBMY1 or HGPRBMY2 gene, such as bone marrow or spleen cells, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the HGPRBMY1 or HGPRBMY2 gene. Such analyses may reveal both quantitative and qualitative aspects of the expression pattern of the HGPRBMY1 or HGPRBMY2 gene, including activation or inactivation of HGPRBMY1 or HGPRBMY2 gene expression.

In one embodiment of such a detection scheme, cDNAs are synthesized from the RNAs of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the HGPRBMY1 or HGPRBMY2 nucleic acid reagents described in Section 5.1. The preferred lengths of such nucleic acid reagents are at least 9-30 nucleotides. For detection of the amplified product, the nucleic acid amplification may be performed using radioactively or non-radioactively labeled nucleic acids. Alternatively, enough amplified product may be made such that the product may be visualized by standard ethidium bromide staining or by utilizing any other suitable nucleic acid staining method.

Additionally, it is possible to perform such HGPRBMY1 or HGPRBMY2 gene expression assays “in situ”, i.e., directly upon tissue sections (fixed and/or frozen) of patient tissue obtained from biopsies or resections, such that no nucleic acid purification is necessary. Nucleic acid reagents such as those described in Section 5.1 may be used as probes and/or primers for such in situ procedures (See, for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of the HGPRBMY1 or HGPRBMY2 gene.

5.4.2. Detection of the HGPRBMY1 and HGPRBMY2 Gene Products

Antibodies directed against wild type or mutant HGPRBMY1 or HGPRBMY2 gene products or conserved variants of the polypeptides or peptides, which are discussed, above, in Section 5.3, may also be used as immune related disorder diagnostics and prognostics, as described herein. Such diagnostic methods, may be used to detect abnormalities in the level of HGPRBMY1 or HGPRBMY2 gene expression, or abnormalities in the structure and/or temporal, tissue, cellular, or subcellular location of the HGPRBMY1 or HGPRBMY2, and may be performed in vivo or in vitro, such as, for example, on biopsy tissue.

For example, antibodies directed to epitopes of the HGPRBMY1 or HGPRBMY2 ECD can be used in vivo to detect the pattern and level of expression of the HGPRBMY1 or HGPRBMY2 in the body. Such antibodies can be labeled, e.g., with a radio-opaque or other appropriate compound and injected into a subject in order to visualize binding to the HGPRBMY1 or HGPRBMY2 expressed in the body using methods such as X-rays, CAT-scans, or MRI. Labeled antibody fragments, e.g., the Fab or single chain antibody comprising the smallest portion of the antigen binding region, are preferred for maximum labeling of HGPRBMY1 or HGPRBMY2 expressed in the bone marrow or spleen.

Additionally, any HGPRBMY1 or HGPRBMY2 fusion polypeptide or HGPRBMY1 or HGPRBMY2 conjugated polypeptide whose presence can be detected, can be administered. For example, HGPRBMY1 or HGPRBMY2 fusion or conjugated polypeptides labeled with a radio-opaque or other appropriate compound can be administered and visualized in vivo for labeled antibodies. Further such agonist or antagonist fusion polypeptides as AP-GPCR on GPCR-Ap fusion polypeptides can be utilized for in vitro diagnostic procedures. Alternatively, immunoassays or fusion polypeptide detection assays, can be utilized on biopsy and autopsy samples in vitro to permit assessment of the expression pattern of the HGPRBMY1 or HGPRBMY2. Such assays are not confined to the use of antibodies that define the HGPRBMY1 or HGPRBMY2 ECD, but can include the use of antibodies directed to epitopes of any of the domains of the HGPRBMY1 or HGPRBMY2, e.g., the ECD, the TM and/or CD. The use of each or all of these labeled antibodies will yield useful information regarding translation and intracellular transport of the HGPRBMY1 or HGPRBMY2 to the cell surface, and can identify defects in processing.

The tissue or cell type to be analyzed will generally include those which are known, or suspected, to express the HGPRBMY1 or HGPRBMY2 gene, such as, for example, bone marrow or spleen cells. The polypeptide isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the HGPRBMY1 or HGPRBMY2 gene. For example, antibodies, or fragments of antibodies, such as those described, above, in Section 5.3, useful in the present invention may be used to quantitatively or qualitatively detect the presence of HGPRBMY1 or HGPRBMY2 gene products or conserved variants of the polypeptides or peptides. This can be accomplished, for example, by immunofluorescence techniques employing a fluorescently labeled antibody (see below, this Section) coupled with light microscopic, flow cytometric, or fluorimetric detection. Such techniques are especially preferred if such HGPRBMY1 or HGPRBMY2 gene products are expressed on the cell surface.

The antibodies (or fragments thereof) or agonist or antagonist fusion or conjugated polypeptides useful in the present invention may, additionally, be employed histologically, as in immunofluorescence, immunoelectron microscopy or non-immuno assays, for in situ detection of HGPRBMY1 or HGPRBMY2 gene products or conserved variants of the polypeptides or peptides, or for agonist or antagonist binding (in the case of labeled agonist or antagonist fusion polypeptide).

In situ detection may be accomplished by removing a histological specimen from a patient, and applying thereto a labeled antibody or fusion polypeptide of the present invention. The antibody (or fragment) or fusion polypeptide is preferably applied by overlaying the labeled antibody (or fragment) onto a biological sample. Through the use of such a procedure, it is possible to determine not only the presence of the HGPRBMY1 or HGPRBMY2 gene product, or conserved variants of the polypeptides or peptides, or agonist or antagonist binding, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for HGPRBMY1 or HGPRBMY2 gene products or conserved variants of the polypeptides or peptides will typically comprise incubating a sample, such as a biological fluid, a tissue extract, freshly harvested cells, or lysates of cells which have been incubated in cell culture, in the presence of a detectably labeled antibody capable of identifying HGPRBMY1 or HGPRBMY2 gene products or conserved variants of the polypeptides or peptides, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble polypeptides. The support may then be washed with suitable buffers followed by treatment with the detectably labeled HGPRBMY1 or HGPRBMY2 antibody or agonist or antagonist fusion polypeptide. The solid phase support may then be washed with the buffer a second time to remove unbound antibody or fusion polypeptide. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation. The binding activity of a given lot of HGPRBMY1 or HGPRBMY2 antibody or agonist or antagonist fusion polypeptide may be determined according to well known methods.

Those skilled in the art will be able to determine optimal assay conditions for each determination by employing routine experimentation.

With respect to antibodies, one of the ways in which the HGPRBMY1 or HGPRBMY2 antibody can be detectably labeled is by linking the same to an enzyme and used in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo).

The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alphaglycerophosphate, dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect HGPRBMY1 or HGPRBMY2 through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic polypeptide increases the efficiency of the chemiluminescent reaction. The presence of a bioluminescent polypeptide is determined by detecting the presence of luminescence. Important bioluminescent compounds for purposes of labeling are luciferin, luciferase and aequorin.

5.5. Screening Assays for Compounds that Modulate HGPRBMY1 and HGPRBMY2

The following assays are designed to identify compounds that interact with (e.g., bind to) HGPRBMY1 or HGPRBMY2 (including, but not limited to the ECD or CD of HGPRBMY1 or HGPRBMY2), compounds that interact with (e.g., bind to) intracellular polypeptides that interact with HGPRBMY1 or HGPRBMY2 (including, but not limited to, the TM and CD of HGPRBMY1 or HGPRBMY2), compounds that interfere with the interaction of HGPRBMY1 or HGPRBMY2 with transmembrane or intracellular polypeptides involved in HGPRBMY1 or HGPRBMY2-mediated signal transduction, and to compounds which modulate the activity of HGPRBMY1 or HGPRBMY2 gene (i.e., modulate the level of HGPRBMY1 or HGPRBMY2 gene expression) or modulate the level of HGPRBMY1 or HGPRBMY2. Assays may additionally be utilized which identify compounds which bind to HGPRBMY1 or HGPRBMY2 gene regulatory sequences (e.g., promoter sequences) and which may modulate HGPRBMY1 or HGPRBMY2 gene expression. See e.g., Platt, K. A., 1994, J. Biol. Chem. 269:28558-28562, which is incorporated herein by reference in its entirety.

The compounds which may be screened in accordance with the invention include, but are not limited to peptides, antibodies and fragments thereof, and other organic compounds (e.g., peptidomimetics) that bind to the ECD of the HGPRBMY1 or HGPRBMY2 and either mimic the activity triggered by the natural ligand (i.e., agonists) or inhibit the activity triggered by the natural ligand (i.e., antagonists); as well as peptides, antibodies or fragments thereof, and other organic compounds that mimic the ECD of the HGPRBMY1 or HGPRBMY2 (or a portion thereof) and bind to and “neutralize” natural ligand.

Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include but are not limited to small organic molecules which may gain entry into an appropriate cell (e.g., in the bone marrow or spleen) and affect the expression of the HGPRBMY1 gene or some other gene involved in the HGPRBMY1 signal transduction pathway (e.g., by interacting with the regulatory region or transcription factors involved in gene expression); or such compounds that affect the activity of the HGPRBMY1 (e.g., by inhibiting or enhancing the enzymatic activity of the CD) or the activity of some other intracellular factor involved in the HGPRBMY1 signal transduction pathway, such as, for example, gp130.

Other compounds which can be screened in accordance with the invention include but are not limited to small organic molecules which may gain entry into an appropriate cell (e.g., in the heart) and affect the expression of the HGPRBMY2 gene or some other gene involved in the HGPRBMY2 signal transduction pathway (e.g., by interacting with the regulatory region or transcription factors involved in gene expression); or such compounds that affect the activity of the HGPRBMY2 (e.g., by inhibiting or enhancing the enzymatic activity of the CD) or the activity of some other intracellular factor involved in the HGPRBMY2 signal transduction pathway, such as, for example, gp130.

Computer modelling and searching technologies permit identification of compounds, or the improvement of already identified compounds, that can modulate HGPRBMY1 or HGPRBMY2 expression or activity. Having identified such a compound or composition, the active sites or regions are identified. Such active sites might typically be ligand binding sites, such as the interaction domains of agonist or antagonist with HGPRBMY1 or HGPRBMY2 itself. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modelling can be used to complete the structure or improve its accuracy. Any recognized modelling method may be used, including parameterized models specific to particular biopolymers such as polypeptides or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a seach can be manual, but is preferably computer assisted. These compounds found from this search are potential HGPRBMY1 or HGPRBMY2 modulating compounds.

Alternatively, these methods can be used to identify improved modulating compounds from an already known modulating compound or ligand. The composition of the known compound can be modified and the structural effects of modification can be determined using the experimental and computer modelling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of agonist or antagonist, HGPRBMY1 or HGPRBMY2, and related transduction and transcription factors will be apparent to those of skill in the art.

Examples of molecular modelling systems are the CHARMM and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific-polypeptides, such as Rotivinen, et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 141-162; and, with respect to a model receptor for nucleic acid components, Askew, et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular polypeptides, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including polypeptides, for compounds which are inhibitors or activators.

Compounds identified via assays such as those described herein may be useful, for example, in elaborating the biological function of the HGPRBMY1 gene product, and for ameliorating immune disorders. Assays for testing the effectiveness of compounds, identified by, for example, techniques such as those described in Section 5.5.1 through 5.5.3, are discussed, below, in Section 5.5.4.

Compounds identified via assays such as those described herein may be useful, for example, in elaborating the biological function of the HGPRBMY2 gene product, and for ameliorating cardiovascular disorders. Assays for testing the effectiveness of compounds, identified by, for example, techniques such as those described in Section 5.5.1 through 5.5.3, are discussed, below, in Section 5.5.4.

The human HGPRBMY1 or HGPRBMY2 polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a HGPRBMY1 or HGPRBMY2 polypeptide, or a bindable peptide fragment, of this invention, comprising providing a plurality of compounds, combining the HGPRBMY1 or HGPRBMY2 polypeptide, or a bindable peptide fragment, with each of a plurality of compounds for a time sufficient to allow binding under suitable conditions and detecting binding of the HGPRBMY1 or HGPRBMY2 polypeptide or peptide to each of the plurality of test compounds, thereby identifying the compounds that specifically bind to the HGPRBMY1 or HGPRBMY2 polypeptide or peptide.

Methods of identifying compounds that modulate the activity of the novel human HGPRBMY1 or HGPRBMY2 polypeptides and/or peptides are provided by the present invention and comprise combining a potential or candidate compound or drug modulator of G-protein coupled receptor biological activity with an HGPRBMY1 or HGPRBMY2 polypeptide or peptide, for example, the HGPRBMY1 or HGPRBMY2 amino acid sequence as set forth in SEQ ID NO:2 or SEQ ID NO:14, and measuring an effect of the candidate compound or drug modulator on the biological activity of the HGPRBMY1 or HGPRBMY2 polypeptide or peptide. Such measurable effects include, for example, physical binding interaction; the ability to cleave a suitable G-protein coupled receptor substrate; effects on native and cloned HGPRBMY1 or HGPRBMY2-expressing cell line; and effects of modulators or other G-protein coupled receptor-mediated physiological measures.

Another method of identifying compounds that modulate the biological activity of the novel HGPRBMY1 or HGPRBMY2 polypeptides of the present invention comprises combining a potential or candidate compound or drug modulator of a G-protein coupled receptor biological activity with a host cell that expresses the HGPRBMY1 or HGPRBMY2 polypeptide and measuring an effect of the candidate compound or drug modulator on the biological activity of the HGPRBMY1 or HGPRBMY2 polypeptide. The host cell can also be capable of being induced to express the HGPRBMY1 or HGPRBMY2 polypeptide, e.g., via inducible expression. Physiological effects of a given modulator candidate on the HGPRBMY1 or HGPRBMY2 polypeptide can also be measured. Thus, cellular assays for particular G-protein coupled receptor modulators may be either direct measurement or quantification of the physical biological activity of the HGPRBMY1 or HGPRBMY2 polypeptide, or they may be measurement or quantification of a physiological effect. Such methods preferably employ a HGPRBMY1 or HGPRBMY2 polypeptide as described herein, or an overexpressed recombinant HGPRBMY1 or HGPRBMY2 polypeptide in suitable host cells containing an expression vector as described herein, wherein the HGPRBMY1 or HGPRBMY2 polypeptide is expressed, overexpressed, or undergoes upregulated expression.

Another aspect of the present invention embraces a method of screening for a compound that is capable of modulating the biological activity of a HGPRBMY1 or HGPRBMY2 polypeptide, comprising providing a host cell containing an expression vector harboring a nucleic acid sequence encoding a HGPRBMY1 or HGPRBMY2 polypeptide, or a functional peptide or portion thereof (e.g., SEQ ID NOS:2); determining the biological activity of the expressed HGPRBMY1 or HGPRBMY2 polypeptide in the absence of a modulator compound; contacting the cell with the modulator compound and determining the biological activity of the expressed HGPRBMY1 or HGPRBMY2 polypeptide in the presence of the modulator compound. In such a method, a difference between the activity of the HGPRBMY1 or HGPRBMY2 polypeptide in the presence of the modulator compound and in the absence of the modulator compound indicates a modulating effect of the compound.

Essentially any chemical compound can be employed as a potential modulator or ligand in the assays according to the present invention. Compounds tested as G-protein coupled receptor modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel HGPRBMY1 or HGPRBMY2 polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without imitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptides (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C & E N, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a HGPRBMY1 or HGPRBMY2 polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a HGPRBMY1 or HGPRBMY2 polypeptide or peptide to measure a biological binding or ligand binding activity, the source may be a whole cell lysate that can be prepared by successive freeze-thaw cycles (e.g., one to three) in the presence of standard protease inhibitors. The HGPRBMY1 or HGPRBMY2 polypeptide may be partially or completely purified by standard protein purification methods, e.g., affinity chromatography using specific antibody described infra, or by ligands specific for an epitope tag engineered into the recombinant HGPRBMY1 or HGPRBMY2 polypeptide molecule, also as described herein. Binding activity can then be measured as described.

Compounds which are identified according to the methods provided herein, and which modulate or regulate the biological activity or physiology of the HGPRBMY1 or HGPRBMY2 polypeptides according to the present invention are a preferred embodiment of this invention. It is contemplated that such modulatory compounds may be employed in treatment and therapeutic methods for treating a condition that is mediated by the novel HGPRBMY1 or HGPRBMY2 polypeptides by administering to an individual in need of such treatment a therapeutically effective amount of the compound identified by the methods described herein.

In addition, the present invention provides methods for treating an individual in need of such treatment for a disease, disorder, or condition that is mediated by the HGPRBMY1 or HGPRBMY2 polypeptides of the invention, comprising administering to the individual a therapeutically effective amount of the HGPRBMY1 or HGPRBMY2 modulating compound identified by a method provided herein.

5.5.1. In Vitro Screening Assays for Compounds that Bind to HGPRBMY1 or HGPRBMY2

In vitro systems may be designed to identify compounds capable of interacting with (e.g., binding to) HGPRBMY1 or HGPRBMY2 (including, but not limited to, the ECD or CD of HGPRBMY1 or HGPRBMY2). Compounds identified may be useful, for example, in modulating the activity of wild type and/or mutant HGPRBMY1 or HGPRBMY2 gene products; may be useful in elaborating the biological function of the HGPRBMY1 or HGPRBMY2; may be utilized in screens for identifying compounds that disrupt normal HGPRBMY1 or HGPRBMY2 interactions; or may in themselves disrupt such interactions.

The principle of the assays used to identify compounds that bind to the HGPRBMY1 or HGPRBMY2 involves preparing a reaction mixture of the HGPRBMY1 or HGPRBMY2 and the test compound under conditions and for a time sufficient to allow the two components to interact and bind, thus forming a complex which can be removed and/or detected in the reaction mixture. The HGPRBMY1 or HGPRBMY2 species used can vary depending upon the goal of the screening assay. For example, where agonists of the natural ligand are sought, the full length HGPRBMY1 or HGPRBMY2, or a soluble truncated HGPRBMY1 or HGPRBMY2, e.g., in which the TM and/or CD is deleted from the molecule, a peptide corresponding to the ECD or a fusion polypeptide containing the HGPRBMY1 or HGPRBMY2 ECD fused to a polypeptide or peptide that affords advantages in the assay system (e.g., labeling, isolation of the resulting complex, etc.) can be utilized. Where compounds that interact with the cytoplasmic domain are sought to be identified, peptides corresponding to the HGPRBMY1 or HGPRBMY2 CD and fusion polypeptides containing the HGPRBMY1 or HGPRBMY2 CD can be used.

The screening assays can be conducted in a variety of ways. For example, one method to conduct such an assay would involve anchoring the HGPRBMY1 or HGPRBMY2 polypeptide, peptide or fusion polypeptide or the test substance onto a solid phase and detecting HGPRBMY1 or HGPRBMY2/test compound complexes anchored on the solid phase at the end of the reaction. In one embodiment of such a method, the HGPRBMY1 or HGPRBMY2 reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the polypeptide and drying. Alternatively, an immobilized antibody, preferably a monoclonal antibody, specific for the polypeptide to be immobilized may be used to anchor the polypeptide to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways.

Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for HGPRBMY1 or HGPRBMY2 polypeptide, peptide or fusion polypeptide or the test compound to anchor any complexes formed in solution, and a labeled antibody specific for the other component of the possible complex to detect anchored complexes.

Alternatively, cell-based assays can be used to identify compounds that interact with HGPRBMY1 or HGPRBMY2. To this end, cell lines that express HGPRBMY1 or HGPRBMY2, or cell lines (e.g., COS cells, CHO cells, fibroblasts, etc.) that have been genetically engineered to express HGPRBMY1 or HGPRBMY2 (e.g., by transfection or transduction of HGPRBMY1 or HGPRBMY2 DNA) can be used. Interaction of the test compound with, for example, the ECD of HGPRBMY1 or HGPRBMY2 expressed by the host cell can be determined by comparison or competition with native agonist or antagonist.

5.5.2. Assays for Polypeptides that Interact with the HGPRBMY1 or HGPRBMY2

Any method suitable for detecting polypeptide-polypeptide interactions may be employed for identifying transmembrane polypeptides or intracellular polypeptides that interact with HGPRBMY1 or HGPRBMY2. Among the traditional methods which may be employed are co-immunoprecipitation, crosslinking and co-purification through gradients or chromatographic columns of cell lysates or polypeptides obtained from cell lysates and the HGPRBMY1 or HGPRBMY2 to identify polypeptides in the lysate that interact with the HGPRBMY1 or HGPRBMY2. For these assays, the HGPRBMY1 or HGPRBMY2 component used can be a full length HGPRBMY1 or HGPRBMY2, a soluble derivative lacking the membrane-anchoring region (e.g., a truncated HGPRBMY1 or HGPRBMY2 in which the TM is deleted resulting in a truncated molecule containing the ECD fused to the CD), a peptide corresponding to the CD or a fusion polypeptide containing the CD of HGPRBMY1 or HGPRBMY2. Once isolated, such an intracellular polypeptide can be identified and can, in turn, be used, in conjunction with standard techniques, to identify polypeptides with which it interacts. For example, at least a portion of the amino acid sequence of an intracellular polypeptide which interacts with the HGPRBMY1 or HGPRBMY2 can be ascertained using techniques well known to those of skill in the art, such as via the Edman degradation technique (See, e.g. Creighton, 1983, “Proteins: Structures and Molecular Principles”, W. H. Freeman & Co., N.Y., pp.34-49). The amino acid sequence obtained may be used as a guide for the generation of oligonucleotide mixtures that can be used to screen for gene sequences encoding such intracellular polypeptides. Screening may be accomplished, for example, by standard hybridization of PCR techniques. Techniques for the generation of oligonucleotide mixtures and the screening are well-known (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the transmembrane or intracellular polypeptides interacting with HGPRBMY1 or HGPRBMY2. These methods include, for example, probing expression, libraries, in a manner similar to the well known technique of antibody probing of λgt11 libraries, using labeled HGPRBMY1 or HGPRBMY2 polypeptide, or an HGPRBMY1 or HGPRBMY2 polypeptide, peptide or fusion polypeptide, e.g., an HGPRBMY1 or HGPRBMY2 polypeptide or HGPRBMY1 or HGPRBMY2 domain fused to a marker (e.g., an enzyme, fluor, luminescent polypeptide, or dye), or an Ig-Fc domain.

One method which detects polypeptide interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid polypeptides: one plasmid consists of nucleic acids encoding the DNA-binding domain of a transcription activator polypeptide fused to an HGPRBMY1 or HGPRBMY2 nucleic acid sequence encoding HGPRBMY1 or HGPRBMY2, an HGPRBMY1 or HGPRBMY2 polypeptide, peptide or fusion polypeptide, and the other plasmid consists of nucleic acids encoding the transcription activator polypeptide's activation domain fused to a cDNA encoding an unknown polypeptide which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid polypeptide alone cannot activate transcription of the reporter gene: the DNA-binding domain hybrid cannot because it does not provide activation function and the activation domain hybrid cannot because it cannot localize to the activator's binding sites. Interaction of the two hybrid polypeptides reconstitutes the functional activator polypeptide and results in expression of the reporter gene, which is detected by an assay for the reporter gene product.

The two-hybrid system or related methodology may be used to screen activation domain libraries for polypeptides that interact with the “bait” gene product. By way of example, and not by way of limitation, HGPRBMY1 or HGPRBMY2 may be used as the bait gene product. Total genomic or cDNA sequences are fused to the DNA encoding an activation domain. This library and a plasmid encoding a hybrid of a bait HGPRBMY1 or HGPRBMY2 gene product fused to the DNA-binding domain are cotransformed into a yeast reporter strain, and the resulting transformants are screened for those that express the reporter gene. For example, and not by way of limitation, a bait HGPRBMY1 or HGPRBMY2 gene sequence, such as the open reading frame of HGPRBMY1 or HGPRBMY2 (or a domain of HGPRBMY1 or HGPRBMY2), as depicted in FIG. 1 can be cloned into a vector such that it is translationally fused to the DNA encoding the DNA-binding domain of the GAL4 polypeptide. These colonies are purified and the library plasmids responsible for reporter gene expression are isolated. DNA sequencing is then used to identify the polypeptides encoded by the library plasmids.

A cDNA library of the cell line from which polypeptides that interact with bait HGPRBMY1 or HGPRBMY2 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait HGPRBMY1 or HGPRBMY2 gene-GAL4 fusion plasmid into a yeast strain which contains a lacZ gene driven by a promoter which contains GAL4 activation sequence. A cDNA encoded polypeptide, fused to GAL4 transcriptional activation domain, that interacts with bait HGPRBMY1 or HGPRBMY2 gene product will reconstitute an active GAL4 polypeptide and thereby drive expression of the HIS3 gene. Colonies which express HIS3 can be detected by their growth on petri dishes containing semi-solid agar based media lacking histidine. The cDNA can then be purified from these strains, and used to produce and isolate the bait HGPRBMY1 or HGPRBMY2 gene-interacting polypeptide using techniques routinely practiced in the art.

Additional assays for identifying polypeptides that bind to and potentially modulate the HGPRBMY1 or HGPRBMY2 polypeptides are described elsewhere herein. More specifically, peptides have been identified that have been shown to bind to and potentially modulate the HGPRBMY2 polypeptide.

5.5.3. Assays for Other Compounds

The macromolecules that interact with the HGPRBMY1 are referred to, for purposes of this discussion, as “binding partners”. These binding partners are likely to be involved in the HGPRBMY1 signal transduction pathway, and therefore, in the role of HGPRBMY1 in immune related regulation. Therefore, it is desirable to identify compounds that interfere with or disrupt the interaction of such binding partners with agonist or antagonist which may be useful in regulating the activity of the HGPRBMY1 and control immune disorders associated with HGPRBMY1 activity.

The macromolecules that interact with the HGPRBMY2 are referred to, for purposes of this discussion, as “binding partners”. These binding partners are likely to be involved in the HGPRBMY2 signal transduction pathway, and therefore, in the role of HGPRBMY2 in cardiovascular regulation. Therefore, it is desirable to identify compounds that interfere with or disrupt the interaction of such binding partners with agonist or antagonist which may be useful in regulating the activity of the HGPRBMY2 and control cardiovascular or neural disorders associated with HGPRBMY2 activity.

The basic principle of the assay systems used to identify compounds that interfere with the interaction between the HGPRBMY1 or HGPRBMY2 and its binding partner or partners involves preparing a reaction mixture containing HGPRBMY1 or HGPRBMY2 polypeptide, peptide or fusion polypeptide as described in Sections 5.5.1 and 5.5.2 above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the HGPRBMY1 or HGPRBMY2 moiety and its binding partner. Control reaction mixtures are incubated without the test compound or with a placebo. The formation of any complexes between the HGPRBMY1 or HGPRBMY2 moiety and the binding partner is then detected. The formation of a complex in the control reaction, but not in the reaction mixture containing the test compound, indicates that the compound interferes with the interaction of the. HGPRBMY1 or HGPRBMY2 and the interactive binding partner. Additionally, complex formation within reaction mixtures containing the test compound and normal HGPRBMY1 or HGPRBMY2 polypeptide may also be compared to complex formation within reaction mixtures containing the test compound and a mutant HGPRBMY1 or HGPRBMY2. This comparison may be important in those cases wherein it is desirable to identify compounds that disrupt interactions of mutant but not normal HGPRBMY 1 or HGPRBMY2.

The assay for compounds that interfere with the interaction of the HGPRBMY 1 or HGPRBMY2 and binding partners can be conducted in a heterogeneous or homogeneous format. Heterogeneous assays involve anchoring either the HGPRBMY1 or HGPRBMY2 moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the HGPRBMY1 or HGPRBMY2 moiety and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the HGPRBMY1 or HGPRBMY2 moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the HGPRBMY1 or HGPRBMY2 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the HGPRBMY1 or HGPRBMY2 moiety and the interactive binding partner is prepared in which either the HGPRBMY1 or HGPRBMY2 or its binding partners is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt HGPRBMY1 or HGPRBMY2/intracellular binding partner interaction can be identified.

In a particular embodiment, an HGPRBMY1 or HGPRBMY2 fusion can be prepared for immobilization. For example, the HGPRBMY1 or HGPRBMY2 polypeptides or peptides, e.g., corresponding to the CD, can be fused to a glutathione-S-transferase (GST) gene using a fusion vector, such as pGEX-5X-1, in such a manner that its binding activity is maintained in the resulting fusion polypeptide. The interactive binding partner can be purified and used to raise a monoclonal antibody, using methods routinely practiced in the art and described above, in Section 5.3. This antibody can be labeled with the radioactive isotope ¹²⁵I, for example, by methods routinely practiced in the art. In a heterogeneous assay, e.g., the GST-HGPRBMY1 or HGPRBMY2 fusion polypeptide can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the HGPRBMY1 or HGPRBMY2 gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-HGPRBMY1 or HGPRBMY2 fusion polypeptide and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the HGPRBMY1 or HGPRBMY2/binding partner interaction can be detected by adding the labeled antibody and measuring the radioactivity associated with the beads.

In another embodiment of the invention, these same techniques can be employed using polypeptides or peptides that correspond to the binding domains of the HGPRBMY1 or HGPRBMY2 and/or the interactive or binding partner (in cases where the binding partner is a polypeptide), in place of one or both of the full length polypeptides. Any number of methods routinely practiced in the art can be used to identify and isolate the binding sites. These methods include, but are not limited to, mutagenesis of the gene encoding one of the polypeptides and screening for disruption of binding in a co-immunoprecipitation assay. compensating mutations in the gene encoding the second species in the complex can then be selected. Sequence analysis of the genes encoding the respective polypeptides will reveal the mutations that correspond to the region of the polypeptide involved in interactive binding.

Alternatively, one polypeptide can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express polypeptides or peptides of the invention, which can then be tested for binding activity and purified or synthesized.

For example, and not by way of limitation, an HGPRBMY1 or HGPRBMY2 gene product can be anchored to a solid material by making a GST-HGPRBMY1 or HGPRBMY2 fusion polypeptide and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-HGPRBMY1 or HGPRBMY2 fusion polypeptide and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative polypeptides using recombinant DNA technology.

5.5.4. Assays for Identification of Compounds that Ameliorate Immune Disorders

Compounds, including but not limited to binding compounds identified via assay techniques such as those described, above, in Sections 5.5.1 through 5.5.3, can be tested for the ability to ameliorate immune related disorder symptoms, including immunodeficiency. The assays described above can identify compounds which affect HGPRBMY1 activity (e.g., compounds that bind to the HGPRBMY1, inhibit binding of the natural ligand, and either activate signal transduction (agonists) or block activation (antagonists), and compounds that bind to the natural ligand of the HGPRBMY1 and neutralize ligand activity); or compounds that affect HGPRBMY1 gene activity (by affecting HGPRBMY1 gene expression, including molecules, e.g., polypeptides or small organic molecules, that affect or interfere with splicing events so that expression of the full length or the truncated form of the HGPRBMY1 can be modulated). However, it should be noted that the assays described can also identify compounds that modulate HGPRBMY1 signal transduction (e.g., compounds which affect downstream signaling events, such as inhibitors or enhancers of tyrosine kinase or phosphatase activities which participate in transducing the signal activated by agonist or antagonist binding to the HGPRBMY1). The identification and use of such compounds which affect another step in the HGPRBMY1 signal transduction pathway in which the HGPRBMY1 gene and/or HGPRBMY1 gene product is involved and, by affecting this same pathway may modulate the effect of HGPRBMY1 on the development of immune disorders are within the scope of the invention. Such compounds can be used as part of a therapeutic method for the treatment of immune disorders.

The invention features cell-based and animal model-based assays for the identification of compounds exhibiting such an ability to ameliorate immune related disorder symptoms. Such cell-based assay systems can also be used as a standard to assay for purity and potency of the natural ligand, agonist or antagonist, including recombinantly or synthetically produced agonist or antagonist and agonist or antagonist mutants.

Cell-based systems can be used to identify compounds which may act to ameliorate immune related disorder symptoms. Such cell systems can include, for example, recombinant or non-recombinant cells, such as cell lines, which express the HGPRBMY1 gene. For example bone marrow or spleen cells, or cell lines derived from bone marrow or spleen can be used. In addition, expression host cells (e.g., COS cells, CHO cells, fibroblasts) genetically engineered to express a functional HGPRBMY1 and to respond to activation by the natural agonist or antagonist ligand, e.g., as measured by a chemical or phenotypic change, induction of another host cell gene, change in ion flux (e.g., Ca⁺⁺), tyrosine phosphorylation of host cell polypeptides, etc., can be used as an end point in the assay.

In utilizing such cell systems, cells may be exposed to a compound suspected of exhibiting an ability to ameliorate immune related disorder symptoms, at a sufficient concentration and for a time sufficient to elicit such an amelioration of immune related disorder symptoms in the exposed cells. After exposure, the cells can be assayed to measure alterations in the expression of the HGPRBMY1 gene, e.g., by assaying cell lysates for HGPRBMY1 mRNA transcripts (e.g., by Northern analysis) or for HGPRBMY1 polypeptide expressed in the cell; compounds which regulate or modulate expression of the HGPRBMY1 gene are good candidates as therapeutics. Alternatively, the cells are examined to determine whether one or more immune related disorder-like cellular phenotypes has been altered to resemble a more normal or more wild type, non-immune related disorder phenotype, or a phenotype more likely to produce a lower incidence or severity of disorder symptoms.

Still further, the expression and/or activity of components of the signal transduction pathway of which HGPRBMY1 is a part, or the activity of the HGPRBMY1 signal transduction pathway itself can be assayed. For example, after exposure, the cell lysates can be assayed for the presence of tyrosine phosphorylation of host cell polypeptides, as compared to lysates derived from unexposed control cells. The ability of a test compound to inhibit tyrosine phosphorylation of host cell polypeptides in these assay systems indicates that the test compound inhibits signal transduction initiated by HGPRBMY1 activation. The cell lysates can be readily assayed using a Western blot format; i.e., the host cell polypeptides are resolved by gel electrophoresis, transferred and probed using a anti-phosphorylated amino acid detection antibody (e.g., an anti-phosphotyrosine antibody labeled with a signal generating compound, such as radiolabel, fluor, enzyme, etc.) (See, e.g., Glenney et al., 1988, J. Immunol. Methods 109:277-285; Frackelton et al., 1983, Mol. Cell. Biol. 3:1343-1352). Alternatively, an ELISA format could be used in which a particular host cell polypeptide involved in the HGPRBMY1 signal transduction pathway is immobilized using an anchoring antibody specific for the target host cell polypeptide, and the presence or absence of phosphorlyated amino acid residues, for example on tyrosine, on the immobilized host cell polypeptide is detected using a labeled anti-phosphotyrosine antibody (See, King et al., 1993, Life Sciences 53:1465-1472). In yet another approach, ion flux, such as calcium ion flux, can be measured as an end point for HGPRBMY1 stimulated signal transduction.

In addition, animal-based immune related disorder systems may for example be used to identify compounds capable of ameliorating immune related disorder-like symptoms. Such animal models may be used as test substrates for the identification of drugs, pharmaceuticals, therapies and interventions which may be effective in treating such disorders. For example, animal models may be exposed to a compound, suspected of exhibiting an ability to ameliorate immune related disorder symptoms, at a sufficient concentration and for a time sufficient to elicit such an amelioration of immune related disorder symptoms in the exposed animals. The response of the animals to the exposure may be monitored by assessing the reversal of disorders associated with immune disorders such as immunodeficiency. With regard to intervention, any treatments which reverse any aspect of immune related disorder-like symptoms should be considered as candidates for human immune related disorder therapeutic intervention. Dosages of test agents may be determined by deriving dose-response curves, as discussed in Section 5.7.1, below.

5.5.4b. Assays for Identification of Compounds that Ameliorate Cardiovascular Disorders

Compounds, including but not limited to binding compounds identified via assay techniques such as those described, above, in Sections 5.5.1 through 5.5.3, can be tested for the ability to ameliorate cardiovascular disorder symptoms, including congestive heart failure. The assays described above can identify compounds which affect HGPRBMY2 activity (e.g., compounds that bind to the HGPRBMY2, inhibit binding of the natural ligand, and either activate signal transduction (agonists) or block activation (antagonists), and compounds that bind to the natural ligand of the HGPRBMY2 and neutralize ligand activity); or compounds that affect HGPRBMY2 gene activity (by affecting HGPRBMY2 gene expression, including molecules, e.g., polypeptides or small organic molecules, that affect or interfere with splicing events so that expression of the full length or the truncated form of the HGPRBMY2 can be modulated). However, it should be noted that the assays described can also identify compounds that modulate HGPRBMY2 signal transduction (e.g., compounds which affect downstream signalling events, such as inhibitors or enhancers of tyrosine kinase or phosphatase activities which participate in transducing the signal activated by agonist or antagonist binding to the HGPRBMY2). The identification and use of such compounds which affect another step in the HGPRBMY2 signal transduction pathway in which the HGPRBMY2 gene and/or HGPRBMY2 gene product is involved and, by affecting this same pathway may modulate the effect of HGPRBMY2 on the development of cardiovascular disorders are within the scope of the invention. Such compounds can be used as part of a therapeutic method for the treatment of cardiovascular disorders.

The invention encompasses cell-based and animal model-based assays for the identification of compounds exhibiting such an ability to ameliorate cardiovascular disorder symptoms. Such cell-based assay systems can also be used as a standard to assay for purity and potency of the natural ligand, agonist or antagonist, including recombinantly or synthetically produced agonist or antagonist and agonist or antagonist mutants.

Cell-based systems can be used to identify compounds which may act to ameliorate cardiovascular disorder symptoms. Such cell systems can include, for example, recombinant or non-recombinant cells, such as cell lines, which express the HGPRBMY2 gene. For example heart cells, or cell lines derived from heart can be used. In addition, expression host cells (e.g., COS cells, CHO cells, fibroblasts) genetically engineered to express a functional HGPRBMY2 and to respond to activation by the natural agonist or antagonist ligand, e.g., as measured by a chemical or phenotypic change, induction of another host cell gene, change in ion flux (e.g., Ca⁺⁺), tyrosine phosphorylation of host cell polypeptides, etc., can be used as an end point in the assay.

In utilizing such cell systems, cells may be exposed to a compound suspected of exhibiting an ability to ameliorate cardiovascular disorder symptoms, at a sufficient concentration and for a time sufficient to elicit such an amelioration of cardiovascular disorder symptoms in the exposed cells. After exposure, the cells can be assayed to measure alterations in the expression of the HGPRBMY2 gene, e.g., by assaying cell lysates for HGPRBMY2 mRNA transcripts (e.g., by Northern analysis) or for HGPRBMY2 polypeptide expressed in the cell; compounds which regulate or modulate expression of the HGPRBMY2 gene are good candidates as therapeutics. Alternatively, the cells are examined to determine whether one or more cardiovascular disorder-like cellular phenotypes has been altered to resemble a more normal or more wild type, non-cardiovascular disorder phenotype, or a phenotype more likely to produce a lower incidence or severity of disorder symptoms. Still further, the expression and/or activity of components of the signal transduction pathway of which HGPRBMY2 is a part, or the activity of the HGPRBMY2 signal transduction pathway itself can be assayed. For example, after exposure, the cell lysates can be assayed for the presence of tyrosine phosphorylation of host cell polypeptides, as compared to lysates derived from unexposed control cells. The ability of a test compound to inhibit tyrosine phosphorylation of host cell polypeptides in these assay systems indicates that the test compound inhibits signal transduction initiated by HGPRBMY2 activation. The cell lysates can be readily assayed using a Western blot format; i.e., the host cell polypeptides are resolved by gel electrophoresis, transferred and probed using a anti-phosphotyrosine detection antibody (e.g., an anti-phosphotyrosine antibody labeled with a signal generating compound, such as radiolabel, fluor, enzyme, etc.) (See, e.g., Glenney et al., 1988, J. Immunol. Methods 109:277-285; Frackelton et al., 1983, Mol. Cell. Biol. 3:1343-1352). Alternatively, an ELISA format could be used in which a particular host cell polypeptide involved in the HGPRBMY2 signal transduction pathway is immobilized using an anchoring antibody specific for the target host cell polypeptide, and the presence or absence of phosphotyrosine on the immobilized host cell polypeptide is detected using a labeled anti-phosphotyrosine antibody. (See, King et al., 1993, Life Sciences 53:1465-1472). In yet another approach, ion flux, such as calcium ion flux, can be measured as an end point for HGPRBMY2 stimulated signal transduction.

In addition, animal-based cardiovascular disorder systems may for example be used to identify compounds capable of ameliorating cardiovascular disorder-like symptoms. Such animal models may be used as test substrates for the identification of drugs, pharmaceuticals, therapies and interventions which may be effective in treating such disorders. For example, animal models may be exposed to a compound, suspected of exhibiting an ability to ameliorate cardiovascular disorder symptoms, at a sufficient concentration and for a time sufficient to elicit such an amelioration of cardiovascular disorder symptoms in the exposed animals. The response of the animals to the exposure may be monitored by assessing the reversal of disorders associated with cardiovascular disorders such as congestive heart failure. With regard to intervention, any treatments which reverse any aspect of cardiovascular disorder-like symptoms should be considered as candidates for human cardiovascular disorder therapeutic intervention. Dosages of test agents may be determined by deriving dose-response curves, as discussed in Section 5.7. 1, below.

5.6. The Treatment of Immune Related, Including Immune Disorders

The invention features methods and compositions for modifying immune related disorders and treating immune disorders, including but not limited to immunodeficiency. Because a loss of normal HGPRBMY1 gene product function results in the development of immune related disease, an increase in HGPRBMY1 gene product activity, or activation of the HGPRBMY1 pathway (e.g., downstream activation) would facilitate progress towards a normal immune related state in individuals exhibiting a deficient level of HGPRBMY1 gene expression and/or HGPRBMY1 activity.

Alternatively, symptoms of certain immune disorders such as, for example, immunodeficiency may be ameliorated by modulating (increasing or decreasing) the level of HGPRBMY1 gene expression, and/or HGPRBMY1 gene activity, and/or modulating activity of the HGPRBMY1 pathway (e.g., by targeting downstream signaling events). Different approaches are discussed below.

HGPRBMY1 is expressed in bone marrow, spleen and thymus tissues, thus HGPRBMY1 nucleic acids, polypeptides, and modulators thereof can be used to modulate the proliferation, development, differentiation, and/or function of immune cells, e.g. B-cells, dendritic cells, natural killer cells and monocytes, and/or immune function. HGPRBMY1 nucleic acids, polypeptides and modulators thereof can be utilized to modulate immune-related processes, e.g., the host immune response by, for example, modulating the formation of and/or binding to immune complexes, detection and defense against surface antigens and bacteria, and immune surveillance for rapid removal or pathogens.

HGPRBMY1 nucleic acids, polypeptides and modulators thereof can be utilized to modulate or treat immune disorders that include, but are not limited to, immune proliferative disorders (e.g., carcinoma, lymphoma, e.g., follicular lymphoma), and disorders associated with fighting pathogenic infections, (e.g., bacterial (e.g., chlamydia) infection, parasitic infection, and viral infection (e.g., HSV or HIV infection)), and pathogenic disorders (e.g., immunodeficiency disorders, such as HIV), autoimmune disorders, such as arthritis, multiple sclerosis, Grave's disease, or Hashimoto's disease, T cell disorders (e.g., AIDS) and inflammatory disorders, such as septicemia, cerebral malaria, inflammatory bowel disease, arthritis (e.g., rheumatoid arthritis, osteoarthritis), and allergic inflammatory disorders (e.g., asthma, psoriasis), apoptotic disorders (e.g., rheumatoid arthritis, systemic lupus erythematosus, insulin-dependent diabetes mellitus), cytotoxic disorders, septic shock, and cachexia.

HGPRBMY1 nucleic acids, polypeptides and modulators thereof can be utilized to regulate immune activation to suppress rejection of a grafted organ or grafted tissue in a graft recipient (e.g., to prevent allograft rejection).

HGPRBMY1 nucleic acids, polypeptides and modulators thereof can be utilized to modulate immune activation. For example, antagonists to HGPRBMY1 action, such as peptides, antibodies or small molecules that decrease or block HGPRBMY1 activity, e.g., binding to extracellular matrix components, e.g., integrins, or that prevent HGPRBMY1 signaling, can be used as immune system activation blockers. In another example, agonists that mimic or partially mimic HGPRBMY1 activity, such as peptides, antibodies or small molecules, can be used to induce immune system activation. Antibodies may activate or inhibit the cell adhesion, proliferation and activation, and may help in treating infection, autoimmunity, inflammation, and cancer by affecting these cellular processes.

HGPRBMY1 nucleic acids, polypeptides and modulators thereof can also be utilized to modulate intercellular signaling in the immune system, e.g., modulate intercellular signal transduction in immune stimulation or suppression and modulate immune cell membrane adhesion to extra-cellular matrix components.

As HGPRBMY1 is expressed in bone marrow, HGPRBMY1 nucleic acids, polypeptides, and modulators thereof can be used to diagnose disorders associated with cells in the bone marrow and/or modulate the proliferation, differentiation, and/or function of cells that appear in the bone marrow, e.g., stem cells (e.g., hematopoietic stem cells), and blood cells, e.g., erythrocytes, platelets, and leukocytes. Thus HGPRBMY1 nucleic acids, polypeptides, and modulators thereof can be used to treat bone marrow, blood, and hematopoietic associated diseases and disorders, e.g., acute myeloid leukemia, hemophilia, leukemia, anemia (e.g., sickle cell anemia), and thalassemia.

As HGPRBMY1 is expressed in the thymus, HGPRBMY1 nucleic acids, polypeptides, and modulators thereof can be used to diagnose thymus associated disorders. HGPRBMY1 nucleic acids, polypeptides, and modulators thereof can also be used modulate the proliferation, development, differentiation, maturation and/or function of thymocytes, e.g., modulate development and maturation of T-lymphocytes. HGPRBMY1 nucleic acids, polypeptides and modulators thereof can be utilized to modulate immune-related processes such as the ability to modulate host immune response by, e.g., modulating the formation of and/or binding to immune complexes, and modulating the positive and negative selection of thymocytes. Such HGPRBMY1 compositions and modulators thereof can be utilized, e.g., to ameliorate incidence of any symptoms associated with disorders that involve such immune-related processes, including, but not limited to infection and autoimmune disorders (e.g., insulin-dependent mellitus, multiple sclerosis, systemic lupus, erythematosus, sjogren's syndrome, autoimmune thyroiditis, idiotpathic Addison's disease, vitiligo, Grave's disease, idiopathic thrombocytopenia purpura, rheumatoid arthritis, and scleroderma). HGPRBMY1 nucleic acids, polypeptides and modulators thereof can also be utilized to treat viral infections, inflammatory immune disorders and immune-related cancers including but not limited to, leukemia (e.g., acute leukemia, chronic leukemia, Hodgkin's disease non-Hodgkin's lymphoma ,and multiple myeloma).

HGPRBMY1 has structural homology with the receptor for the serine protease, thrombin. As such HGPRBMY1 nucleic acids, polypeptides and modulators thereof can be utilized to modulate activities, processes or disorders associated with protease activity, e.g., serine protease activity. For example, HGPRBMY1 nucleic acids, polypeptides or modulators thereof can be used to modulate serine protease activities, such as those activities associated with such serine proteases (or, where appropriate, human homologues thereof), e.g., adipsin (complement factor D), acrosin, thrombin, plasminogen, protein C, cathepsin G, chymotrypsin, complement components and signaling, cytotoxic cell proteases, duodenase I, elastases 1, 2, 3A, 3B and medullasin, enterokinase, hepatocyte growth factor activator, hepsin, kallikreins, gamma-renin, prostate specific antigen, mast cell proteases, myeloblastin, Alzheimer's plaque-related proteases, tryptases, ancrod, batroxobin, cerastobin, flavoxobin, apolipoprotein, blood fluke cercarial protease, Drosophila trypsin like protease (e.g., alpha, easter, and snake locus), Drosophila protease stubble, or major mite fecal antigen.

HGPRBMY1 nucleic acids, polypeptides and modulators thereof can be used to modulate processes and/or diseases involved with serine protease response activity. For example, such processes and/or diseases can include, but are not limited to cellular activation, cellular proliferation, motility and differentiation, the alternative complement pathway, e.g., disturbances of the complement regulation system, such as complement regulator deficiencies, which include, for example, hereditary angioedema (an allergic disorder) and proxysmal nocturnal hemoglobinuria (the presence of hemoglobin in the urine), modulate body weight or body weight disorders, e.g., obesity or cachexia, systemic energy balance and diabetes.

In addition, assays can be developed to measure the biological activity of polypeptides or peptides of the invention. In particular, HGPRBMY1 or modulators thereof, biological activities include, e.g., (1) the ability to modulate development, differentiation, proliferation and/or activity of immune cells (e.g., leukocytes and macrophages), endothelial cells and smooth muscle cells; (2) the ability to modulate the host immune response; (3) the ability to modulate intracellular signaling cascades (e.g., signal transduction cascades); (4) the ability to modulate the development of organs, tissues and/or cells of the embryo and/or fetus; (5) the ability to modulate cell-cell interactions and/or cell-extracellular matrix interactions; (6) the ability to modulate atherosclerosis, e.g., the initiation and progression of atherosclerosis; (7) the ability to modulate atherogenesis; (8) the ability to modulate inflammatory functions e.g., by modulating leukocyte adhesion to extracellular matrix and/or endothelial cells; (9) the ability to bind and phagocytose cells, e.g., aged and apoptotic cells; (10) the ability to remove debris, e.g., apoptotic cells, from blood vessel walls; (11) the ability to modulate, e.g., inhibit, the expression of molecules, e.g., adhesion molecules (e.g., leukocyte adhesion molecules) and growth factors (e.g., smooth-muscle growth factors); (12) the ability to alter, e.g., increase, expression in response to stimuli, e.g., TNF, shear stress, and pathophysiological stimuli relevant to disorders (e.g., atherosclerosis and inflammation); and (13) the ability to form, e.g., stabilize, promote, facilitate, inhibit, or disrupt, cell to cell and cell to blood product interaction, e.g., between leukocytes and platelets or leukocytes and vascular endothelial cells.

5.6b. The Treatment of Cardiovascular, Including Cardiovascular Disorders

The invention encompasses methods and compositions for modifying cardiovascular and treating cardiovascular disorders, including but not limited to congestive heart failure. Because a loss of normal HGPRBMY2 gene product function results in the development of cardiovascular disease, an increase in HGPRBMY2 gene product activity, or activation of the HGPRBMY2 pathway (e.g., downstream activation) would facilitate progress towards a normal cardiovascular state in individuals exhibiting a deficient level of HGPRBMY2 gene expression and/or HGPRBMY2 activity. Alternatively, symptoms of certain cardiovascular disorders such as, for example, congestive heart failure may be ameliorated by modulating (increasing or decreasing) the level of HGPRBMY2 gene expression, and/or HGPRBMY2 gene activity, and/or modulating activity of the HGPRBMY2 pathway (e.g., by targeting downstream signalling events). Different approaches are discussed below.

5.6c. The Treatment of Neurological Disorders and Diseases

Nervous system diseases, disorders, and/or conditions, which can be treated, prevented, and/or diagnosed with the compositions of the invention (e.g., HGPRBMY2 polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B 12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the HGPRBMY2 polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The HGPRBMY2 compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

5.6.1. Modulation of HGPRBMY1 Expression or Activity

Any method which neutralizes an agonist or antagonist or modulates expression of the HGPRBMY1 gene (e.g., by either activating or decreasing transcription or translation) can be used to prevent HGPRBMY1 immune disorders.

For example, the administration of soluble peptides, polypeptides, fusion polypeptides, or antibodies (including anti-idiotypic antibodies) that bind to a circulating agonist or antagonist, the natural ligand for the HGPRBMY1, can be used to prevent or treat immune disorders. To this end, peptides corresponding to the ECD of HGPRBMY1, soluble deletion mutants of HGPRBMY1 (e.g., ATM-HGPRBMY1 mutants), or either of these HGPRBMY1 domains or mutants fused to another polypeptide (e.g., an IgFc polypeptide) can be utilized. Alternatively, anti-idiotypic antibodies or Fab fragments of antiidiotypic antibodies that mimic the HGPRBMY1 ECD and neutralize agonists or antagonists can be used (see Section 5.3, supra). Such HGPRBMY1 polypeptides, peptides, fusion polypeptides, anti-idiotypic antibodies or Fabs are administered to a subject in amounts sufficient to neutralize agonist or antagonist and to prevent or treat immune disorders.

Fusion of the HGPRBMY1, the HGPRBMY1 ECD or the ΔTMHGPRBMY1 to an IgFc polypeptide should not only increase the stability of the preparation, but will increase the half-life and activity of the HGPRBMY1-Ig fusion polypeptide in vivo. The Fc region of the Ig portion of the fusion polypeptide may be further modified to reduce immunoglobulin effector function. In an alternative embodiment for neutralizing circulating agonist or antagonist, cells that are genetically engineered to express such soluble or secreted forms of HGPRBMY1 may be administered to a patient, whereupon they will serve as “bioreactors” in vivo to provide a continuous supply of the agonist or antagonist neutralizing polypeptide. Such cells may be obtained from the patient or an MHC compatible donor and can include, but are not limited to fibroblasts, blood cells (e.g., lymphocytes), adipocytes, muscle cells, endothelial cells etc. The cells are genetically engineered in vitro using recombinant DNA techniques to introduce the coding sequence for the HGPRBMY1 ECD, ΔTMHGPRBMY1, or for HGPRBMY1-Ig fusion polypeptide (e.g., HGPRBMY1-, ECD- or ΔTMHGPRBMY1-IgFc fusion polypeptides) into the cells, etc. by transduction (using viral vectors, and preferably vectors that integrate the transgene into the cell genome) or transfection procedures, including but not limited to the use of plasmids, cosmids, YACs, electroporation, liposomes, etc. The HGPRBMY1 coding sequence can be placed under the control of a strong constitutive or inducible promoter or promoter/enhancer to achieve expression and secretion of the HGPRBMY1 peptide or fusion polypeptide. The engineered cells which express and secrete the desired HGPRBMY1 product can be introduced into the patient systemically, e.g., in the circulation, intraperitoneally, at the heart. Alternatively, the cells can be incorporated into a matrix and implanted in the body, e.g., genetically engineered fibroblasts can be implanted as part of a skin graft; genetically engineered endothelial cells can be implanted as part of a vascular graft (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

In an alternate embodiment, immune disorder therapy can be designed to reduce the level of endogenous HGPRBMY1 gene expression, e.g., using antisense or ribozyme approaches to inhibit or prevent translation of HGPRBMY1 mRNA transcripts; triple helix approaches to inhibit transcription of the HGPRBMY1 gene; or targeted homologous recombination to inactivate or “knock out” the HGPRBMY1 gene or its endogenous promoter. Alternatively, the antisense, ribozyme or DNA constructs described herein could be administered directly to the site containing the target cells; e.g., the bone marrow or spleen.

Antisense approaches involve the design of oligonucleotides (either DNA or RNA) that are complementary to HGPRBMY1 mRNA. The antisense oligonucleotides will bind to the complementary HGPRBMY1 mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Thus, oligonucleotides complementary to either the 5′- or 3′-non-translated, non-coding regions of the HGPRBMY1 shown in SEQ ID NO:1, could be used in an antisense approach to inhibit translation of endogenous HGPRBMY1 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of HGPRBMY1 mRNA, antisense nucleic acids should be at least six nucleotides in length, and are preferably oligonucleotides ranging from 6 to about 50 nucleotides in length. In specific aspects the oligonucleotide is at least nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or polypeptide with that of an internal control RNA or polypeptide. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents (See, e.g., Krol et al., 1988, BioTechniques 6:958-976) or intercalating agents (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc. While antisense nucleic acids complementary to the HGPRBMY1 coding region sequence could be used, those complementary to the transcribed untranslated region are most preferred. For example, antisense oligonucleotides having the following sequences can be utilized in accordance with the invention:

a) 5′-CATCCGCCTTATTACAT-3′ (SEQ ID NO:28) which is complementary to nucleotides −14 to +3 as shown in SEQ ID NO:1;

b) 5′-CATCCGCCTTATTACATCTTTTT-3′ (SEQ ID NO:29) which is complementary to nucleotides −20 to +3 in SEQ ID NO:1.

The antisense molecules should be delivered to cells which express the HGPRBMY1 in vivo, e.g., the bone marrow or spleen. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous HGPRBMY1 transcripts and thereby prevent translation of the HGPRBMY1 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced directly into the tissue site; e.g., the bone marrow or spleen. Alternatively, viral vectors can be used which selectively infect the desired tissue; (e.g., for bone marrow or spleen, herpesvirus vectors may be used or alternatively, in dividing bone marrow cells retroviruses may be used), in which case administration may be accomplished by another route (e.g., systemically).

Ribozyme molecules-designed to catalytically cleave HGPRBMY1 mRNA transcripts can also be used to prevent translation of HGPRBMY1mRNA and expression of HGPRBMY1. (See, e.g., PCT International Publication WO90/11364, published Oct. 4, 1990; Sarver et al., 1990, Science 247:1222-1225). While ribozymes that cleave mRNA at site specific recognition sequences can be used to destroy HGPRBMY1 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are hundreds of potential hammerhead ribozyme cleavage sites within the nucleic acid sequence of human HGPRBMY1 cDNA. Preferably the ribozyme is engineered so that the cleavage recognition site is located near the 5′ tend of the HGPRBMY1 mRNA; i.e., to increase efficiency and minimize the intracellular accumulation of non-functional mRNA transcripts. For example, hammerhead ribozymes can be utilized in accordance with the invention.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent-application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention features those Cech-type ribozymes which target eight base-pair active site sequences that are present in HGPRBMY1.

As in the antisense approach, the ribozymes can be composed of modified oligonucleotides (e.g. for improved stability, targeting, etc.) and should be delivered to cells which express the HGPRBMY1 in vivo, e.g., bone marrow or spleen. A preferred method of delivery involves using a DNA construct “encoding” the ribozyme under the control of a strong constitutive pol III or pol II promoter, so that transfected cells will produce sufficient quantities of the ribozyme to destroy endogenous HGPRBMY1 messages and inhibit translation. Because ribozymes unlike antisense molecules, are catalytic, a lower intracellular concentration is required for efficiency.

Endogenous HGPRBMY1 gene expression can also be reduced by inactivating the HGPRBMY1 gene or its promoter using targeted homologous recombination (e.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional HGPRBMY1, or unrelated sequences, which are flanked by DNA homologous to the endogenous HGPRBMY1 gene locus can be used with or without a selectable marker and/or a negative selectable marker, to transfect cells that express HGPRBMY1 in vivo. Insertion of the DNA construct, via targeted homologous recombination, results in inactivation of the HGPRBMY1 gene. Such approaches are particularly suited in the agricultural field where modifications to ES (embryonic stem) cells can be used to generate animal offspring with an inactive HGPRBMY1 (e.g., see Thomas & Capecchi 1987 and Thompson 1989, supra). However this approach can be adapted for use in humans provided the recombinant DNA constructs are directly administered or targeted to the required site in vivo using appropriate viral vectors, e.g., herpes virus vectors for delivery to tissue; e.g., bone marrow or spleen.

Alternatively, endogenous HGPRBMY1 gene expression can be reduced by targeting deoxyribonucleotide sequences complementary to the regulatory region of the HGPRBMY1 gene (i.e., the HGPRBMY1 promoter and/or enhancers) to form triple helical structures that prevent transcription of the HGPRBMY1 gene in target cells in the body. (See generally, Helene, C. 1991, Anticancer Drug Des., 6(6):569-84; Helene, C., et al., 1992, Ann, N. Y. Accad. Sci., 660:27-36; and Maher, L. J., 1992, Bioassays 14(12):807-15).

In yet another embodiment of the invention, the activity of HGPRBMY1 can be reduced using a “dominant negative” approach to prevent or treat immune disorders. To this end, constructs which encode defective HGPRBMY1 can be used in gene therapy approaches to diminish the activity of the HGPRBMY1 in appropriate target cells. For example, nucleic acid sequences that direct host cell expression of HGPRBMY1 in which the CD is deleted or mutated can be introduced into cells in the bone marrow or spleen (either by in vivo or ex vivo gene therapy methods described above). Alternatively, targeted homologous recombination can be utilized to introduce such deletions or mutations into the subject's endogenous HGPRBMY1 gene in the bone marrow or spleen. The engineered cells will express non-functional receptors (i.e., an anchored receptor that is capable of binding its natural ligand, but incapable of signal transduction). Such engineered cells present in the bone marrow or spleen should demonstrate a diminished response to the endogenous agonist or antagonist ligand, resulting in immune disorders.

With respect to an increase in the level of normal HGPRBMY 1 gene expression and/or HGPRBMY1 gene product activity, HGPRBMY1 nucleic acid sequences can be utilized for the treatment of immune disorders, including immunodeficiency. Where the cause of immunodeficiency is a defective HGPRBMY1, treatment can be administered, for example, in the form of gene replacement therapy.

In another embodiment, the expression characteristics of an endogenous gene (e.g., HGPRBMY1 genes) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., HGPRBMY1 genes) and controls, modulates or activates. For example, endogenous HGPRBMY1 genes which are normally “transcriptionally silent”, i.e., a HGPRBMY1 genes which is normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous HGPRBMY1 genes may be activated by insertion of a promiscuous regulatory element that works across cell types.

A heterologous regulatory element may be inserted into a stable cell line or cloned microorganism, such that it is operatively linked with and activates expression of endogenous HGPRBMY1 genes, using techniques, such as targeted homologous recombination, which are well known to those of skill in the art, and described e.g., in Chappel, U.S. Pat. No.5,272,071; PCT publication No. WO 91/06667, published May 16, 1991; Skoultchi U.S. Pat. No.5,981,214; Treco et al U.S. Pat. No. 5,968,502 and PCT publication No. WO 94/12650, published Jun. 9, 1994. Alternatively, non-targeted e.g., non-homologous recombination techniques which are well-known to those of skill in the art and described, e.g., in PCT publication No. WO 99/15650, published Apr. 1, 1999, may be used.

Specifically, one or more copies of a normal HGPRBMY1 gene or a portion of the HGPRBMY1 gene that directs the production of an HGPRBMY1 gene product exhibiting normal function, may be inserted into the appropriate cells within a patient or animal subject, using vectors which include, but are not limited to adenovirus, adeno-associated virus, retrovirus and herpes virus vectors, in addition to other particles that introduce DNA into cells, such as liposomes.

Because the HGPRBMY1 gene is expressed in the bone marrow, spleen and thymus, such gene replacement therapy techniques should be capable of delivering HGPRBMY1 gene sequences to these cell types within patients. Thus, the techniques for delivery of the HGPRBMY1 gene sequences should be designed to readily involve direct administration of such HGPRBMY1 gene sequences to the site of the cells in which the HGPRBMY1 gene sequences are to be expressed. Alternatively, targeted homologous recombination can be utilized to correct the defective endogenous HGPRBMY1 gene in the appropriate tissue; e.g., bone marrow or spleen cells (particularly B-cells). In animals, targeted homologous recombination can be used to correct the defect in ES cells in order to generate offspring with a corrected trait.

Additional methods which may be utilized to increase the overall level of HGPRBMY1 gene expression and/or HGPRBMY1 activity include the introduction of appropriate HGPRBMY1-expressing cells, preferably autologous cells, into a patient at positions and in numbers which are sufficient to ameliorate the symptoms of immune disorders, including immunodeficiency. Such cells may be either recombinant or non-recombinant. Among the cells which can be administered to increase the overall level of HGPRBMY1 gene expression in a patient are normal cells, preferably bone marrow or spleen cells, cells which express the HGPRBMY1 gene. The cells can be administered at the anatomical site in the body, or as part of a tissue graft located at a different site in the body. Such cell-based gene therapy techniques are well known to those skilled in the art, see, e.g., Anderson, et al., U.S. Pat. No. 5,399,349; Mulligan & Wilson, U.S. Pat. No. 5,460,959.

Finally, compounds, identified in the assays described above, that stimulate or enhance the signal transduced by activated HGPRBMY1, e.g., by activating downstream signaling polypeptides in the HGPRBMY1 cascade and thereby by-passing the defective HGPRBMY1, can be used to ameliorate immune related disease. The formulation and mode of administration will depend upon the physico-chemical properties of the compound.

5.6.1b. Modulation of HGPRBMY2 Expression or HGPRBMY2 Activity to Prevent Heart Failure

Any method which neutralizes an agonist or antagonist or modulates expression of the HGPRBMY2 gene (either transcription or translation) can be used to prevent heart failure or heart disease. Such approaches can be used to treat any cardiovascular disorder.

For example, the administration of soluble peptides, polypeptides, fusion polypeptides, or antibodies that bind to the natural ligand for the HGPRBMY2, can be used to prevent or treat heart disease. To this end, peptides corresponding to the ECD of HGPRBMY2, soluble deletion mutants of HGPRBMY2 (e.g., ATM-HGPRBMY2 mutants), or either of these HGPRBMY2 domains or mutants fused to another polypeptide (e.g., an IgFc polypeptide) can be utilized. Alternatively, anti-idiotypic antibodies or fragments thereof that mimic the HGPRBMY2 ECD and neutralize agonists or antagonists can be used (see Section 5.3, supra). Such HGPRBMY2 polypeptides, peptides, fusion polypeptides, and/or antibodies are administered to a subject in amounts sufficient to bind the ligand and to prevent or treat heart disease.

Fusion of the HGPRBMY2, the HGPRBMY2-ECD to an IgFc polypeptide should not only increase the stability of the preparation, but will increase the half-life and activity of the HGPRBMY2-Ig fusion polypeptide in vivo. The Fc region of the Ig portion of the fusion polypeptide may be further modified to reduce immunoglobulin effector function. In an alternative embodiment for neutralizing circulating agonist or antagonist, cells that are genetically engineered to express such soluble or secreted forms of HGPRBMY2 may be administered to a patient to provide a continuous supply of the agonist or antagonist neutralizing polypeptide. Such cells may be obtained from the patient or an MHC compatible donor and can include, but are not limited to fibroblasts, blood cells (e.g., lymphocytes), adipocytes, muscle cells, endothelial cells etc. The cells are genetically engineered in vitro using recombinant DNA techniques to introduce the coding sequence for the HGPRBMY2 ECD, ΔTMHGPRBMY2, or for HGPRBMY2-Ig fusion polypeptide (e.g., HGPRBMY2-, ECD- or ΔTMHGPRBMY2-IgFc fusion polypeptides) into the cells, etc. by transduction (using viral vectors, and preferably vectors that integrate the transgene into the cell genome) or transfection procedures, including but not limited to the use of plasmids, cosmids, YACs, electroporation, liposomes, etc.

The HGPRBMY2 coding sequence can be placed under the control of a strong constitutive or inducible promoter or promoter/enhancer to achieve expression and secretion of the HGPRBMY2 peptide or fusion polypeptide. The engineered cells which express and secrete the desired HGPRBMY2 product can be introduced into the patient systemically, e.g., in the circulation, intraperitoneally, at the heart. Alternatively, the cells can be incorporated into a matrix and implanted in the body, e.g., genetically engineered fibroblasts can be implanted as part of a skin graft; genetically engineered endothelial cells can be implanted as part of a vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

In an alternate embodiment, heart failure therapy can be designed to reduce the level of endogenous HGPRBMY2 gene expression, e.g., using antisense or ribozyme approaches to inhibit or prevent translation of HGPRBMY2 mRNA transcripts; triple helix approaches to inhibit transcription of the HGPRBMY2 gene; or targeted homologous recombination to inactivate or “knock out” the HGPRBMY2 gene or its endogenous promoter. Alternatively, the antisense, ribozyme or DNA constructs described herein could be administered directly to the site containing the target cells; e.g., the heart.

Antisense approaches involve the design of oligonucleotides (either DNA or RNA) that are complementary to HGPRBMY2 mRNA. The antisense oligonucleotides will bind to the complementary HGPRBMY2 mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Thus, oligonucleotides complementary to either the 5′- or 3′-non-translated, non-coding regions of the HGPRBMY2 shown in SEQ ID NO:13, could be used in an antisense approach to inhibit translation of endogenous HGPRBMY2 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of HGPRBMY2 mRNA, antisense nucleic acids should be at least six nucleotides in length, and are preferably oligonucleotides ranging from 6 to about 50 nucleotides in length. In specific aspects the oligonucleotide is at least nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or polypeptide with that of an internal control RNA or polypeptide. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyarninomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleic acids complementary to the HGPRBMY2 coding region sequence could be used, those complementary to the transcribed untranslated region are most preferred. For example, antisense oligonucleotides having the following sequences can be utilized in accordance with the invention:

a) 5′-CATGCGGGGCAGCGAGG-3′ (SEQ ID NO:30) which is complementary to nucleotides −14 to +3 as shown in SEQ ID NO:13;

b) 5′-CATGCGGGGCAGCGAGGGCTTCGG-3′ (SEQ ID NO:31) which is complementary to nucleotides −20 to +3 in SEQ ID NO:13.

The antisense molecules should be delivered to cells which express HGPRBMY2 in vivo, e.g., the heart. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

Alternatively, an antisense nucleic acid is delivered via a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous HGPRBMY2 transcripts and thereby prevent translation of the HGPRBMY2 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art.

Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced directly into the tissue site; e.g., the heart. Alternatively, viral vectors can be used which selectively infect the desired tissue; (e.g., for heart, herpesvirus vectors may be used), in which case administration may be accomplished by another route (e.g., systemically).

Ribozyme molecules-designed to catalytically cleave HGPRBMY2 mRNA transcripts can also be used to prevent translation of HGPRBMY2mRNA and expression of HGPRBMY2. (See, e.g., PCT International Publication WO90/11364, published Oct. 4, 1990; Sarver et al., 1990, Science 247:1222-1225). While ribozymes that cleave mRNA at site specific recognition sequences can be used to destroy HGPRBMY2 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are hundreds of potential hammerhead ribozyme cleavage sites within the nucleic acid sequence of human HGPRBMY2 cDNA. Preferably the ribozyme is engineered so that the cleavage recognition site is located near the 5′ tend of the HGPRBMY2 mRNA; i.e., to increase efficiency and minimize the intracellular accumulation of non-functional mRNA transcripts. For example, hammerhead ribozymes can be utilized in accordance with the invention.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent-application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in HGPRBMY2.

As in the antisense approach, the ribozymes can be composed of modified oligonucleotides (e.g. for improved stability, targeting, etc.) and should be delivered to cells which express the HGPRBMY2 in vivo, e.g., heart. A preferred method of delivery involves using a DNA construct “encoding” the ribozyme under the control of a strong constitutive pol III or pol II promoter, so that transfected cells will produce sufficient quantities of the ribozyme to destroy endogenous HGPRBMY2 messages and inhibit translation. Because ribozymes unlike antisense molecules, are catalytic, a lower intracellular concentration is required for efficiency.

Endogenous HGPRBMY2 gene expression can also be reduced by inactivating or “knocking out” the HGPRBMY2 gene or its promoter using targeted homologous recombination (e.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional HGPRBMY2 flanked by DNA homologous to the endogenous HGPRBMY2 gene locus, coding or regulatory, can be used with or without a selectable marker and/or a negative selectable marker to transfect cells that express HGPRBMY2 in vivo. Insertion of the DNA construct, via targeted homologous recombination, results in inactivation of the HGPRBMY2 gene. Such approaches are particularly suited in the agricultural field where modifications to ES (embryonic stem) cells can be used to generate animal offspring with an inactive HGPRBMY2 (e.g., see Thomas & Capecchi 1987 and Thompson 1989, supra). However this approach can be adapted for use in humans provided the recombinant DNA constructs are directly administered or targeted to the required site in vivo using appropriate viral vectors, e.g., herpes virus vectors for delivery to tissue; e.g., heart.

Alternatively, endogenous HGPRBMY2 gene expression can be reduced by targeting deoxyribonucleotide sequences complementary to the regulatory region of the HGPRBMY2 gene (i.e., the HGPRBMY2 promoter and/or enhancers) to form triple helical structures that prevent transcription of the HGPRBMY2 gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

In yet another embodiment of the invention, the activity of HGPRBMY2 can be reduced using a “dominant negative” approach to prevent or treat heart failure. To this end, constructs which encode defective HGPRBMY2 can be used in gene therapy approaches to diminish the activity of the HGPRBMY2 in appropriate target cells. For example, nucleic acid sequences that direct host cell expression of HGPRBMY2 in which the CD is deleted or mutated can be introduced into cells in the heart (either by in vivo or ex vivo gene therapy methods described above). Alternatively, targeted homologous recombination can be utilized to introduce such deletions or mutations into the subject's endogenous HGPRBMY2 gene in the heart. The engineered cells will express non-functional receptors (i.e., an anchored receptor that is capable of binding its natural ligand, but incapable of signal transduction). Such engineered cells present in the heart should demonstrate a diminished response to the endogenous agonist or antagonist ligand, resulting in heart failure.

With respect to an increase in the level of normal HGPRBMY2 gene expression and/or HGPRBMY2 gene product activity, HGPRBMY2 nucleic acid sequences can be utilized for the treatment of cardiovascular disorders, including congestive heart failure. here the cause of congestive heart failure is a defective HGPRBMY2, treatment can be administered, for example, in the form of gene replacement therapy.

In another embodiment, the expression characteristics of an endogenous gene (e.g., HGPRBMY2 genes) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., HGPRBMY2 genes) and controls, modulates or activates. For example, endogenous HGPRBMY2 genes which are normally “transcriptionally silent”, i.e., a HGPRBMY2 genes which is normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous HGPRBMY2 genes may be activated by insertion of a promiscuous regulatory element that works across cell types.

A heterologous regulatory element may be inserted into a stable cell line or cloned microorganism, such that it is operatively linked with and activates expression of endogenous HGPRBMY2 genes, using techniques, such as targeted homologous recombination, which are well known to those of skill in the art, and described e.g., in Chappel, U.S. Pat. No. 5,272,071; PCT publication No. WO 91/06667, published May 16, 1991; Skoultchi U.S. Pat. No. 5,981,214; Treco et al U.S. Pat. No. 5,968,502 and PCT publication No. WO 94/12650, published Jun. 9, 1994. Alternatively, non-targeted e.g., non-homologous recombination techniques which are well-known to those of skill in the art and described, e.g., in PCT publication No. WO 99/15650, published Apr. 1, 1999, may be used.

Specifically, one or more copies of a normal HGPRBMY2 gene or a portion of the HGPRBMY2 gene that directs the production of an HGPRBMY2 gene product exhibiting normal function, may be inserted into the appropriate cells within a patient or animal subject, using vectors which include, but are not limited to adenovirus, adeno-associated virus, retrovirus and herpes virus vectors, in addition to other particles that introduce DNA into cells, such as liposomes.

Because the HGPRBMY2 gene is expressed in the heart and thymus, such gene replacement therapy techniques should be capable of delivering HGPRBMY2 gene sequences to these cell types within patients. Thus, the techniques for delivery of the HGPRBMY2 gene sequences should be designed to readily involve direct administration of such HGPRBMY2 gene sequences to the site of the cells in which the HGPRBMY2 gene sequences are to be expressed. Alternatively, targeted homologous recombination can be utilized to correct the defective endogenous HGPRBMY2 gene in the appropriate tissue; e.g., heart. In animals, targeted homologous recombination can be used to correct the defect in ES cells in order to generate offspring with a corrected trait.

Additional methods which may be utilized to increase the overall level of HGPRBMY2 gene expression and/or HGPRBMY2 activity include the introduction of appropriate HGPRBMY2-expressing cells, preferably autologous cells, into a patient at positions and in numbers which are sufficient to ameliorate the symptoms of cardiovascular disorders, including congestive heart failure. Such cells may be either recombinant or non-recombinant. Among the cells which can be administered to increase the overall level of HGPRBMY2 gene expression in a patient are normal cells, preferably heart cells, cells which express the HGPRBMY2 gene. The cells can be administered at the anatomical site in the body, or as part of a tissue graft located at a different site in the body. Such cell-based gene therapy techniques are well known to those skilled in the art, see, e.g., Anderson, et al., U.S. Pat. No. 5,399,349; Mulligan & Wilson, U.S. Pat. No. 5,460,959.

Finally, compounds, identified in the assays described above, that stimulate or enhance the signal transduced by activated HGPRBMY2, e.g., by activating downstream signalling polypeptides in the HGPRBMY2 cascade and thereby by-passing the defective HGPRBMY2, can be used to ameliorate cardiovascular disease. The formulation and mode of administration will depend upon the physico-chemical properties of the compound.

5.7. Pharmaceutical Preparations and Methods of Administration

The compounds that are determined to affect HGPRBMY1 gene expression or HGPRBMY1 activity can be administered to a patient at therapeutically effective doses to treat or ameliorate bone marrow or spleen disorders. A therapeutically effective dose refers to that amount of the compound sufficient to result in amelioration of symptoms of immune disorders.

The compounds that are determined to affect HGPRBMY2 gene expression or HGPRBMY2 activity can be administered to a patient at therapeutically effective doses to treat or ameliorate heart disorders. A therapeutically effective dose refers to that amount of the compound sufficient to result in amelioration of symptoms of cardiovascular or neural disorders.

5.7.1. Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀ Compounds which exhibit large therapeutic indices are preferred.

While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

5.7.2. Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### REFERENCES

Draetta, G. Mammalian G1 cyclins. Curr. Opin. Cell Biol. 6, 842-846 (1994).

Schafer, K A. The cell cycle: a review. Vet Pathol 1998 35, 461-478 (1998).

Medema, R. H.; Kops, G. J. P. L.; Bos, J. L.; Burgering, B. M. T. AFX-like forkhead transcription factors mediate cell-cycle regulation by Ras and PKB through p27(kip1). Nature 40, 782-787 (2000).

Lee M H, Yang H Y. Negative regulators of cyclin-dependent kinases and their roles in cancers. Cell Mol Life Sci. 2001 58, 1907-1922 (2001).

Desdouets C. and Brechot C. p27: a pleiotropic regulator of cellular phenotype and a target for cell cycle dysregulation in cancer. Pathol Biol (Paris) 48, 203-210 (2000).

Sgambato A, Cittadini A, Faraglia B, Weinstein I B. Multiple functions of p27(Kip1) and its alterations in tumor cells: a review. J Cell Physiol. 183, 18-27 (2000).

### EXAMPLES

### Example 1

HGPRBMY1 Bioinformatics Analysis

G-protein coupled receptor sequences were used as a probe to search the Incyte and public domain EST databases. All the G-protein coupled receptor sequences available at the GPCRdb GPCR Database (http://www.gpcr.org/7tm) were used as queries. The search program used was gapped BLAST (Altschul, et al., 1997, Nucleic Acids Res 25:3389-3402). The top EST hits from the BLAST results were searched back against the non-redundant polypeptide and patent sequence databases. From this analysis, ESTs encoding a potential novel GPCRs were identified based on sequence homology. The public domain EST (ATCC® CloneID: 145375) was selected as potential novel GPCR candidate, HGPRBMY1 for subsequent analysis.

This EST was sequenced over its full length and was shown to contain a coding region bearing distinctive characteristics of a G-protein coupled receptor (GPCR). More specifically, the complete polypeptide sequence of HGPRBMY1 was analyzed for potential transmembrane domains. The TMPRED program was used for transmembrane prediction (K Hofmann and W Stoffel, 1993, Biol. Chem. Hoppe-Seyler 347:166). The program predicted seven transmembrane domains and the predicted domains match with the predicted transmembrane domains of related GPCRs at the sequence level. Based on sequence, structure and known GPCR signature sequences, the orphan polypeptide, HGPRBMY1, is likely a novel human GPCR.

### Example 2

Cloning of the Novel Human GPCR HGPRBMY1

A PCR primer pair, designed from the DNA sequence of ATCC® clone was used to amplify a piece of DNA from the same clone in which the antisense strand of the amplified fragment was biotinylated on the 3′ end. This biotinylated piece of double stranded DNA was denatured and incubated with a mixture of single-stranded covalently closed circular cDNA libraries which contain DNA corresponding to the sense strand.

Hybrids between the biotinylated DNA and the circular cDNA were captured on streptavidin magnetic beads. Upon thermal release of the cDNA from the biotinlyated DNA, the single stranded cDNA was converted into double strands using a primer homologous to a sequence on the cDNA cloning vector. The double stranded cDNA was introduced into E. coli by electroporation and the resulting colonies were screen by PCR, using the original primer pair, to identify the proper cDNA.

### Example 3

Expression Profiling of Novel Human GPCR, HGPRBMY1

A PCR primer was designed from the ATCC® clone and was used to measure the steady state levels of mRNA by quantitative PCR. The sequence of the primer pair was as follows:

5′-GATCCCCGTCGGTCATCTT-3′ (SEQ ID NO:3)

5′-GGTCACCACGTTGCAAAGC-3′ (SEQ ID NO:4)

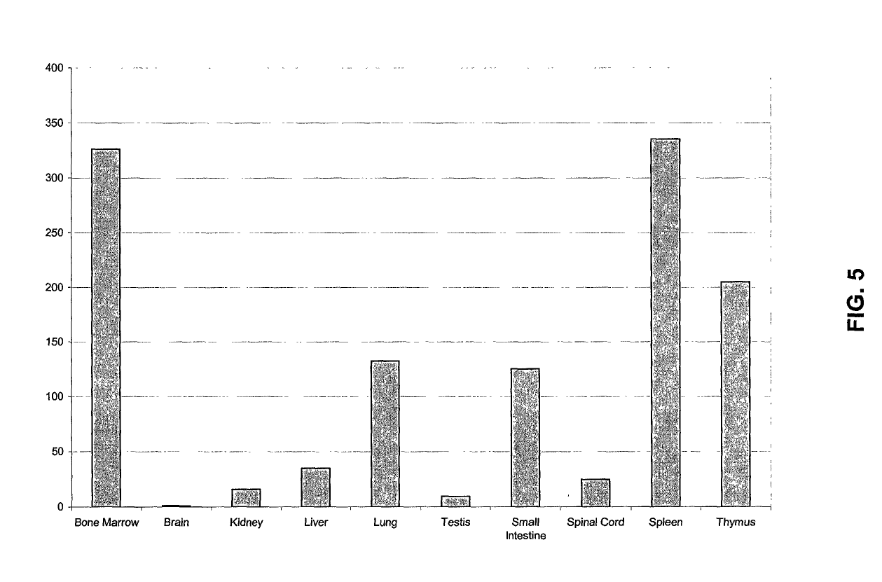

Briefly, first strand cDNA was made from commercially available mRNA. The relative amount of cDNA used in each assay was determined by performing a parallel experiment using a primer pair for a gene expressed in equal amounts in all tissues, cyclophilin. The cyclophilin primer pair detected small variations in the amount of cDNA in each sample and these data were used for normalization of the data obtained with the primer pair for this gene. The PCR data was converted into a relative assessment of the difference in transcript abundance amongst the tissues tested and the data is presented in FIG. 5.

Transcripts corresponding to the orphan GPCR, HGPRBMY1, are expressed highly in bone marrow and spleen, and to a lesser extent in the thymus.

### Example 4

Complementary Polynucleotides and Association of HGPRBMY1 to Cell Cycle and Apoptosis Regulation

Antisense molecules or nucleic acid sequences complementary to the HGPRBMY1 protein-encoding sequence, or any part thereof, is used to decrease or to inhibit the expression of naturally occurring HGPRBMY1. Although the use of antisense or complementary oligonucleotides comprising about 15 to 35 base-pairs is described, essentially the same procedure is used with smaller or larger nucleic acid sequence fragments. An oligonucleotide based on the coding sequence of HGPRBMY1 protein, as shown in FIG. 1, or as depicted in SEQ ID NO:1, for example, is used to inhibit expression of naturally occurring HGPRBMY1. The complementary oligonucleotide is typically designed from the most unique 5′ sequence and is used either to inhibit transcription by preventing promoter binding to the coding sequence, or to inhibit translation by preventing the ribosome from binding to the HGPRBMY1 protein-encoding transcript, among others. However, other regions may also be targeted.

Using an appropriate portion of the signal and 5′ sequence of SEQ ID NO:1, an effective antisense oligonucleotide includes any of about 15-35 nucleotides spanning the region which translates into the signal or 5′ coding sequence, among other regions, of the polypeptide as shown in FIG. 2 (SEQ ID NO:2). Appropriate oligonucleotides are designed using OLIGO 4.06 software and the HGPRBMY1 protein coding sequence (SEQ ID NO:1). Preferred oligonucleotides are deoxynucleotide-, or chimeric deoxynucleotide/ribonucleotide-based and are provided below. The oligonucleotides were synthesized using chemistry essentially as described in U.S. Pat. No. 5,849,902; which is hereby incorporated herein by reference in its entirety.

ID# Sequence

| | | |
|---|---|---|
| 15214 | ACAGGAUGCAACGCUUAAGUCGACG | (SEQ ID NO:5) |
| 15215 | AGAUUUGGAAAGGCAACACGCUGGC | (SEQ ID NO:6) |
| 15216 | CUUGAGGACGUCGAAGCAGGUGAUG | (SEQ ID NO:7) |
| 15217 | GCCUCCUCCGUGCGCAACAGCUUGA | (SEQ ID NO:8) |
| 15218 | CUGAUACAGGUCAUGGUGAGGAUGC | (SEQ ID NO:9) |

The HGPRBMY1 polypeptide has been shown to be involved in the regulation of mammalian cell cycle pathways. Subjecting cells with an effective amount of a pool of all five of the above antisense oligoncleotides (SEQ ID NO:5 thru 9) resulted in a significant increase in p27 expression/activity providing convincing evidence that HGPRBMY1 at least regulates the activity and/or expression of p27 either directly, or indirectly. Moreover, the results suggest that HGPRBMY1 is involved in the negative regulation of p27 activity and/or expression, either directly or indirectly. The p27 assay used is described below and was based upon the analysis of p27 activity as a downstream marker for proliferative signal transduction events.

Moreover, the HGPRBMY1 polypeptide has also been shown to be involved in the regulation of mammalian NF-□B and apoptosis pathways. Subjecting cells with an effective amount of a pool of all five of the above antisense oligoncleotides (SEQ ID NO:5 thru 9) resulted in a significant increase in I□B□ expression/activity providing convincing evidence that HGPRBMY1 at least regulates the activity and/or expression of I□B□ either directly, or indirectly. Moreover, the results suggest that HGPRBMY1 is involved in the negative regulation of NF-□B/I□B□ activity and/or expression, either directly or indirectly. The I□B□ assay used is described below and was based upon the analysis of I□B□ activity as a downstream marker for proliferative signal transduction events.

Based upon the regulation of p27 and IkB, antagonists directed against HGPRBMY1 would be useful for upregulating P27 and IkB, which would be beneficial to cancer patients by stopping proliferation and inducing apoptosis of a cell comprising a tumor.

Transfection of Post-quiescent A549 Cells with AntiSense Oligonucleotides.

Materials Needed:

A549 cells maintained in DMEM with high glucose (Gibco-BRL) supplemented with 10% Fetal Bovine Serum, 2 mM L-Glutamine, and 1X penicillin/streptomycin.

Opti-MEM (Gibco-BRL)

Lipofectamine 2000 (Invitrogen)

Antisense oligomers (Sequitur)

Polystyrene tubes.

Tissue culture treated plates.

Quiescent cells were prepared as follows:

Day 0: 300,000 A549 cells were seeded in a T75 tissue culture flask in 10 ml of A549 media, and incubated in at 37° C., 5% CO₂ in a humidified incubator for 48 hours.

Day 2: The T75 flasks were rocked to remove any loosely adherent cells, and the A549 growth media removed and replenished with 10 ml of fresh A549 media. The cells were cultured for six days without changing the media to create a quiescent cell population.

Day 8: Quiescent cells were plated in multi-well format and transfected with antisense oligonucleotides.

A549 cells were transfected according to the following:

1. Trypsinize T75 flask containing quiescent population of A549 cells.

2. Count the cells and seed 24-well plates with 60K quiescent A549 cells per well.

3. Allow the cells to adhere to the tissue culture plate (approximately 4 hours).

4. Transfect the cells with antisense and control oligonucleotides according to the following:

a. A 10× stock of lipofectamine 2000 (10 ug/ml is 10×) was prepared, and diluted lipid was allowed to stand at RT for 15 minutes. Stock solution of lipofectamine 2000 was 1 mg/ml. 10× solution for transfection was 10 ug/ml. To prepare 10× solution, dilute 10 ul of lipofectamine 2000 stock per 1 ml of Opti-MEM (serum free media).

b. A 10× stock of each oligomer was prepared to be used in the transfection. Stock solutions of oligomers were at 100 uM in 20 mM HEPES, pH 7.5. 10×concentration of oligomer was 0.25 uM. To prepare the 10× solutions, dilute 2.5 ul of oligomer per 1 ml of Opti-MEM.

c. Equal volumes of the 10× lipofectamine 2000 stock and the 10× oligomer solutions were mixed well, and incubated for 15 minutes at RT to allow complexation of the oligomer and lipid. The resulting mixture was 5×.

d. After the 15 minute complexation, 4 volumes of full growth media was added to the oligomer/lipid complexes (solution was 1×).

e. The media was aspirated from the cells, and 0.5 ml of the 1× oligomer/lipid complexes added to each well.

f. The cells were incubated for 16-24 hours at 37° C. in a humidified CO₂ incubator.

g. Cell pellets were harvested for RNA isolation and TaqMan analysis of downstream marker genes.

TaqMan Reactions—p27 Reactions

Quantitative RT-PCR analysis was performed on total RNA preps that had been treated with DNaseI or poly A selected RNA. The Dnase treatment may be performed using methods known in the art, though preferably using a Qiagen RNeasy kit to purify the RNA samples, wherein DNAse I treatment is performed on the column.

Briefly, a master mix of reagents was prepared according to the following table:

Dnase I Treatment

| Reagent | Per r′xn (in uL) |
|---|---|
| 10x Buffer | 2.5 |
| Dnase I (1 unit/ul @ 1 unit per ug | 2 |
| sample) | |
| DEPC H₂O | 0.5 |
| RNA sample @ 0.1 | 20 |
| ug/ul | |
| (2-3 ug total) | |
| Total | 25 |

Next, 5 ul of master mix was aliquoted per well of a 96-well PCR reaction plate (PE part # N801-0560). RNA samples were adjusted to 0.1 ug/ul with DEPC treated H₂O (if necessary), and 20 ul was added to the aliquoted master mix for a final reaction volume of 25 ul.

The wells were capped using strip well caps (PE part # N801-0935), placed in a plate, and briefly spun in a centrifuge to collect all volume in the bottom of the tubes. Generally, a short spin up to 500 rpm in a Sorvall RT is sufficient.

The plates were incubated at 37° C. for 30 mins. Then, an equal volume of 0.1 mM EDTA in 10 mM Tris was added to each well, and heat inactivated at 70° C. for 5 min. The plates were stored at −80° C. upon completion.

RT Reaction

A master mix of reagents was prepared according to the following table:

RT reaction

| | Γ | No RT |
|---|---|---|
| Reagent | x′n (in ul) | x′n (in ul) |
| 10x RT buffer | 5 | 2.5 |
| MgCl₂ | 11 | 5.5 |
| D0132NP | | |
| DNTP mixture | 10 | 5 |
| Random Hexamers | 2.5 | 1.25 |
| Rnase inhibitors | 1.25 | 0.625 |
| RT enzyme | 1.25 | — |
| Total RNA 500 ng (100 ng | 19.0 max | 10.125 max |
| no RT) | | |
| DEPC H₂O | — | — |
| Total | 50 uL | 25 uL |

Samples were adjusted to a concentration so that 500 ng of RNA was added to each RT rx′n (100 ng for the no RT). A maximum of 19 ul can be added to the RT rx′n mixture (10.125 ul for the no RT.) Any remaining volume up to the maximum values was filled with DEPC treated H₂O, so that the total reaction volume was 50 ul (RT) or 25 ul (no RT).

On a 96-well PCR reaction plate (PE part # N801-0560), 37.5 ul of master mix was aliquoted (22.5 ul of no RT master mix), and the RNA sample added for a total reaction volume of 50 ul (25 ul, no RT). Control samples were loaded into two or even three different wells in order to have enough template for generation of a standard curve.

The wells were capped using strip well caps (PE part # N801-0935), placed in a plate, and spin briefly in a centrifuge to collect all volume in the bottom of the tubes. Generally, a short spin up to 500 rpm in a Sorvall RT is sufficient.

For the RT-PCR reaction, the following thermal profile was used:

25° C. for 10 min

48° C. for 30 min

95° C. for 5 min

4° C. hold (for 1 hour)

Store plate @−20° C. or lower upon completion.

TaqMan Reaction (Template Comes from RT Plate.)

A master nix was prepared according to the following table:

TaqMan reaction (per well)

| Reagent | Per Rx′n (in ul) |
|---|---|
| TaqMan Master Mix | 4.17 |
| 100 uM Probe | .025 |
| (SEQ ID | |
| NO:12) | |
| 100 uM | .05 |
| Forward | |
| primer (SEQ | |
| ID NO:10) | |
| 100 uM | .05 |
| Reverse | |
| primer (SEQ | |
| ID NO:11) | |
| Template | — |
| DEPC H₂O | 18.21 |
| Total | 22.5 |

The primers used for the RT-PCR reaction is as follows:

P27 primer and probes:

| | |
|---|---|
| Forward Primer: | |
| CCCGGTGGACCACGAA | (SEQ ID NO:10) |
| Reverse Primer: | |
| GGCTCGCCTCTTCCATGTC | (SEQ ID NO:11) |
| TaqMan Probe: | |
| AACCCGGGACTTGGAGAAGCACTGC | (SEQ ID NO:12) |

TaqMan Reactions—IkB Reactions

Quantitative RT-PCR analysis was performed on total RNA preps that had been treated with DNaseI or poly A selected RNA. The Dnase treatment may be performed using methods known in the art, though preferably using a Qiagen RNeasy kit to purify the RNA samples, wherein DNAse I treatment is performed on the column.

Briefly, a master mix of reagents was prepared according to the following table:

Dnase I Treatment

| Reagent | Per r′xn in uL |
|---|---|
| 10x Buffer | 2.5 |
| Dnase I (1 unit/ul @ 1 unit per ug | 2 |
| sample) | |
| DEPC H₂O | 0.5 |
| RNA sample @ 0.1 | 20 |
| ug/ul | |
| (2-3 ug total) | |
| Total | 25 |

Next, 5 ul of master mix was aliquoted per well of a 96-well PCR reaction plate (PE part # N801-0560). RNA samples were adjusted to 0.1 ug/ul with DEPC treated H₂O (if necessary), and 20 ul was added to the aliquoted master mix for a final reaction volume of 25 ul.

The wells were capped using strip well caps (PE part # N801-0935), placed in a plate, and briefly spun in a centrifuge to collect all volume in the bottom of the tubes. Generally, a short spin up to 500 rpm in a Sorvall RT is sufficient

The plates were incubated at 37° C. for 30 mins. Then, an equal volume of 0.1 mM EDTA in 10 mM Tris was added to each well, and heat inactivated at 70° C. for 5 min. The plates were stored at −80° C. upon completion.

RT Reaction

A master mix of reagents was prepared according to the following table:

RT reaction

| | Γ | No RT |
|---|---|---|
| Reagent | x′n (in ul) | x′n (in ul) |
| 10x RT buffer | 5 | 2.5 |
| MgCl₂ | 11 | 5.5 |
| DNTP mixture | 10 | 5 |
| Random Hexamers | 2.5 | 1.25 |
| Rnase inhibitors | 1.25 | 0.625 |
| RT enzyme | 1.25 | — |
| Total RNA 500 ng (100 ng | 19.0 max | 10.125 max |
| no RT) | | |
| DEPC H₂O | — | — |
| Total | 50 uL | 25 uL |

Samples were adjusted to a concentration so that 500 ng of RNA was added to each RT rx′n (100 ng for the no RT). A maximum of 19 ul can be added to the RT rx′n mixture (10.125 ul for the no RT.) Any remaining volume up to the maximum values was filled with DEPC treated H₂O, so that the total reaction volume was 50 ul (RT) or 25 ul (no RT).

On a 96-well PCR reaction plate (PE part # N801-0560), 37.5 ul of master mix was aliquoted (22.5 ul of no RT master mix), and the RNA sample added for a total reaction volume of 50 ul (25 ul, no RT). Control samples were loaded into two or even three different wells in order to have enough template for generation of a standard curve.

The wells were capped using strip well caps (PE part # N801-0935), placed in a plate, and spin briefly in a centrifuge to collect all volume in the bottom of the tubes. Generally, a short spin up to 500 rpm in a Sorvall RT is sufficient.

For the RT-PCR reaction, the following thermal profile was used:

25° C. for 10 min

48° C. for 30 min

95° C. for 5 min

4° C. hold (for 1 hour)

Store plate @−20° C. or lower upon completion.

TaqMan Reaction (Template Comes from RT Plate.)

A master mix was prepared according to the following table:

TaqMan reaction (per well)

| Reagent | Per Rx′n in ul |
|---|---|
| TaqMan Master Mix | 4.17 |
| 100 uM Probe | .025 |
| (SEQ ID | |
| NO:15) | |
| 100 uM | .05 |
| Forward | |
| primer (SEQ | |
| ID NO:13) | |
| 100 uM | .05 |
| Reverse | |
| primer (SEQ | |
| ID NO:14) | |
| Template | — |
| DEPC H₂O | 18.21 |
| Total | 22.5 |

The primers used for the RT-PCR reaction is as follows:

IkB primer and probes:

| | |
|---|---|
| Forward Primer: | |
| GAGGATGAGGAGAGCTATGACACA | (SEQ ID NO:13) |
| Reverse Primer: | |
| CCCTTTGCACTCATAACGTCAG | (SEQ ID NO:14) |
| TaqMan Probe: | |
| AAACACACAGTCATCATAGGGCAGCTCGT | (SEQ ID NO:15) |

Using a Gilson P-10 repeat pipetter, 22.5 ul of master mix was aliquouted per well of a 96-well optical plate. Then, using P-10 pipetter, 2.5 ul of sample was added to individual wells. Generally, RT samples are run in triplicate with each primer/probe set used, and no RT samples are run once and only with one primer/probe set, often gapdh (or other internal control).

A standard curve is then constructed and loaded onto the plate. The curve has five points plus one no template control (NTC, =DEPC treated H₂O). The curve was made with a high point of 50 ng of sample (twice the amount of RNA in unknowns), and successive samples of 25, 10, 5, and 1 ng. The curve was made from a control sample(s) (see above).

The wells were capped using optical strip well caps (PE part # N801-0935), placed in a plate, and spun in a centrifuge to collect all volume in the bottom of the tubes. Generally, a short spin up to 500 rpm in a Sorvall RT is sufficient.

Plates were loaded onto a PE 5700 sequence detector making sure the plate is aligned properly with the notch in the upper right hand corner. The lid was tightened down and run using the 5700 and 5700 quantitation program and the SYBR probe using the following thermal profile:

50° C. for 2 min

95° C. for 10 min

and the following for 40 cycles:

95° C. for 15 sec

60° C. for 1 min

Change the reaction volume to 25 ul.

Once the reaction was complete, a manual threshold of around 0.1 was set to minimize the background signal. Additional information relative to operation of the GeneAmp 5700 machine may be found in reference to the following manuals: “GeneAmp 5700 Sequence Detection System Operator Training CD”; and the “User's Manual for 5700 Sequence Detection System”; available from Perkin-Elmer and hereby incorporated by reference herein in their entirety.

### Example 5

HGPRBMY2 Bioinformatics Analysis

G-protein coupled receptor sequences were used as a probe to search the Incyte and public domain EST databases. All the G-protein coupled receptor sequences available at the GPCRdb GPCR Database (http://www.gpcr.org/7tm) were used as queries. The search program used was gapped BLAST (Altschul, et al., 1997, Nucleic Acids Res 25:3389-3402). The top EST hits from the BLAST results were searched back against the non-redundant polypeptide and patent sequence databases. From this analysis, ESTs encoding a potential novel GPCRs were identified based on sequence homology. The public domain EST (ATCC CloneID: 3293096) was selected as potential novel GPCR candidate, HGPRBMY2 for subsequent analysis.

This EST was sequenced and the full-length clone of this GPCR was obtained using the EST sequence information. The complete polypeptide sequence of HGPRBMY2 was analyzed for potential transmembrane domains. The TMPRED program was used for transmembrane prediction (K Hofmann and W Stoffel, 1993, Biol. Chem. Hoppe-Seyler 347:166). The program predicted seven transmembrane domains and the predicted domains match with the predicted transmembrane domains of related GPCRs at the sequence level. Based on sequence, structure and known GPCR signature sequences, the orphan polypeptide, HGPRBMY2, is likely a novel human GPCR.

### Example 6

Cloning of the Novel Human GPCR HGPRBMY2

A PCR primer pair, designed from the DNA sequence of ATCC clone was used to amplify a piece of DNA from the same clone in which the antisense strand of the amplified fragment was biotinylated on the 3′ end. This biotinylated piece of double stranded DNA was denatured and incubated with a mixture of single-stranded covalently closed circular cDNA libraries which contain DNA corresponding to the sense strand.

Hybrids between the biotinylated DNA and the circular cDNA were captured on streptavidin magnetic beads. Upon thermal release of the cDNA from the biotinlyated DNA, the single stranded cDNA was converted into double strands using a primer homologous to a sequence on the CDNA cloning vector. The double stranded cDNA was introduced into E. coli by electroporation and the resulting colonies were screen by PCR, using the original primer pair, to identify the proper cDNA.

### Example 7

Expression Profiling of Novel Human GPCR, HGPRBMY2

A PCR primer was designed from the ATCC clone and was used to measure the steady state levels of mRNA by quantitative PCR. The sequence of the primer pair was as follows:

5′-TTTCTGGATCGTCAGCTTGCT-3′(SEQ ID NO:15)

5′-ACAGGGCTGGTCCACTCTTCT-3′(SEQ ID NO:16)

Briefly, first strand cDNA was made from commercially available mRNA. The relative amount of CDNA used in each assay was determined by performing a parallel experiment using a primer pair for a gene expressed in equal amounts in all tissues, cyclophilin. The cyclophilin primer pair detected small variations in the amount of CDNA in each sample and these data were used for normalization of the data obtained with the primer pair for this gene. The PCR data was converted into a relative assessment of the difference in transcript abundance amongst the tissues tested and the data is presented in FIG. 10.

Transcripts corresponding to the orphan GPCR, HGPRBMY2, are expressed highly in heart, testes and to a lesser degree in thymus.

### Example 8

Method of Assessing the Expression Profile of the Novel HGPRBMY2 Polypeptides of the Present Invention Using Expanded mRNA Tissue and Cell Sources

Total RNA from tissues was isolated using the TriZol protocol (Invitrogen) and quantified by determining its absorbance at 260 nM. An assessment of the 18s and 28s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For HGPRBMY2, the primer probe sequences were as follows

| | |
|---|---|
| Forward Primer | |
| 5′-CACCAACCGAAGGGCTTTC-3′ | (SEQ ID NO:25) |
| Reverse Primer | |
| 5′-CCACATGGGTGATCCTACGAT-3′ | (SEQ ID NO:26) |
| TaqMan Probe | |
| 5′-ACTGCCACCAGCCAGACCACACCTA-3′ | (SEQ ID NO:27) |

DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+ RNA. If not the RNA was not used in actual experiments.

Reverse Transcription Reaction and Sequence Detection

100 ng of Dnase-treated total RNA was annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 500 μM of each dNTP, buffer and 5U AmpliTaq Gold™. The PCR reaction was then held at 94° C. for 12 min, followed by 40 cycles of 94° C. for 15 sec and 60° C. for 30 sec.

Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}

The expanded expression profile of the HGPRBMY2 polypeptide, is provided in FIG. 16 and are described elsewhere herein.

### Example 9

Functional Characterization of the Novel Human GPCR, HGPRBMY2

The use of mammalian cell reporter assays to demonstrate functional coupling of known GPCRs (G Protein Coupled Receptors) has been well documented in the literature (Gilman, 1987, Boss et al., 1996; Alam & Cook, 1990; George et al., 1997; Selbie & Hill, 1998; Rees et al., 1999). In fact, reporter assays have been successfully used for identifying novel small molecule agonists or antagonists against GPCRs as a class of drug targets (Zlokarnik et al., 1998; George et al., 1997; Boss et al., 1996; Rees et al, 2001). In such reporter assays, a promoter is regulated as a direct consequence of activation of specific signal transduction cascades following agonist binding to a GPCR (Alam & Cook 1990; Selbie & Hill, 1998; Boss et al., 1996; George et al., 1997; Gilman, 1987).

A number of response element-based reporter systems have been developed that enable the study of GPCR function. These include cAMP response element (CRE)-based reporter genes for G alpha i/o, G alpha s-coupled GPCRs, Nuclear Factor Activator of Transcription (NFAT)-based reporters for G alpha q/11—coupled receptors and MAP kinase reporter genes for use in Galpha i/o coupled receptors (Selbie & Hill, 1998; Boss et al., 1996; George et al., 1997; Gilman, 1987; Rees et al., 2001). Transcriptional response elements that regulate the expression of Beta-Lactamase within a CHO K1 cell line (Cho/NFAT-CRE: Aurora Biosciences™) (Zlokarnik et al., 1998) have been implemented to characterize the function of the orphan HGPRBMY2 polypeptide of the present invention. The system enables demonstration of constitutive G-protein coupling to endogenous cellular signaling components upon intracellular overexpression of orphan receptors. Overexpression has been shown to represent a physiologically relevant event. For example, it has been shown that overexpression occurs in nature during metastatic carcinomas, wherein defective expression of the monocyte chemotactic protein 1 receptor, CCR2, in macrophages is associated with the incidence of human ovarian carcinoma (Sica, et al.,2000; Salcedo et al., 2000). Indeed, it has been shown that overproduction of the Beta 2 Adrenergic Receptor in transgenic mice leads to constitutive activation of the receptor signaling pathway such that these mice exhibit increased cardiac output (Kypson et al., 1999; Dorn et al., 1999). These are only a few of the many examples demonstrating constitutive activation of GPCRs whereby many of these receptors are likely to be in the active, R*, conformation (J. Wess 1997).

Materials and Methods:

DNA Constructs:

The putative GPCR HGPRBMY2 cDNA was PCR amplified using PFU™ (Stratagene). The primers used in the PCR reaction were specific to the HGPRBMY2 polynucleotide and were ordered from Gibco BRL (5 prime primer: 5′-CCCAAGCTTATGCAGGCGCTTAACATTACCCCG-3′ (SEQ ID NO:17), 3 prime primer: 5′-CGGGATCCTTAATGCCACTGTCTAAAGGAAGA-3′ (SEQ ID NO:18). The following 3 prime primer was used to add a Flag-tag epitope to the HGPRBMY2 polypeptide for immunocytochemistry: 5′-CGGGATCCTTACTTGTCGTCGTCGTCCTTGTAGTCCATATGCCCACTGTCTAA AGGAGAATTCTCAAC-3′(SEQ ID NO:19). The product from the PCR reaction was isolated from a 0.8% Agarose gel (Invitrogen) and purified using a Gel Extraction Kit™ from Qiagen.

The purified product was then digested overnight along with the pcDNA3.1 Hygro™ mammalian expression vector from Invitrogen using the HindIII and BamHI restriction enzymes (New England Biolabs). These digested products were then purified using the Gel Extraction Kit™ from Qiagen and subsequently ligated to the pcDNA3.1 Hygro™ expression vector using a DNA molar ratio of 4 parts insert: 1 vector. All DNA modification enzymes were purchased from NEB. The ligation was incubated overnight at 16 degrees Celsius, after which time, one microliter of the mix was used to transform DH5 alpha cloning efficiency competent E. coli™ (Gibco BRL). A detailed description of the pcDNA3.1 Hygro™ mammalian expression vector is available at the Invitrogen web site (www.Invitrogen.com). The plasmid DNA from the ampicillin resistant clones were isolated using the Wizard DNA Miniprep System™ from Promega. Positive clones were then confirmed and scaled up for purification using the Qiagen Maxiprep™ plasmid DNA purification kit.

Cell Line Generation:

The pcDNA3.1hygro vector containing the orphan HGPRBMY2 cDNA were used to transfect Cho/NFAT-CRE (Aurora Biosciences) cells using Lipofectamine 2000™ according to the manufacturers specifications (Gibco BRL). Two days later, the cells were split 1:3 into selective media (DMEM 11056, 600 ug/ml Hygromycin, 200 ug/ml Zeocin, 10% FBS). All cell culture reagents were purchased from Gibco BRL-Invitrogen.

The Cho/NFAT-CRE cell lines, transiently or stably transfected with the orphan HGPRBMY2 GPCR, were analyzed using the FACS Vantage SE™ (BD), fluorescence microscopy (Nikon), and the LJL Analyst™ (Molecular Devices). In this system, changes in real-time gene expression, as a consequence of constitutive G-protein coupling of the orphan HGPRBMY2 GPCR, is examined by analyzing the fluorescence emission of the transformed cells at 447 nm and 518 nm. The changes in gene expression can be visualized using Beta-Lactamase as a reporter, that, when induced by the appropriate signaling cascade, hydrolyzes an intracellularly loaded, membrane-permeant ester substrate (CCF2/AM™ Aurora Biosciences; Zlokarnik, et al., 1998). The CCF2/AM™ substrate is a 7-hydroxycoumarin cephalosporin with a fluorescein attached through a stable thioether linkage. Induced expression of the Beta-Lactamase enzyme is readily apparent since each enzyme molecule produced is capable of changing the fluorescence of many CCF2/AM™ substrate molecules. A schematic of this cell based system is shown below.

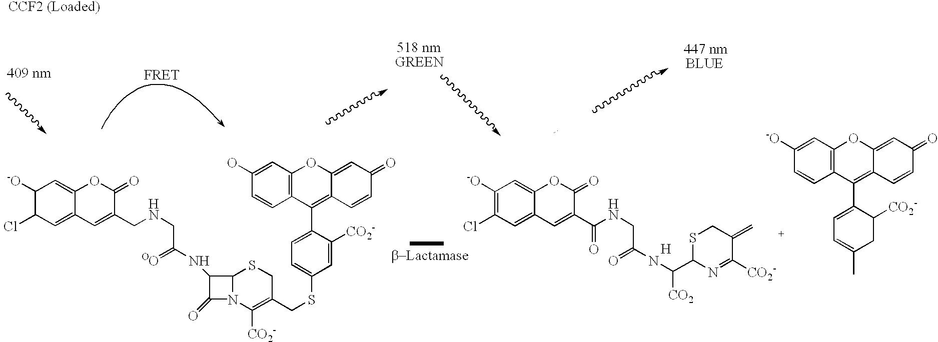

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only thus giving rise to blue fluorescent emission at 447 nm.

Fluorescent emissions were detected using a Nikon-TE300 microscope equipped with an excitation filter (D405/10×-25), dichroic reflector (430DCLP), and a barrier filter for dual DAPI/FITC (510 mM) to visually capture changes in Beta-Lactamase expression. The FACS Vantage SE is equiped with a Coherent Enterprise II Argon Laser and a Coherent 302C Krypton laser. In flow cytometry, UV excitation at 351-364 nm from the Argon Laser or violet excitation at 407 nm from the Krypton laser are used. The optical filters on the FACS Vantage SE are HQ460/50 m and HQ535/40 m bandpass separated by a 490 dichroic mirror.

Prior to analyzing the fluorescent emissions from the cell lines as described above, the cells were loaded with the CCF2/AM substrate. A 6×CCF2/AM loading buffer was prepared whereby 1 mM CCF2/AM (Aurora Biosciences) was dissolved in 100% DMSO (Sigma). 12 ul of this stock solution was added to 60 ul of 100 mg/ml Pluronic F127 (Sigma) in DMSO containing 0.1% Acetic Acid (Sigma). This solution was added while vortexing to 1 mL of Sort Buffer (PBS minus calcium and magnesium-Gibco-25 mM HEPES-Gibco- pH 7.4, 0.1% BSA). Cells were placed in serum-free media and the 6×CCF2/AM was added to a final concentration of 1×. The cells were then loaded at room temperature for one to two hours, and then subjected to fluorescent emission analysis as described herein. Additional details relative to the cell loading methods and/or instrument settings may be found by reference to the following publications: see Zlokarnik, et al., 1998; Whitney et al., 1998; and BD Biosciences,1999.

Immunocytochemistry:

The cell lines transfected and selected for expression of Flag-epitope tagged orphan GPCRs were analyzed by immunocytochemistry. The cells were plated at 1×10ˆ 3 in each well of a glass slide (VWR). The cells were rinsed with PBS followed by acid fixation for 30 minutes at room temperature using a mixture of 5% Glacial Acetic Acid/90% ETOH. The cells were then blocked in 2% BSA and 0.1% Triton in PBS, incubated for 2 h at room temperature or overnight at 4° C. A monoclonal anti-Flag FITC antibody was diluted at 1:50 in blocking solution and incubated with the cells for 2 h at room temperature. Cells were then washed three times with 0.1%Triton in PBS for five minutes. The slides were overlayed with mounting media dropwise with Biomedia-Gel Mount™ (Biomedia; Containing Anti-Quenching Agent). Cells were examined at 10× magnification using the Nikon TE300 equiped with FITC filter (535 nm).

Results—HGPRBMY2 Constitutively Activates Gene Expression Through the NFAT Response Element.

There is strong evidence that certain GPCRs exhibit a cDNA concentration-dependent constitutive activity through cAMP response element (CRE) luciferase reporters (Chen et al., 1999). In an effort to demonstrate functional coupling of HGPRBMY2 to known GPCR second messenger pathways, the HGPRBMY2 polypeptide was expressed at high constitutive levels in the Cho-NFAT/CRE cell line. To this end, the HGPRBMY2 cDNA was PCR amplified and subcloned into the pcDNA3.1 hygro™ mammalian expression vector as described herein. Early passage Cho-NFAT/CRE cells were then transfected with the resulting pcDNA3.1 hygro™/HGPRBMY2 construct. Transfected and non-transfected Cho-NFAT/CRE cells (control) were loaded with the CCF2 substrate and stimulated with 10 nM PMA, and 1 uM Thapsigargin (NFAT stimulator) or 10 uM Forskolin (CRE stimulator) to fully activate the NFAT/CRE element. The cells were then analyzed for fluorescent emission by FACS.

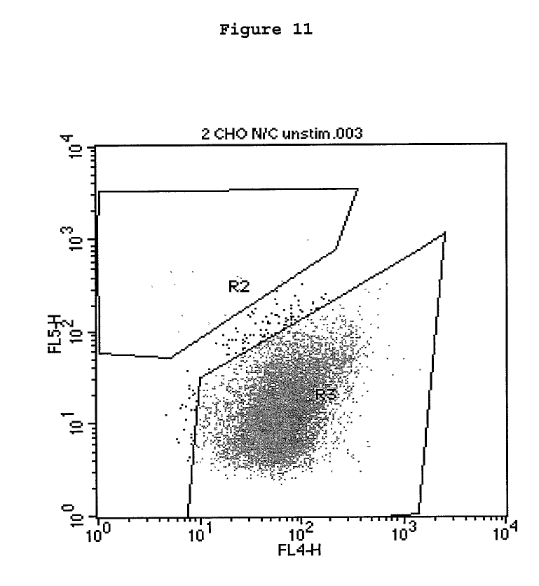

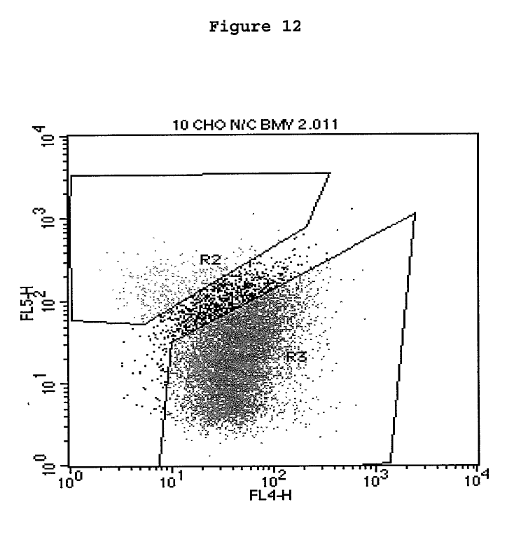

The FACS profile demonstrates the constitutive activity of HGPRBMY2 in the Cho-NFAT/CRE line as evidenced by the significant population of cells with blue fluorescent emission at 447 nm (see FIG. 12: Blue Cells). As expected, the NFAT/CRE response element in the untransfected control cell line was not activated (i.e., beta lactamase not induced), enabling the CCF2 substrate to remain intact, and resulting in the green fluorescent emission at 518 nM (see FIG. 11—Green Cells). A very low level of leaky Beta Lactamase expression was detectable as evidenced by the small population of cells emitting at 447 nm. Analysis of a stable pool of cells transfected with HGPRBMY2 revealed constitutive coupling of the cell population to the NFAT/CRE response element, activation of Beta Lactamase and cleavage of the substrate (FIG. 12—Blue Cells). These results demonstrate that overexpression of HGPRBMY2 leads to constitutive coupling of signaling pathways known to be mediated by Gq/11 or Gs coupled receptors that converge to activate either the NFAT or CRE response elements respectively (Boss et al., 1996; Chen et al., 1999).

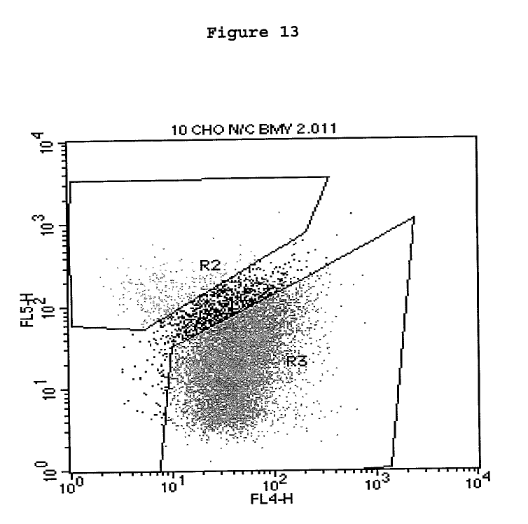

In an effort to further characterize the observed functional coupling of the HGPRBMY2 polypeptide, its ability to couple to the cAMP response element (CRE) independent of the NFAT response element was examined. To this end, HEK-CRE cell line that contained only the integrated 3XCRE linked to the Beta-Lactamase reporter was transfected with the pcDNA3.1 hygro™/HGPRBMY2 construct. Analysis of the fluorescence emission from this stable pool showed that HGPRBMY2 does not constitutively couple to the cAMP mediated second messenger pathways (see FIG. 13). Experiments have shown that known Gs coupled receptors do demonstrate constitutive activation when overexpressed in the HEK-CRE cell line. For example, direct activation of adenylate cyclase using 10 uM Forskolin has been shown to activate CRE and the subsequent induction of Beta-Lactamase in the HEK-CRE cell line (data not shown). In conclusion, the results are consistent with HGPRBMY2 representing a functional GPCR analogous to known Gq coupled receptors. Therefore, constitutive expression of HGPRBMY2 in the CHO Nfat/CRE cell line leads to NFAT activation through accumulation of intracellular Ca²⁺ as has been demonstrated for the M3 muscarinic receptor (Boss et al., 1996).

In preferred embodiments, the HGPRBMY2 polynucleotides and polypeptides, including agonists, antagonists, and fragments thereof, are useful for modulating intracellular Ca²⁺ levels, modulating Ca²⁺ sensitive signaling pathways, and modulating NFAT element associated signaling pathways.

Demonstration of Cell Surface Expression:

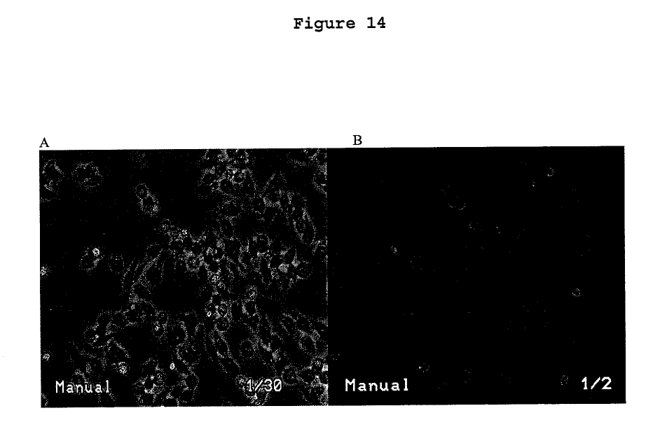

HGPRBMY2 was tagged at the C-terminus using the Flag epitope and inserted into the pcDNA3.1 hygro™ expression vector, as described herein. Immunocytochemistry of Cho Nfat-CRE cell lines transfected with the Flag-tagged HGPRBMY2 construct with FITC conjugated Anti Flag monoclonal antibody demonstrated that HGPRBMY2 is indeed a cell surface receptor. The immunocytochemistry also confirmed expression of the HGPRBMY2 in the Cho Nfat-CRE cell lines. Briefly, Cho Nfat-CRE cell lines were transfected with pcDNA3.1 hygro™/HGPRBMY2-Flag vector, fixed with 70% methanol, and permeablized with 0.1% Triton×100. The cells were then blocked with 1% Serum and incubated with a FITC conjugated Anti Flag monoclonal antibody at 1:50 dilution in PBS-Triton. The cells were then washed several times with PBS-Triton, overlayed with mounting solution, and fluorescent images were captured (see FIG. 14). The control cell line, non-transfected ChoNfat CRE cell line, exhibited no detectable background fluorescence (Data not shown). The BMY2 -FLAG tagged expressing Cho Nfat CRE line exhibited specific plasma membrane expression as indicated (Panel B). These data provide clear evidence that BMY2 is expressed at the plasma membrane. Plasma membrane localization in consistent with HGPRBMY2 representing a 7 transmembrane domain containing GPCR. Taken together, the data indicates that HGPRBMY2 is a cell surface GPCR that functions through increases in Ca2+ signal transduction pathways.

Screening Paradigm

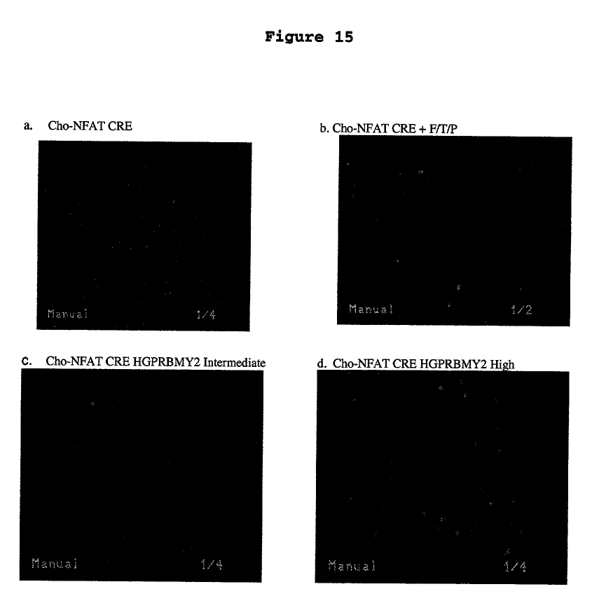

The Aurora Beta-Lactamase technology provides a clear path for identifying agonists and antagonists of the HGPRBMY2 polypeptide. Cell lines that exhibit a range of constitutive coupling activity have been identified by sorting through HGPRBMY2 transfected cell lines using the FACS Vantage SE (see FIG. 15). For example, cell lines have been sorted that have an intermediate level of HGPRBMY2 expression, which also correlates with an intermediate coupling response, using the LJL analyst. Such cell lines will provide the opportunity to screen, indirectly, for both agonists and antagonists of HGPRBMY2 by looking for inhibitors that block the beta lactamase response, or agonists that increase the beta lactamase response. As described herein, modulating the expression level of beta lactamase directly correlates with the level of cleaved CCR2 substrate. For example, this screening paradigm has been shown to work for the identification of modulators of a known GPCR, 5HT6, that couples through Adenylate Cyclase, in addition to, the identification of modulators of the 5HT2c GPCR, that couples through changes in [Ca²⁺]i. The data shown below represent cell lines that have been engineered with the desired pattern of HGPRBMY2 expression to enable the identification of potent small molecule agonists and antagonists. HGPRBMY2 modulator screens may be carried out using a variety of high throughput methods known in the art, though preferably using the fully automated Aurora UHTSS system. The uninduced, HGPRBMY2 transfected Cho Nfat-CRE cell line represents the relative background level of beta lactamase expression (FIG. 15; panel a). Following treatment with a cocktail of 10 nM Forskolin, 1 uM Thapsigargin, and 100 nM PMA (FIG. 15; F/T/P; panel b), the cells fully activate the CRE-NFAT response element demonstrating the dynamic range of the assay. Panel C (FIG. 15) represents a HGPRBMY2 transfected Cho Nfat-CRE cell line that shows an intermediate level of beta lactamase expression post F/T/P stimulation, while panel D (FIG. 15) represents a HGPRBMY2 transfected Cho Nfat-CRE cell line that shows a high level of beta lactamase expression post F/T/P stimulation.

In preferred embodiments, the HGPRBMY2 transfected Cho Nfat-CRE cell lines of the present invention are useful for the identification of agonists and antagonists of the HGPRBMY2 polypeptide. Representative uses of these cell lines would be their inclusion in a method of identifying HGPRBMY2 agonists and antagonists. Preferably, the cell lines are useful in a method for identifying a compound that modulates the biological activity of the HGPRBMY2 polypeptide, comprising the steps of (a) combining a candidate modulator compound with a host cell expressing the HGPRBMY2 polypeptide having the sequence as set forth in SEQ ID NO:14; and (b) measuring an effect of the candidate modulator compound on the activity of the expressed HGPRBMY2 polypeptide. Representative vectors expressing the HGPRBMY2 polypeptide are referenced herein (e.g., pcDNA3.1 hygro™) or otherwise known in the art.

The cell lines are also useful in a method of screening for a compound that is capable of modulating the biological activity of HGPRBMY2 polypeptide, comprising the steps of: (a) determining the biological activity of the HGPRBMY2 polypeptide in the absence of a modulator compound; (b) contacting a host cell expression the HGPRBMY2 polypeptide with the modulator compound; and (c) determining the biological activity of the HGPRBMY2 polypeptide in the presence of the modulator compound; wherein a difference between the activity of the HGPRBMY2 polypeptide in the presence of the modulator compound and in the absence of the modulator compound indicates a modulating effect of the compound. Additional uses for these cell lines are described herein or otherwise known in the art

1. Rees, S., Brown, S., Stables, J.: Reporter gene systems for the study of G Protein Coupled Receptor signalling in mammalian cells. In Milligan G. (ed.): Signal Transduction: A practical approach. Oxford: Oxford University Press, 1999: 171-221.

2. Alam, J., Cook, J. L.: Reporter Genes: Application to the study of mammalian gene transcription. Anal. Biochem. 1990; 188: 245-254.

3. Selbie, L. A. and Hill, S. J.: G protein-coupled receptor cross-talk: The fine-tuning of multiple receptor-signaling pathways. TiPs. 1998; 19: 87-93.

4. Boss, V., Talpade, D. J., and Murphy, T. J.: Induction of NFAT mediated transcription by Gq-coupled Receptors in lympoid and non-lymphoid cells. JBC. 1996; 271: 10429-10432.

5. George, S. E., Bungay, B. J., and Naylor, L. H.: Functional coupling of endogenous serotonin (5-HT1B) and calcitonin (Cla) receptors in Cho cells to a cyclic AMP-responsive responsive luciferase reporter gene. J. Neurochem. 1997; 69: 1278-1285.

6. Suto, C M, Igna D M: Selection of an optimal reporter for cell-based high throughput screening assays. J. Biomol. Screening. 1997; 2: 7-12.

7. Zlokarnik, G., Negulescu, P. A., Knapp, T. E., More, L., Burres, N., Feng, L., Whitney, M., Roemer, K., and Tsien, R. Y. Quantitation of transcription and clonal selection of single living cells with a B-Lactamase Reporter. Science. 1998; 279: 84-88.

8. S. Fiering et. al., Genes Dev. 4, 1823 (1990).

9. J. Karttunen and N. Shastri, PNAS 88, 3972 (1991).

10. Hawes, B. E., Luttrell. L. M., van Biesen, T., and Lefkowitz, R. J. (1996) JBC 271, 12133-12136.

11. Gilman, A.G. (1987) Annul. Rev. Biochem. 56, 615-649.

12. Maniatis et al.,

13. Salcedo, R., Ponce, M. L., Young, H. A., Wasserman, K., Ward, J. M., Kleinman, H. K., Oppenheim, J. J., Murphy, W. J. Human endothelial cells express CCR2 and respond to MCP-1: direct role of MCP-1 in angiogenesis and tumor progression. Blood. 2000; 96 (1): 34-40.

14. Sica, A., Saccani, A., Bottazzi, B., Bernasconi, S., Allavena, P., Gaetano, B., LaRossa, G., Scotton, C., Balkwill F., Mantovani, A. Defective expression of the monocyte chemotactic protein 1 receptor CCR2 in macrophages associated with human ovarian carcinoma. J. Immunology. 2000; 164: 733-8.

15. Kypson, A., Hendrickson, S., Akhter, S., Wilson, K., McDonald, P., Lilly, R., Dolber, P., Glower, D., Lefkowitz, R., Koch, W. Adenovirus-mediated gene transfer of the B2 AR to donor hearts enhances cardiac function. Gene Therapy. 1999; 6: 1298-304.

16. Dorn, G. W., Tepe, N. M., Lorenz, J. N., Kock, W. J., Ligget, S. B. Low and high level transgenic expression of B2AR differentially affect cardiac hypertrophy and function in Galpha q-overexpressing mice. PNAS. 1999; 96: 6400-5.

17. J. Wess. G protein coupled receptor: molecular mechanisms involved in receptor activation and selectivity of G-protein recognition.

18. Whitney, M, Rockenstein, E, Cantin, G., Knapp, T., Zlokarnik, G., Sanders, P., Durick, K., Craig, F. F., and Negulescu, P. A. A genome-wide functional assay of signal transduction in living mammalian cells. 1998. Nature Biotech. 16: 1329-1333.

19. BD Biosciences: FACS Vantage SE Training Manual. Part Number 11-11020-00 Rev. A. August 1999.

20. Chen, G., Jaywickreme, C., Way, J., Armour S., Queen K., Watson., C., Ignar, D., Chen, W. J., Kenakin, T. Constitutive Receptor systems for drug discovery. J. Pharmacol. Toxicol. Methods 1999; 42: 199-206.

### Example 10

Phage Display Methods for Identifying Peptide Ligands or Modulators of Orphan GPCRs

Creation of Peptide Libraries.

Two types of libraries may be created: i.) libraries of 12- and 15 mer peptides for finding peptides that may function as (ant-)agonists and ii.) libraries of peptides with 23-33 random residues that are for finding natural ligands through database searches.

The 15 mer library may be i.) an aliquot of the fUSE5-based 15 mer library originally constructed by G P Smith (Scott, J K and Smith, G P. 1990, Science 249, 386-390). Such a library may be made essentially as described therein, or ii.) a library that is constructed at Bristol-Myers Squibb in vector M13KE (New England Biolabs) using a single-stranded library oligonucleotide extension method (S. S. Sidhu, H. B. Lowman, B. C. Cunningham, J. A. Wells: Methods Enzymol., 2000, vol 328, 333-363).

The 12 mer library is an aliquot of the M13KE-based ‘PhD’ 12 mer library (New England Biolabs).

The libraries with 27-33 random residues are also constructed at Bristol-Myers Squibb in vector M13KE (New England Biolabs) using the method described in (S. S. Sidhu, H. B. Lowman, B. C. Cunningham, J. A. Wells: Methods Enzymol., 2000, vol 328, 333-363).

All libraries in vector M13KE utilize the standard NNK motif to encode the specified number of random residues, where N=A+G+C+T and where K=G+T.

Sequencing of Bound Phage:

Standard procedure. Phage in eluates are infected into E. coli host strain (TG1 for fUSE5-based 15 mer library; ER2738 (New England Biolabs) for all M13KE-based libraries) and are plated for single colonies (fUSE5 vector) or plaques (all M13KE-based libraries). Colonies are grown in liquid and sequenced by standard procedure which involves 1.) generating PCR product with suitable primers that anneal adjacent to the library segments in the vectors and 2.) sequencing of the PCR products using one primer of each PCR primer pair. Sequences are analyzed for homologies by visual inspection or by using the Vector NTI alignment tool.

Peptide Synthesis

Peptides are synthesized on Fmoc-Knorr amide resin [N-(9-fluorenyl)methoxycarbonyl-Knorr amide-resin, Midwest Biotech, Fishers, Ind.] with an Applied Biosystems (Foster City, Calif.) model 433A synthesizer and theFastMoc chemistry protocol (0.25 mmol scale) supplied with the instrument. Amino acids are double coupled as their N-alpha-Fmoc- derivatives and reactive side chains are protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group is removed by the multi-step treatment with piperidine in N-Methylpyrrolidone described by the manufacturer. The N-terminal free amines are then treated with 10% acetic anhydride, 5% Diisopropylamine in N-Methylpyrrolidone to yield the N-acetyl-derivative. The protected peptidyl-resins are simultaneously deprotected and removed from the resin by standard methods. The lyophilized peptides are purified on C₁₈ to apparent homogeneity as judged by RP-HPLC analysis. Predicted peptide molecular weights are verified by electrospray mass spectrometry. (J. Biol. Chem. vol. 273, pp.12041-12046, 1998)

Cyclic analogs are prepared from the crude linear products. The cystine disulfide may be formed using one of the following methods:

Method 1: A sample of the crude peptide is dissolved in water at a concentration of 0.5 mg/mL and the pH adjusted to 8.5 with NH₄OH. The reaction is stirred, open to room air, and monitored by RP-HPLC.

Once complete, the reaction is brought to pH 4 with acetic acid and lyophilized. The product is purified and characterized as above.

Method 2: A sample of the crude peptide is dissolved at a concentration of 0.5 mg/mL in 5% acetic acid. The pH is adjusted to 6.0 with NH₄OH. DMSO (20% by volume) is added and the reaction is stirred overnight. After analytical RP-HPLC analysis, the reaction is diluted with H₂O and triple lyophilized to remove DMSO. The crude product is purified by preparative RP-HPLC. (JACS. vol. 113, 6657, 1991)

HGPRBMY2 Peptide Modulators of the Present Invention.

| | |
|---|---|
| GDFWYEACESSCAFW | (SEQ ID NO:32) |
| LEWGSDVFYDVYDCC | (SEQ ID NO:33) |
| CLRSGTGCAFQLYRF | (SEQ ID NO:34) |
| FAGQIIWYDALDTLM | (SEQ ID NO:35) |

Assessing Affect of Peptides on GPCR Function.

The effect of any one of these peptides on the function of the GPCR of the present invention may be determined by adding an effective amount of each peptide to each functional assay. Representative functional assays are described more specifically herein.

Uses of the Peptide Modulators of the Present Invention.

The aforementioned peptides of the present invention are useful for a variety of purposes, though most notably for modulating the function of the GPCR of the present invention, and potentially with other GPCRs of the same G-protein coupled receptor subclass (e.g., peptide receptors, adrenergic receptors, purinergic receptors, etc.), and/or other subclasses known in the art. For example, the peptide modulators of the present invention may be useful as HGPRBMY2 agonists. Alternatively, the peptide modulators of the present invention may be useful as HGPRBMY2 antagonists of the present invention. In addition, the peptide modulators of the present invention may be useful as competitive inhibitors of the HGPRBMY2 cognate ligand(s), or may be useful as non-competitive inhibitors of the HGPRBMY2 cognate ligand(s).

Furthermore, the peptide modulators of the present invention may be useful in assays designed to either deorphan the HGPRBMY2 polypeptide of the present invention, or to identify other agonists or antagonists of the HGPRBMY2 polypeptide of the present invention, particularly small molecule modulators.

### Example 11

Alternative Method of Assessing the Ability of HGPRBMY1 or HGPRBMY2 to Serve as a GPCR Receptor.

The activity of the HGPRBMY1 or HGPRBMY2 polypeptides may be measured using an assay based upon the property of some known GPCRs to support proliferation in vitro of fibroblasts and tumor cells under serum-free conditions (Chiquet Ehrismann, R. et al. (1986) Cell 47: 131-139). Briefly, wells in 96 well cluster plates (Falcon, Fisher Scientific, Santa Clara Calif.) are coated with HGPRBMY1 or HGPRBMY2 polypeptides by incubation with solutions at 50-100 Rg/ml for 15 min at ambient temperature. The coating solution is aspirated, and the wells washed with Dulbecco's medium before cells are plated. Rat fibroblast cultures or rat mammary tumor cells are prepared as described and plated at a density of 104-105 cells/ml in Dulbecco's medium supplemented with 10% fetal calf serum (FCS).

After three days the media are removed, and the cells washed three times with phosphatebuffered saline (PBS) before the addition of serum-free Dulbecco's medium containing 0.25 mg/ml bovine serum albumin (BSA, Fraction V, Sigma Chemical, St. Louis, Mo.). After 2 days the medium is aspirated, and 100 il of [3H] thymidine (NEN) at 2 IlCi/ml in fresh Dulbecco's medium containing 0.25 mg/ml BSA added. Parallel plates are fixed and stained to determine cell numbers. After 16 hr, the medium is aspirated, the cell layer washed with PBS, and the 10% trichloroacetic acid-precipitable counts in the cell layer determined by liquid scintillation counting of radioisotope (normalized to relative cell numbers; Chiquet-Ehrismann, R. et al. (1986) supra). The rates of cell proliferation and [3H] thymidine uptake are proportional to the levels of GCRP in the sample.

Alternatively, the assay for HGPRBMY1 or HGPRBMY2 polypeptide activity based upon the property of CD97/Emrl GPCR family proteins to modulate G protein-activated second messenger signal transduction pathways (e. g., cAMP; Gaudin, P. et al. (1998) J. Biol. Chem. 273: 4990-4996). A plasmid encoding the full length HGPRBMY1 or HGPRBMY2 polypeptide is transfected into a mammalian cell line (e. g., COS-7 or Chinese hamster ovary (CHO-K1) cell lines) using methods well-known in the art. Transfected cells are grown in 12-well trays in culture medium containing 2% FCS for 48 hours, the culture medium is discarded, then the attached cells are gently washed with PBS. The cells are then incubated in culture medium with 10% FCS or 2% FCS for 30 minutes, then the medium is removed and cells lysed by treatment with 1 M perchloric acid. The cAMP levels in the lysate are measured by radioimmunoassay using methods well-known in the art. Changes in the levels of cAMP in the lysate from 10% FCS-treated cells compared with those in 2% FCS-treated cells are proportional to the amount of the HGPRBMY1 or HGPRBMY2 polypeptide present in the transfected cells.

### Example 12

Method of Assessing the Physiological Function of the HGPRBMY1 or HGPRBMY2 Polypeptide at the Cellular Level.

The physiological function of the HGPRBMY1 or HGPRBMY2 polypeptide may be assessed by expressing the sequences encoding HGPRBMY1 or HGPRBMY2 at physiologically elevated levels in mammalian cell culture systems cDNA is subcloned into a mammalian expression vector containing a strong promoter that drives high levels of cDNA expression (examples are provided elsewhere herein). Vectors of choice include pCMV SPORT (Life Technologies) and pCR3.1 (Invitrogen, Carlsbad Calif.), both of which contain the cytomegalovirus promoter 5-10, ug of recombinant vector are transiently transfected into a human cell line, preferably of endothelial or hematopoietic origin, using either liposome formulations or electroporation 1-2ug of an additional plasmid containing sequences encoding a marker protein are cotransfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of HGPRBMY1 or HGPRBMY2 polypeptides on gene expression can be assessed using highly purified populations of cells transfected with sequences encoding HGPRBMY1 or HGPRBMY2 and either CD64 or CD64-GFP, CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding HGPRBMY1 or HGPRBMY2 polypeptides and other genes of interest can be analyzed by northern analysis or microarray techniques.

### Example 13

Method of Assessing the Physiological Function of the HGPRBMY1 or HGPRBMY2 Polypeptides in Xenopus Oocytes.

Capped RNA transcripts from linearized plasmid templates encoding the receptor cDNAs of the invention are synthesized in vitro with RNA polymerases in accordance with standard procedures.

In vitro transcripts are suspended in water at a final concentration of 0.2 mg/ml. Ovarian lobes are removed from adult female toads, Stage V defolliculatedoocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca2+ free Barth's medium at room temperature.

In a preferred embodiment, such a system can be used to screen known ligands and tissue/cell extracts for activating ligands. A number of GPCR ligands are known in the art and are encompassed by the present invention (see, for example, The G-Protein Linked Receptor Facts Book, referenced elsewhere herein).

### Example 14

Method of Assessing the Physiological Function of the HGPRBMY1 or HGPRBMY2 Polypeptides Using Microphysiometric Assays.

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor that is coupled to an energy utilizing intracellular signaling pathway such as the G-protein coupled receptor of the present invention.

### Example 15

Method of Assessing the Physiological Function of the HGPRBMY1 or HGPRBMY2 Polypeptides Using Calcium and Camp Functional Assays.

A well known observation in the art relates to the fact that GPCR receptors which are expressed in HEK 293 cells have been shown to be functionally couple—leading to subsequent activation of phospoholipase C (PLC) and calcium mobilization, and/or cAMP stimuation or inhibition.

Based upon the above, calcium and cAMP assays may be useful in assessing the ability of HGPRBMY1 or HGPRBMY2 to serve as a GPCR. Briefly, basal calcium levels in the HEK 293 cells in HGPRBMY1 or HGPRBMY2-transfected or vector control cells can be observed to determine whether the levels fall within a normal physiological range, 100 nM to 200 nM. HEK 293 cells expressing recombinant receptors are then loaded with fura 2 and in a single day selected GPCR ligands or tissue/cell extracts are evaluated for agonist induced calcium mobilization. Similarly, HEK 293 cells expressing recombinant HGPRBMY1 or HGPRBMY2 receptors are evaluated for the stimulation or inhibition of cAMP production using standard cAMP quantitation assays. Agonists presenting a calcium transient or cAMP flucuation are tested in vector control cells to determine if the response is unique to the transfected cells expressing the HGPRBMY1 or HGPRBMY2 receptor.

### Example 16

Method of Screening for Compounds that Interact with the HGPRBMY1 or HGPRBMY2 Polypeptide.

The following assays are designed to identify compounds that bind to the HGPRBMY1 or HGPRBMY2 polypeptide, bind to other cellular proteins that interact with the HGPRBMY1 or HGPRBMY2 polypeptide, and to compounds that interfere with the interaction of the HGPRBMY1 or HGPRBMY2 polypeptide with other cellular proteins.

Such compounds can include, but are not limited to, other cellular proteins. Specifically, such compounds can include, but are not limited to, peptides, such as, for example, soluble peptides, including, but not limited to Ig-tailed fusion peptides, comprising extracellular portions of HGPRBMY1 or HGPRBMY2 polypeptide transmembrane receptors, and members of random peptide libraries (see, e.g., Lam, K. S. et al., 1991, Nature 354:82-84; Houghton, R. et al., 1991, Nature 354:84-86), made of D-and/or L-configuration amino acids, phosphopeptides (including, but not limited to, members of random or partially degenerate phosphopeptide libraries; see, e.g., Songyang, Z., et al., 1993, Cell 72:767-778), antibodies (including, but not limited to, polyclonal, monoclonal, humanized, anti-idiotypic, chimeric or single chain antibodies, and FAb, F(ab′).sub.2 and FAb expression libary fragments, and epitope-binding fragments thereof), and small organic or inorganic molecules.

Compounds identified via assays such as those described herein can be useful, for example, in elaborating the biological function of the HGPRBMY1 or HGPRBMY2 polypeptide, and for ameliorating symptoms of tumor progression, for example. In instances, for example, whereby a tumor progression state or disorder results from a lower overall level of HGPRBMY1 or HGPRBMY2 expression, HGPRBMY1 or HGPRBMY2 polypeptide, and/or HGPRBMY1 or HGPRBMY2 polypeptide activity in a cell involved in the tumor progression state or disorder, compounds that interact with the HGPRBMY1 or HGPRBMY2 polypeptide can include ones which accentuate or amplify the activity of the bound HGPRBMY1 or HGPRBMY2 polypeptide. Such compounds would bring about an effective increase in the level of HGPRBMY1 or HGPRBMY2 polypeptide activity, thus ameliorating symptoms of the tumor progression disorder or state. In instances whereby mutations within the HGPRBMY1 or HGPRBMY2 polypeptide cause aberrant HGPRBMY1 or HGPRBMY2 polypeptides to be made which have a deleterious effect that leads to tumor progression, compounds that bind HGPRBMY1 or HGPRBMY2 polypeptide can be identified that inhibit the activity of the bound HGPRBMY1 or HGPRBMY2 polypeptide. Assays for testing the effectiveness of such compounds are known in the art and discussed, elsewhere herein.

### Example 17

Method of Screening, in Vitro, Compounds that Bind to the HGPRBMY1 or HGPRBMY2 Polypeptide.

In vitro systems can be designed to identify compounds capable of binding the HGPRBMY1 or HGPRBMY2 polypeptide of the invention. Compounds identified can be useful, for example, in modulating the activity of wild type and/or mutant HGPRBMY1 or HGPRBMY2 polypeptide, preferably mutant HGPRBMY1 or HGPRBMY2 polypeptide, can be useful in elaborating the biological function of the HGPRBMY1 or HGPRBMY2 polypeptide, can be utilized in screens for identifying compounds that disrupt normal HGPRBMY1 or HGPRBMY2 polypeptide interactions, or can in themselves disrupt such interactions.

The principle of the assays used to identify compounds that bind to the HGPRBMY1 or HGPRBMY2 polypeptide involves preparing a reaction mixture of the HGPRBMY1 or HGPRBMY2 polypeptide and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring HGPRBMY1 or HGPRBMY2 polypeptide or the test substance onto a solid phase and detecting HGPRBMY1 or HGPRBMY2 polypeptide/test compound complexes anchored on the solid phase at the end of the reaction. In one embodiment of such a method, the HGPRBMY1 or HGPRBMY2 polypeptide can be anchored onto a solid surface, and the test compound, which is not anchored, can be labeled, either directly or indirectly.

In practice, microtitre plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for HGPRBMY1 or HGPRBMY2 polypeptide or the test compound to anchor any complexes formed in solution, and a labeled antibody specific for the other component of the possible complex to detect anchored complexes.

### Example 18

Method for Identifying a Putative Ligand for the HGCRBMY1 or HGPRBMY2 Polypeptide.

Ligand binding assays provide a direct method for ascertaining receptor pharmacology and are adaptable to a high throughput format. A panel of known GPCR purified ligands may be radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

A number of GPCR ligands are known in the art and are encompassed by the present invention (see, for example, The G-Protein Linked Receptor Facts Book, referenced elsewhere herein).

Alternatively, the HGPRBMY1 or HGPRBMY2 polypeptide of the present invention may also be functionally screened (using calcium, cAMP, microphysiometer, oocyte electrophysiology, etc., functional screens) against tissue extracts to identify natural ligands. Extracts that produce positive functional responses can be sequencially subfractionated until an activating ligand is isolated identified using methods well known in the art, some of which are described herein.

### Example 19

Method of Identifying Compounds that Interfere with HGPRBMY1 or HGPRBMY2 Polypeptide/Cellular Product Interaction.

The HGPRBMY1 or HGPRBMY2 polypeptide of the invention can, in vivo, interact with one or more cellular or extracellular macromolecules, such as proteins. Such macromolecules include, but are not limited to, polypeptides, particularly GPCR ligands, and those products identified via screening methods described, elsewhere herein. For the purposes of this discussion, such cellular and extracellular macromolecules are referred to herein as “binding partner(s)”. For the purpose of the present invention, “binding partner” may also encompass polypeptides, small molecule compounds, polysaccarides, lipids, and any other molecule or molecule type referenced herein. Compounds that disrupt such interactions can be useful in regulating the activity of the HGPRBMY1 or HGPRBMY2 polypeptide, especially mutant HGPRBMY1 or HGPRBMY2 polypeptide. Such compounds can include, but are not limited to molecules such as antibodies, peptides, and the like described in elsewhere herein.

The basic principle of the assay systems used to identify compounds that interfere with the interaction between the HGPRBMY1 or HGPRBMY2 polypeptide and its cellular or extracellular binding partner or partners involves preparing a reaction mixture containing the HGPRBMY1 or HGPRBMY2 polypeptide, and the binding partner under conditions and for a time sufficient to allow the two products to interact and bind, thus forming a complex. In order to test a compound for inhibitory activity, the reaction mixture is prepared in the presence and absence of the test compound. The test compound can be initially included in the reaction mixture, or can be added at a time subsequent to the addition of HGPRBMY1 or HGPRBMY2 polypeptide and its cellular or extracellular binding partner. Control reaction mixtures are incubated without the test compound or with a placebo. The formation of any complexes between the HGPRBMY1 or HGPRBMY2 polypeptide and the cellular or extracellular binding partner is then detected. The formation of a complex in the control reaction, but not in the reaction mixture containing the test compound, indicates that the compound interferes with the interaction of the HGPRBMY1 or HGPRBMY2 polypeptide and the interactive binding partner. Additionally, complex formation within reaction mixtures containing the test compound and normal HGPRBMY1 or HGPRBMY2 polypeptide can also be compared to complex formation within reaction mixtures containing the test compound and mutant HGPRBMY1 or HGPRBMY2 polypeptide. This comparison can be important in those cases wherein it is desirable to identify compounds that disrupt interactions of mutant but not normal HGPRBMY1 or HGPRBMY2 polypeptide.

The assay for compounds that interfere with the interaction of the HGPRBMY1 or HGPRBMY2 polypeptide and binding partners can be conducted in a heterogeneous or homogeneous format. Heterogeneous assays involve anchoring either the HGPRBMY1 or HGPRBMY2 polypeptide or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the HGPRBMY1 or HGPRBMY2 polypeptide and the binding partners, e.g., by competition, can be identified by conducting the reaction in the presence of the test substance; i.e., by adding the test substance to the reaction mixture prior to or simultaneously with the HGPRBMY1 or HGPRBMY2 polypeptide and interactive cellular or extracellular binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the HGPRBMY1 or HGPRBMY2 polypeptide or the interactive cellular or extracellular binding partner, is anchored onto a solid surface, while the non-anchored species is labeled, either directly or indirectly. In practice, microtitre plates are conveniently utilized. The anchored species can be immobilized by non-covalent or covalent attachments. Non-covalent attachment can be accomplished simply by coating the solid surface with a solution of the HGPRBMY1 or HGPRBMY2 polypeptide or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the HGPRBMY1 or HGPRBMY2 polypeptide and the interactive cellular or extracellular binding partner product is prepared in which either the HGPRBMY1 or HGPRBMY2 polypeptide or their binding partners are labeled, but the signal generated by the label is quenched due to complex formation (see, e.g., U.S. Pat. No. 4,109,496 by Rubenstein which utilizes this approach for immunoassays).

The addition of a test substance that competes with and displaces one of the species from the preformed complex will result in the generation of a signal above background. In this way, test substances which disrupt HGPRBMY1 or HGPRBMY2 polypeptide-cellular or extracellular binding partner interaction can be identified.

In a particular embodiment, the HGPRBMY1 or HGPRBMY2 polypeptide can be prepared for immobilization using recombinant DNA techniques known in the art. For example, the HGPRBMY1 or HGPRBMY2 polypeptide coding region can be fused to a glutathione-S-transferase (GST) gene using a fusion vector such as pGEX-5X-1, in such a manner that its binding activity is maintained in the resulting fusion product. The interactive cellular or extracellular product can be purified and used to raise a monoclonal antibody, using methods routinely practiced in the art and described above. This antibody can be labeled with the radioactive isotope .sup.125 I, for example, by methods routinely practiced in the art. In a heterogeneous assay, e.g., the GST-HGPRBMY1 or HGPRBMY2 polypeptide fusion product can be anchored to glutathione-agarose beads. The interactive cellular or extracellular binding partner product can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the HGPRBMY1 or HGPRBMY2 polypeptide and the interactive cellular or extracellular binding partner can be detected by measuring the amount of radioactivity that remains associated with the glutathione-agarose beads. A successful inhibition of the interaction by the test compound will result in a decrease in measured radioactivity.

Alternatively, the GST-HGPRBMY1 or HGPRBMY2 polypeptide fusion product and the interactive cellular or extracellular binding partner product can be mixed together in liquid in the absence of the solid glutathione-agarose beads. The test compound can be added either during or after the binding partners are allowed to interact. This mixture can then be added to the glutathione-agarose beads and unbound material is washed away. Again the extent of inhibition of the binding partner interaction can be detected by adding the labeled antibody and measuring the radioactivity associated with the beads.

In another embodiment of the invention, these same techniques can be employed using peptide fragments that correspond to the binding domains of the HGPRBMY1 or HGPRBMY2 polypeptide product and the interactive cellular or extracellular binding partner (in case where the binding partner is a product), in place of one or both of the full length products.

Any number of methods routinely practiced in the art can be used to identify and isolate the protein's binding site. These methods include, but are not limited to, mutagenesis of one of the genes encoding one of the products and screening for disruption of binding in a co-immunoprecipitation assay. Compensating mutations in the gene encoding the second species in the complex can be selected. Sequence analysis of the genes encoding the respective products will reveal the mutations that correspond to the region of the product involved in interactive binding. Alternatively, one product can be anchored to a solid surface using methods described in this Section above, and allowed to interact with and bind to its labeled binding partner, which has been treated with a proteolytic enzyme, such as trypsin. After washing, a short, labeled peptide comprising the binding domain can remain associated with the solid material, which can be isolated and identified by amino acid sequencing. Also, once the gene coding for the cellular or extracellular binding partner product is obtained, short gene segments can be engineered to express peptide fragments of the product, which can then be tested for binding activity and purified or synthesized.

### Example 20

Isolation of a Specific Clone from the Deposited Sample.

The deposited material in the sample assigned the ATCC Deposit Number cited herein for any given cDNA clone also may contain one or more additional plasmids, each comprising a cDNA clone different from that given clone. Thus, deposits sharing the same ATCC Deposit Number contain at least a plasmid for each cDNA clone identified herein. Typically, each ATCC deposit sample cited herein comprises a mixture of approximately equal amounts (by weight) of about 1-10 plasmid DNAs, each containing a different cDNA clone and/or partial cDNA clone; but such a deposit sample may include plasmids for more or less than 2 cDNA clones.

Two approaches can be used to isolate a particular clone from the deposited sample of plasmid DNA(s) of the present invention. First, a plasmid is directly isolated by screening the clones using a polynucleotide probe corresponding to SEQ ID NO:1 or SEQ ID NO:13.

Particularly, a specific polynucleotide with 30-40 nucleotides is synthesized using an Applied Biosystems DNA synthesizer according to the sequence reported. The oligonucleotide is labeled, for instance, with 32P-(-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host, as indicated above (such as XL-1 Blue (Stratagene)) using techniques known to those of skill in the art, such as those provided by the vector supplier or in related publications or patents cited above. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:1 or SEQ ID NO:13 (i.e., within the region of SEQ ID NO:1 or SEQ ID NO:13 bounded by the 5′ NT and the 3′ NT of the clone defined herein) are synthesized and used to amplify the desired CDNA using the deposited cDNA plasmid as a template. The polymerase chain reaction is carried out under routine conditions, for instance, in 25 ul of reaction mixture with 0.5 ug of the above cDNA template. A convenient reaction mixture is 1.5-5 mM MgCl2, 0.01% (w/v) gelatin, 20 uM each of dATP, dCTP, dGTP, dTTP, 25 pmol of each primer and 0.25 Unit of Taq polymerase. Thirty five cycles of PCR (denaturation at 94 degree C. for 1 min; annealing at 55 degree C. for 1 min; elongation at 72 degree C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

The polynucleotide(s) of the present invention, the polynucleotide encoding the polypeptide of the present invention, or the polypeptide encoded by the deposited clone may represent partial, or incomplete versions of the complete coding region (i.e., full-length gene). Several methods are known in the art for the identification of the 5′ or 3′ non-coding and/or coding portions of a gene which may not be present in the deposited clone. The methods that follow are exemplary and should not be construed as limiting the scope of the invention. These methods include but are not limited to, filter probing, clone enrichment using specific probes, and protocols similar or identical to 5′ and 3′ “RACE” protocols that are well known in the art. For instance, a method similar to 5′ RACE is available for generating the missing 5′ end of a desired full-length transcript. (Fromont-Racine et al., Nucleic Acids Res. 21(7):1683-1684 (1993)).

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full-length gene.

This above method starts with total RNA isolated from the desired source, although poly-A+ RNA can be used. The RNA preparation can then be treated with phosphatase if necessary to eliminate 5′ phosphate groups on degraded or damaged RNA that may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene. Moreover, it may be advantageous to optimize the RACE protocol to increase the probability of isolating additional 5′ or 3′ coding or non-coding sequences. Various methods of optimizing a RACE protocol are known in the art, though a detailed description summarizing these methods can be found in B. C. Schaefer, Anal. Biochem., 227:255-273, (1995).

An alternative method for carrying out 5′ or 3′ RACE for the identification of coding or non-coding sequences is provided by Frohman, M. A., et al., Proc.Nat'l.Acad.Sci.USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation, therefor. The following briefly describes a modification of this original 5′ RACE procedure. Poly A+ or total RNAs reverse transcribed with Superscript II (Gibco/BRL) and an antisense or I complementary primer specific to the cDNA sequence. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoIJ Sail and ClaI) at the 5′ end and a primer containing just these restriction sites. This double-stranded cDNA is PCR amplified for 40 cycles with the same primers as well as a nested cDNA-specific antisense primer. The PCR products are size-separated on an ethidium bromide-agarose gel and the region of gel containing cDNA products the predicted size of missing protein-coding DNA is removed. cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length genes. A second kit is available from Clontech which is a modification of a related technique, SLIC (single-stranded ligation to single-stranded cDNA), developed by Dumas et al., Nucleic Acids Res., 19:5227-32(1991). The major differences in procedure are that the RNA is alkaline hydrolyzed after reverse transcription and RNA ligase is used to join a restriction site-containing anchor primer to the first-strand cDNA. This obviates the necessity for the dA-tailing reaction which results in a polyT stretch that is difficult to sequence past.

An alternative to generating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

RNA Ligase Protocol for Generating the 5′ or 3 ′ End Sequences to Obtain Full Length Genes

Once a gene of interest is identified, several methods are available for the identification of the 5′ or 3′ portions of the gene which may not be present in the original cDNA plasmid. These methods include, but are not limited to, filter probing, clone enrichment using specific probes and protocols similar and identical to 5′ and 3′ RACE. While the full-length gene may be present in the library and can be identified by probing, a useful method for generating the 5′ or 3′ end is to use the existing sequence information from the original cDNA to generate the missing information. A method similar to 5′ RACE is available for generating the missing 5′ end of a desired full-length gene. (This method was published by Fromont-Racine et al., Nucleic Acids Res., 21(7): 1683-1684 (1993)). Briefly, a specific RNA oligonucleotide is ligated to the 5′ ends of a population of RNA presumably 30 containing full-length gene RNA transcript and a primer set containing a primer specific to the ligated RNA oligonucleotide and a primer specific to a known sequence of the gene of interest, is used to PCR amplify the 5′ portion of the desired full length gene which may then be sequenced and used to generate the full length gene. This method starts with total RNA isolated from the desired source, poly A RNA may be used but is not a prerequisite for this procedure. The RNA preparation may then be treated with phosphatase if necessary to eliminate 5′ phosphate groups on degraded or damaged RNA which may interfere with the later RNA ligase step. The phosphatase if used is then inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a gene specific oligonucleotide. The first strand synthesis reaction can then be used as a template for PCR amplification of the desired 5′ end using a primer specific to the ligated RNA oligonucleotide and a primer specific to the known sequence of the apoptosis related of interest. The resultant product is then sequenced and analyzed to confirm that the 5′ end sequence belongs to the relevant apoptosis related.

### Example 21

Tissue Distribution of Polypeptide.

Tissue distribution of mRNA expression of polynucleotides of the present invention is determined using protocols for Northern blot analysis, described by, among others, Sambrook et al. For example, a cDNA probe produced by the method described in Example 20 is labeled with p32 using the rediprime(tm) DNA labeling system (Amersham Life Science), according to manufacturer's instructions. After labeling, the probe is purified using CHROMA SPINO-100 column (Clontech Laboratories, Inc.) according to manufacturer's protocol number PT1200-1. The purified labeled probe is then used to examine various tissues for mRNA expression.

Tissue Northern blots containing the bound mRNA of various tissues are examined with the labeled probe using ExpressHybtm hybridization solution (Clonetech according to manufacturers protocol number PT 1190-1. Northern blots can be produced using various protocols well known in the art (e.g., Sambrook et al). Following hybridization and washing, the blots are mounted and exposed to film at −70 C. overnight, and the films developed according to standard procedures.

### Example 22

Chromosomal Mapping of the Polynucleotides.

An oligonucleotide primer set is designed according to the sequence at the 5′ end of SEQ ID NO:1 or SEQ ID NO:13. This primer preferably spans about 100 nucleotides. This primer set is then used in a polymerase chain reaction under the following set of conditions: 30 seconds, 95 degree C.; 1 minute, 56 degree C.; 1 minute, 70 degree C. This cycle is repeated 32 times followed by one 5 minute cycle at 70 degree C. Mammalian DNA, preferably human DNA, is used as template in addition to a somatic cell hybrid panel containing individual chromosomes or chromosome fragments (Bios, Inc). The reactions are analyzed on either 8% polyacrylamide gels or 3.5% agarose gels. Chromosome mapping is determined by the presence of an approximately 100 bp PCR fragment in the particular somatic cell hybrid.

### Example 23

Bacterial Expression of a Polypeptide.

A polynucleotide encoding a polypeptide of the present invention is amplified using PCR oligonucleotide primers corresponding to the 5′ and 3′ ends of the DNA sequence, as outlined in Example 20, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacd repressor, clearing the P/O leading to increased gene expression.

Cells are grown for an extra 3 to 4 hours. Cells are then harvested by centrifugation (20 mins at 600×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 24

Purification of a Polypeptide from an Inclusion Body.

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 25

Cloning and Expression of a Polypeptide in a Baculovirus Expression System.

In this example, the plasmid shuttle vector pAc373 is used to insert a polynucleotide into a baculovirus to express a polypeptide. A typical baculovirus expression vector contains the strong polyhedrin promoter of the Autographa califomica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in Example 20, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described in Example 20. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 26

Expression of a Polypeptide in Mammalian Cells.

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the manmmalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### Example 27

Method of Creating N- and C-terminal Deletion Mutants Corresponding to the HGPRBMY1 and HGPRBMY2 Polypeptides of the Present Invention.

As described elsewhere herein, the present invention encompasses the creation of N- and C-terminal deletion mutants, in addition to any combination of N- and C-terminal deletions thereof, corresponding to the HGPRBMY1 or HGPRBMY2 polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length HGPRBMY1 or HGPRBMY2 polypeptide sequence (as described herein, for example), appropriate primers of about 15-25 nucleotides derived from the desired 5′ and 3′ positions of SEQ ID NO:1 or SEQ ID NO:13 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the M76 to H431 HGPRBMY2 N-terminal deletion mutant, the following primers could be used to amplify a cDNA fragment corresponding to this deletion mutant:

| | | |
|---|---|---|
| 5′ Primer | 5′-GCAGCA GCGGCCGC ATGCGCACCGTCACCAACATC-3′ | (SEQ ID NO:20) |
| | NotI | |
| 3′ Primer | 5′-GCAGCA GTCGAC ATGCCCACTGTCTAAAGGAGAATTC-3′ | (SEQ ID NO:21) |
| | SalI | |

For example, in the case of the M1 to Y305 HGPRBMY2 C-terminal deletion mutant, the following primers could be used to amplify a cDNA fragment corresponding to this deletion mutant:

| | | |
|---|---|---|
| 5′ Primer | 5′-GCAGCA GCGGCCGC CGGCGCATGGGGCCCAGATCCCCG-3′ | (SEQ ID NO:22) |
| | NotI | |
| 3′ Primer | 5′-GCAGCA GTCGAC GAACACACTCTCCTGCCTCTGGAGG-3′ | (SEQ ID NO:23) |
| | SalI | |

For example, in the case of the R50 to F359 HGPRBMY1 N-terminal deletion mutant, the following primers could be used to amplify a cDNA fragment corresponding to this deletion mutant:

| | | |
|---|---|---|
| 5′ Primer | 5′-GCAGCA GCGGCCGC ATGCAGGTCCCGAACAGCACCGGCC-3′ | (SEQ ID NO:52) |
| | NotI | |
| 3′ Primer | 5′-GCAGCA GTCGAC CTTGTACACGTGGTAGTAGCTCTTG-3′ | (SEQ ID NO:53) |
| | SalI | |

For example, in the case of the M1 to K276 HGPRBMY1 C-terminal deletion mutant, the following primers could be used to amplify a cDNA fragment corresponding to this deletion mutant:

| | | |
|---|---|---|
| 5′ Primer | 5′-GCAGCA GCGGCCGC ATGCAGGCGCTTAACATTACCCCGG-3′ | (SEQ ID NO:54) |
| | NotI | |
| 3′ Primer | 5′-GCAGCA GTCGAC ATATTCCTTTTCAAAATTACTG-3′ | (SEQ ID NO:55) |
| | SalI | |

Representative PCR amplification conditions are provided below, although the skilled artisan would appreciate that other conditions may be required for efficient amplification. A 100 ul PCR reaction mixture may be prepared using 10 ng of the template DNA (cDNA clone of HGPRBMY2), 200 uM 4 dNTPs, 1 uM primers, 0.25 U Taq DNA polymerase (PE), and standard Taq DNA polymerase buffer. Typical PCR cycling condition are as follows:

| | | |
|---|---|---|
| 20-25 | cycles: | 45 sec, 93 degrees |
| | | 2 min, 50 degrees |
| | | 2 min, 72 degrees |
| 1 | cycle: | 10 min, 72 degrees |

After the final extension step of PCR, 5 U Kienow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula:

(S+(X*3)) to ((S+(X*3))+25),

wherein ‘S’ is equal to the nucleotide position of the initiating start codon of the HGPRBMY1 or HGPRBMY2 gene (SEQ ID NO:1 or SEQ ID NO:13 ), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:1 or SEQ ID NO:13 . Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25),

wherein ‘S’ is equal to the nucleotide position of the initiating start codon of the HGPRBMY1 or HGPRBMY2 gene (SEQ ID NO:1 or SEQ ID NO:13 ), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1 or SEQ ID NO:13. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

### Example 28

Protein Fusions.

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example described herein; see also EP A 394,827; Traunecker, et al., Nature 331:84-86 (1988).) Similarly, fusion to IgG-1, IgG-3, and albumin increases the half-life time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector. Note that the polynucleotide is cloned without a stop codon, otherwise a fusion protein will not be produced.

The naturally occurring signal sequence may be used to produce the protein (if applicable). Alternatively, if the naturally occurring signal sequence is not used, the vector can be modified to include a heterologous signal sequence. (See, e.g., WO 96/34891 and/or U.S. Pat. No. 6,066,781, supra.)

Human IgG Fc Region:

(SEQ ID NO:24)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCAAGCGACATCGCCGTG
GAGTGGGAGAGCAATGGGCAGCCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAACGCAGAAGAGCCTCTCCCTGTCTCCGGGTA
AATGAGTGCGACGGCCGCGACTCTAGAGGAT

### Example 29

Production of an Antibody from a Polypeptide.

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 26

Method of Enhancing the Biological Activity/Functional Characteristics of Invention Through Molecular Evolution.

Although many of the most biologically active proteins known are highly effective for their specified function in an organism, they often possess characteristics that make them undesirable for transgenic, therapeutic, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “terror-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments -further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatmann) or could be purified using Microcon concentrators (Amicon) of the appropriate molecular weight cutoff, or could use oligonucleotide purification columns (Qiagen), in addition to other methods known in the art. If using DE81 ion-exchange paper, the 10-50 bp fragments could be eluted from said paper using 1M NaCl, followed by ethanol precipitation.

The resulting purified fragments would then be subjected to a PCR assembly reaction by re-suspension in a PCR mixture containing: 2 mM of each DNTP, 2.2 mM MgCl2, 50 mM KCl, 10 mM Tris.HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C. for 60 s; 94 C. for 30 s, 50-55 C. for 30 s, and 72 C. for 30 s using 30-45 cycles, followed by 72 C. for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C for 30 s, 50 C. for 30 s, and 72 C. for 30 s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 30

Method of Determining Alterations in a Gene Corresponding to a Polynucleotide.

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95 degrees C. for 30 seconds; 60-120 seconds at 52-58 degrees C.; and 60-120 seconds at 70 degrees C., using buffer solutions described in Sidransky et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products is cloned into T-tailed vectors as described in Holton et al., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 2 are nick-translated with digoxigenindeoxy-uridine 5′ -triphosphate (Boehringer Manheim), and FISH performed as described in Johnson et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 31

Method of Detecting Abnormal Levels of a Polypeptide in a Biological Sample.

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/ml. The antibodies are either monoclonal or polyclonal and are produced by the method described elsewhere herein. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 32

Formulation.

The invention also provides methods of treatment and/or prevention diseases, disorders, and/or conditions (such as, for example, any one or more of the diseases or disorders disclosed herein) by administration to a subject of an effective amount of a Therapeutic. By therapeutic is meant a polynucleotides or polypeptides of the invention (including fragments and variants), agonists or antagonists thereof, and/or antibodies thereto, in combination with a pharmaceutically acceptable carrier type (e.g., a sterile carrier).

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kglday for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Therapeutics can be administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention may also be suitably administered by sustained-release systems. Suitable examples of sustained-release Therapeutics include suitable polymeric materials (such as, for example, semi-permeable polymer matrices in the form of shaped articles, e.g., films, or microcapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2- hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D- (−)-3-hydroxybutyric acid (EP 133,988).

Sustained-release Therapeutics also include liposomally entrapped Therapeutics of the invention (see, generally, Langer, Science 249:1527-1533 (1990); Treat et al., in Liposomes in the Therapy of Infectious Disease and Cancer, Lopez-Berestein and Fidler (eds.), Liss, N.Y., pp. 317 -327 and 353-365 (1989)). Liposomes containing the Therapeutic are prepared by methods known per se: DE 3,218,121; Epstein et al., Proc. Natl. Acad. Sci. (USA) 82:3688-3692 (1985); Hwang et al., Proc. Natl. Acad. Sci.(USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The Therapeutic will typically be formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

The Therapeutics of the invention may be administered alone or in combination with adjuvants. Adjuvants that may be administered with the Therapeutics of the invention include, but are not limited to, alum, alum plus deoxycholate (ImmunoAg), MTP-PE (Biocine Corp.), QS21 (Genentech, Inc.), BCG, and MPL. In a specific embodiment, Therapeutics of the invention are administered in combination with alum. In another specific embodiment, Therapeutics of the invention are administered in combination with QS-21. Further adjuvants that may be administered with the Therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the Therapeutics of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The Therapeutics of the invention may be administered alone or in combination with other therapeutic agents. Therapeutic agents that may be administered in combination with the Therapeutics of the invention, include but not limited to, other members of the TNF family, chemotherapeutic agents, antibiotics, steroidal and non-steroidal anti-inflammatories, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the Therapeutics of the invention are administered in combination with members of the TNF family. TNF, TNF-related or TNF-like molecules that may be administered with the Therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-1BB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892),TR10 (International Publication No. WO 98/54202), 312C2 (International Publication No. WO 98/06842), and TR12, and soluble forms CD154, CD70, and CD153.

In certain embodiments, Therapeutics of the invention are administered in combination with antiretroviral agents, nucleoside reverse transcriptase inhibitors, non-nucleoside reverse transcriptase inhibitors, and/or protease inhibitors. Nucleoside reverse transcriptase inhibitors that may be administered in combination with the Therapeutics of the invention, include, but are not limited to, RETROVIR (zidovudine/AZT), VIDEX (didanosine/ddI), HIVID (zalcitabine/ddC), ZERIT (stavudine/d4T), EPIVIR (lamivudine/3TC), and COMBIVIR (zidovudine/lamivudine). Non-nucleoside reverse transcriptase inhibitors that may be administered in combination with the Therapeutics of the invention, include, but are not limited to, VIRAMUNE (nevirapine), RESCRIPTOR (delavirdine), and SUSTIVA (efavirenz). Protease inhibitors that may be administered in combination with the Therapeutics of the invention, include, but are not limited to, CRIXIVAN (indinavir), NORVIR (ritonavir), INVIRASE (saquinavir), and VIRACEPT (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In other embodiments, Therapeutics of the invention may be administered in combination with anti-opportunistic infection agents. Anti-opportunistic agents that may be administered in combination with the Therapeutics of the invention, include, but are not limited to, TRIMETHOPRIM-SULFAMETHOXAZOLE, DAPSONE, PENTAMIDINE, ATOVAQUONE, ISONIAZID, RIFAMPIN, PYRAZINAMIDE, ETHAMBUTOL, RIFABUTIN, CLARITHROMYCIN, AZITHROMYCIN, GANCICLOVIR, FOSCARNET, CIDOFOVIR, FLUCONAZOLE, ITRACONAZOLE, KETOCONAZOLE, ACYCLOVIR, FAMCICOLVIR, PYRIMETHAMINE, LEUCOVORIN, NEUPOGEN (filgrastim/G-CSF), and LEUKINE (sargramostim/GM-CSF). In a specific embodiment, Therapeutics of the invention are used in any combination with TRIMETHOPRI-SULFAMETHOXAZ(OLE, DAPSONE, PENTAMIDINE, and/or ATOVAQUONE to prophylactically treat or prevent an opportunistic Pneumocystis carinii pneumonia infection. In another specific embodiment, Therapeutics of the invention are used in any combination with ISONIAZID, RIFAMPIN, PYRAZINAMIDE, and/or ETHAMBUTOL to prophylactically treat or prevent an opportunistic Mycobacterium avium complex infection. In another specific embodiment, Therapeutics of the invention are used in any combination with RIFABUTIN, CLARITHROMYCIN, and/or AZITHROMYCIN to prophylactically treat or prevent an opportunistic Mycobacterium tuberculosis infection. In another specific embodiment, Therapeutics of the invention are used in any combination with GANCICLOVIR, FOSCARNET, and/or CIDOFOVIR to prophylactically treat or prevent an opportunistic cytomegalovirus infection. In another specific embodiment, Therapeutics of the invention are used in any combination with FLUCONAZOLE, ITRACONAZOLE, and/or KETOCONAZOLE to prophylactically treat or prevent an opportunistic fungal infection. In another specific embodiment, Therapeutics of the invention are used in any combination with ACYCLOVIR and/or FAMCICOLVIR to prophylactically treat or prevent an opportunistic herpes simplex virus type I and/or type II infection. In another specific embodiment, Therapeutics of the invention are used in any combination with PYRIMETHAMINE and/or LEUCOVORIN to prophylactically treat or prevent an opportunistic Toxoplasma gondii infection. In another specific embodiment, Therapeutics of the invention are used in any combination with LEUCOVORIN and/or NEUPOGEN to prophylactically treat or prevent an opportunistic bacterial infection.

In a further embodiment, the Therapeutics of the invention are administered in combination with an antiviral agent. Antiviral agents that may be administered with the Therapeutics of the invention include, but are not limited to, acyclovir, ribavirin, amantadine, and remantidine.

In a further embodiment, the Therapeutics of the invention are administered in combination with an antibiotic agent. Antibiotic agents that may be administered with the Therapeutics of the invention include, but are not limited to, amoxicillin, beta-lactamases, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

Conventional nonspecific immunosuppressive agents, that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, Therapeutics of the invention are administered in combination with immunosuppressants. Immunosuppressants preparations that may be administered with the Therapeutics of the invention include, but are not limited to, ORTHOCLONE (OKT3), SANDIMMUNE/NEORAL/SANGDYA (cyclosporin), PROGRAF (tacrolimus), CELLCEPT (mycophenolate), Azathioprine, glucorticosteroids, and RAPAMUNE (sirolimus). In a specific embodiment, immunosuppressants may be used to prevent rejection of organ or bone marrow transplantation.

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR, IVEEGAM, SANDOGLOBULIN, GAMMAGARD S/D, and GAMIMUNE. In a specific embodiment, Therapeutics of the invention are administered in combination with intravenous immune globulin preparations in transplantation therapy (e.g., bone marrow transplant).

In an additional embodiment, the Therapeutics of the invention are administered alone or in combination with an anti-inflammatory agent. Anti-inflammatory agents that may be administered with the Therapeutics of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the Therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, Therapeutics of the invention are administered in combination with CHOP (cyclophosphamide, doxorubicin, vincristine, and prednisone) or any combination of the components of CHOP. In another embodiment, Therapeutics of the invention are administered in combination with Rituximab. In a further embodiment, Therapeutics of the invention are administered with Rituxmab and CHOP, or Rituxmab and any combination of the components of CHOP.

In an additional embodiment, the Therapeutics of the invention are administered in combination with cytokines. Cytokines that may be administered with the Therapeutics of the invention include, but are not limited to, IL2, IL3, IL4, IL5, IL6, IL7, IL10, IL12, IL13, IL15, anti-CD40, CD40L, IFN-gamma and TNF-alpha. In another embodiment, Therapeutics of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21.

In an additional embodiment, the Therapeutics of the invention are administered in combination with angiogenic proteins. Angiogenic proteins that may be administered with the Therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-682110; Platelet Derived Growth Factor-B (PDGF-B), as disclosed in European Patent Number EP-282317; Placental Growth Factor (PlGF), as disclosed in International Publication Number WO 92/06194; Placental Growth Factor-2 (P1GF-2), as disclosed in Hauser et al., Gorwth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the Therapeutics of the invention are administered in combination with hematopoietic growth factors. Hematopoietic growth factors that may be administered with the Therapeutics of the invention include, but are not limited to, LEUKINE (SARGRAMOSTE) and NEUPOGEN (FILGRASTIM).

In an additional embodiment, the Therapeutics of the invention are administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In a specific embodiment, formulations of the present invention may further comprise antagonists of P-glycoprotein (also referred to as the multiresistance protein, or PGP), including antagonists of its encoding polynucleotides (e.g., antisense oligonucleotides, ribozymes, zinc-finger proteins, etc.). P-glycoprotein is well known for decreasing the efficacy of various drug administrations due to its ability to export intracellular levels of absorbed drug to the cell exterior. While this activity has been particularly pronounced in cancer cells in response to the administration of chemotherapy regimens, a variety of other cell types and the administration of other drug classes have been noted (e.g., T-cells and anti-HIV drugs). In fact, certain mutations in the PGP gene significantly reduces PGP function, making it less able to force drugs out of cells. People who have two versions of the mutated gene—one inherited from each parent—have more than four times less PGP than those with two normal versions of the gene. People may also have one normal gene and one mutated one. Certain ethnic populations have increased incidence of such PGP mutations. Among individuals from Ghana, Kenya, the Sudan, as well as African Americans, frequency of the normal gene ranged from 73% to 84%. In contrast, the frequency was 34% to 59% among British whites, Portuguese, Southwest Asian, Chinese, Filipino and Saudi populations. As a result, certain ethnic populations may require increased administration of PGP antagonist in the formulation of the present invention to arrive at the an efficacious dose of the therapeutic (e.g., those from african descent). Conversly, certain ethnic populations, particularly those having increased frequency of the mutated PGP (e.g., of caucasian descent, or non-african descent) may require less pharmaceutical compositions in the formulation due to an effective increase in efficacy of such compositions as a result of the increased effective absorption (e.g., less PGP activity) of said composition.

In additional embodiments, the Therapeutics of the invention are administered in combination with other therapeutic or prophylactic regimens, such as, for example, radiation therapy.

### Example 33

Method of Treating Decreased Levels of the Polypeptide.

The present invention relates to a method for treating an individual in need of an increased level of a polypeptide of the invention in the body comprising administering to such an individual a composition comprising a therapeutically effective amount of an agonist of the invention (including polypeptides of the invention). Moreover, it will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a Therapeutic comprising an amount of the polypeptide to increase the activity level of the polypeptide in such an individual.

For example, a patient with decreased levels of a polypeptide receives a daily dose 0.1-100 ug/kg of the polypeptide for six consecutive days. Preferably, the polypeptide is in the secreted form. The exact details of the dosing scheme, based on administration and formulation, are provided herein.

### Example 34

Method of Treating Increased Levels of the Polypeptide.

The present invention also relates to a method of treating an individual in need of a decreased level of a polypeptide of the invention in the body comprising administering to such an individual a composition comprising a therapeutically effective amount of an antagonist of the invention (including polypeptides and antibodies of the invention).

In one example, antisense technology is used to inhibit production of a polypeptide of the present invention. This technology is one example of a method of decreasing levels of a polypeptide, preferably a secreted form, due to a variety of etiologies, such as cancer. For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided herein.

### Example 35

Method of Treatment Using Gene Therapy-Ex Vivo.

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 20 using primers and having appropriate restriction sites and initiation/stop codons, if necessary. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of conirmning that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 36

Gene Therapy Using Endogenous Genes Corresponding to Polynucleotides of the Invention.

Another method of gene therapy according to the present invention involves operably associating the endogenous polynucleotide sequence of the invention with a promoter via homologous recombination as described, for example, in U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous polynucleotide sequence, flanking the promoter. The targeting sequence will be sufficiently near the 5′ end of the polynucleotide sequence so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous polynucleotide sequence. This results in the expression of polynucleotide corresponding to the polynucleotide in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×106 cells/ml. Electroporation should be performed immediately following resuspension.

Plasmid DNA is prepared according to standard techniques. For example, to construct a plasmid for targeting to the locus corresponding to the polynucleotide of the invention, plasmid pUC18 (MBI Fermentas, Amherst, N.Y.) is digested with HindIII. The CMV promoter is amplified by PCR with an XbaI site on the 5′ end and a BamHI site on the 3′ end. Two non-coding sequences are amplified via PCR: one non-coding sequence (fragment 1) is amplified with a HindIII site at the 5′ end and an Xba site at the 3′end; the other non-coding sequence (fragment 2) is amplified with a BamHI site at the 5′end and a HindIII site at the 3′end. The CMV promoter and the fragments (1 and 2) are digested with the appropriate enzymes (CMV promoter—XbaI and BamHI; fragment 1—XbaI; fragment 2—BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5×106 cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 degree C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 37

Method of Treatment Using Gene Therapy—in Vivo.

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata et al., Cardiovasc. Res. 35(3):470-479 (1997); Chao et al., Pharmacol. Res. 35(6):517-522 (1997); Wolff, Neuromuscul. Disord. 7(5):314-318 (1997); Schwartz et al., Gene Ther. 3(5):405A411 (1996); Tsurumi et al., Circulation 94(12):3281-3290 (1996) (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 38

Transgenic Animals.

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of trarisgenic animals. Such techniques include, but are not limited to, pronuclear microinjection (Paterson et al., Appl. Microbiol. Biotechnol. 40:691-698 (1994); Carver et al., Biotechnology (NY) 11: 1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR(RT-PCR) . . . Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 39

Knock-Out Animals.

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 40

Production of an Antibody.

a) Hybridoma Technology

The antibodies of the present invention can be prepared by a variety of methods. (See, Current Protocols, Chapter 2.) As one example of such methods, cells expressing HGPRBMY1 or HGPRBMY2 are administered to an animal to induce the production of sera containing polyclonal antibodies. In a preferred method, a preparation of HGPRBMY1 or HGPRBMY2 protein is prepared and purified to render it substantially free of natural contaminants. Such a preparation is then introduced into an animal in order to produce polyclonal antisera of greater specific activity.

Monoclonal antibodies specific for protein HGPRBMY1 or HGPRBMY2 are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (preferably a mouse) is immunized with HGPRBMY1 or HGPRBMY2 polypeptide or, more preferably, with a secreted HGPRBMY1 or HGPRBMY2 polypeptide-expressing cell. Such polypeptide-expressing cells are cultured in any suitable tissue culture medium, preferably in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the HGPRBMY1 or HGPRBMY2 polypeptide.

Alternatively, additional antibodies capable of binding to HGPRBMY1 or HGPRBMY2 polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the HGPRBMY1 or HGPRBMY2 protein-specific antibody can be blocked by HGPRBMY1 or HGPRBMY2. Such antibodies comprise anti-idiotypic antibodies to the HGPRBMY1 or HGPRBMY2 protein-specific antibody and are used to immunize an animal to induce formation of further HGPRBMY1 or HGPRBMY2 protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation of Antibody Fragments Directed Against HGPRBMY1 or HGPRBMY2 From A Library of scFvs

Naturally occurring V-genes isolated from human PBLs are constructed into a library of antibody fragments which contain reactivities against HGPRBMY1 or HGPRBMY2 to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to inoculate 50 ml of 2×TY-AMP-GLU, 2×108 TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 Mm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0 M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 41

Assays Detecting Stimulation or Inhibition of B Cell Proliferation and Differentiation.

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL10, IL-13, IL-14 and IL-15. Interestingly, these signals are by themselves weak effectors but can, in combination with various co-stimulatory proteins, induce activation, proliferation, differentiation, homing, tolerance and death among B cell populations. One of the best studied classes of B-cell co-stimulatory proteins is the TNF-superfamily. Within this family CD40, CD27, and CD30 along with their respective ligands CD154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In Vitro Assay—Purified polypeptides of the invention, or truncated forms thereof, is assessed for its ability to induce activation, proliferation, differentiation or inhibition and/or death in B-cell populations and their precursors. The activity of the polypeptides of the invention on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 105 B-cells suspended in culture medium (RPMI 1640 containing 10% FBS, 5×10-5M 2ME, 100 U/ml penicillin, 10 ug/ml streptomycin, and 10-5 dilution of SAC) in a total volume of 150 ul. Proliferation or inhibition is quantitated by a 20 h pulse (1uCi/well) with 3H-thymidine (6.7 Ci/mM) beginning 72 h post factor addition. The positive and negative controls are IL2 and medium respectively.

In Vivo Assay-BALB/c mice are injected (i.p.) twice per day with buffer only, or 2 mg/Kg of a polypeptide of the invention, or truncated forms thereof. Mice receive this treatment for 4 consecutive days, at which time they are sacrificed and various tissues and serum collected for analyses. Comparison of H&E sections from normal spleens and spleens treated with polypeptides of the invention identify the results of the activity of the polypeptides on spleen cells, such as the diffusion of peri-arterial lymphatic sheaths, and/or significant increases in the nucleated cellularity of the red pulp regions, which may indicate the activation of the differentiation and proliferation of B-cell populations. Immunohistochemical studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from mice treated with polypeptide is used to indicate whether the polypeptide specifically increases the proportion of ThB+, CD45R(B220)dull B cells over that which is observed in control mice.

Likewise, a predicted consequence of increased mature B-cell representation in vivo is a relative increase in serum Ig titers. Accordingly, serum IgM and IgA levels are compared between buffer and polypeptide-treated mice.

One skilled in the art could easily modify the exemplified studies to test the activity of polynucleotides of the invention (e.g., gene therapy), agonists, and/or antagonists of polynucleotides or polypeptides of the invention.

### Example 42

T Cell Proliferation Assay.

A CD3-induced proliferation assay is performed on PBMCs and is measured by the uptake of 3H-thymidine. The assay is performed as follows. Ninety-six well plates are coated with 100 (1/well of mAb to CD3 (HIT3a, Pharmingen) or isotype-matched control mAb (B33. 1) overnight at 4 degrees C. (1 (g/ml in 0.05M bicarbonate buffer, pH 9.5), then washed three times with PBS. PBMC are isolated by F/H gradient centrifugation from human peripheral blood and added to quadruplicate wells (5×104/well) of mAb coated plates in RPMI containing 10% FCS and P/S in the presence of varying concentrations of polypeptides of the invention (total volume 200 ul). Relevant protein buffer and medium alone are controls. After 48 hr. culture at 37 degrees C., plates are spun for 2 min. at 1000 rpm and 100 (1 of supernatant is removed and stored −20 degrees C. for measurement of IL-2 (or other cytokines) if effect on proliferation is observed. Wells are supplemented with 100 ul of medium containing 0.5 uCi of 3H-thymidine and cultured at 37 degrees C. for 18-24 hr. Wells are harvested and incorporation of 3H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of polypeptides of the invention.

One skilled in the art could easily modify the exemplified studies to test the activity of polynucleotides of the invention (e.g., gene therapy), agonists, and/or antagonists of polynucleotides or polypeptides of the invention.

### Example 43

Effect of Polypeptides of the Invention on the Expression of MHC Class II, Costimulatory and Adhesion Molecules and Cell Differentiation of Monocytes and Monocyte-Derived Human Dendritic Cells.

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and MHC class II antigens). Treatment with activating factors, such as TNF-, causes a rapid change in surface phenotype (increased expression of MHC class I and II, costimulatory and adhesion molecules, downregulation of FC(RII, upregulation of CD83). These changes correlate with increased antigen-presenting capacity and with functional maturation of the dendritic cells.

FACS analysis of surface antigens is performed as follows. Cells are treated 1-3 days with increasing concentrations of polypeptides of the invention or LPS (positive control), washed with PBS containing 1% BSA and 0.02 mM sodium azide, and then incubated with 1:20 dilution of appropriate FITC- or PE-labeled monoclonal antibodies for 30 minutes at 4 degrees C. After an additional wash, the labeled cells are analyzed by flow cytometry on a FACScan (Becton Dickinson).

Effect on the production of cytokines. Cytokines generated by dendritic cells, in particular IL-12, are important in the initiation of T-cell dependent immune responses. IL-12 strongly influences the development of Thl helper T-cell immune response, and induces cytotoxic T and NK cell function. An ELISA is used to measure the IL-12 release as follows. Dendritic cells (106/ml) are treated with increasing concentrations of polypeptides of the invention for 24 hours. LPS (100 ng/ml) is added to the cell culture as positive control. Supernatants from the cell cultures are then collected and analyzed for IL-12 content using commercial ELISA kit(e.g., R & D Systems (Minneapolis, Minn.)). The standard protocols provided with the kits are used.

Effect on the expression of MHC Class II, costimulatory and adhesion molecules. Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of polypeptides of the invention or LPS (positive control), washed with PBS containing 1% BSA and 0.02 mM sodium azide, and then incubated with 1:20 dilution of appropriate FITC- or PE-labeled monoclonal antibodies for 30 minutes at 4 degrees C. After an additional wash, the labeled cells are analyzed by flow cytometry on a FACScan (Becton Dickinson).

Monocyte activation and/or increased survival. Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. Polypeptides, agonists, or antagonists of the invention can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×106/ml in PBS containing PI at a final concentration of 5 (g/ml, and then incubated at room temperature for 5 minutes before FACScan analysis. PI uptake has been demonstrated to correlate with DNA fragmentation in this experimental paradigm.

Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×105 cells/ml with increasing concentrations of the a polypeptide of the invention and under the same conditions, but in the absence of the polypeptide. For IL-12 production, the cells are primed overnight with IFN (100 U/ml) in presence of a polypeptide of the invention. LPS (10 ng/ml) is then added. Conditioned media are collected after 24 h and kept frozen until use. Measurement of TNF-alpha, IL-10, MCP-1 and IL-8 is then performed using a commercially available ELISA kit(e.g., R & D Systems (Minneapolis, Minn.)) and applying the standard protocols provided with the kit.

Oxidative burst. Purified monocytes are plated in 96-w plate at 2-1×105 cell/well. Increasing concentrations of polypeptides of the invention are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 nM PMA). The plates are incubated at 37(C. for 2 hours and the reaction is stopped by adding 20 μl 1N NaOH per well. The absorbance is read at 610 nm. To calculate the amount of H2O2 produced by the macrophages, a standard curve of a H2O2 solution of known molarity is performed for each experiment.

One skilled in the art could easily modify the exemplified studies to test the activity of polynucleotides of the invention (e.g., gene therapy), agonists, and/or antagonists of polynucleotides or polypeptides of the invention.

### Example 44

Biological Effects of HGPRBMY2 Polypeptides of the Invention.

Astrocyte and Neuronal Assays.

Recombinant polypeptides of the invention, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate a polypeptide of the invention's activity on these cells.

Moreover, previous reports describing the biological effects of FGF-2 (basic FGF) on cortical or hippocampal neurons in vitro have demonstrated increases in both neuron survival and neurite outgrowth (Walicke et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of a polypeptide of the invention to induce neurite outgrowth can be compared to the response achieved with FGF-2 using, for example, a thymidine incorporation assay.

Fibroblast and Endothelial Cell Assays.

Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated or one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE2 assays, the human lung fibroblasts are ultured at 5,000 cells/well in a 96-well plate for one day. After a medium change to 0.1% BSA basal medium, the cells are incubated with FGF-2 or polypeptides of the invention with or without IL-1( for 24 hours. The supernatants are collected and assayed for PGE2 by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or with or without polypeptides of the invention IL-1( for 24 hours. The supernatants are collected and assayed for IL-6 by ELISA kit (Endogen, Cambridge, Mass.).

Human lung fibroblasts are cultured with FGF-2 or polypeptides of the invention for 3 days in basal medium before the addition of Alamar Blue to assess effects on growth of the fibroblasts. FGF-2 should show a stimulation at 10-2500 ng/ml which can be used to compare stimulation with polypeptides of the invention.

Parkinson Models.

The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP+) and released. Subsequently, MPP+ is actively accumulated in dopaminergic neurons by the high-affinity reuptake transporter for dopamine. MPP+ is then concentrated in mitochondria by the electrochemical gradient and selectively inhibits nicotidamide adenine disphosphate: ubiquinone oxidoreductionase (complex 1), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, polypeptides of the invention can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of a polypeptide of the invention is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm2 on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopaminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if a polypeptide of the invention acts to prolong the survival of dopaminergic neurons, it would suggest that the polypeptide may be involved in Parkinson's Disease.

One skilled in the art could easily modify the exemplified studies to test the activity of polynucleotides of the invention (e.g., gene therapy), agonists, and/or antagonists of polynucleotides or polypeptides of the invention.

### Example 45

Stimulation of Nitric Oxide Production By Endothelial Cells.

Nitric oxide released by the vascular endothelium is believed to be a mediator of vascular endothelium relaxation. Thus, activity of a polypeptide of the invention can be assayed by determining nitric oxide production by endothelial cells in response to the polypeptide.

Nitric oxide is measured in 96-well plates of confluent microvascular endothelial cells after 24 hours starvation and a subsequent 4 hr exposure to various levels of a positive control (such as VEGF-1) and the polypeptide of the invention. Nitric oxide in the medium is determined by use of the Griess reagent to measure total nitrite after reduction of nitric oxide-derived nitrate by nitrate reductase. The effect of the polypeptide of the invention on nitric oxide release is examined on HUVEC.

Briefly, NO release from cultured HUVEC monolayer is measured with a NO specific polarographic electrode connected to a NO meter (Iso-NO, World Precision Instruments Inc.) (1049). Calibration of the NO elements is performed according to the following equation:

2KNO2+2KI+2H2SO4 6 2 NO+I2+2H2O+2K2SO4

The standard calibration curve is obtained by adding graded concentrations of KNO2 (0, 5, 10, 25, 50, 100, 250, and 500 nmol/L) into the calibration solution containing KI and H2SO4. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×106 endothelial cells. All values reported are means of four to six measurements in each group (number of cell culture wells). See, Leak et al. Biochem. and Biophys. Res. Comm. 217:96-105 (1995).

One skilled in the art could easily modify the exemplified studies to test the activity of polynucleotides of the invention (e.g., gene therapy), agonists, and/or antagonists of polynucleotides or polypeptides of the invention.

### Example 46

Effect of Polypeptides of the Invention on Vasodilation.

Since dilation of vascular endothelium is important in reducing blood pressure, the ability of polypeptides of the invention to affect the blood pressure in spontaneously hypertensive rats (SHR) is examined. Increasing doses (0, 10, 30, 100, 300, and 900 mg/kg) of the polypeptides of the invention are administered to 13-14 week old spontaneously hypertensive rats (SHR). Data are expressed as the mean +/−SEM. Statistical analysis are performed with a paired t-test and statistical significance is defined as p<0.05 vs. the response to buffer alone.

One skilled in the art could easily modify the exemplified studies to test the activity of polynucleotides of the invention (e.g., gene therapy), agonists, and/or antagonists of polynucleotides or polypeptides of the invention.

### Example 47

Peripheral Arterial Disease Model.

Angiogenic therapy using a polypeptide of the invention is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) a polypeptide of the invention, in a dosage range of 20 mg-500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of expression of a polypeptide of the invention and histology. Biopsy is also performed on the other side of normal muscle of the contralateral hindlimb.

One skilled in the art could easily modify the exemplified studies to test the activity of polynucleotides of the invention (e.g., gene therapy), agonists, and/or antagonists of polynucleotides or polypeptides of the invention.

What is claimed is:

1. An isolated nucleic acid molecule consisting of a polynucleotide having a nucleotide sequence selected from the group consisting of:
(a) a polynucleotide encoding a polypeptide of SEQ ID NO:2;
(b) an isolated polynucleotide consisting of nucleotides 250 to 1323 of SEQ ID NO:1, wherein said nucleotides encode a polypeptide corresponding to amino cids 2 to 359 of SEQ ID NO:2 minus the start codon;
(c) an isolated polynucleotide consisting of nucleotides 247 to 1323 of SEQ ID NO:1, wherein said nucleotides encode a polypeptide corresponding to amino acids 1 to 329 of SEQ ID NO:2 including the start codon;
(d) a polynucleotide encoding the HGPRBMY1 polypeptide encoded by the cDNA clone contained in ATCC Deposit No. XXXXX;
(e) a polynucleotide which represents the complimentary sequence (antisense) of SEQ ID NO:1;
(a) a polynucleotide encoding a polypeptide of SEQ ID NO:14;
(b) an isolated polynucleotide consisting of nucleotides 362 to 1651 of SEQ ID NO:13, wherein said nucleotides encode a polypeptide corresponding to amino acids 2 to 431 of SEQ ID NO:14 minus the start codon;
(c) an isolated polynucleotide consisting of nucleotides 359 to 1651 of SEQ ID NO:13, wherein said nucleotides encode a polypeptide corresponding to amino acids 1 to 431 of SEQ ID NO:13 including the start codon;
(d) a polynucleotide encoding the HGPRBMY2 polypeptide encoded by the cDNA clone contained in ATCC Deposit No. XXXXX; and
(e) a polynucleotide which represents the complimentary sequence (antisense) of SEQ ID NO:13.

2. The isolated nucleic acid molecule of claim 1, wherein the polynucleotide comprises a nucleotide sequence encoding a human G-protein coupled receptor protein.

3. A recombinant vector comprising the isolated nucleic acid molecule of claim 2.

4. A recombinant host cell comprising the recombinant vector of claim 3.

5. An isolated polypeptide consisting of an amino acid sequence selected from the group consisting of:
(a) a full length protein of SEQ ID NO:2;
(b) a polypeptide corresponding to amino acids 2 to 359 of SEQ ID NO:2, wherein said amino acids 2 to 359 comprise a polypeptide of SEQ ID NO:2 minus the start methionine;
(c) a polypeptide corresponding to amino acids 1 to 359 of SEQ ID NO:2;
(d) a polypeptide encoded by the cDNA contained in ATCC Deposit No. XXXXX;
(e) a full length protein of SEQ ID NO:14;
(f) a polypeptide corresponding to amino acids 2 to 431 of SEQ ID NO:14, wherein said amino acids 2 to 431 comprise a polypeptide of SEQ ID NO:14 minus the start methionine;
(g) a polypeptide corresponding to amino acids 1 to 431 of SEQ ID NO:14; and
(h) a polypeptide encoded by the cDNA contained in ATCC Deposit No. XXXXX.

6. A cell comprising the polypeptide of claim 4 and a member selected from the group consisting of NFAT/CRE, and NFAT G alpha 15.

7. A method of screening for candidate compounds capable of modulating activity of a G-protein coupled receptor-encoding polypeptide, comprising:
(a) contacting a test compound with the cell according to claim 6; and
(b) selecting as candidate modulating compounds those test compounds that modulate activity of the G-protein coupled receptor polypeptide.

8. A polypeptide that binds to the polypeptide of claim 1 selected from the group consisting of SEQ Id NO:32, 33, 34, and 35.

9. The polypeptide of claim 8 wherein said polypeptide modulates the G-protein coupled receptor of claim 5.

10. A method of screening for candidate compounds capable of modulating activity of a G-protein coupled receptor-encoding polypeptide, comprising:
(a). contacting the modulating polypeptide of claim 9 with the cell according to claim 4;
(b). contacting a test compound with said cell; and
(c). selecting as candidate modulating compounds those test compounds that modulate activity of the G-protein coupled receptor polypeptide.

11. A method of modulating the G-protein coupled receptor activity of the polypeptide of claim 5 comprising an effective amount of the polypeptide of claim 9.

12. The method for the treatment of immune-related disorders comprising a member of the group consisting of:
(a) administering to the subject a therapeutically effective amount of a HGPRBMY1 polypeptide;
(b) modulating the activity of a HGPRBMY1 polypeptide;
(c) administering to the subject a therapeutically effective amount of a HGPRBMY1 polypeptide wherein the HGPRBMY1 polypeptide is contained in a pharmaceutical composition;
(d) modulating the activity of a HGPRBMY1 polypeptide wherein the HGPRBMY1 polypeptide is HGPRBMY1 or a functionally equivalent derivative thereof;
(e) wherein the HGPRBMY1 polypeptide is HGPRBMY1 or a functionally equivalent derivative thereof wherein the HGPRBMY1 polypeptide is HGPRBMY1 or a functionally equivalent derivative thereof wherein the method comprises administering an effective amount of a compound that agonizes or antagonizes the activity of the HGPRBMY1 polypeptide;
(f) administering an effective amount of a compound that decreases expression of a HGPRBMY1 gene;
(g) administering an effective amount of a compound that decreases expression of a HGPRBMY1 gene in which the compound is an oligonucleotide encoding an antisense or ribozyme molecule that targets HGPRBMY1 transcripts and inhibits translation; and
(h) administering an effective amount of a compound that increases expression of a HGPRBMY1 gene.

13. The method for the treatment of heart-related disorders comprising a member of the group consisting of:
(i) administering to the subject a therapeutically effective amount of a HGPRBMY2 polypeptide;
(j) modulating the activity of a HGPRBMY2 polypeptide;
(k) administering to the subject a therapeutically effective amount of a HGPRBMY2 polypeptide wherein the HGPRBMY2 polypeptide is contained in a pharmaceutical composition;
(l) modulating the activity of a HGPRBMY2 polypeptide wherein the HGPRBMY2 polypeptide is HGPRBMY2 or a functionally equivalent derivative thereof;
(m) wherein the HGPRBMY2 polypeptide is HGPRBMY2 or a functionally equivalent derivative thereof wherein the HGPRBMY2 polypeptide is HGPRBMY2 or a functionally equivalent derivative thereof wherein the method comprises administering an effective amount of a compound that agonizes or antagonizes the activity of the HGPRBMY2 polypeptide;
(n) administering an effective amount of a compound that decreases expression of a HGPRBMY2 gene;
(o) administering an effective amount of a compound that decreases expression of a HGPRBMY2 gene in which the compound is an oligonucleotide encoding an antisense or ribozyme molecule that targets HGPRBMY2 transcripts and inhibits translation; and
(p) administering an effective amount of a compound that increases expression of a HGPRBMY2 gene.

14. A method for preventing, treating, or ameliorating a medical condition, comprising the step of administering to a mammalian subject a therapeutically effective amount of a member of the group consisting of:
(a) a polynucleotide of claim 1;
(b) a polypeptide of claim 5;
(c) an antagonist of the polypeptide of claim 5;
(d) an agonist of the polypeptide of claim 5;
(e) an antagonist of the polynucleotide of claim 1;
(f) a agonist of the polynucleotide of claim 1;
(g) the modulatory polypeptide of claim 9; and
(h) an antibody directed against the polypeptide of claim 5.

15. The method of preventing, treating, or ameliorating a medical condition of claim 14, wherein the medical condition is selected from the group consisting of an immune disorder, a hematopoietic disorder, a pulmonary disorder, and a gastrointestinal disorder.

16. The method of preventing, treating, or ameliorating a medical condition of claim 14, wherein the medical condition is selected from the group consisting of a cardiovascular disorder, a neural disorder, and a reproductive disorder.

17. The method of preventing, treating, or ameliorating a medical condition of claim 14, wherein the medical condition is a cell cycle defect, a disorder related to aberrant phosphorylation, a disorder related to aberrant signal transduction, a proliferating disorder, an autoimmune disorder, a disorder related to hyper immune activity, an inflammatory condition, a disorder related to aberrant acute phase responses, a hypercongenital condition, a birth defect, a necrotic lesion, a wound, organ transplant rejection, and a condition related to organ transplant rejection.

18. The method of preventing, treating, or ameliorating a medical condition of claim 14, wherein the medical condition is a metabolic disorder, obesity, and pain.

19. The method of preventing, treating, or ameliorating a medical condition of claim 14, wherein the condition is a condition related to aberrant cell cycle regulation, aberrant p27 regulation, aberrant apoptosis regulation, aberrant IkB regulation, aberrant NFkB regulation, aberrant DNA repair, and aberrant cellular phophorylation.

20. The isolated polypeptide of claim 5, wherein the full length protein comprises sequential amino acid deletions from either the C-terminus or the N-terminus.
